(19) United States
(12) Patent Application Publication
(10) Pub. No.: US 2025/0019439 A1
(43) Pub. Date: Jan. 16, 2025

(54) ANTI-IDIOTYPE ANTIBODIES

(71) Applicant: ADIMAB, LLC, Lebanon, NH (US)

(72) Inventor: William George ROACH, Lebanon, NH (US)

(21) Appl. No.: 18/712,739

(22) PCT Filed: Nov. 22, 2022
(86) PCT No.: PCT/US2022/080328

§ 371 Date: May 23, 2024

Related U.S. Application Data
(60) Provisional application No. 63/282,949, filed on Nov. 24, 2021.
(60) Provisional application No. 63/425,163, filed on Nov. 14, 2022.

(51) Int. Cl. C07K 16/28

(52) U.S. Cl. CPC C07K 16/2809; C07K 2317/24; C07K 2317/622; C07K 2317/92

(57) ABSTRACT

The present disclosure relates to anti-idiotypic antibodies binding to anti-CD3 antibodies, and uses thereof, e.g., in assays and methods of treatment.

### CROSS-REFERENCE TO RELATED APPLICATIONS

This application claims priority to: U.S. Provisional Application No. 63/282,949, filed on Nov. 24, 2021, entitled “Anti-Idiotype Antibodies”; and U.S. Provisional Application No. 63/425,163, filed on Nov. 14, 2022, entitled “Anti-Idiotype Antibodies.” The contents of said applications are incorporated by reference in their entirety herein.

### BACKGROUND

Cell proliferative disorders, such as cancer, are characterized by the uncontrolled growth of cell subpopulations. They are the leading cause of death in the developed world and the second leading cause of death in developing countries. Treatments being developed include those recruiting and activating T cells to kill tumor cells through CD3 targeting (Staerz et al. Nature 1985 314: 628-32). Bispecific antibodies have been developed for such treatments with CD3-specific affinity for T-cell recruitment and activation and secondary binding sites targeting disease-associated antigens, such as those produced by tumor cells. CD3 bispecific antibodies trigger the CD3 surface receptor on T cells by binding to their second target protein expressed on tumors such that available T cells can bind to target-expressing cells via bridging by the CD3 bispecific antibody, irrespective of the peptide/MHC specificity of their T-cell receptor. (See, e.g., Bassan, 2012, Blood 120:5094-95). Bridging of T cells and tumor cells using CD3 bispecific antibodies can induce dramatic regression of advanced-stage malignancies and, in some cases, lead to complete remission.

A variety of anti-CD3 antibodies are known in the art, including monoclonal and bispecific antibody formats. See, e.g., U.S. Pat. Nos. 7,262,276; 7,635,472; 7,862,813; 9,587,021; and 10,174,124; U.S. Publication No. US 2020/0190189; and International Publication Nos. WO2020247929 and WO2020247932. There remains a need in the art for anti-idiotypic antibodies that specifically bind to anti-CD3 antibodies for a variety of applications. The present disclosure meets this need by providing related compositions and methods of using them.

### SUMMARY

In one aspect, the present disclosure provides antibodies and antigen-binding fragments thereof, e.g., those that binds to a target antibody such as an anti-CD3 antibody.

In some embodiments, the present disclosure provides an antibody or antigen-binding fragment thereof comprising: (A) a heavy chain variable domain (VH) polypeptide comprising a set of VH complementarity determining regions: VH (CDR)1 (CDRH1), VH CDR2 (CDRH2), and VH CDR3 (CDRH3); and (B) a light chain variable domain (VL) polypeptide comprising a set of VL complementarity determining regions: VL CDR1 (CDRL1), VL CDR2 (CDRL2), and VL CDR3 (CDRL3).

In certain embodiments, at least three, four, five or all six of said CDRs may comprise: (i) the amino acid sequence of a corresponding VH or VL CDR contained in any of the variable domain sequences listed in Table 3A or 3B; and/or (ii) the amino acid sequence of a corresponding VH or VL CDR selected from any of the VH or VL CDRs listed in Table 4A or 4B.

In certain embodiments, (A) the VH polypeptide comprises a set of VH CDRs: CDRH1, CDRH2, and CDRH3 of a specific antibody selected from those listed in Table 4A; and/or (B) the VL polypeptide comprises a set of VL CDRs: CDRL1, CDRL2, and CDRL3 of a specific antibody selected from those listed in Table 4B.

In some embodiments, the present disclosure provides an antibody or antigen-binding fragment thereof comprising: (A) a VH polypeptide comprising: (a) a CDRH1 comprising the amino acid sequence of: (i) the CDRH1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the CDRH1 contained in any one of SEQ ID NOS: 640, 880, 560, 580, 600, 620, 660, 680, 700, 720, 740, 760, 780, 800, 820, 840, 860, and 900; and/or (iii) any one of SEQ ID NOS: 642, 882, 562, 582, 602, 622, 662, 682, 702, 722, 742, 762, 782, 802, 822, 842, 862, 902, 922, and 942; (b) a CDRH2 comprising the amino acid sequence of: (i) the CDRH2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the CDRH2 contained in any one of SEQ ID NOS: 640, 880, 560, 580, 600, 620, 660, 680, 700, 720, 740, 760, 780, 800, 820, 840, 860, and 900; and/or (iii) any one of SEQ ID NOS: 644, 884, 564, 584, 604, 624, 664, 684, 704, 724, 744, 764, 784, 804, 824, 844, 864, 904, 924, and 944; and/or (c) a CDRH3 comprising the amino acid sequence of: (i) the CDRH3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the CDRH3 contained in any one of SEQ ID NOS: 640, 880, 560, 580, 600, 620, 660, 680, 700, 720, 740, 760, 780, 800, 820, 840, 860, and 900; and/or (iii) any one of SEQ ID NOS: 646, 886, 566, 586, 606, 626, 666, 686, 706, 726, 926, 746, 766, 786, 806, 826, 846, 866, 906, and 946; and/or (B) a VL polypeptide comprising: (a) a CDRL1 comprising the amino acid sequence of: (i) the CDRL1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the CDRL1 contained in any one of SEQ ID NOS: 650, 890, 570, 590, 610, 630, 670, 690, 710, 730, 750, 770, 790, 810, 830, 850, 870, and 910; (iii) (iii-1) RASQSX₁SSX₂YLX₃ (SEQ ID NO: 932), wherein X₁ is V or I, X₂ is D, S, or deletion (no amino acid), and X₃ is A or N, or (iii-2) RSSQSLLX₁SNGYNYLD (SEQ ID NO: 952), wherein X₁=H or Y; and/or (iv) any one of SEQ ID NOS: 652, 892, 572, 592, 612, 632, 672, 692, 712, 732, 752, 772, 792, 812, 832, 852, 872, and 912; (b) a CDRL2 comprising the amino acid sequence of: (i) the CDRL2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the CDRL2 contained in any one of SEQ ID NOS: 650, 890, 570, 590, 610, 630, 670, 690, 710, 730, 750, 770, 790, 810, 830, 850, 870, and 910; (iii) X₁ASX₂X₃X₄X₅ (SEQ ID NO: 934), wherein X₁ is G or A, X₂ is S or N, X₃ is R or L, X₄ is A or Q, and X₅ is T or S; and/or (iv) any one of SEQ ID NOS: 654, 894, 574, 594, 614, 634, 674, 694, 714, 734, 754, 774, 794, 814, 834, 854, 874, 914, and 954; (c) a CDRL3 comprising the amino acid sequence of: (i) the CDRL3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the CDRL3 contained in any one of SEQ ID NOS: 650, 890, 570, 590, 610, 630, 670, 690, 710, 730, 750, 770, 790, 810, 830, 850, 870, and 910; (iii) (iii-1) X₁QX₂X₃X₄X₅X₆X₇T (SEQ ID NO: 936), wherein X₁ is Q or L, X₂ is F, T, or A, X₃ is G, L, or H, X₄ is R, S, H, P, or T, X₅ is V, D, S, T, Y, F, or A, X₆ is P or L, and X₇ is deletion (no amino acid), I, or W, or (iii-2) MQVX₁X₂X₃PWT (SEQ ID NO: 956), wherein X₁ is L, R, K, or S, X₂ is E, Q, D, or G, and X₃ is L, T, or I; and/or (iv) any one of SEQ ID NOS: 656, 896, 576, 596, 616, 636, 676, 696, 716, 736, 756, 776, 796, 816, 836, 856, 876, and 916, or an antibody or antigen-binding fragment thereof which comprises a combination of one or more of the foregoing CDRs.

In certain embodiments, the antibody or antigen-binding fragment thereof may comprise: (A) a VH polypeptide comprising said CDRH1, said CDRH2, and said CDRH3; and/or (B) a VL polypeptide comprising said CDRL1, said CDRL2, and said CDRL3.

In certain embodiments, the antibody or antigen-binding fragment thereof may comprise:(A) a VH polypeptide comprising said CDRH1, said CDRH2, and said CDRH3; and (B) a VL polypeptide comprising said CDRL1, said CDRL2, and said CDRL3.

In certain embodiments, the antibody or antigen-binding fragment thereof may comprise: (I) (i) the CDRL1, the CDRL2, and the CDRL3 contained in ADI-34134; or (ii) a CDRL1 comprising SEQ ID NO: 652, a CDRL2 comprising SEQ ID NO: 654, and a CDRL3 comprising SEQ ID NO: 656; or (II) (i) the CDRL1, the CDRL2, and the CDRL3 contained in ADI-34145; or (ii) a CDRL1 comprising SEQ ID NO: 892, a CDRL2 comprising SEQ ID NO: 894, and a CDRL3 comprising SEQ ID NO: 896.

In particular embodiments, the antibody or antigen-binding fragment thereof may comprise: (I) (i) the CDRH1, the CDRH2, the CDRH3, the CDRL1, the CDRL2, and the CDRL3 contained in ADI-34134; or (ii) a CDRH1 comprising SEQ ID NO: 642, a CDRH2 comprising SEQ ID NO: 644, a CDRH3 comprising SEQ ID NO: 646, a CDRL1 comprising SEQ ID NO: 652, a CDRL2 comprising SEQ ID NO: 654, and a CDRL3 comprising SEQ ID NO: 656.

In particular embodiments, the antibody or antigen-binding fragment thereof may comprise: (II) (i) the CDRH1, the CDRH2, the CDRH3, the CDRL1, the CDRL2, and the CDRL3 contained in ADI-34145; or (ii) a CDRH1 comprising SEQ ID NO: 882, a CDRH2 comprising SEQ ID NO: 884, a CDRH3 comprising SEQ ID NO: 886, a CDRL1 comprising SEQ ID NO: 892, a CDRL2 comprising SEQ ID NO: 894, and a CDRL3 comprising SEQ ID NO: 896.

In particular embodiments, the antibody or antigen-binding fragment thereof may comprise: (III) (i) the CDRH1, the CDRH2, the CDRH3, the CDRL1, the CDRL2, and the CDRL3 contained in ADI-30242; or (ii) a CDRH1 comprising SEQ ID NO: 562, a CDRH2 comprising SEQ ID NO: 564, a CDRH3 comprising SEQ ID NO: 566, a CDRL1 comprising SEQ ID NO: 572, a CDRL2 comprising SEQ ID NO: 574, and a CDRL3 comprising SEQ ID NO: 576.

In particular embodiments, the antibody or antigen-binding fragment thereof may comprise: (IV) (i) the CDRH1, the CDRH2, the CDRH3, the CDRL1, the CDRL2, and the CDRL3 contained in ADI-34131; or (ii) a CDRH1 comprising SEQ ID NO: 582, a CDRH2 comprising SEQ ID NO: 584, a CDRH3 comprising SEQ ID NO: 586, a CDRL1 comprising SEQ ID NO: 592, a CDRL2 comprising SEQ ID NO: 594, and a CDRL3 comprising SEQ ID NO: 596.

In particular embodiments, the antibody or antigen-binding fragment thereof may comprise: (V) (i) the CDRH1, the CDRH2, the CDRH3, the CDRL1, the CDRL2, and the CDRL3 contained in ADI-34132; or (ii) a CDRH1 comprising SEQ ID NO: 602, a CDRH2 comprising SEQ ID NO: 604, a CDRH3 comprising SEQ ID NO: 606, a CDRL1 comprising SEQ ID NO: 612, a CDRL2 comprising SEQ ID NO: 614, and a CDRL3 comprising SEQ ID NO: 616.

In particular embodiments, the antibody or antigen-binding fragment thereof may comprise: (VI) (i) the CDRH1, the CDRH2, the CDRH3, the CDRL1, the CDRL2, and the CDRL3 contained in ADI-34133; or (ii) a CDRH1 comprising SEQ ID NO: 622, a CDRH2 comprising SEQ ID NO: 624, a CDRH3 comprising SEQ ID NO: 626, a CDRL1 comprising SEQ ID NO: 632, a CDRL2 comprising SEQ ID NO: 634, and a CDRL3 comprising SEQ ID NO: 636.

In particular embodiments, the antibody or antigen-binding fragment thereof may comprise: (VII) (i) the CDRH1, the CDRH2, the CDRH3, the CDRL1, the CDRL2, and the CDRL3 contained in ADI-34135; or (ii) a CDRH1 comprising SEQ ID NO: 662, a CDRH2 comprising SEQ ID NO: 664, a CDRH3 comprising SEQ ID NO: 666, a CDRL1 comprising SEQ ID NO: 672, a CDRL2 comprising SEQ ID NO: 674, and a CDRL3 comprising SEQ ID NO: 676.

In particular embodiments, the antibody or antigen-binding fragment thereof may comprise: (VIII) (i) the CDRH1, the CDRH2, the CDRH3, the CDRL1, the CDRL2, and the CDRL3 contained in ADI-34136; or (ii) a CDRH1 comprising SEQ ID NO: 682, a CDRH2 comprising SEQ ID NO: 684, a CDRH3 comprising SEQ ID NO: 686, a CDRL1 comprising SEQ ID NO: 692, a CDRL2 comprising SEQ ID NO: 694, and a CDRL3 comprising SEQ ID NO: 696.

In particular embodiments, the antibody or antigen-binding fragment thereof may comprise: (IX) (i) the CDRH1, the CDRH2, the CDRH3, the CDRL1, the CDRL2, and the CDRL3 contained in ADI-34137; or (ii) a CDRH1 comprising SEQ ID NO: 702, a CDRH2 comprising SEQ ID NO: 704, a CDRH3 comprising SEQ ID NO: 706, a CDRL1 comprising SEQ ID NO: 712, a CDRL2 comprising SEQ ID NO: 714, and a CDRL3 comprising SEQ ID NO: 716.

In particular embodiments, the antibody or antigen-binding fragment thereof may comprise: (X) (i) the CDRH1, the CDRH2, the CDRH3, the CDRL1, the CDRL2, and the CDRL3 contained in ADI-34138; or (ii) a CDRH1 comprising SEQ ID NO: 722, a CDRH2 comprising SEQ ID NO: 724, a CDRH3 comprising SEQ ID NO: 726, a CDRL1 comprising SEQ ID NO: 732, a CDRL2 comprising SEQ ID NO: 734, and a CDRL3 comprising SEQ ID NO: 736.

In particular embodiments, the antibody or antigen-binding fragment thereof may comprise: (XI) (i) the CDRH1, the CDRH2, the CDRH3, the CDRL1, the CDRL2, and the CDRL3 contained in ADI-30254; or (ii) a CDRH1 comprising SEQ ID NO: 742, a CDRH2 comprising SEQ ID NO: 744, a CDRH3 comprising SEQ ID NO: 746, a CDRL1 comprising SEQ ID NO: 752, a CDRL2 comprising SEQ ID NO: 754, and a CDRL3 comprising SEQ ID NO: 756.

In particular embodiments, the antibody or antigen-binding fragment thereof may comprise: (XII) (i) the CDRH1, the CDRH2, the CDRH3, the CDRL1, the CDRL2, and the CDRL3 contained in ADI-34139; or (ii) a CDRH1 comprising SEQ ID NO: 762, a CDRH2 comprising SEQ ID NO: 764, a CDRH3 comprising SEQ ID NO: 766, a CDRL1 comprising SEQ ID NO: 772, a CDRL2 comprising SEQ ID NO: 774, and a CDRL3 comprising SEQ ID NO: 776.

In particular embodiments, the antibody or antigen-binding fragment thereof may comprise: (XIII) (i) the CDRH1, the CDRH2, the CDRH3, the CDRL1, the CDRL2, and the CDRL3 contained in ADI-34140; or (ii) a CDRH1 comprising SEQ ID NO: 782, a CDRH2 comprising SEQ ID NO: 784, a CDRH3 comprising SEQ ID NO: 786, a CDRL1 comprising SEQ ID NO: 792, a CDRL2 comprising SEQ ID NO: 794, and a CDRL3 comprising SEQ ID NO: 796.

In particular embodiments, the antibody or antigen-binding fragment thereof may comprise: (XIV) (i) the CDRH1, the CDRH2, the CDRH3, the CDRL1, the CDRL2, and the CDRL3 contained in ADI-34141; or (ii) a CDRH1 comprising SEQ ID NO: 802, a CDRH2 comprising SEQ ID NO: 804, a CDRH3 comprising SEQ ID NO: 806, a CDRL1 comprising SEQ ID NO: 812, a CDRL2 comprising SEQ ID NO: 814, and a CDRL3 comprising SEQ ID NO: 816.

In particular embodiments, the antibody or antigen-binding fragment thereof may comprise: (XV) (i) the CDRH1, the CDRH2, the CDRH3, the CDRL1, the CDRL2, and the CDRL3 contained in ADI-34142; or (ii) a CDRH1 comprising SEQ ID NO: 822, a CDRH2 comprising SEQ ID NO: 824, a CDRH3 comprising SEQ ID NO: 826, a CDRL1 comprising SEQ ID NO: 832, a CDRL2 comprising SEQ ID NO: 834, and a CDRL3 comprising SEQ ID NO: 836.

In particular embodiments, the antibody or antigen-binding fragment thereof may comprise: (XVI) (i) the CDRH1, the CDRH2, the CDRH3, the CDRL1, the CDRL2, and the CDRL3 contained in ADI-34143; or (ii) a CDRH1 comprising SEQ ID NO: 842, a CDRH2 comprising SEQ ID NO: 844, a CDRH3 comprising SEQ ID NO: 846, a CDRL1 comprising SEQ ID NO: 852, a CDRL2 comprising SEQ ID NO: 854, and a CDRL3 comprising SEQ ID NO: 856.

In particular embodiments, the antibody or antigen-binding fragment thereof may comprise: (XVII) (i) the CDRH1, the CDRH2, the CDRH3, the CDRL1, the CDRL2, and the CDRL3 contained in ADI-34144; or (ii) a CDRH1 comprising SEQ ID NO: 862, a CDRH2 comprising SEQ ID NO: 864, a CDRH3 comprising SEQ ID NO: 866, a CDRL1 comprising SEQ ID NO: 872, a CDRL2 comprising SEQ ID NO: 874, and a CDRL3 comprising SEQ ID NO: 876.

In particular embodiments, the antibody or antigen-binding fragment thereof may comprise: (XVIII) (i) the CDRH1, the CDRH2, the CDRH3, the CDRL1, the CDRL2, and the CDRL3 contained in ADI-34146; or (ii) a CDRH1 comprising SEQ ID NO: 902, a CDRH2 comprising SEQ ID NO: 904, a CDRH3 comprising SEQ ID NO: 906, a CDRL1 comprising SEQ ID NO: 912, a CDRL2 comprising SEQ ID NO: 914, and a CDRL3 comprising SEQ ID NO: 916.

In certain embodiments, in the antibody or antigen-binding fragment thereof: (A) the VH polypeptide may comprise: (a) a VH framework region 1 (FRH1) comprising the amino acid sequence of: (i) the FRH1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRH1 contained in any one of SEQ ID NOS: 640, 880, 560, 580, 600, 620, 660, 680, 700, 720, 740, 760, 780, 800, 820, 840, 860, and 900; and/or (iii) any one of SEQ ID NOS: 641, 881, 561, 581, 601, 621, 661, 681, 701, 721, 741, 761, 781, 801, 821, 841, 861, 901, 921, and 941; (b) a VH framework region 2 (FRH2) comprising the amino acid sequence of: (i) the FRH2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRH2 contained in any one of SEQ ID NOS: 640, 880, 560, 580, 600, 620, 660, 680, 700, 720, 740, 760, 780, 800, 820, 840, 860, and 900; and/or (iii) any one of SEQ ID NOS: 643, 883, 563, 583, 603, 623, 663, 683, 703, 723, 743, 763, 783, 803, 823, 843, 863, 903, 923, and 943; (c) a VH framework region 3 (FRH3) comprising the amino acid sequence of: (i) the FRH3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRH3 contained in any one of SEQ ID NOS: 640, 880, 560, 580, 600, 620, 660, 680, 700, 720, 740, 760, 780, 800, 820, 840, 860, and 900; and/or (iii) any one of SEQ ID NOS: 645, 885, 565, 585, 605, 625, 665, 685, 705, 725, 745, 765, 785, 805, 825, 845, 865, 905, 925, and 945; and/or (d) a VH framework region 4 (FRH4) comprising the amino acid sequence of: (i) the FRH4 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRH4 contained in any one of SEQ ID NOS: 640, 880, 560, 580, 600, 620, 660, 680, 700, 720, 740, 760, 780, 800, 820, 840, 860, and 900; and/or (iii) any one of SEQ ID NOS: 647, 887, 567, 587, 607, 627, 667, 687, 707, 727, 747, 767, 787, 807, 827, 847, 867, 907, 927, and 947; and/or (B) the VL may polypeptide comprise:(a) a VL framework region 1 (FRL1) comprising the amino acid sequence of: (i) the FRL1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRL1 contained in any one of SEQ ID NOS: 650, 890, 570, 590, 610, 630, 670, 690, 710, 730, 750, 770, 790, 810, 830, 850, 870, and 910; (iii) X₁IX₂X₃TQSPX₄X₅LSX₆SX₇GX₅RX₉TX₁₀X₁₁C (SEQ ID NO: 931), wherein X₁ is E or D, X₂ is V or Q, X₃ is L or M, X₄ is G or S, X₅ is T or S, X₆ is L or A, X₇ is P or V, X₅ is E or D, X₉ is A or V, X₁₀ is L or I, and X₁₁ is S or T; and/or (iv) any one of SEQ ID NOS: 651, 891, 571, 591, 611, 631, 671, 691, 711, 731, 751, 771, 791, 811, 831, 851, 871, 911, and 951; (b) a VL framework region 2 (FRL2) comprising the amino acid sequence of: (i) the FRL2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRL2 contained in any one of SEQ ID NOS: 650, 890, 570, 590, 610, 630, 670, 690, 710, 730, 750, 770, 790, 810, 830, 850, 870, and 910; (iii) WYQQKPGX₁APX₂LLIY (SEQ ID NO: 933), wherein X₁ is Q or K, and X₂ is R or K; and/or (iv) any one of SEQ ID NOS: 653, 893, 573, 593, 613, 633, 673, 693, 713, 733, 753, 773, 793, 813, 833, 853, 873, 913, and 953; (c) a VL framework region 3 (FRL3) comprising the amino acid sequence of: (i) the FRL3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRL3 contained in any one of SEQ ID NOS: 650, 890, 570, 590, 610, 630, 670, 690, 710, 730, 750, 770, 790, 810, 830, 850, 870, and 910; (iii) GX₁PX₂RFSGSGSGTDFTLTISX₃LX₄PEDFAX5YYC (SEQ ID NO: 935), wherein X₁ is I or V, X₂ is D or S, X₃ is R or S, X₄ is E or Q, and X₅ is V or T; and/or (iv) any one of SEQ ID NOS: 655, 895, 575, 595, 615, 635, 675, 695, 715, 735, 755, 775, 795, 815, 835, 855, 875, 915, and 955; and/or (d) a VL framework region 4 (FRL4) comprising the amino acid sequence of: (i) the FRL4 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146; (ii) the FRL4 contained in any one of SEQ ID NOS: 650, 890, 570, 590, 610, 630, 670, 690, 710, 730, 750, 770, 790, 810, 830, 850, 870, and 910; and/or (iii) any one of SEQ ID NOS: 657, 897, 577, 597, 617, 637, 677, 697, 717, 737, 757, 777, 797, 817, 837, 857, 877, 917, 937, and 957. In certain embodiments, the antibody or antigen-binding fragment thereof may comprise a VH and/or a VL that comprises any combination of the foregoing VH and VL framework regions.

In certain embodiments, the antibody or antigen-binding fragment thereof may comprise: (I) (i) the FRH1, the FRH2, the FRH3, the FLRH4, the FRL1, the FRL2, the FRL3, and the FRL4 contained in any one of ADI-34134, ADI-30242, ADI-34131, ADI-34133, ADI-34136, and ADI-34137; and/or (ii) a FRH1 comprising any one of SEQ ID NOS: 641, 561, 581, 621, 681, and 701, a FRH2 comprising any one of SEQ ID NOS: 643, 563, 583, 623, 683, and 703, a FRH3 comprising any one of SEQ ID NOS: 645, 565, 585, 625, 685, and 705, a FRH4 comprising any one of SEQ ID NOS: 647, 567, 587, 627, 687, and 707, a FRL1 comprising any one of SEQ ID NOS: 651, 571, 591, 631, 691, and 711, a FRL2 comprising any one of SEQ ID NOS: 653, 573, 593, 633, 693, and 713, a FRL3 comprising any one of SEQ ID NOS: 655, 575, 595, 635, 695, and 715, and a FRL4 comprising any one of SEQ ID NOS: 657, 577, 597, 637, 697, and 717.

In certain embodiments, the antibody or antigen-binding fragment thereof may comprise: (II) (i) the FRH1, the FRH2, the FRH3, the FLRH4, the FRL1, the FRL2, the FRL3, and the FRL4 contained in any one of ADI-34145, ADI-30254, ADI-34139, ADI-34140, ADI-34141, ADI-34142, ADI-34143, ADI-34144, and ADI-34146; and/or (ii) a FRH1 comprising any one of SEQ ID NOS: 881, 741, 761, 781, 801, 821, 841, 861, and 901, a FRH2 comprising any one of SEQ ID NOS: 883, 743, 763, 783, 803, 823, 843, 863, and 903, a FRH3 comprising any one of SEQ ID NOS: 885, 745, 765, 785, 805, 825, 845, 865, and 905, a FRH4 comprising any one of SEQ ID NOS: 887, 747, 767, 787, 807, 827, 847, 867, and 907, a FRL1 comprising any one of SEQ ID NOS: 891, 751, 771, 791, 811, 831, 851, 871, and 911, a FRL2 comprising any one of SEQ ID NOS: 893, 753, 773, 793, 813, 833, 853, 873, and 913, a FRL3 comprising any one of SEQ ID NOS: 895, 755, 775, 795, 815, 835, 855, 875, and 915, and a FRL4 comprising any one of SEQ ID NOS: 897, 757, 777, 797, 817, 837, 857, 877, and 917.

In certain embodiments, the antibody or antigen-binding fragment thereof may comprise: (III) (i) the FRH1, the FRH2, the FRH3, the FLRH4, the FRL1, the FRL2, the FRL3, and the FRL4 contained in ADI-34135 or ADI-34138; and/or (ii) a FRH1 comprising SEQ ID NO: 661 or 721, a FRH2 comprising SEQ ID NO: 663 or 723, a FRH3 comprising SEQ ID NO: 665 or 725, a FRH4 comprising SEQ ID NO: 667 or 727, a FRL1 comprising SEQ ID NO: 671 or 731, a FRL2 comprising SEQ ID NO: 673 or 733, a FRL3 comprising SEQ ID NO: 675 or 735, and a FRL4 comprising SEQ ID NO: 677 or 737.

In certain embodiments, the antibody or antigen-binding fragment thereof may comprise: (IV) (i) the FRH1, the FRH2, the FRH3, the FLRH4, the FRL1, the FRL2, the FRL3, and the FRL4 contained in ADI-34132; and/or (ii) a FRH1 comprising SEQ ID NO: 601, a FRH2 comprising SEQ ID NO: 603, a FRH3 comprising SEQ ID NO: 605, a FRH4 comprising SEQ ID NO: 607, a FRL1 comprising SEQ ID NO: 611, a FRL2 comprising SEQ ID NO: 613, a FRL3 comprising SEQ ID NO: 615, and a FRL4 comprising SEQ ID NO: 617.

In particular embodiments, the antibody or antigen-binding fragment thereof may comprise: (I) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34134; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 641, 642, 643, 644, 645, 646, 647, 651, 652, 653, 654, 655, 656, and 657, respectively.

In particular embodiments, the antibody or antigen-binding fragment thereof may comprise: (II) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34145; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 881, 882, 883, 884, 885, 886, 887, 891, 892, 893, 894, 895, 896, and 897, respectively.

In particular embodiments, the antibody or antigen-binding fragment thereof may comprise: (III) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-30242; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 561, 562, 563, 564, 565, 566, 567, 571, 572, 573, 574, 575, 576, and 577, respectively.

In particular embodiments, the antibody or antigen-binding fragment thereof may comprise: (IV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34131; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 581, 582, 583, 584, 585, 586, 587, 591, 592, 593, 594, 595, 596, and 597, respectively.

In particular embodiments, the antibody or antigen-binding fragment thereof may comprise: (V) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34132; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 601, 602, 603, 604, 605, 606, 607, 611, 612, 613, 614, 615, 616, and 617, respectively.

In particular embodiments, the antibody or antigen-binding fragment thereof may comprise: (VI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34133; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 621, 622, 623, 624, 625, 626, 627, 631, 632, 633, 634, 635, 636, and 637, respectively.

In particular embodiments, the antibody or antigen-binding fragment thereof may comprise: (VII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34135; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 661, 662, 663, 664, 665, 666, 667, 671, 672, 673, 674, 675, 676, and 677, respectively.

In particular embodiments, the antibody or antigen-binding fragment thereof may comprise: (VIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34136; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 681, 682, 683, 684, 685, 686, 687, 691, 692, 693, 694, 695, 696, and 697, respectively.

In particular embodiments, the antibody or antigen-binding fragment thereof may comprise: (IX) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34137; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 701, 702, 703, 704, 705, 706, 707, 711, 712, 713, 714, 715, 716, and 717, respectively.

In particular embodiments, the antibody or antigen-binding fragment thereof may comprise: (X) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34138; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 721, 722, 723, 724, 725, 726, 727, 731, 732, 733, 734, 735, 736, and 737, respectively.

In particular embodiments, the antibody or antigen-binding fragment thereof may comprise: (XI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-30254; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 741, 742, 743, 744, 745, 746, 747, 751, 752, 753, 754, 755, 756, and 757, respectively.

In particular embodiments, the antibody or antigen-binding fragment thereof may comprise: (XII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34139; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 761, 762, 763, 764, 765, 766, 767, 771, 772, 773, 774, 775, 776, and 777, respectively.

In particular embodiments, the antibody or antigen-binding fragment thereof may comprise: (XIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34140; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 781, 782, 783, 784, 785, 786, 787, 791, 792, 793, 794, 795, 796, and 797, respectively.

In particular embodiments, the antibody or antigen-binding fragment thereof may comprise: (XIV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34141; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 801, 802, 803, 804, 805, 806, 807, 811, 812, 813, 814, 815, 816, and 817, respectively.

In particular embodiments, the antibody or antigen-binding fragment thereof may comprise: (XV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34142; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 821, 822, 823, 824, 825, 826, 827, 831, 832, 833, 834, 835, 836, and 837, respectively.

In particular embodiments, the antibody or antigen-binding fragment thereof may comprise: (XVI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34143; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 841, 842, 843, 844, 845, 846, 847, 851, 852, 853, 854, 855, 856, and 857, respectively.

In particular embodiments, the antibody or antigen-binding fragment thereof may comprise: (XVII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34144; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 861, 862, 863, 864, 865, 866, 867, 871, 872, 873, 874, 875, 876, and 877, respectively.

In particular embodiments, the antibody or antigen-binding fragment thereof may comprise: (XVIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34146; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 901, 902, 903, 904, 905, 906, 907, 911, 912, 913, 914, 915, 916, and 917, respectively.

In certain embodiments, the antibody or antigen-binding fragment thereof may comprise any of the above-described CDR combinations (the combination of three VH CDRs, the combination of three V LCDRs, or the combination of six CDRs) and further meet the following:
In (I), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 640; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 650.

In (II), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 880; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 890.

In (III), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 560; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 570.

In (IV), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 580; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 590.

In (V), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 600; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 610.

In (VI), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 620; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 630.

In (VII), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 660; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 670.

In (VIII), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 680; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 690.

In (IX), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 700; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 710.

In (X), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 720; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 730.

In (XI), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 740; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 750.

In (XII), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 760; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 770.

In (XIII), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 780; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 790.

In (XIV), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 800; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 810.

In (XV), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 820; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 830.

In (XVI), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 840; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 850.

In (XVII), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 860; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 870.

In (XVIII), (A) the VH polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 900; and/or (B) the VL polypeptide may comprise an amino acid sequence at least 80%, at least 85%, at least 90%, at least 92%, at least 94%, at least 95%, at least 96%, at least 97%, at least 98%, at least 99%, or 100% identical to SEQ ID NO: 910.

In particular embodiments, the VH polypeptide and the VL polypeptide may comprise the amino acid sequences of: (I) SEQ ID NOS: 640 and 650, respectively; (II) SEQ ID NOS: 880 and 890, respectively; (III) SEQ ID NOS: 560 and 570, respectively; (IV) SEQ ID NOS: 580 and 590, respectively; (V) SEQ ID NOS: 600 and 610, respectively; (VI) SEQ ID NOS: 620 and 630, respectively; (VII) SEQ ID NOS: 660 and 670, respectively; (VIII) SEQ ID NOS: 680 and 690, respectively; (IX) SEQ ID NOS: 700 and 710, respectively; (X) SEQ ID NOS: 720 and 730, respectively; (XI) SEQ ID NOS: 740 and 750, respectively; (XII) SEQ ID NOS: 760 and 770, respectively; (XIII) SEQ ID NOS: 780 and 790, respectively; (XIV) SEQ ID NOS: 800 and 810, respectively; (XV) SEQ ID NOS: 820 and 830, respectively; (XVI) SEQ ID NOS: 840 and 850, respectively; (XVII) SEQ ID NOS: 860 and 870, respectively; or (XVIII) SEQ ID NOS: 900 and 910, respectively.

In certain embodiments, the antibody or antigen-binding fragment thereof may comprise any one or more of the following: (i) one or more antibody constant regions, a CH1 domain, a hinge, a CH2 domain, and/or a CH3 domain, which optionally is/are individually of, or derived from an IgG or human IgG, further optionally of or derived from a human IgG1, IgG4, IgG2, or IgG3; (ii) a fragment crystallizable (Fc) region, optionally of, or derived from: (1) a human IgG1, further optionally comprising one or more of the following amino acid modifications: N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, or any combination thereof, according to EU numbering; (2) a human IgG4, further optionally comprising one or more of the following amino acid modifications: E233P, F234V, L235A, G237A, E318A, S228P, L236E, S241P, L248E, T394D, M252Y, S254T, T256E, N297A, N297Q, or any combination thereof, according to EU numbering; (3) a human IgG2, further optionally comprising one or more of the following amino acid modifications: P238S, V234A, G237A, H268A, H268Q, H268E, V309L, N297A, N297Q, A330S, P331S, C232S, C233S, M252Y, S254T, T256E, or any combination thereof, according to EU numbering; and/or (4) a human IgG3, further optionally comprising E235Y, according to EU numbering; (iii) an IgG, IgA, IgE, IgD, or IgM, optionally IgG1, IgG4, IgG2, or IgG3; and/or (iv) an antibody fragment selected from the group consisting of: a fragment antigen-binding (Fab); an Fab₂; an Fab₃; an Fab′ fragment; an F(ab′)₂; a variable fragment (Fv); a single-chain Fv (scFv) fragment; a diabody; a triabody; a minibody; a scFv-Fc; a scFv2-Fc2; scFv-IgG; and/or a monovalent IgG (or a half IgG).

In some embodiments, the antibody or antigen-binding fragment thereof of according to the present disclosure may bind to a target antibody.

In certain embodiments, the target antibody may comprise an antigen-binding domain that binds Cluster of Differentiation 3 (CD3).

In certain embodiments, the target antibody may be or may comprise (1) an IgG, optionally IgG1, or (2) a Fab.

In certain embodiments, the antibody or antigen-binding fragment thereof may bind to the target antibody with an equilibrium dissociation constant (Kd) value of about 1.0×10⁶ (M) or lower, about 5.0×10⁷ (M) or lower, about 1.0×10⁷ (M) or lower, about 5.0×10⁸ (M) or lower, about 1.0×10⁸ (M) or lower, about 9.0×10⁹ (M) or lower, about 8.0×10⁹ (M) or lower, about 7.0×10⁹ (M) or lower, about 6.0×10⁹ (M) or lower, about 5.0×10⁹ (M) or lower, about 4.0×10⁹ (M) or lower, about 3.0×10⁹ (M) or lower, about 2.0×10⁹ (M) or lower, about 1.0×10⁹ (M) or lower, about 9.0×10¹⁰ (M) or lower, about 8.0×10¹⁰ (M) or lower, about 7.0×10¹⁰ (M) or lower, about 6.0×10¹⁰ (M) or lower, about 5.0×10¹⁰ (M) or lower, about 4.0×10¹⁰ (M) or lower, about 3.0×10¹⁰ (M) or lower, about 2.0×10¹⁰ (M) or lower, about 1.0×10¹⁰ (M) or lower, about 9.0×10¹¹ (M) or lower, about 8.0×10¹¹ (M) or lower, about 7.0×10¹¹ (M) or lower, about 6.0×10¹¹ (M) or lower, about 5.0×10¹¹ (M) or lower, or about 4.0×10¹¹ (M) or lower, optionally measured via bio-layer interferometry (BLI), optionally using an OCTET® system or via surface plasmon resonance (SPR), optionally using a CARTERRA® system.

In certain embodiments, the antibody or antigen-binding fragment thereof may exhibit a PSR score lower than 0.33, lower than about 0.30, lower than about 0.20, lower than about 0.15, lower than about 0.12, lower than about 0.10, lower than about 0.08, lower than about 0.06, lower than about 0.05, lower than about 0.04, lower than about 0.03, lower than about 0.02, lower than about 0.01, or of about 0.00; and/or a PSR score of about >0.10 and <0.33 or a PSR score of about <0.10.

In certain embodiments, the antibody or antigen-binding fragment thereof may exhibit a hydrophobic interaction chromatography (HIC) retention time of <about 10.5 minutes, <about 10.0 minutes, or <about 9.5 minutes.

In certain embodiments, the target antibody may comprise: (A) a VH polypeptide comprising: (a) a FRH1 comprising the amino acid sequence of: (i) the FRH1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRH1 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; (b) a CDRH1 comprising the amino acid sequence of: (i) the CDRH1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; (ii) the CDRH1 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; and/or (iii) any one of SEQ ID NOS: 181, 191, 201, 211, 221, 231, 241, 251, 261, 271, 281, 291, 301, 311, 321, 331, 341, 351, 361, 371, 381, 391,401, 411, 421, 431,441,451,461,471, 481, 491, 501, 511, 521, and 531; (c) a FRH2 comprising the amino acid sequence of: (i) the FRH2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRH2 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; (d) a CDRH2 comprising the amino acid sequence of: (i) the CDRH2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; (ii) the CDRH2 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; and/or (iii) any one of SEQ ID NOS: 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, and 532; (e) a FRH3 comprising the amino acid sequence of: (i) the FRH3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRH3 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; (f) a CDRH3 comprising the amino acid sequence of: (i) the CDRH3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; (ii) the CDRH3 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; and/or (iii) any one of SEQ ID NOS: 183, 193, 203, 213, 223, 233, 243, 253, 263, 273, 283, 293, 303, 313, 323, 333, 343, 353, 363, 373, 383, 393, 403, 413, 423, 433, 443, 453, 463, 473, 483, 493, 503, 513, 523, and 533; and/or (g) a FRH4 comprising the amino acid sequence of: (i) the FRH4 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRH4 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530.

In certain embodiments, the target antibody may comprise: (B) a VL polypeptide comprising: (a) a FRL1 comprising the amino acid sequence of: (i) the FRL1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRL1 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; (b) a CDRL1 comprising the amino acid sequence of: (i) the CDRL1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; (ii) the CDRL1 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; and/or (iii) any one of SEQ ID NOS: 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, and 536; (c) a FRL2 comprising the amino acid sequence of: (i) the FRL2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRL2 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; (d) a CDRL2 comprising the amino acid sequence of: (i) the CDRL2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; (ii) the CDRL2 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; and/or (iii) any one of SEQ ID NOS: 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, and 537; (e) a FRL3 comprising the amino acid sequence of: (i) the FRL3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRL3 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; (f) a CDRL3 comprising the amino acid sequence of: (i) the CDRL3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; (ii) the CDRL3 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; and/or (iii) any one of SEQ ID NOS: 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, 528, and 538; and/or (g) a FRL4 comprising the amino acid sequence of: (i) the FRL4 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or (ii) the FRL4 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435,445, 455, 465, 475, 485, 495, 505, 515, 525, and 535.

In certain embodiments, the target antibody may comprise: (A) a VH polypeptide comprising a set of a CDRH1, a CDRH2, and a CDRH3 according to any of those listed in Table 2A; and/or (B) a VL polypeptide comprising a set of a CDRL1, a CDRL2, and a CDRL3 according to any of those listed in Table 2B.

In certain embodiments, the target antibody may comprise: (A) a VH polypeptide comprising an amino acid sequence at least 80%, at least 85%, at least 90%, at least 92%, at least 94%, at least 95%, at least 96%, at least 97%, at least 98%, at least 99%, or 100% identical to any of those listed in Table 1A; and/or (B) a VL polypeptide comprising an amino acid sequence at least 80%, at least 85%, at least 90%, at least 92%, at least 94%, at least 95%, at least 96%, at least 97%, at least 98%, at least 99%, or 100% identical to any of those listed in Table 1B.

In particular embodiments, the target antibody may comprise a VH polypeptide and a VL polypeptide comprising the amino acid sequences of: 110 and 115, respectively; 120 and 125, respectively; 130 and 135, respectively; 140 and 145, respectively; 150 and 155, respectively; 160 and 165, respectively; 170 and 175, respectively; 180 and 185, respectively; 190 and 195, respectively; 200 and 205, respectively; 210 and 215, respectively; 220 and 225, respectively; 230 and 235, respectively; 240 and 245, respectively; 250 and 255, respectively; 260 and 265, respectively; 270 and 275, respectively; 280 and 285, respectively; 290 and 295, respectively; 300 and 305, respectively; 310 and 315, respectively; 320 and 325, respectively; 330 and 335, respectively; 340 and 345, respectively; 350 and 355, respectively; 360 and 365, respectively; 370 and 375, respectively; 380 and 385, respectively; 390 and 395, respectively; 400 and 405; respectively, 410 and 415, respectively; 420 and 425, respectively; 430 and 435, respectively; 440 and 445, respectively; 450 and 455, respectively; 460 and 465, respectively; 470 and 475, respectively; 480 and 485, respectively; 490 and 495, respectively; 500 and 505, respectively; 510 and 515, respectively; 520 and 525, respectively; 530 and 535, respectively.

In certain embodiments, the target antibody may comprise any one or more of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240 or an antigen-binding fragment thereof.

In some embodiments, the target antibody may comprise or may be comprised in a multispecific antibody or antibody fragment comprising at least: (a) a first antigen-binding region that binds to CD3; and (b) a second antigen-binding region that binds to a second antigen, optionally wherein the multispecific antibody or antibody fragment is a bispecific antibody or antibody fragment.

In certain embodiments, the second antigen may be or may comprise one or more of: an oncology target; a target molecule expressed on cancer cells; an immune-oncology target; a target molecule expressed on immune cells; a neurodegenerative disease target; an autoimmune disorder target (optionally a self-reactive immune molecule or a target molecule expressed on an immune cell expressing a self-reactive immune molecule); an inflammatory disease target (optionally an inflammatory cytokine or chemokine or a receptor thereof); an infectious disease target (optionally a target molecule of a virus, bacterium, or a fungus); a target molecule expressed on infected cells (optionally infected with a virus, a bacterium, or fungus); a metabolic disease target; a cognitive disorder target; a blood-brain barrier target; and/or a blood disease target.

In certain embodiments, the second antigen may be or may comprise one or more of: 17-IA, 4-1BB, 4Dc, 6-keto-PGFla, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTSS, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen (CCA), Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD3ε, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor Ila, Factor VII, Factor VIIIc, Factor IX, fibroblast activation protein (FAP), Fas, FcRl, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alphal, GFR-alpha2, GFR-alpha3, GITR, Glucagon, Glut 4, glycoprotein Ilb/IIIa (GP Ilb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309 antigen, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCK-2, MCP, M-CSF, MDC, Mer, metalloproteases, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian inhibiting substance, Mug, MuSK, NAIP, NAP, NCAD, N-Cadherin, NCA 90, NCAM, NCAM, Neprilysin, Neurotrophin-3,-4, or-6, Neurturin, Neuronal growth factor (NGF), NGFR, NGF-beta, nNOS, NO, NOS, Npn, NRG-3, NT, NTN, OB, OGGI, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PlGF, PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta Rllb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF1OA (TRAIL R1 Apo-2, DR4), TNFRSFIOB (TRAIL R2 DR5, KILLER, TRICK-2A, TRICK-B), TNFRSF1OC (TRAIL R3 DcRl, LIT, TRID), TNFRSF1OD (TRAIL R4 DcR2, TRUNDD), TNFRSF11 A (RANK ODF R, TRANCE R), TNFRSFllB (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-lBB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF1O (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSFIA (TNF-a Conectin, DIF, TNFSF2), TNFSFlB (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD3β Ligand CD153), TNFSF9 (4-lBB Ligand CD137 Ligand), TP-1, t-PA, Tpo, TRAIL, TRAIL R, TRAIL-R1, TRAIL-R2, TRANCE, transferrin receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (fit-4), VEGI, VIM, Viral antigens, VLA, VLA-1, VLA-4, VNR integrin, von Willebrand's factor, WIF-1, WNTi, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCRI, XCRI, XEDAR, XIAP, XPD, CTLA4 (cytotoxic T lymphocyte antigen-4), PD-1 (programmed cell death protein 1), PD-L1 (programmed cell death ligand 1), LAG-3 (lymphocyte activation gene-3), TIM-3 (T cell immunoglobulin and mucin protein-3), a hormone receptor, and a growth factor.

In certain embodiments, the second antigen may be or may comprise one or more of: BCMA, CTLA4 (cytotoxic T lymphocyte antigen-4), PD-1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFα, TGFO, a cytokine receptor, IL-2R, a chemokine, a chemokine receptor, a growth factor, VEGF, HGF, and a hormone receptor.

In certain embodiments, the multispecific antibody or antibody fragment may comprise a multispecific format according to one or more of a Fab-Fc-scFv, scFv2-Fc2, scFv-IgG “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

In certain embodiments, the target antibody may include a chimeric antigen receptor (CAR). The CAR may optionally include a transmembrane domain, an intracellular domain from a T-cell receptor, a CD3 subunit, and/or a co-stimulatory domain. In certain embodiments, the target antibody may include an scFv2-Fc2 and/or scFv-IgG; an IgG constant domain.

Another aspect of the present disclosure provides a nucleic acid encoding an antibody or antigen-binding fragment thereof according to any of those described herein.

Another aspect of the present disclosure provides a vector that includes the nucleic acid.

Another aspect of the present disclosure provides a cell that includes the nucleic acid. The cell may be a mammalian cell or a yeast cell.

Another aspect of the present disclosure provides a composition that includes (a) an excipient and (b) (i) one or more of an antibody or antigen-binding fragment thereof according to any of those described herein; (ii) a nucleic acid according to any of those described herein; (iii) a vector according to any of those described herein; and/or (iv) a cell according to any of those described herein.

Another aspect of the present disclosure provides a pharmaceutical composition that includes (a) a pharmaceutically acceptable excipient and (b) (i) one or more of an antibody or antigen-binding fragment thereof according to any of those described herein; (ii) a nucleic acid according to any of those described herein; (iii) a vector according to any of those described herein; and/or (iv) a cell according to any of those described herein.

Another aspect of the present disclosure provides a method of treating a subject in need of such treatment, the method including administering an antibody or antigen-binding fragment thereof according to any of those described herein or a composition comprising the antibody or antigen-binding fragment thereof such as any of the compositions described herein.

In some embodiments, the subject may comprise a target antibody (e.g., the subject is administered with the target antibody), wherein the target antibody may include an antigen-binding region that binds CD3 (i.e., the target antibody may be an anti-CD3 antibody), and wherein administering the antibody or antigen-binding fragment thereof partially or completely neutralizes the target antibody and/or inhibits or prevents the binding of the target antibody to CD3 or CD3-expressing cells.

In some embodiments, the antibody or antigen-binding fragment thereof may be administered in combination with a target antibody, wherein the target antibody may include an antigen-binding region that binds CD3 (i.e., the target antibody may be an anti-CD3 antibody). In certain embodiments, t antibody or antigen-binding fragment thereof may be conjugated with the target antibody, optionally via a linker, further optionally a cleavable linker.

In certain embodiments of any of the above-described methods, the antibody or antigen-binding fragment thereof may reduce an effect of the target antibody in at least one cell or tissue. In certain embodiments of any of the above-described methods, the target antibody may be administered to treat a target cell or target tissue and the antibody or antigen-binding fragment thereof may reduce an effect of the target antibody in a non-target cell or non-target tissue. In certain embodiments of any of the above-described methods, the antibody or antigen-binding fragment thereof may be used as a masking agent of the target antibody.

In some embodiments of any of the above-described methods, the antibody or antigen-binding fragment thereof may be bound to the target antibody prior to administration. In certain embodiments he antibody or antigen-binding fragment thereof may dissociate from the target antibody after administration. In particular embodiments, dissociation of the antibody or antigen-binding fragment thereof from the target antibody may be induced by one or more of, for example, change in pH, association with an inhibitor, presence of a competitive inhibitor, and presence of a protease.

In some embodiments of any of the methods described above, the antibody or antigen-binding fragment thereof may be administered to the subject to treat a disorder. In certain embodiments, the disorder may be related to a prior treatment of the subject with a target antibody, wherein the target antibody may include an antigen-binding domain that binds CD3 (i.e., the target antibody may be an anti-CD3 antibody). In certain embodiments, the disorder may include an inflammatory disorder. In certain embodiments, the disorder may include cytokine release syndrome.

In some embodiments of any of the methods described above, the method may include administering an additional therapeutic agent.

In some embodiments of any of the methods described above, the subject may be a mammal. In certain embodiments, the subject may be human.

Another aspect of the present disclosure provides a method of isolating a target antibody, the method including contacting the target antibody with an antibody or antigen-binding fragment thereof described herein.

Another aspect of the present disclosure provides a method of detecting a target antibody, and the method may comprise contacting the target antibody with any of the antibodies and antigen-binding fragments thereof described herein. In some embodiments, the contacting may occur in vitro. In certain embodiments, the target antibody may be contained in or derived from a biological sample, optionally from a subject (e.g., a subject who is/was administered with the target antibody). In some embodiments, the contacting may occur in vivo. In certain embodiments, the method may comprise administering the antibody or antigen-binding fragment thereof to a subject, optionally a subject administered with or comprising the target antibody. In certain embodiments, such an in vivo method may be for determining the distribution and/or the amount of the target antibody in the subject.

Another aspect of the present disclosure provides a method of characterizing a target antibody, wherein the target antibody may include an antigen-binding region that binds CD3 (i.e., the target antibody may be an anti-CD3 antibody), the method including the use of an antibody or antigen-binding fragment thereof according to any of those described herein. In some embodiments, the antibody or antigen-binding fragment thereof may be used to characterize a pharmacokinetic and/or pharmacodynamic property associated with the target antibody.

Another aspect of the present disclosure provides a method of determining the presence or absence of an anti-drug antibody in a sample, the method including the use of an antibody or antigen-binding fragment thereof according to any of those described herein. In some embodiments, the method may be for determining the presence or absence of an anti-drug antibody which competes with said antibody or antigen-binding fragment thereof for binding to the target antibody.

Another aspect of the present disclosure provides a method of making any of the antibodies and antigen-binding fragments thereof described herein, and the method may comprise (a) culturing cells comprising a nucleic acid (one or more nucleic acids) encoding the antibody or antigen-binding fragment in a condition that allows for expression of said antibody or antigen-binding fragment, and (b) harvesting and/or purifying the antibody or antigen-binding fragment from the cell culture from (a).

Another aspect of the present disclosure provides a method of manufacturing a cell according to the present disclosure or a population of such cells, comprising introducing a nucleic acid according to the present disclosure and/or a vector according to the present disclosure into one or more cells. In some embodiments, the introducing occurs in vitro. In some embodiments, the introducing occurs ex vivo. In certain embodiments, such cells manufactured may be ex vivo may be administered to a subject. In some cases the subject may be the one the cells were derived from (i.e., cells are manufactured for autologous administration/transplant). In some cases, the subject may be different from the one the cells were derived from (e.g., cells are manufactured for allogeneic or xenogeneic administration/transplant). In some embodiments, the introducing occurs in vivo.

Another aspect of the present disclosure provides kits comprising: (a) an antibody or antigen-binding fragment according to the present disclosure; and (b) a target antibody to which the antibody or antigen-binding fragment of (a) is specific for. In some embodiments, the kits may be used for in vitro assays, e.g., for measuring the amount of the target antibody in a sample. In certain embodiments, the target antibody of (b) may be used for preparing a control sample, such as for generating a standard curve.

In one aspect, any of the antibody or antigen-binding fragment thereof provided herein may be for use in any of the methods described herein.

In one aspect, any of the antibody or antigen-binding fragment thereof provided herein may be for use in the manufacture of a medicament. In some embodiments, the medicament may be for a treatment method according to the present disclosure.

In another aspect, the present disclosure provides use of any of the antibodies and antigen-binding fragments thereof according to the present disclosure in any of the method described herein.

### DETAILED DESCRIPTION

In some embodiments, the present disclosure provides anti-idiotypic antibodies. An antibody “idiotype” is a classification used to group antibodies by antigen specificity. As used herein, the term “anti-idiotypic antibody” or “anti-ID antibody” refers to an antibody that binds to antibodies of a specific idiotype. Anti-idiotypic antibodies may bind to variable domain regions of target antibodies and may, in some cases, bind to variable domain complementarity determining regions (CDRs) of target antibodies.

In some embodiments, the present disclosure provides anti-ID antibodies that bind to target antibodies that bind Cluster of Differentiation 3 (CD3). “Cluster of Differentiation 3” or “CD3”, generally refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3E or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP-000724), which is 207 amino acids in length, and human CD3γ protein (NCBI RefSeq No. NP-000064), which is 182 amino acids in length. “CD3εN27” and “CD3εN13” refer to the N-terminal 27 amino acids and the N-terminal 13 amino acids, respectively, of CD3, and optionally containing chemical modifications or conjugations made thereto. Antibodies or an antigen-binding fragments capable of binding to CD3 are referred to herein as “anti-CD3 antibodies.”

### Compounds and Compositions

In some embodiments, compounds and compositions of the present disclosure include antibodies. The term “antibody” is used herein in the broadest sense and encompasses various antibody structures, including but not limited to monoclonal antibodies, polyclonal antibodies, multispecific antibodies (e.g., bispecific antibodies), whole antibodies, antibody fragments (e.g., “antigen-binding fragments” or “antigen-binding antibody fragments, which means fragments of an antibody that retain antigen-binding activity), and various recombinant antibody formats (e.g., scFv formats).

A “monoclonal antibody” or “mAb” refers to an antibody obtained from a population of substantially homogeneous antibodies, i.e., the individual antibodies of the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

With regard to multispecific antibodies (e.g., bispecific, trispecific, tetraspecific, and so on), such antibodies include at least two different antigen-binding regions which recognize and specifically bind to at least two different antigens or epitopes. With regard to bispecific antibodies, such antibodies include two different antigen-binding regions which recognize and specifically bind to at least two different antigens or epitopes. Therefore, a “bispecific antibody” is a type of multispecific antibody. The at least two epitopes may or may not be within the same antigen. A bispecific antibody may target, for example, two different surface receptors on the same or different (e.g., an immune cell and a cancer cell) cells.

A “different antigen” may refer to different and/or distinct proteins, polypeptides, or molecules; as well as different and/or distinct epitopes, which epitopes may be contained within one protein, one polypeptide, or one molecule.

An “antigen-binding region” or “antigen-binding domain” refers to a portion of an antibody or antigen-binding fragment with specificity for an antigen.

The term “epitope” refers to an antigenic determinant that interacts with a specific antigen-binding region in the variable region of an antibody molecule known as a “paratope.” A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The terms “intact antibody” and “whole antibody” or the like are used herein interchangeably and refer to an antibody having a structure substantially similar to a native antibody. In some instances, an antibody includes heavy (H) and light (L) chains interconnected by disulfide bonds. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and β, respectively. For example, an intact IgG (or IgD or IgE) antibody comprises two immunoglobulin heavy chains and two immunoglobulin light chains. Therefore, in some instances, an antibody according to the present disclosure may comprise two pairs of heavy and light chains interconnected by disulfide bonds, or an antigen-binding fragment(s) thereof. Some intact antibody comprises multiple units each comprising two pairs of heavy and light chains interconnected by disulfide bonds. For example, an intact IgA comprises two units and an intact IgM comprises five units. Therefore, in other instances, an antibody according to the present disclosure may instead comprise multiple (e.g., two, three, four, five, and so on) units each comprising two pairs of heavy and light chains interconnected by disulfide bonds, or an antigen-binding fragment(s) thereof.

Each heavy chain is comprised of a heavy chain variable domain (“VH”) and a heavy chain constant region (“CH”), which is typically comprised of domains CH1, CH2 and CH3. Each light chain is comprised of a light chain variable domain (“VL”) and a light chain constant domain (“CL”). The VH and VL can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. CDRs in a heavy chain are designated “CDRH1,” “CDRH2,” and “CDRH3,” respectively, and the CDRs in a light chain are designated “CDRL1,” “CDRL2,” and “CDRL3.” In certain embodiments of the disclosure, the FRs of the antibody (or antigen-binding fragment thereof) may be identical to the human germline-encoded sequences or may be naturally or artificially modified. An amino acid consensus sequence may be defined based on a side-by-side analysis of two or more CDRs.

The numbering of amino acid residues in antibody variable and/or constant domains may be performed by any appropriate numbering schemes, methods, and definitions, e.g., based on numbering schemes such as EU numbering (as described in Kabat et al., Sequences of Proteins of Immunological Interest, 5th Ed. Public Health Service, National Institutes of Health, Bethesda, Md. (1991)), IMGT numbering, Kabat numbering, Chothia numbering, Martin numbering, Gelfand numbering, or Honneger's numbering); or structurally (see e.g., NCBI online tool, IgBlast; Dondelinger et al. Front Immunol. 2018 Oct 16;92278).

According to IMGT (the international ImMunoGeneTics information system for immunoglobulins or antibodies, T cell receptors, MH, immunoglobulin superfamily IgSF and MhSF), the CH1 domain, the hinge region, the CH2 domain, and the CH3 domain correspond to the amino acid positions 118-215, 216-230, 231-340, and 341-446, respectively (EU numbering). The terms “CH1 domain”, “hinge”, “CH2 domain”, and “CH3” are used in a broad sense herein to encompass any naturally occurring, corresponding heavy chain constant domain and/or region allotypes and variants thereof, which may comprise fewer or more amino acids (e.g., a CH1 domain may comprise a portion of a hinge region) and/or amino acid modification(s).

An exemplary CH1 domain of a human IgG1 may comprise the amino acid sequence of SEQ ID NO: 541 or 542; an exemplary hinge of a human IgG1 may comprise the amino acid sequence of SEQ ID NO: 543; and a CH2 domain of a human IgG1 may comprise the amino acid sequences of SEQ ID NOS: 544. An exemplary CH3 domain of a human IgG1 may comprise the amino acid sequence of SEQ ID NO: 545, 546, 547, or 548, and a C-terminal K may be added to any of such CH3 sequences. Any variants of such exemplary sequences may be used in conjunction with anti-CD3 variable sequences described herein.

“Fc region” is a C-terminal region of an immunoglobulin heavy chain that contains at least a portion of the constant region, including native sequence Fc regions and variant Fc regions. A human IgG heavy chain Fc region can extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

There are two major light chain isotypes, kappa (κ) and lambda (λ), and the corresponding light chain constant domains are called kappa CL domain (CLλdomain) and lambda CL domain (CLλ domain), respectively.

According to IMGT, the CLκdomain is the amino acid positions 108-214 (EU numbering). An exemplary CLκdomain of a human IgG may comprise the amino acid sequence of SEQ ID NO: 551. According to IMGT, the CLλ domain is the amino acid positions 107-215 (EU numbering). An exemplary CLλ domain of a human IgG may comprise the amino acid sequence of SEQ ID NO: 552.

The terms “CLκdomain” “CLλ domain” are used in a broad sense herein to encompass any naturally occurring, corresponding light chain constant domain and/or region allotypes and variants thereof, which may comprise fewer or more amino acids and/or amino acid modification(s).

Various standard sequences (corresponding to different allotypes) of the constant domains of human IgG1, IgG2, IgG3, and IgG4 are known in the field and may be found for example in Vidarsson et al., Front Immunol. 2014 Oct. 20; 5:520 and U.S. Pat. No. 9,150,663, the disclosures of which are hereby incorporated by reference herein in their entirety herein. Again these reference sequences are intended to be exemplary as Applicant intends for human IgG1, IgG2, IgG3, and IgG4 sequences to include any naturally occurring human IgG1, IgG2, IgG3, and IgG4 allotype.

An “antigen-binding fragment” refers to a portion of an intact antibody or to a combination of portions derived from an intact antibody or from intact antibodies and binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex, including an antibody fragment. Exemplary antigen-binding fragments include, but are not limited to, Fv, Fab, Fab′, Fab′-SH, F(ab′)₂; diabodies; linear antibodies; single-chain antibody molecules (e.g., single-chain variable fragment (scFv) or VH only or VL only); and multispecific antibodies formed from antibody fragments. In some embodiments, the antigen-binding fragments of antibodies described herein are or comprise scFvs. The term “half molecule” or “half antibody” when referring to IgG, IgE, or IgD, which may also be referred to as “half IgG”, “half IgE”, or “half IgD”, respectively, refers to a set of one heavy chain and one light chain of the referenced antibody.

Multispecific antibodies and antibody fragments that include anti-CD3 antibodies or antigen-binding fragments thereof disclosed herein may be prepared according to a variety of techniques including, but not limited to, recombinant co-expression of two immunoglobulin heavy chain-light chain pairs having different specificities (see, Milstein and Cuello, Nature 305: 537 (1983)), WO 93/08829, and Traunecker et al., EMBO J. 10: 3655 (1991)), “knob-in-hole” engineering (see, e.g., U.S. Pat. No. 5,731,168); immunoglobulin crossover (also known as Fab domain exchange or CrossMab format) technology (see, e.g., WO2009/080253; Schaefer et al., Proc. Natl. Acad. Sci. USA, 108:11187-11192 (2011)); engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); leucine zippers (see, e.g., Kostelny et al., J Immunol, 148(5):1547-1553 (1992)); “diabody” technology (see, e.g., Hollinger et al., Proc. Natl. Acad. Sci. USA, 90:6444-6448 (1993)); single-chain Fv (scFv) dimers (see, e.g. Gruber et al., J Immunol, 152:5368 (1994)); and trispecific antibodies as described, e.g., in Tutt et al. J Immunol 147: 60 (1991).

A variety of multispecific antibody formats may be used in the context of a multispecific antibodies and antibody fragments described herein. Non-limiting examples of multispecific and bispecific formats include, e.g., Fab-Fc-scFv (“bottle-opener”) (XENCOR), Mab-scFv (XENCOR), Mab-Fv (XENCOR), Dual scFv (XENCOR), central Fv (XENCOR), central scFv (XENCOR), one-arm central scFv (XENCOR), Fab-Fab (XENCOR), Fab-Fv (XENCOR), mAb-Fv (XENCOR), mAb-Fab (XENCOR), DART (MACROGENICS), BiTE (AMGEN/MICROMET), KiTE, common light chain-IgG (Genentech), TandAb (SFFIMED), Cross-Mab (ROCHE), SEED (EMD SERONO), BEAT (GLENMARK), TrioMab (TRION PHARMA/FRESENIUS BIOTECH), DuetMab (MEDIMMUNE), and others, as disclosed, e.g., in (WO2021067404; WO2022150787; WO2022150785; WO 95/09917; WO 2008/119566; WO 2008/119567; WO2011/121110; WO 2010/037835; WO 2007/042261; WO 2007/110205; WO 2011/121110; WO 2012/055961; WO 2012/16067; WO 2016/086189; WO 2016/182751; WO 2015/006749; WO 2014/049003; WO 2013/177101; WO 2015/128509; U.S. Pat. No. 7,951,917; US 2009/0252729; US 2014/0348839; U.S. Pat. No. 7,183,076; Mazor et al., Mabs, Vol. 7, pages 377-389 (2015); Muda et al., Protein Engineering, Design, & Selection, Vol. 24, pages 447-454 (2011); and Del Bano et al., Antibodies, Vol. 5, pages 1-23 (2016). In some embodiments, scFv fragments described herein include one or more variable domains of a multispecific (e.g., bispecific), antibody.

### Anti-CD3 Antibodies (Exemplary Target Antibodies)

In some embodiments, antibodies and antigen-binding fragments according to the present disclosure may specifically bind to one or more target antibodies, which may include anti-CD3 antibodies. A variety of anti-CD3 antibodies are known in the art, including, but not limited to, any of those described in U.S. Pat. Nos. 7,262,276, 7,635,472, 7,862,813, 9,587,021, and 10,174,124; U.S. Publication No. US 2020/0190189; U.S. Provisional Application No. 63/245,499; International Application No. PCT/US22/76522; and International Publication Nos. WO2020247929 and WO2020247932, the contents of each of which are herein incorporated by reference in their entirety.

### Anti-CD3 Antibody Sequences (Exemplary Target Antibody Sequences)

In some embodiments, anti-CD3 antibodies of the present disclosure may include a variable domain amino acid sequence that includes one or more of the variable domain amino acid sequences listed in Table 1A or 1B or a fragment or variant thereof. In certain embodiments, the antibodies may include a VH and VL pair with amino acid sequences according to any one of those associated with any of the antibodies identified by the ADI Numbers as listed in Table 1A and/or 1B.

In some embodiments, anti-CD3 antibodies of the present disclosure may include one or more of the complementarity determining region (CDR) amino acid sequences listed in Tables 2A and 2B or a fragment or variant thereof. In some embodiments, anti-CD3 antibodies of the present disclosure include a set of CDR sequences (e.g., a set of three VH CDRs and/or a set of three VL CDRs, or a set of six CDRs) associated with a specific antibody identified by the ADI Number listed in Tables 2A and/or 2B.

In some embodiments, anti-CD3 antibodies may include variable domain amino acid sequences that include CDR amino acid sequences according to any of the CDR amino acid sequences of Tables 2A and/or 2B and/or framework (FR) amino sequences according to any of those of the antibody variable domains shown in Tables 1A and 1B (FR sequences are defined by the sequence spans without an underline). In some embodiments, anti-CD3 antibodies may include variable domain amino acid sequences that include CDR amino acid sequences according to any of the CDR amino acid sequences of Tables 2A and/or 2B and any appropriate FR amino sequences including any known variable domain germline sequences (e.g., mammalian germline sequence, e.g., mouse, human, or non-human germline sequence) or variants thereof.

In some embodiments, an anti-CD3 antibody binds to CD3 with a dissociation constant (K_{D}) of about 100×10⁻⁹ M or less. In some embodiments, K_{D} is measured by surface plasmon resonance (SPR) using, e.g., a CARTERRA® LSA® instrument (CARTERRA®) or a BIACORE® system (Cytiva, previously GE Healthcare), biolayer interferometry (BLI) measurements using, e.g., a FORTEBIO OCTET® HTX instrument (Pall Life Sciences), or solution-affinity ELISA. In some embodiments, the K_{D} is measured using an scFv fragment of the anti-CD3 antibody. In some embodiments, the monovalent K_{D} is measured. In some embodiments, the anti-CD3 antibody binds to an epitope of CD3 that is conserved among CD3 from different species, e.g., human and cynomolgus monkey cross-reactive.

In certain embodiments, the anti-CD3 antibodies and/or antigen-binding fragments thereof as described herein may be, may comprise, or may be contained in a multispecific antibody (in particular, a bispecific antibody) that has binding specificity for a second antigen. Such a second antigen may be a different target altogether than the first antigen (i.e., CD3), or a different epitope present on the same target (i.e., CD3). In some embodiments, the binding specificities are to two different epitopes of CD3 (e.g., CD3ε or CD3γ). In other embodiments, one of the binding specificities is for CD3 (e.g., CD3ε or CD3γ) and the other is for a different biological molecule (e.g., a cell surface antigen, e.g., a tumor antigen).

Non-limiting examples of second antigens targeted by multispecific (e.g., bispecific) anti-CD3 antibodies and/or antigen-binding fragments thereof described herein include: 17-IA, 4-1BB, 4Dc, 6-keto-PGFla, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-l-antitrypsin, alpha-V/beta-1 antagonist, ANG, Ang, APAF-1, APE, APJ, APP, APRIL, AR, ARC, ART, Artemin, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD3ε, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor Ila, Factor VII, Factor VIIIc, Factor IX, fibroblast activation protein (FAP), Fas, FcRl, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alphal, GFR-alpha2, GFR-alpha3, GITR, Glucagon, Glut 4, glycoprotein Ilb/IIIa (GP Ilb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, metalloproteases, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian inhibiting substance, Mug, MuSK, NAIP, NAP, NCAD, N-Cadherin, NCA 90, NCAM, NCAM, Neprilysin, Neurotrophin-3,-4, or-6, Neurturin, Neuronal growth factor (NGF), NGFR, NGF-beta, nNOS, NO, NOS, Npn, NRG-3, NT, NTN, OB, OGGI, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta Rllb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF1OA (TRAIL RI Apo-2, DR4), TNFRSFIOB (TRAIL R2 DR5, KILLER, TRICK-2A, TRICK-B), TNFRSF1OC (TRAIL R3 DcR1, LIT, TRID), TNFRSF1OD (TRAIL R4 DcR2, TRUNDD), TNFRSF11A (RANK ODF R, TRANCE R), TNFRSFllB (OPG OCIF, TRI), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-lBB CD137, ILA), TNFRSF21 (DR6), TNFRSF22 (DcTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD3β Ligand CD153), TNFSF9 (4-lBB Ligand CD137 Ligand), TP-1, t-PA, Tpo, TRAIL, TRAIL R, TRAIL-RI, TRAIL-R2, TRANCE, transferrin receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD-1 (programmed cell death protein 1), PD-L1 (programmed cell death ligand 1), LAG-3 (lymphocyte activation gene-3), TIM-3 (T cell immunoglobulin and mucin protein-3), a hormone receptor, and a growth factor.

Further examples of second antigens targeted by multispecific (e.g., bispecific) anti-CD3 antibodies and/or antigen-binding fragments thereof include:: BCMA, CTLA4 (cytotoxic T lymphocyte antigen-4), PD-1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFα, TGFO, cytokine receptors, IL-2R, chemokines, chemokine receptors, growth factors, VEGF, HGF, and hormone receptors.

The present disclosure also contemplates modification of anti-CD3 antibodies disclosed herein. Such modifications may include one or more amino acid substitutions, insertions and/or deletions in the FR and/or CDR regions of the heavy and/or light chain variable domains. Once obtained, such derivative antibodies and/or antigen-binding fragments thereof can be tested for one or more desired properties such as improved binding specificity, increased binding affinity, improved developability, etc.

In some embodiments, anti-CD3 antibodies and/or antigen-binding fragments thereof include VH amino acid sequences, VL amino acid sequences, and/or CDR amino acid sequences described herein (e.g., in Table 1A or 1B or Table 2A or 2B). In some embodiments, anti-CD3 antibodies and/or antigen-binding fragments thereof may have amino acid sequence identities of at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 80%, at least about 75%, at least about 70%, at least about 65%, at least about 60%, at least about 55%, at least about 50%; and/or all sequence identity percentages in between to corresponding sequences of anti-CD3 antibodies disclosed in Table 1A or 1B or Table 2A or 2B. In some embodiments, percent identity is measured by any well-known algorithm of sequence identity, such as FASTA, BLAST or GAP.

### Anti-ID Antibodies

In some embodiments, the present disclosure provides anti-ID antibodies. Anti-ID antibodies are antibodies that bind to antigen-binding regions of another antibody, referred to herein as a “target antibody.”

In some embodiments, the target antibody may comprise: (A) a VH comprising: one or more of VH CDRs contained in any of the VH in Table 1A; one or more of VH CDRs in Table 2A; and/or a set of CDRH1, CDRH2, and CDRH3 of an antibody in Table 2A; and/or (B) a VL comprising: one or more of VL CDRs contained in any of the VL in Table 1B; one or more of VL CDRs in Table 2B; and/or a set of CDRL1, CDRL2, and CDRL3 of an antibody in Table 2B.

In some embodiments, anti-ID antibodies of the present disclosure may bind to anti-CD3 target antibodies and may bind specifically to anti-CD3 antigen-binding regions of such target antibodies. Anti-ID antibodies binding to anti-CD3 target antibodies may bind to anti-CD3 antibody variable domains, including, but not limited to, any of those described herein (e.g., any of those listed in Table 1A or 1B) or variants thereof and any of those set forth in the following patent applications and publications or variants thereof: U.S. Publication No. US 2020/0190189; U.S. Provisional Application No. 63/245,499; International Application No. PCT/US22/76522; and International Publication Nos. WO2020247929 and WO2020247932, the contents of each of which are herein incorporated by reference in their entirety. Anti-ID antibodies binding to anti-CD3 target antibodies may bind specifically to one or more anti-CD3 target antibody CDRs and/or FRs, including, but not limited to, any of those described herein (e.g., any of those listed in Table 2A or 2B or contained in Table 1A or 1B) or variants thereof and any of those set forth in the following patent applications and publications or variants thereof: U.S. Publication No. US 2020/0190189; U.S. Provisional Application No. 63/245,499; International Application No. PCT/US22/76522; and International Publication Nos. WO2020247929 and WO2020247932, the contents of each of which are herein incorporated by reference in their entirety.

Anti-CD3 target antibody antigen-binding regions may include: a VH that includes an amino acid sequence according to any of those listed in Table 1A; a VL that includes an amino acid sequence according to any of those listed in Table 1B; a VH that includes a CDR sequence set according to any of those listed in Table 2A; and/or a VL that includes a CDR sequence set according to any of those listed in Table 2B. Target antibodies may include multispecific antibodies; bispecific antibodies, and/or scFvs. Multispecific target antibodies may include antigen-binding regions that bind to oncology targets; immune-oncology targets; neurodegenerative disease targets; autoimmune disorder targets; infectious disease targets; metabolic disease targets; cognitive disorder targets; blood-brain barrier targets; and/or blood disease targets.

Multispecific target antibodies may comprise a first antigen-binding region specific to a first antigen (e.g., CD3) and a second antigen-binding region specific to a second antigen, which may be or comprise any of the exemplary second antigens described herein. Target antibodies bound by anti-ID antibodies described herein may include chimeric antigen receptors (CARs). Such target antibodies may include transmembrane domains, intracellular domains from T-cell receptors, CD3(subunits, and/or co-stimulatory domains.

Target antibodies bound by anti-ID antibodies described herein may include an scFv2-Fc2 and/or scFv-IgG; an IgG constant domain; and/or a multispecific antibody format according to one or more of a Fab-Fc-scFv, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

### Anti-ID Antibody Sequences

In some embodiments, anti-ID antibodies of the present disclosure may include a variable domain amino acid sequence that includes one or more of the variable domain amino acid sequences listed in Table 3A or 3B or a fragment or variant thereof. The antibodies may include a VH and VL pair with amino acid sequences according to any one of the VH and VL pairs associated with any of the antibodies identified by the ADI Names listed.

In some embodiments, anti-ID antibodies of the present disclosure may include one or more of the CDR amino acid sequences, or a fragment or variant thereof, listed in Table 4A or 4B. In some embodiments, anti-ID antibodies of the present disclosure include a set of CDR sequences (for example, a set including a CDRH1, CDRH2, and CDRH3, a set including a CDRL1, CDRL2, and CDRL3, or a set including a CDRH1, CDRH2, CDRH3, CDRL1, CDRL2, and CDRL3) associated with a specific antibody identified by the ADI Name listed in Tables 4A and/or 4B.

In some embodiments, anti-ID antibodies may include variable domain amino acid sequences that include CDR amino acid sequences according to any of the CDR amino acid sequences of Table 4A or 4B and FR amino sequences according to any of those of the antibody variable domains shown in Table 4C or 4D. In some embodiments, anti-ID antibodies may include variable domain amino acid sequences that include CDR amino acid sequences according to any of the CDR amino acid sequences of Table 4A or 4B and any appropriate FR amino sequences including FR sequences according to any known variable domain germline sequence (e.g., mammalian germline sequence, e.g., mouse, human, or non-human germline sequence) or variants thereof.

The present disclosure also contemplates modification of anti-ID antibodies disclosed herein. Such modifications may include one or more amino acid substitutions, insertions and/or deletions in the FR and/or CDR regions of the heavy and/or light chain variable domains. Once obtained, such derivative antibodies and/or antigen-binding fragments thereof can be tested for one or more desired properties such as improved binding specificity, increased binding affinity, improved developability, etc.

In some embodiments, anti-ID antibodies and/or antigen-binding fragments thereof include VH amino acid sequences, VL amino acid sequences, and/or CDR amino acid sequences described herein (e.g., in Table 3A or 3B or Table 4A or 4B). In some embodiments, anti-ID antibodies and/or antigen-binding fragments thereof have amino acid sequence identities of at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 80%, at least about 75%, at least about 70%, at least about 65%, at least about 60%, at least about 55%, at least about 50%; and/or all sequence identity percentages in between to corresponding sequences of anti-ID antibodies disclosed in Table 3A or 3B or Table 4A or 4B. In some embodiments, percent identity is measured by any well-known algorithm of sequence identity, such as FASTA, BLAST or GAP.

In some embodiments, the present disclosure provides nucleic acids encoding anti-ID antibodies or antigen-binding fragments thereof according to any of those described herein. Such an antibody or antigen-binding fragment thereof may be encoded in one nucleic acid or multiple (e.g., two) nucleic acids, e.g., one nucleic acid encoding a VH-containing polypeptide and one nucleic acid encoding a VL-containing polypeptide. The present disclosure also includes vectors that include such nucleic acids. An antibody or antigen-binding fragment thereof may be encoded in one vector or multiple (e.g., two) vectors, e.g., one vector comprising a nucleic acid encoding a VH-containing polypeptide and one vector comprising a nucleic acid encoding a VL-containing polypeptide. The present disclosure also includes cells that include the nucleic acids and/or vectors described above. The cells may be mammalian or yeast cells.

In some embodiments, the present disclosure provides compositions that include an excipient and one or more of. an antibody or antigen-binding fragment thereof according to any of those described herein; a nucleic acid according to any of those described herein; a vector according to any of those described herein; and a cell according to any of those described herein. Such compositions may be pharmaceutical compositions that include pharmaceutically acceptable excipients. A “pharmaceutical composition” or “pharmaceutical formulation” refers to a preparation in such form as to permit the biological activity of an active ingredient contained therein, such as antibodies described herein, to be effective and which preferably contains no additional components which are unacceptably toxic to a subject to which the formulation would be administered.

Antibody modification and optimization

The present disclosure also contemplates modified versions of antibodies disclosed herein, such as anti-ID antibodies and target antibodies (e.g, anti-CD3 antibodies). Such modifications may include one or more amino acid substitutions, insertions and/or deletions in the FR and/or CDR regions of the heavy and/or light chain variable domains. Once obtained, such derivative antibodies and/or antigen-binding fragments thereof can be tested for one or more desired properties such as improved binding specificity, increased binding affinity, improved developability, etc.

In some embodiments, antibodies of the present disclosure include modified versions of VH amino acid sequences, VL amino acid sequences, and/or CDR amino acid sequences described herein (e.g., in Table 1A or 1B, Table 2A or 2B, Table 3A or 3B, or Table 4A or 4B). Such antibodies may include amino acid sequence identities of at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 80%, at least about 75%, at least about 70%, at least about 65%, at least about 60%, at least about 55%, at least about 50%; and/or all sequence identity percentages in between to corresponding sequences of antibodies disclosed in Table 1A or 1B, Table 2A or 2B, Table 3A or 3B, or Table 4A or 4B. In some embodiments, percent identity is measured by any well-known algorithm of sequence identity, such as FASTA, BLAST or GAP.

In some embodiments, residue positions that are not identical may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. (See, e.g., Pearson (1994) Methods Mol. Biol. 24: 307-331). Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine. Alternatively, in some embodiments, a conservative replacement comprises any change having a positive value in the PAM250 log-likelihood matrix disclosed in Gonnet et al. (1992) Science 256: 1443 45. In some embodiments, a “moderately conservative” replacement comprises any change having a nonnegative value in a PAM250 log-likelihood matrix.

Substitution of one or more CDR residues or omission of one or more CDRs is also possible. Antibodies have been described in which one or two CDRs can be dispensed to alter binding in the scientific literature. Padlan et al. (1995 FASEB J. 9:133-139) analyzed contact regions between antibodies and their antigens, based on published crystal structures, and concluded that only about one fifth to one third of CDR residues actually contact their associated antigen. Padlan also found many antibodies in which one or two CDRs had zero amino acids in contact with an antigen (see also, Vajdos et al. 2002 J Mol Biol 320:415-428). CDR residues not contacting an antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of an antibody molecule include fusion to the N- or C-terminus of the antibody to an enzyme (e.g., for ADEPT) or a polypeptide which increases serum half-life of the antibody.

Antibodies and antigen-binding fragments described herein may possess favorable developability and may be, thus, relatively developable.

The term “developable” refers to the extent to which one or more polypeptides in a plurality of polypeptides possess desirable characteristics, such as, e.g., desirable expression, for example, in mammalian cells; solubility; viscosity; aggregation; chemical and/or physical stability; desirable shelf-life; melting temperature; pharmacokinetic profiles; circulation half-life; and clearance characteristics. Such characteristics may serve as indicia, independently, as combinations of sub-sets of such indicia, or in totality, for the likelihood that such one or more polypeptides may be successfully developed as a therapeutic candidate, and ultimately an approved drug. Accordingly, as understood in the art, generally, polypeptides with desirable developability characteristics possess, e.g., relatively high solubility, relatively low viscosity, relatively low propensity for aggregation, relatively high chemical stability, relatively high physical stability, relatively long shelf life, relatively high melting temperature, relatively long circulation half-life, relatively long clearance time, and the like. Polypeptides with undesirable developability characteristics possess, e.g., relatively low solubility, relatively high viscosity, relatively high propensity for aggregation, relatively poor chemical stability, relatively poor physical stability, relatively short shelf life, relatively low melting temperature, relatively short circulation half-life, relatively short clearance time, and the like.

Methods and assays that may be employed to ascertain the degree to which polypeptides, such as antibodies and/or antigen-binding fragments thereof described herein, possess desirable developability characteristics are available in the art, and include, for example; PSR assays (WO 2014/179363 and Xu et al., Protein Eng Des Sel, Vol. 26, pages 663-670 (2013)); SMP and SCP assays and the like; cross interaction chromatography (CIC); self-interaction chromatography (SIC); dynamic light scattering (DLS) spectroscopy; size exclusion chromatography (SEC), dynamic light scattering (DLS) spectroscopy; photon correlation spectroscopy; quasi-elastic light scattering, circular dichroism (CD), viscosity measurements; whole cell binding; tissue micro array methodologies; BVP ELISA assays; AC-SINS assays (Liu et al; MAbs, Vol. 6, pages 483-492 (2014); differential scanning calorimetry; and the like (see, e.g., He et al., J. Pharm. Sci., Vol. 100(4), pp. 1330-1340 (2011); Wagner et al., Pharm. Develop. & Technol (posted online 2012; hyper-text transfer protocol: informahealthcare.com/doi/abs/10.3109/10837450.2011.649851); Hotzel et al., MAbs, Vol. 4(6), pages 753-7601 (2012); Weiqiang et al., J Pharm. Sci., Vol. 101(5), pp. 1701-1720 (2012); Banks et al., J. Pharm. Sci., Vol. 101(8), pp. 2720-2732 (2012); Lie et al., J. Pharm. Sci., Vol. 94(9), pp. 1928-1948 (2005); and Payne et al., Biopolymers, Vol. 85(5), pp. 527-533 (2006)).

In some embodiments, antibodies that are identified as possessing decreased developability are so detected by virtue of their interaction with a polyspecificity reagent (“PSR”) and, as such, are referred to as “polyspecific” polypeptides. Such polyspecific antibodies may be referred to as relatively “undevelopable” or relatively “non-developable”.

A “developability profile” refers to an index that may be assigned to antibodies upon assessing their developability. A developability profile is a measure or metric by which developability of antibodies may be assessed, compared, and/or ranked. Such developability profiles serve as a measure of the degree of antibody interactions. Degree of interaction may be assessed by any number of means available in the art that provides an output value that correlates with a strength or affinity of a polypeptide for a moiety to which it is bound. Exemplary means include flow cytometry means, such as FACS; ELISA; quantitative immunoaffinity assays or immunoprecipitation assays; mammalian two-hybrid or yeast two-hybrid assays, and the like. In the context of FACS, as demonstrated in the Examples, a degree of interaction between polypeptides in the plurality and the PSR may be ascertained by generating a mean fluorescence intensity (MFI) for each polypeptide-PSR interaction that is detected, and then ordering the MFI in either ascending or descending order, thereby ranking the polypeptides in the plurality according to the relative degree of interaction between each detected polypeptide and the PSR. Such a ranking provides for a ranking of polypeptides of the plurality such that those polypeptides possessing enhanced developability are readily ascertained, as are those polypeptides possessing decreased developability.

A developability profile may also take the form of a normalized score, for example, by normalizing developability of antibodies described herein to the developability of a standard (or control) antibody.

In certain embodiments, antibodies of the present disclosure may be optimized for enhanced developability profile. The developability profile may be obtained by performing one or more of a PSR assay; an SCP assay; AC-SINS; an ELISA; a DSF assay; a Tm assay; a HIC assay; a CIC assay; and combinations thereof.

In some embodiments, antibodies of the present disclosure may have or be optimized for improved poly-specificity reagent (PSR) score. The PSR score may be between about 0.0 and about 0.45. In some embodiments, the PSR is between about 0.0 and about 0.4. In some embodiments, the PSR is between about 0.0 and about 0.35. In some embodiments, the PSR is between about 0.0 and about 0.3. In some embodiments, the PSR is between about 0.0 and about 0.25. In some embodiments, the PSR is between about 0.0 and about 0.2. In some embodiments, the PSR is between about 0.0 and about 0.15. In some embodiments, the PSR is between about 0.0 and about 0.1, also referred to as a “clean PSR.” In some embodiments, the PSR score is a “low PSR” of between about 0.1 and 0.33. In some embodiments, the PSR score is a “medium PSR” of between about 0.33 to 0.66. In some embodiments, the PSR score is a “high PSR” of between about 0.66 and 1.00. In some embodiments, a high PSR score is indicative of decreased (or poor) developability. Generally, the lower the PSR score the more favorable the developability of the antibody.

In some embodiments, antibodies of the present disclosure display a HIC score of less than about 10.5 minutes (a clean to low HIC score). In some embodiments, a HIC score is between about 10.5 minutes and 11.5 minutes (a medium HIC score). In some embodiments, a HIC score is greater than about 11.5 minutes (a high HIC score). Generally, the lower the HIC score the more favorable the developability of the antibody.

In some embodiments, antibodies of the present disclosure are assessed by size exclusion chromatography (SEC) to confirm that antibodies are monomeric and not aggregating.

In some embodiments, antibodies of the present disclosure display a Tm of less than about 65° C.

In some embodiments, antibodies of the present disclosure may be further modified to minimize effector function, e.g., by modification with a silent Fc.

“Effector function” refers to biological activities attributable to the Fc region of an antibody, which varies by antibody isotype. Exemplary effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

In certain embodiments, the antibodies and/or antigen-binding fragments thereof described herein may be affinity detuned and/or modified to reduce the likelihood or severity of cytokine release syndrome induced by the antibody. Non-limiting exemplary modifications may include silent Fc regions (e.g., removing the Fc completely or modifying the Fc region to reduce or eliminate effector function), and/or masking (e.g., a polypeptide mask that is positioned such that it reduces or inhibits the ability of the antibody or antigen-binding fragment thereof to specifically bind CD3).

In certain embodiments, one or more amino acid modifications may be introduced into the Fc region of an antibody of the disclosure, thereby generating an Fc region variant (see, e.g., US 2012/0251531). An Fc region variant may include a human Fc region sequence (e.g., a human IgG1, IgG2, IgG3 or IgG4 Fc region) including an amino acid modification (e.g., a substitution) at one or more amino acid positions.

In certain embodiments, the disclosure contemplates antibody modifications to include some but not all effector functions, which may be desirable, for example, where in vivo half-life optimization is needed, but where certain effector functions (e.g., ADCC and CDC) are unnecessary or deleterious. In vitro and/or in vivo cytotoxicity assays can be conducted to confirm reduction/depletion of CDC and/or ADCC activities. For example, Fc receptor (FcR) binding assays can be conducted to ensure that an antibody lacks FcγR binding (hence likely lacking ADCC activity) but retains FcRn binding ability. The primary cells for mediating ADCC (e.g., NK cells), express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'lAcad. Sci. USA 83:7059-7063 (1986)) and Hellstrom, I et al., Proc. Nat'lAcad. Sci. USA 82:1499-1502 (1985); U.S. Pat. No. 5,821,337 (see, Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (Cell Technology, Inc. Mountain View, Calif); and CYTOTOX 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity may be assessed in vivo, e.g., in an animal model such as that disclosed in Clynes et al. Proc. Nat'l Acad. Sci. USA 95:652-656 (1998). C1q binding assays may also be carried out to confirm that an antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. ImmunolMethods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol 18(12):1759-1769 (2006)).

In some embodiments, antibodies with reduced effector function include those with substitution of one or more of Fc region residues 238, 265, 269, 270, 297, 327 and 329 (e.g., see U.S. Pat. Nos. 6,737,056 and 8,219,149). In some embodiments, Fc mutants include Fc mutants with substitutions at two or more of amino acid positions 265, 269, 270, 297 and 327, including the so-called “DANA” Fc mutant with substitution of residues 265 and 297 to alanine (e.g., see U.S. Pat. Nos. 7,332,581 and 8,219,149).

In some embodiments, antibodies of the present disclosure may be modified to include a masking agent, e.g., a polypeptide mask, attached via a cleavable linker.

In some embodiments, antibodies described herein may be conjugated to a therapeutic moiety thereby forming an immunoconjugate. An “immunoconjugate” is an antibody conjugated to one or more heterologous molecule(s) such as, e.g., an antibiotic, anti-CD3 antibody, anti-ID anti-CD3 antibody, vaccine, toxoid, or any other therapeutic moiety.

In certain embodiments, antibodies described herein may be altered to increase or decrease the extent of glycosylation. Addition or deletion of glycosylation sites may be conveniently accomplished by altering the amino acid sequence such that one or more glycosylation sites is created or removed.

In certain embodiments, antibodies described herein may be further modified to contain additional non-proteinaceous moieties that are known in the art and are readily available. Moieties suitable for derivatization of an antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, antibodies described herein are associated with detectable labels. Detectable labels may be detected directly (such as with fluorescent, chromophoric, electron-dense, chemiluminescent, and radioactive labels) or indirectly (such as with enzymes or ligands). Non-limiting exemplary detectable labels include, radioisotopes such as 32P, 14C, 1251, 3H, and 1311; fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase; heterocyclic oxidases such as uricase and xanthine oxidase, coupled with an enzyme that employs hydrogen peroxide to oxidize a dye precursor such as HRP, lactoperoxidase, or microperoxidase; biotin/avidin; spin labels; bacteriophage labels; stable free radicals; and the like.

### Antibody Production and Composition Preparation

Antibodies and antigen-binding fragments of the present disclosure may be produced using any appropriate methods including but not limited to recombinant methods. For example, one or more isolated nucleic acids encoding antibodies or antigen-binding fragments thereof as described herein are provided. Such nucleic acids may encode amino acid sequences comprising VL and/or VH amino acid sequences (e.g., antibody light and/or heavy chains).

In some embodiments, one nucleic acid molecule may encode both (i) an amino acid sequence comprising the VH and (ii) an amino acid sequence comprising the VL of the antibody. In certain embodiments, (i) and (ii) may be encoded on the same strand of the nucleic acid molecule. In some cases, (i) and (ii) may be encoded under a single promoter. In certain cases, (i) and (ii) may be encoded in the same direction (in some instances, (i) and (ii) may be transcribed into a single transcript, and in some instances (i) and (ii) may be transcribed into two separate transcripts). In certain cases, (i) and (ii) may be encoded in the opposite directions. In some cases, (i) and (ii) may be encoded under separate promoters. In certain embodiments, (i) and (ii) may be encoded on different strands within a nucleic acid molecule.

In some embodiments, the antibody-encoding nucleic acids may comprise: (i) a first nucleic acid encoding an amino acid sequence comprising the VH; and (ii) a second nucleic acid encoding an amino acid sequence comprising the VL.

In further embodiments, vectors (e.g., expression vectors) that include such nucleic acids are provided.

In some embodiments, one vector may comprise a nucleic acid which encodes both (i) an amino acid sequence comprising the VH and (ii) an amino acid sequence comprising the VL of the antibody. In certain embodiments, (i) and (ii) may be encoded on the same strand of the nucleic acid molecule. In some cases, (i) and (ii) may be encoded under a single promoter. In certain cases, (i) and (ii) may be encoded in the same direction (in some instances, (i) and (ii) may be transcribed into a single transcript, and in some instances (i) and (ii) may be transcribed into two separate transcripts). In certain cases, (i) and (ii) may be encoded in the opposite directions. In some cases, (i) and (ii) may be encoded under separate promoters. In certain embodiments, (i) and (ii) may be encoded on different strands within a nucleic acid.

In some embodiments, one or more vectors may comprise: (i) a first vector comprising a nucleic acid encoding an amino acid sequence comprising the VH; and (ii) a second vector comprising a nucleic acid encoding an amino acid sequence comprising the VL.

In further embodiments, cells (e.g., isolated, recombinant, and/or host cells) that include such nucleic acids and/or vectors are provided. In one such embodiment, a host cell comprises (e.g., has been transformed with): (1) a vector comprising a nucleic acid sequence that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody.

In one embodiment, the host cell is eukaryotic, optionally mammalian (e.g., a Chinese Hamster Ovary (CHO) cell, human embryonic kidney (HEK) cell, or lymphoid cell (e.g., Y0, NS0, Sp20 cell)) or yeast.

In one embodiment, a method of making antibodies of the present disclosure is provided, wherein the method comprises culturing a host cell comprising one or more nucleic acids encoding the antibody, as provided above, under conditions suitable for expression of the antibody, and optionally recovering the antibody from the host cell (or host cell culture medium).

The term “host cell” refers to cells into which an exogenous nucleic acid sequence has been introduced, including the progeny of such cells. Host cells include transformants and transformed cells, which include the primary transformed cell and progeny derived therefrom without regard to the number of passages.

For recombinant production of antibodies, nucleic acids encoding an antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody).

Suitable host cells for cloning and/or expression of antibody-encoding vectors include prokaryotic or eukaryotic cells. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523 (See also, Charlton, Methods in Molecular Biology, Vol. 248 (B. K. C. Lo, ed., Humana Press, Totowa, N.J., 2003), pp. 245-254, describing expression of antibody fragments in E. coli). After expression, the antibody may be isolated from the bacterial cell paste in a soluble fraction and can be further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See, e.g., Gerngross, Nat. Biotech. 22:1409-1414 (2004), and Li et al., Nat. Biotech. 24:210-215 (2006); WO 2009/036379; WO 2010/105256; and WO 2012/009568.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CVI line transformed by SV40 (COS-7); human embryonic kidney line (293 or 293 cells as described, e.g., in Graham et al., J Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Antibodies of the present disclosure may be identified, screened for, selected for, or characterized for their physical/chemical properties and/or biological activities by various assays known in the art, e.g., ELISA, Western blot, etc. Competition assays may be used to identify an antibody that competes for antigen binding. In an exemplary competition assay, immobilized antigen is incubated in a solution comprising a first labeled antibody that binds to the antigen and a second unlabeled antibody that is being tested for its ability to compete with the first antibody for antigen binding. The second antibody may be present in a hybridoma supernatant. As a control, immobilized antigen may be incubated in a solution comprising the first labeled antibody but not the second unlabeled antibody. After incubation under conditions permissive for binding of the first antibody to the antigen, excess unbound antibody can be removed, and the amount of label associated with immobilized antigen can be measured. If the amount of label associated with immobilized antigen is substantially reduced in the test sample relative to the control sample, then the second antibody is characterized as competing with the first antibody for antigen binding. See, e.g., Harlow and Lane (1988) Antibodies: A Laboratory Manual. Ch.14 (Cold Spring Harbor Laboratory, Cold Spring Harbor, N.Y.).

Antibodies and/or antigen-binding fragments thereof possessing biological activity may be identified using standard approaches. Biological activity may include, e.g., antigen binding (e.g., binding to CD3 on the surface of a T cell either in vivo, in vitro, or ex vivo). In the case of a multispecific antibody (such as a bispecific antibody with one antigen-binding region that binds to CD3 and another antigen-binding region that binds to a different target, e.g., a cell surface antigen, e.g., a tumor antigen), biological activity may also include effector cell activation (e.g., CD8+ and/or CD4+ T cell activation), effector cell population expansion (i.e., an increase in T cell count), target cell population reduction (i.e., a decrease in the population of cells expressing the second biological molecule on their cell surfaces), and/or target cell killing.

Antibodies may be identified, isolated, and/or engineered for desirable cytokine release syndrome (CRS) risk profiles, in some instances due to engineering for different affinity and pH-dependent antigen binding profiles. Antibodies may be further engineered to include favorable developability profiles and, in particular, favorable polyspecificity profiles (e.g., to reduce polyspecificity).

The tendency of antibodies to bind multiple targets is referred to as “polyspecificity,” which, with target-specific therapeutic antibodies, may be associated with negative clinical outcomes. Antibodies of the present disclosure may exhibit reduced polyspecificity. Such antibodies may be engineered from starting antibodies by substituting various amino acid residues with residues having charged side chains. Residues may be substituted with amino acid residues having negatively charged side chains, e.g., Asp and Glu residues. Residues selected for substitution may be selected from those not predicted to specifically interact with antigen amino acid residues targeted by the antibodies.

In another aspect of the disclosure, articles of manufacture containing materials useful for the treatment, prevention and/or diagnosis of disorders described herein are provided. Articles of manufacture may include a container and a label or package insert on or associated with the container. Suitable containers include, for example, bottles, vials, syringes, IV solution bags, etc. Containers may be formed from a variety of materials such as glass or plastic. Containers may hold a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition may be an antibody of the disclosure. The label or package insert may indicate that the composition is used for treating a condition of choice. Moreover, articles of manufacture may include (a) a first container with a composition contained therein, wherein the composition comprises an antibody of the disclosure; and (b) a second container with a composition contained therein, wherein the composition comprises a cytotoxic or therapeutic agent. The article of manufacture in this embodiment of the disclosure may further include a package insert indicating that the compositions can be used to treat a particular condition. Alternatively, or additionally, the article of manufacture may further include a second (or third) container including a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Accordingly, manufacture and/or preparation of a pharmaceutical compositions including antibodies disclosed herein is also contemplated. The compositions may be used alone or in combination with other active agents to treat a cell proliferative disorder (e.g., cancer) or an autoimmune disorder (e.g., arthritis, rheumatoid arthritis, colitis, inflammatory bowel disease, autoimmune type I diabetes, etc.).

In some embodiments, pharmaceutical compositions of the present disclosure are prepared, e.g., by mixing antibodies having a desired degree of purity with one or more optional pharmaceutically acceptable carriers (Remington's Pharmaceutical Sciences 16th edition, Osol, A. Ed. (1980)), in the form of lyophilized formulations or aqueous solutions, optionally prepared for modified (e.g., sustained) release. Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968.

Such formulations may contain more than one active ingredient as necessary for the particular indication being treated, preferably those with complementary activities that do not adversely affect each other and present in amounts that are effective for the purpose intended. For example, it may be desirable to further provide an additional therapeutic agent (e.g., a chemotherapeutic agent, a cytotoxic agent, a growth inhibitory agent, and/or an anti-hormonal agent).

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

### Methods of Use

### Methods of Treatment

In some embodiments, antibodies described herein, as well as pharmaceutical compositions of such antibodies, may be used in therapeutic methods.

A “disorder” refers to any condition or disease that would benefit from treatment including, but not limited to, chronic and acute disorders or diseases including those pathological conditions which predispose a mammal to the disorder in question.

The terms “cell proliferative disorder” and “proliferative disorder” refer to disorders that are associated with some degree of abnormal cell proliferation. Cell proliferative disorders include cancer, e.g., a tumor.

“Tumor” as used herein refers to all neoplastic cell growth and proliferation, whether malignant or benign, and all pre-cancerous and cancerous cells and tissues.

“Cancer” refers to a physiological condition in mammals characterized by unregulated cell growth. Examples of cancer include, but are not limited to, carcinoma, lymphoma, blastoma, sarcoma, and leukemia or lymphoid malignancies; with more particular examples including squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by antibodies of the disclosure include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In other embodiments, the cancer is selected from a class of mature B-Cell cancers excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, μ Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

As used herein, “treatment” or “treat” or “treating” refer to clinical intervention in an attempt to alter the natural course of an individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “prevent,” “preventing,” and “prevention” refer to the prevention or inhibition of the development or onset of a disorder or disease.

As used herein, the terms “ameliorate” and “alleviate” refer to a reduction or diminishment in the severity of a condition or any symptoms thereof.

In some embodiments, antibodies of the disclosure are used to delay development of a disorder or disease or to delay the progression of a disorder or disease. As used herein, “delaying progression” of a disorder or disease means to defer, hinder, slow, retard, stabilize, and/or postpone development of the disease or disorder (e.g., a cell proliferative disorder, e.g., cancer). The delay can be of varying lengths of time, depending on the history of the disease and/or individual being treated.

An effective amount of such antibody or composition may be administered to an individual suffering from cancer or arthritis, rheumatoid arthritis, colitis, inflammatory bowel disease, autoimmune type I diabetes, etc. An “effective amount” of an antibody disclosed herein or a composition (e.g., pharmaceutical composition) comprising such antibody, is at least the minimum amount required to achieve the desired therapeutic or prophylactic result, e.g., a measurable improvement or prevention of a particular disorder, e.g., a cell proliferative disorder, e.g., cancer, preferably with minimal or no toxic or detrimental effects. An effective amount may vary according to inter alia disease state, age, sex, and weight of the patient, and the ability of the antibody (or antigen-binding fragment thereof) to elicit a desired response in the individual and, in some instances, by co-administering one or more additional therapeutic agents.

In some embodiments, the present disclosure provides methods of treating subjects (e.g., mammals, e.g., humans, non-human primates, rodents, etc.) by administering anti-ID antibodies described herein. Such treatments may be used to neutralize target antibodies and/or reduce target antibody effects. Effects may include negative effects of prior or concomitant treatment with a target antibody. In some embodiments, anti-ID antibodies of the present disclosure may be administered to reduce negative effects associated with anti-CD3 antibody treatment (e.g., an inflammatory effect, for example, cytokine release syndrome).

The term “cytokine release syndrome” (or “CRS”) refers to a pro-inflammatory, positive feedback loop between cytokines and immune cells leading to excessive or uncontrolled release of pro-inflammatory cytokines by cells within the immune system (see, e.g., Lee et al., Blood, Vol. 124, pages 188-195 (2014) and Tisoncik et al., MicrobiolMol Biol Rev, Vol. 76, pages 16-32 (2012). Upon stimulation and activation, T cells release a series of cytokines to a level and degree that generates untoward biological/physiological effects or varying degree and severity, including acute inflammation characterized by, e.g., rubor (redness), swelling or edema, calor (heat), dolor (pain), and “functio laesa” (loss of function). When localized in skin or other tissue, biological/physiological effects comprise increased blood flow, enabling vascular leukocytes and plasma proteins to reach extravascular sites of injury, increasing local temperatures and generation of pain, tissue edema and extravascular pressure and a reduction in tissue perfusion. Other biological/physiological effects comprise organ and system dysfunction, such as cardiac dysfunction, adult respiratory distress syndrome, neurologic toxicity, renal and/or hepatic failure, and disseminated intravascular coagulation. Elevated levels of IFNγ, IL-6, TNFα, TGFbeta, IL-2, granulocyte macrophage-colony-stimulating factor (GM-CSF), IL-10, IL-8, IL-5, and/or fractalkine are implicated as predictive and/or causative of CRS or the propensity to elicit CRS upon T-cell stimulation. In situations where subjects receiving anti-CD3 antibodies develop CRS, anti-ID antibody treatment methods of the present disclosure may be used to reduce or eliminate the disorder. In some cases, the anti-ID antibody may promote clearance of anti-CD3 antibodies. In some cases, the anti-ID antibody may block binding of the anti-CD3 antibodies to CD3 or CD3-expressing cells such as T cells.

Treatment methods of the present disclosure involving administration of anti-ID antibodies may include administration of additional therapeutic agents. Non-limiting exemplary additional therapeutic agents include a chemotherapy agent, an antibody-drug conjugate (ADC), and/or a biological modifier. Chemotherapy agents may be selected from cyclophosphamide, doxorubicin, vincristine, and prednisolone (CHOP). ADC may be selected from an anti-CD79b antibody drug conjugate (such as anti-CD79b-MC-vc-PAB-MMAE or the anti-CD79b antibody drug conjugate described in any one of U.S. Pat. No. 8,088,378 and/or US 2014/0030280, or polatuzumab vedotin), an anti-CD19 antibody drug conjugate, an anti-CD22 antibody drug conjugate, an anti-CD45 antibody drug conjugate, and an anti-CD32 drug conjugate. A biological modifier may be selected from a BCL-2 inhibitor (such as GDC-0199/ABT-199), lenalidomide (REVLIMID®), a P13K-delta inhibitor (such as idelalisib (ZYDELIG®)), a PD-1 axis binding antagonist, an agonist, e.g., agonist antibody, directed against an activating co-stimulatory molecule, e.g., CD40, CD226, CD28, OX40 (e.g., AgonOX), GITR, CD137 (also known as TNFRSF9, 4-1 BB, or ILA), CD27 (e.g., CDX-1127), HVEM, or CD127, an antagonist, e.g., antagonist antibody, directed against an inhibitory co-stimulatory molecule, e.g., CTLA-4 (also known as CD152), PD-1, TIM-3, BTLA, VISTA, LAG-3, B7-H3, B7-H4, IDO (e.g., 1-methyl-D-tryptophan (also known as 1-D-MT)), TIGIT, MICA/B, GITR (e.g., TRX518) or arginase, ipilimumab (also known as MDX-010, MDX-101, or YERVOY®), tremelimumab (also known as ticilimumab or CP-675,206, urelumab (also known as BMS-663513), MGA271, an antagonist directed against a TGF beta, e.g., metelimumab (also known as CAT-192), fresolimumab (also known as GC1008), LY2157299k, and an adoptive transfer of a T cell (e.g., a cytotoxic T cell or CTL) expressing a chimeric antigen receptor (CAR), e.g., adoptive transfer of a T cell comprising a dominant-negative TGF beta receptor, e.g, a dominant-negative TGF beta type II receptor. In certain embodiments, additional therapeutic agents include a chemotherapeutic agent, growth inhibitory agent, cytotoxic agent, agent used in radiation therapy, anti-angiogenesis agent, apoptotic agent, anti-tubulin agent, or other agent, such as a epidermal growth factor receptor (EGFR) antagonist (e.g., a tyrosine kinase inhibitor), HER1/EGFR inhibitor (e.g., erlotinib (TARCEVA™)), platelet derived growth factor inhibitor (e.g., GLEEVAC™ (Imatinib Mesylate)), a COX-2 inhibitor (e.g., celecoxib), interferon, cytokine, antibody (other than the antibodies of the disclosure), such as an antibody that bind to one or more of the following targets ErbB2, ErbB3, ErbB4, PDGFR-beta, BIyS, APRIL, BCMA VEGF, or VEGF receptor(s), TRAIL/Apo2, PD-1, PD-L1, PD-L2, or another bioactive or organic chemical agent.

Anti-ID antibody treatment methods may include anti-ID administration in combination with (e.g., concomitantly (e.g., in a single composition or separate compositions), sequentially, or other timing format) target antibodies. Combination therapies include combined administration (where two or more therapeutic agents are included in the same or separate formulations), and separate administration, in which case, administration occurs prior to, simultaneously with, and/or following, administration of additional therapeutic agents. Administration of anti-ID antibodies and administration of target antibodies and/or additional therapeutic agents may occur within about one month, or within about one, two or three weeks, or within about one, two, three, four, five, or six days, or within about one, two, three, four, five, six, eight, ten, twelve, twenty four, or forty eight hours of each other.

In some embodiments, anti-ID antibodies are used as masking agents. As used herein, the term “masking agent” refers to a compound used in combination with a drug or other active agent to block or suppresses activity of the drug or active agent for a period of time, under certain conditions (e.g., under certain pH levels), or until reaching a specific location (e.g., a target cell or tissue). Anti-ID antibodies may serve as masking agents by binding to target antibodies and blocking target antibody antigen binding. In some embodiments, anti-ID antibodies or their antigen-binding fragments are conjugated to target antibodies, e.g., to keep the compounds together and facilitate binding site masking. The conjugation may be via a linker. Such linkers may be cleavable. Cleavable linkers may be used to promote masking agent release at sites where target antibody activity is desired, e.g., where the linker is cleaved by a protease expressed at the desired site of activity. In some embodiments, release of the anti-ID antibody from the target antibody may be pH dependent. For example, anti-ID antibody may dissociate from target antibodies at tumor sites based on lower pH around the tumor. Tumor cells typically have an extracellular pH of around about 6.3-6.5. Anti-ID antibodies may preferentially bind to anti-CD3 antibodies at physiological pH levels, causing the anti-ID antibodies to dissociate from anti-CD3 antibodies around the tumor microenvironment, thereby limiting anti-CD3 target antibody binding to the tumor site and reducing or eliminating off-target effects.

In some embodiments, anti-ID antibodies may be used to mask target antibodies according to any of the masking strategies described in Lin W-W. et al. J Biomed Sci. 2020 27:76, the contents of which are herein incorporated by reference in their entirety.

In some embodiments, anti-ID antibodies of the present disclosure are administered in combination with anti-CD3 target antibodies to mitigate or reduce or eliminate an effect of the target antibodies in a cell or tissue. The anti-CD3 target antibodies may be administered to treat a target cell or target tissue (e.g., a cancer cell or cancerous tumor or tissue) and the anti-ID antibodies may mitigate or reduce or eliminate an effect of the target antibody in a non-target cell or non-target tissue (e.g., by masking anti-CD3 binding activity). Anti-ID antibodies may be bound to or associated with anti-CD3 target antibodies prior to administration. Such treatments may prevent anti-CD3 antibody binding until a target cell or tissue is reached post-administration. The anti-ID antibodies may be induced to dissociate from the anti-CD3 target antibodies by one or more of, for example, pH change, association with an inhibitor, presence of a competitive inhibitor, and presence of a protease or other factor promoting dissociation.

In some embodiments, methods of the disclosure may further include additional therapies. The additional therapies may include radiation therapy, surgery, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy may be a separate administration of one or more of the therapeutic agents described above.

In some embodiments, antibodies described herein may be used for diagnosis and/or detection. “Detection” as used herein encompasses quantitative or qualitative detection.

### Administration Methods

Antibodies of the disclosure (and/or any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In some embodiments, antibodies are administered by subcutaneous administration. Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the disclosure may be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, and other factors known to medical practitioners. Antibodies need not, but may optionally be, formulated with one or more agents currently used to prevent or treat a disorder in question. The effective amount of such other agents depends on the amount of antibody present in the formulation, the type of disorder or treatment, and other factors discussed above. These are generally used in the same dosages and with administration routes as described herein, or from about 1 to 99% of the dosages described herein, or in any dosage and by any route that is empirically/clinically determined to be appropriate.

For the prevention or treatment of disease, the appropriate dosage of antibodies of the disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. Antibodies may be suitably administered to subjects at one time or over a series of treatments.

As a general proposition, a therapeutically effective amount of antibodies administered to humans will be in the range of about 0.01 to about 100 mg/kg of patient body weight whether by one or more administrations. In some embodiments, an antibody used is administered in about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg daily, for example. In one embodiment, an anti-ID antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, for example, every week or every three weeks (e.g., such that the patient receives from about two to about twenty, or, for example, about six doses of the anti-ID antibody). An initial higher loading dose, followed by one or more lower doses, may be administered. The progress of this therapy is easily monitored by conventional techniques and assays.

### Isolation and Characterization Methods

In some embodiments, the present disclosure provides methods of isolating target antibodies by contacting them with anti-ID antibodies described herein. Such methods may include associating anti-ID antibodies with a substrate (e.g., a in a column or on an ELISA plate) and exposing the substrate to a sample or solution that includes target antibodies (e.g., anti-CD3 antibodies). The sample may be a body fluid sample (e.g., blood sample). In certain embodiments, such method may be for determining the amount of the target antibodies (e.g., time course changes of the amount) in the sample. The solution may include an antibody library.

In some embodiments, the present disclosure provides methods of characterizing target antibodies using anti-ID antibodies disclosed herein. Such methods may include evaluating target antibody affinity (e.g., affinity of anti-CD3 antibody) for anti-ID antibodies by surface plasmon resonance (SPR) analysis or BLI. In some embodiments, anti-ID antibodies may be used to evaluate pharmacokinetic and/or pharmacodynamic properties associated with target antibody treatments (e.g., treatment with an anti-CD3 antibody). Such treatments may include treatments with multispecific anti-CD3 antibodies. Anti-ID antibodies may be used as competitors for evaluating associations between anti-CD3 antibodies and CD3.

In some embodiments, methods of the present disclosure include the use of anti-ID antibodies to determine the presence or absence of anti-drug antibodies in samples. Such methods may include contacting samples with anti-ID antibodies and assessing the presence or absence of antibodies competing for target antibody binding in the samples. The samples may be from subjects that have been administered anti-CD3 antibodies.

### Kit

The present disclosure further provides a kit comprising (a) any of the antibodies and antigen-binding fragments described herein (anti-ID antibodies and antigen-binding fragments); and (b) a target antibody of the antibody or antigen-binding fragment thereof of (a).

In some embodiments, the kit may be for use in a method of analyzing a sample, e.g., a biological sample from a subject. In certain embodiments, the subject may be administered with a target antibody (e.g., an anti-CD3 antibody) and the method may be for determining the amount of the target antibody contained in the sample. In such embodiments, the target antibody comprised in the kit may be used as a control antibody, e.g., for generating a standard curve as a reference for determining the amount of the target antibody in the sample. In some cases, such a method may be for analyzing pharmacokinetics of the target antibody in the subject.

In some embodiments, the kit may be for used in analyzing competition in binding to the target antibody (e.g., anti-CD3 antibody). In certain embodiments, whether an antibody of interest (e.g., another anti-ID antibody) may compete with a reference antibody (e.g., an anti-ID antibody) in binding to the target antibody may be tested. In some cases, for example, one sample comprising the target antibody and one or more samples comprising the target antibody and the reference antibody (e.g., of different concentrations in different samples) may be first provided, and the antibody of interest may be gradually added to each sample. In some cases, for example, one sample comprising the target antibody and one or more samples comprising the target antibody and the antibody of interest (e.g., of different concentrations in different samples) may be first provided, and the reference antibody of interest may be gradually added to each sample. The reference antibody or the antibody of interest may be quantified (e.g., reference antibody or the antibody of interest may comprise a label) to determine whether the reference antibody and the antibody of interest may compete.

In some embodiments, the kit may be for comparing binding. In certain embodiments, the kit may be for testing whether an antibody of interest (e.g., another anti-ID antibody) may bind better (e.g., with higher affinity/avidity) than a reference antibody (e.g., anti-ID contained in the kit) to the target antibody contained in the kit. In certain embodiments, the kit may be for testing whether the antibody or antigen-binding fragment contained in the kit may bind better (e.g., with higher affinity/avidity) to a target antibody of interest (e.g., another anti-CD3 antibody) than the reference target antibody (e.g., anti-CD3 antibody) contained in the kit.

It must also be noted that, unless the context clearly dictates otherwise, the singular forms “a,” “an,” and “the” as used herein and in the appended claims include plural refence. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

It is understood that aspects and embodiments of the disclosure described herein include “comprising,” “consisting,” and “consisting essentially of” aspects and embodiments.

Although various embodiments and examples of the present invention have been described referring to certain molecules, compositions, methods, or protocols, it is to be understood that the present invention is not limited to the particular molecules, compositions, methods, or protocols described herein, as theses may vary. It should be understood that, unless clearly indicated otherwise, in any methods disclosed or claimed herein that comprise more than one step, the order of the steps to be performed is not restricted by the order of the steps cited.

It is also to be understood that the terminology used in the description is for the purpose of describing the particular versions or embodiments only and is not intended to limit the scope of the present invention which will be limited only by the appended claims.

All references cited herein, including patent documents and non-patent documents, are hereby incorporated by reference in their entirety. Nothing herein is to be construed as an admission that the present invention is not entitled to antedate such disclosure by virtue of prior invention.

Examples are provided below to illustrate the present invention. These examples are not meant to constrain the present invention to any particular application or theory of operation.

### EXAMPLES

The following examples unless otherwise indicated were effected using the antibody sequences shown in Tables 1A-1B, 2A-2B, 3A-3B, 4A-4D, 11, and/or 12. These antibody sequences are intended to be exemplary as the invention embraces the construction and use of variants thereof.

### Example 1: Anti-Idiotype Anti-CD3 Antibodies

Anti-idiotype (anti-ID) antibodies were identified based on affinity for anti-CD3 antibody ADI-22523 described in U.S. Publication No. US 2020/0190189, which is incorporated by reference herein in its entirety. ADI-30242 and ADI-30254 were identified during initial analysis and used to prepare light chain variants for additional affinity screening. Additional screening identified ADI-30242 antibody variants ADI-34131-ADI-34138 and ADI-30254 antibody variants ADI-34139-ADI-34146.

Specific affinity values of identified antibodies (produced in yeast) for ADI-22523 (produced in yeast) were determined by measuring kinetic constants (kₒₙ, k_{off}, K_{D}) on ForteBio Octet. ForteBio affinity measurements were performed generally as previously described (Estep et al., MAbs. 2013 5(2):270-8). Briefly, ForteBio affinity measurements were performed by loading antibody IgG or Fabs on-line onto AHC sensors. Sensors were equilibrated off-line in assay buffer for 30 minutes and then monitored on-line for 60 seconds for baseline establishment. For avid binding measurement, sensors with loaded antibodies were exposed to IgG or Fab solutions for 3 minutes, afterwards they were transferred to assay buffer for 3 minutes for off-rate measurement. Kinetics data were fit using a 1:1 binding model in the data analysis software provided by ForteBio to obtain equilibrium dissociation constant (K_{D}=k_{off}/kₒₙ) values. Results associated with anti-ID antibodies analyzed are provided in Table 5. In each column, tip-immobilized antibodies are listed first and solution antibodies second.

All of the identified antibodies demonstrated affinity for ADI-22523 IgG and Fab, with the exception of ADI-34132, which did not demonstrate observable binding to ADI-22523 Fab. Among the ADI-30242 variants shown, ADI-34134 demonstrated the highest affinity for both ADI-22523 IgG and Fab. Among the ADI-30254 variants shown, ADI-34144 and ADI-34145 demonstrated the highest affinity for ADI-22523 IgG, with ADI-34145 demonstrating the highest affinity for ADI-22523 Fab.

Additional assessments were carried out to assess whether the anti-ID antibodies compete for ADI-22523 binding to CD3 by further ForteBio Octet affinity analysis, but with prior association of tip-immobilized ADI-22523 IgG with CD38E-Fc and exposure to anti-ID IgGs in solution. Kinetic values indicated that ADI-30254 and associated variants competed with CD3 for ADI-22523 binding, while ADI-30242 and associated variants did not. ADI-34132 did not demonstrate any observable binding in the assay.

Based on these results, ADI-34134 and ADI-34145 were selected for additional characterization.

ADI-34134 and ADI-34145 (produced in yeast) affinity for additional anti-CD3 antibodies (produced in yeast) described in U.S. Publication No. US 2020/0190189 was determined by ForteBio Octet affinity analysis according to the method described above. Anti-ID antibody affinity for CTL-19613 and CTL-19672 was also determined. CTL-19613 is an IgG1 antibody prepared with anti-CD3 variable domains corresponding with those of clone 12C described in United States Publication Number US 2010/0183615 (e.g., see paragraphs 469-479 and FIG. 25 thereof), which also make up the CD3 binding domain of the bispecific antibody pasotuxizumab. CTL-19672 is an IgG1 antibody prepared with anti-CD3 variable domains corresponding with those associated with clone 38E4v1 as described in U.S. Pat. No. 10,174,124. Resulting kinetic values are provided in the Table 6. In each column, tip-immobilized antibodies are listed first and solution antibodies second. In Table 6, “poor fit” is indicated where antibody affinity was observed, but where associated values could not be adequately fit to a 1:1 binding model.

Anti-ID antibodies tested demonstrated strong affinity for most of the anti-CD3 antibodies tested, with ADI-34134 demonstrating broader affinity than ADI-34145. CTL-19672 IgG was bound at relatively lower affinity by ADI-34134 and not bound by ADI-34145.

Additional affinity measurements with ADI-34134 and ADI-34145 (produced in yeast) for binding to anti-CD3 antibodies (produced in yeast) were obtained using alternative arrangements of tip-associated antibodies and antibodies in solution. Resulting kinetic values are provided in Table 7. In each column, tip-immobilized antibodies are listed first and solution antibodies second. In Table 7, “poor fit” is indicated where antibody affinity was observed, but where associated values could not be adequately fit to a 1:1 binding model.

Additional analysis was conducted using bispecific antibody formats incorporating anti-CD3 antibody variable domains. Bispecific antibodies were prepared using standard IgG formats (bsIgG) with replacement of Fab arms with anti-CD3 scFvs in Vx-VH orientation and with S354C and Y349C mutations for disulfide stabilization. Antibodies used for Fab arm replacement were anti-HER2 IgG antibodies, with one Fab arm from each replaced by a scFv corresponding to a different anti-CD3 antibody. Kinetic values obtained for ADI-34134 and ADI-34145 (produced in yeast) for binding to anti-CD3 antibodies (produced in yeast) are provided in Table 8. In each column, tip-immobilized antibodies are listed first and solution antibodies second. Anti-CD3 antibodies associated with incorporated scFvs are listed by antibody number. In Table 8, “poor fit” is indicated where antibody affinity was observed, but where associated values could not be adequately fit to a 1:1 binding model.

Anti-ID antibodies tested demonstrated affinity for bsIgGs similar to that observed with monovalent anti-CD3 IgGs.

### Example 2. Anti-ID Antibody Developability Assessment

Anti-ID antibody developability was determined using multiple assessments, including polyspecificity and hydrophobic interaction analyses.

Antibodies with high affinity for a target may otherwise fail in clinical settings where they also exhibit binding to multiple non-target entities. Antibody polyspecificity was assessed by measuring interaction with polyspecificity reagent (“PSR Polyspecific reactivity reagent (PSR) was prepared as described in, e.g., WO 2014/179363 and Xu et. al., Protein Eng Des Sel, 26(10):663-670 (2013). In brief, 2.5 liters CHO-S cells were used as starting material. The cells were pelleted at 2,400×g for 5 min in 500 mL centrifuge bottles filled to 400 mL. Cell pellets were combined and then resuspended in 25 ml Buffer B and pelleted at 2,400×g for 3 min. The buffer was decanted and the wash repeated one time. Cell pellets were resuspended in 3× the pellet volume of Buffer B containing 1× protease inhibitors (Roche, Complete, EDTA-free) using a polytron homogenizer with the cells maintained on ice. The homogenate was then centrifuged at 2,400×g for 5 min and the supernatant retained and pelleted one additional time (2,400×g/5 min) to ensure the removal of unbroken cells, cell debris and nuclei; the resultant supernatant is the total protein preparation. The supernatant was then transferred into two Nalgene Oak Ridge 45 mL centrifuge tubes and pelleted at 40,000×g for 40 min at 4° C. The supernatants containing the Separated Cytosolic Proteins (SCPs) were then transferred into clean Oak Ridge tubes, and centrifuged at 40,000×g one more time. In parallel, the pellets containing the membrane fraction (EMF) were retained and centrifuged at 40,000 for 20 min to remove residual supernatant. The EMF pellets were then rinsed with Buffer B. 8 mL Buffer B was then added to the membrane pellets to dislodge the pellets and transfer into a Dounce Homogenizer. After the pellets were homogenized, they were transferred to a 50 mL conical tube and represented the final EMF preparation.

One billion mammalian cells (e.g., CHO, HEK293, Sf9) at ˜10⁶-10⁷ cells/mL were transferred from tissue culture environment into 4×250 mL conical tubes and pelleted at 550×g for 3 min. All subsequent steps were performed at 4° C. or on ice with ice-cold buffers. Cells were washed with 100 mL of PBSF (1× PBS+1 mg/mL BSA) and combined into one conical tube. After removing the supernatant, the cell pellet was then re-suspended in 30 mL Buffer B (50 mM HEPES, 0.15 M NaCl, 2 mM CaCl₂), 5 mM KCl, 5 mM MgCl2, 10% Glycerol, pH 7.2) and pelleted at 550×g for 3 min. Buffer B supernatant was decanted and cells re-suspended in 3× pellet volume of Buffer B plus 2.5× protease inhibitor (Roche, cOmplete, EDTA-free). Protease inhibitors in Buffer B were included from here on forward. Cells were homogenized four times for 30 sec pulses (Polyton homogenizer, PT1200E) and the membrane fraction was pelleted at 40,000×g for 1 hour at 4° C. The pellet is rinsed with 1 mL Buffer B; the supernatant is retained and represents the s. The pellet is transferred into a Dounce homogenizer with 3 mL of Buffer B and re-suspended by moving the pestle slowly up and down for 30-35 strokes. The enriched membrane fraction (EMF) is moved into anew collection tube, rinsing the pestle to collect all potential protein. Determine the protein concentration of the purified EMF using the Dc-protein assay kit (BioRad). To solubilize the EMF, transfer into Solubilization Buffer (50 mM HEPES, 0.15 M NaCl, 2 mM CaCl₂), 5 mM KCl, 5 mM MgCl2, 1% n-Dodecyl-b-D-Maltopyranoside (DDM), 1× protease inhibitor, pH 7.2) to a final concentration of 1 mg/mL. Rotate the mixture overnight at 4° C. rotating followed by centrifugation in a 50 mL Oak Ridge tube (Fisher Scientific, 050529-ID) at 40,000×g for 1 hour. Collect the supernatant which represents the soluble membrane proteins (SMPs) and quantify the protein yield as described above.

For biotinylation, prepare the NHS-LC-Biotin stock solution according to manufacturer's protocol (Pierce, Thermo Fisher). In brief, 20 ul of biotin reagent is added for every 1 mg of EMF sample and incubated at 4° C. for 3 hours with gentle agitation. Adjust the volume to 25 mL with Buffer B and transfer to an Oak Ridge centrifuge tube. Pellet the biotinylated EMF (b-EMF) at 40,000×g for 1 hour, and rinse two times with 3 mL of Buffer C (Buffer B minus the glycerol) without disturbing the pellet. Remove the residual solution. Re-suspended the pellet with a Dounce homogenizer in 3 mL of Buffer C as described previously. The re-suspended pellet now represents biotinylated EMF (b-EMF). Solubilized as described above to prepare b-SMPs.

PSR Binding Analyses. Assays were performed generally as described in, e.g., Xu et al. Protein Eng Des Sel, 26(10):663-670 (2013). To characterize the PSR profile of monoclonal antibodies presented on yeast, two million IgG-presenting yeast were transferred into a 96-well assay plate and pellet at 3000×g for 3 min to remove supernatant. Re-suspend the pellet in 50 ul of freshly prepared 1:10 dilution of stock b-PSRs and incubate on ice for 20 minutes. Wash the cells twice with 200 ul of cold PBSF and pellet re-suspended in 50 ul of secondary labeling mix (Extravidin-R-PE, anti-human LC-FITC, and propidium iodide). Incubate the mix on ice for 20 minutes followed by two washes with 200 ul ice-cold PBSF. Re-suspend the cells in 100 ul of ice-cold PBSF and run the plate on a FACSCanto (BD Biosciences) using HTS sample injector. Flow cytometry data was analyzed for mean fluorescence intensity in the R-PE channel and normalized to proper controls in order to assess non-specific binding. Numerous methods for presentation or display of antibodies or antibody fragments on the surface of yeast have been described previously, all of which are consistent with this protocol (Blaise et al., Gene, 342(2):211-8 (2004), Boder and Wittrup, Nat Biotechnol., 15(6):553-7 (1997), Kuroda and Ueda, Biotechnol Lett., 33(1):1-9 (2011), Orcutt and Wittrup, Springer Protocols: Antibody Engineering, 1:207-233 (2010), Rakestraw et al., Protein Eng Des Sel., 24(6):525-30 (2011), Sazinsky et al., Proc Natl Acad Sci USA., 105(51):20167-72 (2008), Tasumi et al., Proc Natl Acad Sci USA., 106(31):12891-6 (2009).

Hydrophobic interaction chromatography (HIC). IgG1 samples were buffer exchanged into 1 M ammonium sulfate and 0.1 M sodium phosphate at pH 6.5 using a Zeba 40 kDa 0.5 mL spin column (Thermo Pierce, cat #87766). A salt gradient was established on a Dionex ProPac HIC-10 column from 1.8 M ammonium sulfate, 0.1 M sodium phosphate at pH 6.5 to the same condition without ammonium sulfate. The gradient ran for 17 min at a flow rate of 0.75 ml/min. An acetonitrile wash step was added at the end of the run to remove any remaining protein and the column was re-equilibrated over 7 column volumes before the next injection cycle. Peak retention times were monitored at A280 absorbance and concentrations of ammonium sulfate at elution were calculated based on gradient and flow rate.

Final PSR scores and HIC retention times for antibodies (produced in yeast) tested are listed in Table 9.

The majority of antibodies tested demonstrated PSR scores<0.1, a “clean” PSR score, indicating very low levels of non-target-specific binding. Similarly, a majority of antibodies tested demonstrated HIC retention times<10.5, a “clean to low” HIC retention time.

### Example 3. Methods

The following paragraphs include methods used in antibody assessments, including many of those described above.

Hu and Cy CD3εδFc heterodimer antigen production. Recombinant heterodimeric CD3 Fc fusion antigens were produced in HEK 293 cells by co-transfection of plasmids encoding Hu CD3ε Fc (ectodomain, ECD, residues 22-126) and CD3ε Fc-HIS (ECD residues 22-100) or Cy CD3ε Fc (ECD residues 22-117) and CD3ε Fc-HIS (ECD residues 22-100) utilizing a heterologous signal peptide sequence. Chromatographic separations were performed on a computer controlled AKTA Avant 150 preparative chromatography system (GE Healthcare Life Sciences) equipped with an integrated conductivity sensor, enabling in-line salt concentration monitoring during the run. Clarified culture supernatants were purified by Ni Sepharose 6 Fast Flow (GE Healthcare Life Sciences), which removes the CD3εε Fc-HIS homodimer. CD3εδ Fc-HIS heterodimer was resolved from CD366 Fc-HIS homodimer by Mono Q 10/100 GL by a linear Tris-buffered KCl gradient at pH 8.5.

Peptides. C-terminally biotinylated CD3ε N-terminal peptides were obtained from New England Peptide. All peptides were delivered with a purity of ≥95%. Peptides were designed based on the primary sequence of Hu CD3ε and the crystal structure of Hu CD3εδ bound to OKT3 (Kjer-Nielsen L. et al. PNAS 2004). The CD3εN27 peptide has the sequence H2N-QDGNEEMGSITQTPYQVSISGTTVILT[K/SCBiot(dPEG4)]-amide (SEQ ID NO: 104) and the CD3εN13 peptide has the sequence H2N-QDGNEEMGGITQT[K/SCBiot(dPEG4)]-amide (SEQ ID NO: 105).

Antigen biotinylation. CD3 antigens were biotinylated using the EZ-Link Sulfo-NHS-Biotinylation Kit from Pierce. Goat anti-human F(ab′)2 kappa-FITC (LC-FITC), Extravidin-PE (EA-PE) and streptavidin-633 (SA-633) were obtained from Southern Biotech, Sigma and Molecular Probes, respectively. Streptavidin MicroBeads and MACS LC separation columns were purchased from Miltenyi Biotec.

Cell line propagation and cell labeling assays. Human Jurkat CD3+ cells (ATCC TIB-152) and Jurkat CD3cells (ATCC TIB-153) were obtained from ATCC. Cyno HSC-F cells were obtained from the NIH Non-human Primate Reagent Resource. All cell lines were cultured in RPMI 1640 GlutaMax media supplemented with 10% fetal bovine serum (FBS).

Cell labeling was conducted by aliquoting 100,000-200,000 cells per well in a 96-well assay plate. Cells were centrifugated at 500×g for 5 min at 4° C., then resuspended in 100 μl of 100 nM IgG and incubated at room temperature for 20 min. Cells were then washed in buffer (phosphate-buffered saline (PBS)/0.1% bovine serum albumin (BSA) three times and resuspended in secondary reagent, typically goat anti-human R-PE (Southern Biotech). The plate was assayed on a FACSCANTO™ (BD Biosciences) using an HTS sample injector. Flow cytometry data was analyzed for median fluorescence intensity in the R-PE channel.

FACS affinity pressured selection methods. Briefly, yeast cells (at least ˜2×10⁷ cells/labeling condition) were incubated with a volume of biotinylated antigen sufficient to represent a stoichiometric excess with respect to the average IgG presentation number. Antigen labeling conditions are 100 to 1 nM under equilibrium conditions, typically carried out for 20 min to several hours at room temperature in FACS wash buffer (phosphate-buffered saline (PBS)/0.1% bovine serum albumin (BSA)). After washing three times with wash buffer, yeast were then stained with secondary reagents anti-human light chain FITC conjugate (LC-FITC) diluted 1:100 and either streptavidin-633 (SA-633) diluted 1:500 or extravidin-phycoerythrin (EA-PE) diluted 1:50 for 15 min at 4° C. After washing twice with ice-cold wash buffer, the cell pellets were resuspended in wash buffer in a typical volume of at least 1 mL per 1×10⁷ yeast and transferred to strainer-capped sort tubes. Sorting was performed using a FACSARIA™ sorter (BD Biosciences) and sort gates were determined to select for binders. After the final round of sorting, yeast were plated and individual colonies picked for characterization.

Antibody yeast production and purification. Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect or CaptureSelect IgG-CH1 (GE Healthcare LifeSciences).

Antibody HEK production and purification. Mammalian expression of IgG was done by sub-cloning antibodies into a new expression vector followed by transient transfection and expression in HEK cells. Briefly, expression vectors containing the antibody of interest were transfected by complexing with a transfection reagent followed by exposure to HEK cells for one hour followed by dilution of culture media to a final density of 4 million cells per mL. The cells were then cultured for 7 days with fresh feed media every 48 hours. After 7 days, the supernatant was collected following centrifugation and purification was performed using protein A. If necessary, a CHT column purification was added to reach>95% monomer.

Cell binding assays. CD3+ human Jurkat cells (ATCC) and CHO-S cells (Invitrogen/ThermoFisher) were thawed and washed with cold PBSF buffer, pH 7.4 (PBS+0.1% BSA, pH 7.4). About 200,000 cells were aliquoted per well of a 96-well plate (FACS Assay Plate VWR BD 353263) and pelleted by centrifugation (5 minutes at 500× g). The cells were washed with either PBSF pH 7.4 or PBSF pH 6.0 (PBS+0.1% BSA, pH 6.0), and then resuspended in 100 μl in either PBSF pH 7.4 or PBSF pH 6.0 with IgG antibody (100 nM) produced in yeast as described above. The mixture (cells+ antibody) was incubated for 20 minutes on ice, then washed twice with either PBSF pH 7.4 or PBSF pH 6.0. Cells were resuspended in 50 μl of propidium iodide (Roche; 1:500 dilution) and anti-human IgG-RPE (Southern Biotech; 1:100 dilution) prepared in either PBSF pH 7.4 or PBSF pH 6.0, then incubated for 20 minutes on ice in the dark before cells were washed twice with either PBSF pH 7.4 or PBSF pH 6.0. Binding was analyzed on FACS Canto II.

ForteBio K_{D} measurements (Biolayer interferometry; BLI). ForteBio affinity measurements were performed generally as previously described (Estep, P., et al., High throughput solution-based measurement of antibody-antigen affinity and epitope binning. MAbs, 2013. 5(2): p. 270-8.). Briefly, ForteBio affinity measurements were performed by loading IgGs online onto AHC sensors. Sensors were equilibrated off-line in assay buffer for 30 min and then monitored on-line for 60 seconds for baseline establishment. Sensors with loaded IgGs were exposed to 100 nM antigen (e.g., CD3 or anti-CD3 antibodies) for 5 min, afterwards they were transferred to assay buffer for 5 min for off-rate measurement. Kinetics were analyzed using the 1:1 binding model.

ForteBio Kinetics. FortBio Octet HTX instruments were used in 12 channel mode (8 sensors per channel, 96 sensors per experiment) with either AHC, SA, or AHQ sensors. Instrumentation was driven by manufacturer supplied software (versions 8.2 and 9.0). Sample names and concentrations were input into the plate data page, and sensor associated proteins were identified in the “information” column on the sensor data page. Kinetic experiments were collected with either a 90 or 180 s baseline, 180 s association phase, and 180 s dissociation phase. All files were saved into a shared network drive with a naming convention that identifies the format of the experiment.

HIC. IgG1 samples were buffer exchanged into 1 M ammonium sulfate and 0.1 M sodium phosphate at pH 6.5 using a Zeba 40 kDa 0.5 mL spin column (Thermo Pierce, cat #87766). A salt gradient was established on a Dionex ProPac HIC-10 column from 1.8 M ammonium sulfate, 0.1 M sodium phosphate at pH 6.5 to the same condition without ammonium sulfate. The gradient ran for 17 min at a flow rate of 0.75 ml/min. An acetonitrile wash step was added at the end of the run to remove any remaining protein and the column was re-equilibrated over 7 column volumes before the next injection cycle. Peak retention times were monitored at A280 absorbance and concentrations of ammonium sulfate at elution were calculated based on gradient and flow rate.

LCMS. mAb samples were reduced by DTT, followed by middle down LCMS analysis on a Bruker maXis4G mass spectrometer coupled with an Agilent 1100 HPLC (Agilent). A POROS R2 10 μm (2.1×30 mm) reversed phase column was used to remove salt in the samples. A fast LC flow at 2 mL/min allows the separation between sample and salt and elution of samples and regeneration of column to finish within a 2.1 min cycle. A T-junction is used to deliver only 0.15 mL/min sample flow into the mass spectrometer for sample analysis. The Bruker maXis 4G mass spectrometer was run in positive ion mode with detection in the range of 750 to 2500 m/z. The remaining source parameters were set as follows; the capillary was set at 5500V, the Nebulizer at 4.0 Bar, dry gas at 4.0 1/min, and dry temp set at 200° C.

The MS spectra were analyzed using Bruker Data Analysis version 4.1 and the deconvolution was accomplished using maximum entropy deconvolution with a mass range of 20 to 30 kDa.

SEC. Agilent 1260 HPLC was employed to monitor the column chromatography (TSKgel Super SW mAb HTP column). The column was equilibrated with wash buffer (200 mM Sodium Phosphate, 250 mM Sodium Chloride pH 6.8) at a flow rate adjusted to 0.400 mL/min prior to use. Approximately 2-5 μg of an IgG1 or Fab protein sample was injected onto column. Protein migration was monitored at wavelength 280 nm. Total assay time was approximately 6 minutes. Data was analyzed using ChemStation software. Propensity of antibodies to aggregate was assessed based on % monomer in SEC chromatogram. Antibodies with % monomer of 95% or higher were considered to be substantially existing as a monomer, i.e., not aggregating.

Acidic stress tolerability. IgG1 samples were incubated at an acidic pH or a physiological pH and subjected to SEC-HPLC analyses. Briefly, IgG1 samples at 20 mg/mL were buffer exchanged into PBS (200 mM phosphate buffered with 250 mM sodium chloride, pH 7.0) or pH 3.5 buffer (50 mM sodium chloride, 200 mM acetic acid, pH 3.5). After 1 hour at room temperature (25° C.), buffer exchanged samples were diluted to 1 mg/mL in PBS (200 mM phosphate buffered with 250 mM sodium chloride, pH 7.0), and 2 μg of sample was injected into an Agilent 1260 Infinity analytical HPLC (Agilent, Santa Clara, CA) fitted with a TSKgel SuperSW mAb HTP column (TOSOH Bioscience, King of Prussia, PA, Product Code 22855). SEC data was collected and subjected to analysis using Agilent ChemStation software (Agilent, Santa Clara, CA). Tolerance of antibodies to acidic stress was evaluated based on propensity of antibodies to aggregate in an acidic condition assessed by % monomer in SEC chromatogram.

AC-SINS. Self-interaction was be measured in vitro by affinity-capture self-interaction nanoparticle spectroscopy (AC-SINS) using a previously described protocol (Liu y et al., MAbs. Mar-Apr 2014; 6(2):483-92). Briefly, polyclonal goat anti-human IgG Fc antibodies (capture; Jackson ImmunoResearch Laboratories) and polyclonal goat non-specific antibodies (non-capture; Jackson ImmunoResearch Laboratories) were buffer exchanged into 20 mM sodium acetate (pH 4.3) and concentrated to 0.4 mg/ml. A 4:1 volume ratio of capture:non-capture may be prepared and further incubated at a 1:9 volume ratio with 20 nm gold nanoparticles (AuNP; Ted Pella Inc.) for 1 hour at room temperature. Thiolated PEG (Sigma-Aldrich) was then be used to block empty sites on the AuNP and filtered via a 0.22 μm PVDF membrane (Millipore). Coated particles were subsequently added to the test IgG1 antibody solution and incubated for 2 hours at room temperature before measuring absorbance from 510 to 570 nm on a plate reader. Data points were fit with a second-order polynomial in Excel to obtain wavelengths at maximum absorbance. Values were reported as the difference between plasmon wavelengths of the sample and background (Δλmax). Self-interaction levels were determined based on Δλmax. Self-interaction was considered: low when Δλmax<5.0 nm; medium when Δλmax≥5.0 nm and <20.0 nm; and high when Δλmax≥20.0 nm.

DLS. Self-interaction was measured by dynamic light scattering (DLS). Diffusion Interaction Parameter (kD) of monoclonal antibodies, measured at concentrations lower than 12 mg/mL, has strong correlation with their solution behavior in very high concentrations (>100 mg/mL). Positive kD values indicate repulsive interaction among the molecules and has positive correlation with low viscosity at high concentration, in the same formulation buffer. kD values were obtained by measuring mutual diffusion coefficient for a series of different concentrations, by DLS. Specifically, DLS kD measurements at multiple concentrations between 0.5-12 mg/mL, in 10 mM Histidine buffer, pH 6.0 were taken. kD values<20 mL/g were considered as being associated with high viscosity or high opalescence.

DSF. Melting temperature (Tm) was measured by differential scanning fluorometry (DSF) using a CFX96 Real-Time System from Bio-Rad. Briefly, 20 μL of 1 mg/mL sample was mixed with 10 μL of 20× SYPRO orange. The plate was scanned from 40° C. to 95° C. at a rate of 0.5° C./2 min in a C1000 thermocycler (BioRad) to collect Fret signal. The Fab Tm was assigned using the first derivative of the raw data from the Bio-Rad analysis software. Antibodies with a Tm higher than 65° C. were considered to be stable.

### Example 4. Additional Binding Analyses

Binding kinetics associated with anti-ID antibody binding to anti-CD3 antibodies were further analyzed by SPR. Anti-ID antibodies were produced in yeast as described above. For ADI-34134 and ADI-34145, antibodies were also produced in CHO cells. Anti-CD3 antibodies were produced in CHO cells. These included a selection of those described in Example 1 and related variants as well as a set of anti-CD3 antibodies associated with various clinical studies.

Kinetic analysis was conducted at 25° C. in an HBSTE running buffer system (Carterra Cat #3630) using a Carterra LSA (Carterra USA, Salt Lake City, UT). The sample compartment was maintained at 20° C. for the duration of each experiment.

For antibody capture experiments, a goat anti-human Fc antibody (Jackson ImmunoResearch) was covalently coupled to an HC30M sensor chip surface via standard amine coupling (1:1 EDC:NHS) and then blocked with ethanolamine (1.0 M, pH 8.5). Covalent coupling steps were performed in 25 mM MES buffer (Carterra, Cat #3625) plus 0.05% Tween20. The anti-CD3 antibodies (100 nM in running buffer) were flowed over discrete regions of interest on the chip surface for 5 minutes. Five concentrations of the anti-idiotype antibodies ranging from 200.0 to 0.781 nM (4-fold dilutions in running buffer) were injected for 300s over the entire chip surface. Dissociation of the anti-idiotype antibodies was monitored for 300s. Several blank buffer samples were injected over the entire chip surface at the start of each non-regenerative kinetic cycle. The preceding blank buffer injection was used for blank surface subtraction. At the end of each non-regenerative kinetic cycle, all surfaces were regenerated via two 30s injections of 10 mM glycine, pH 1.7.

For data processing and fitting, sensorgrams were double reference subtracted with a baseline drift correction of 4 RU. Sensorgrams were y-aligned and baseline corrected, filtered and cropped to include only the association and dissociation steps. Data was fitted to a 1:1 binding model using Carterra Kinetics Data Analysis software version 1.7.1.3055.

Kinetic values (avidity K_{D} (M)) of yeast-produced ADI-30242 and ADI-30242 variants; yeast-produced ADI-30254 and ADI-30254 variants; and CHO cell-produced ADI-34134 and ADI-34145 for anti-CD3 antibodies (produced in CHO cells) are provided in Tables 10A, 10B, and 10C, respectively. In Tables 10A-10C, anti-CD3 antibodies are listed by ADI number or CTL number in the first column. ADI-48576, ADI-48587, ADI-48592, ADI-48650, ADI-74965, ADI-74966, ADI-74967, and ADI-74968 are pH-dependent anti-CD3 antibodies having increased affinity at pH 6.0 compared with pH 7.4. CTL-62223 is mosunetuzumab, CTL-62227 is odronextamab, CTL-62228 is glofitamab, and CTL-62240 is ONO-4685. “P.F.”, representing poor fit, is indicated where antibody affinity was observed, but where associated values could not be adequately fit to a 1:1 binding model. “W.B.” means weak binding under the conditions of this assay and thus a K_{D} was unable to be assigned. “N.B.” means no binding under the conditions of this assay.

As shown in Tables 10A-10C, anti-ID antibodies demonstrated binding to a variety of anti-CD3 antibodies tested with affinity values varying between anti-ID and anti-CD3 antibody binding pairs.

### Exemplary Embodiments

Described herein below are some exemplary embodiments according to the present disclosure.

Embodiment 1. An antibody or antigen-binding fragment thereof comprising: a complementarity determining region (CDR) comprising an amino acid sequence selected from any of those listed in Table 4A or 4B or Table 17.

Embodiment 2. The antibody or antigen-binding fragment thereof of Embodiment 1 comprising: a heavy chain variable domain (VH) comprising an amino acid sequence selected from any of those listed in Table 3A or Table 16; and/or a light chain variable domain (VL) comprising an amino acid sequence selected from any of those listed in Table 3B or Table 16.

Embodiment 3. The antibody or antigen-binding fragment thereof of Embodiment 2, wherein: the VH comprises the amino acid sequence of SEQ ID NO: 51 and the VL comprises the amino acid sequence of SEQ ID NO: 53; the VH comprises the amino acid sequence of SEQ ID NO: 51 and the VL comprises the amino acid sequence of SEQ ID NO: 54; the VH comprises the amino acid sequence of SEQ ID NO: 51 and the VL comprises the amino acid sequence of SEQ ID NO: 55; the VH comprises the amino acid sequence of SEQ ID NO: 51 and the VL comprises the amino acid sequence of SEQ ID NO: 56; the VH comprises the amino acid sequence of SEQ ID NO: 51 and the VL comprises the amino acid sequence of SEQ ID NO: 57; the VH comprises the amino acid sequence of SEQ ID NO: 51 and the VL comprises the amino acid sequence of SEQ ID NO: 58; the VH comprises the amino acid sequence of SEQ ID NO: 51 and the VL comprises the amino acid sequence of SEQ ID NO: 59; the VH comprises the amino acid sequence of SEQ ID NO: 51 and the VL comprises the amino acid sequence of SEQ ID NO: 60; the VH comprises the amino acid sequence of SEQ ID NO: 51 and the VL comprises the amino acid sequence of SEQ ID NO: 61; the VH comprises the amino acid sequence of SEQ ID NO: 52 and the VL comprises the amino acid sequence of SEQ ID NO: 62; the VH comprises the amino acid sequence of SEQ ID NO: 52 and the VL comprises the amino acid sequence of SEQ ID NO: 63; the VH comprises the amino acid sequence of SEQ ID NO: 52 and the VL comprises the amino acid sequence of SEQ ID NO: 64; the VH comprises the amino acid sequence of SEQ ID NO: 52 and the VL comprises the amino acid sequence of SEQ ID NO: 65; the VH comprises the amino acid sequence of SEQ ID NO: 52 and the VL comprises the amino acid sequence of SEQ ID NO: 66; the VH comprises the amino acid sequence of SEQ ID NO: 52 and the VL comprises the amino acid sequence of SEQ ID NO: 67; the VH comprises the amino acid sequence of SEQ ID NO: 52 and the VL comprises the amino acid sequence of SEQ ID NO: 68; the VH comprises the amino acid sequence of SEQ ID NO: 52 and the VL comprises the amino acid sequence of SEQ ID NO: 69; or the VH comprises the amino acid sequence of SEQ ID NO: 52 and the VL comprises the amino acid sequence of SEQ ID NO: 70.

Embodiment 4. The antibody or antigen-binding fragment thereof of Embodiment 1 comprising: a VH comprising a CDR set according to any of those listed in Table 4A or a CDR set comprised in a VH listed in Table 16; and a VL comprising a CDR set according to any of those listed in Table 4B or a CDR set comprised in a VL listed in Table 16.

Embodiment 5. The antibody or antigen-binding fragment thereof of any one of Embodiments 1-4, wherein the antibody or antigen-binding fragment thereof binds to a target antibody.

Embodiment 6. The antibody or antigen-binding fragment thereof of Embodiment 5, wherein the target antibody comprises an antigen-binding domain that binds Cluster of Differentiation 3 (CD3).

Embodiment 7. The antibody or antigen-binding fragment thereof of Embodiment 5 or 6, wherein the target antibody antigen-binding domain comprises: a VH comprising an amino acid sequence according to any of those listed in Table TA or Table 14; and a VL comprising an amino acid sequence according to any of those listed in Table 1B or Table 14.

Embodiment 8. The antibody or antigen-binding fragment thereof of any one of Embodiments 5-7, wherein the target antibody antigen-binding domain comprises: a VH comprising a CDR sequence set according to any of those listed in Table 2A or a CDR set comprised in a VH listed in Table 14; and/or a VL comprising a CDR sequence set according to any of those listed in Table 2B or a CDR set comprised in a VL listed in Table 14.

Embodiment 9. The antibody or antigen-binding fragment thereof of any one of Embodiments 5-8, wherein the target antibody comprises a multispecific antibody, a bispecific antibody, and/or an scFv.

Embodiment 10. The antibody or antigen-binding fragment thereof of any one of Embodiments 5-9, wherein the target antibody comprises a multispecific antibody comprising a second antigen-binding domain that binds to: an oncology target; an immune-oncology target; a neurodegenerative disease target; an autoimmune disorder target; an infectious disease target; a metabolic disease target; a cognitive disorder target; a blood-brain barrier target; and/or a blood disease target.

Embodiment 11. The antibody or antigen-binding fragment thereof of Embodiment 10, wherein the target antibody second antigen-binding domain binds to any one or more of the second antigens described herein.

Embodiment 12. The antibody or antigen-binding fragment thereof of Embodiment 10, wherein the target antibody second antigen-binding domain binds to one or more of: BCMA, CTLA4 (cytotoxic T lymphocyte antigen-4), PD-1 (programmed cell death protein 1), PD-L1 (programmed cell death ligand 1), LAG-3 (lymphocyte activation gene-3), TIM-3, CD20, CD2, CD19, Her2, EGFR, EpCAM, FcγRIIIa (CD16), FcγRIIa (CD32a), FcγRIIb (CD32b), FcγRI (CD64), Toll-like receptors (TLRs), TLR4, TLR9, cytokines, IL-2, IL-5, IL-13, IL-6, IL-17, IL-12, IL-23, TNFα, TGFO, a cytokine receptor, IL-2R, a chemokine, a chemokine receptor, a growth factor, VEGF, HGF, and a hormone receptor.

Embodiment 13. The antibody or antigen-binding fragment thereof of any one of Embodiments 5-12, wherein the target antibody comprises a chimeric antigen receptor (CAR).

Embodiment 14. The antibody or antigen-binding fragment thereof of Embodiment 13, wherein the CAR optionally comprises a transmembrane domain, an intracellular domain from a T-cell receptor, a CD3 subunit, and/or a co-stimulatory domain.

Embodiment 15. The antibody or antigen-binding fragment thereof of any one of Embodiments 5-12, wherein the target antibody comprises: an scFv2-Fc2 and/or scFv-IgG; an IgG constant domain; and/or a multispecific format according to one or more of a Fab-Fc-scFv, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

Embodiment 16. A nucleic acid encoding an antibody or antigen-binding fragment thereof according to any one of Embodiments 1-15.

Embodiment 17. A construct comprising a nucleic acid sequence according to Embodiment 16.

Embodiment 18. A cell comprising a nucleic acid according to Embodiment 16 and/or a construct according to Embodiment 17, wherein the cell is optionally a mammalian cell or a yeast cell.

Embodiment 19. A composition comprising an excipient and one or more of: an antibody or antigen-binding fragment thereof according to any one of Embodiments 1-15; a nucleic acid according to Embodiment 16; a construct according to Embodiment 17; and a cell according to Embodiment 18.

Embodiment 20. A pharmaceutical composition comprising a pharmaceutically acceptable excipient and one or more of: an antibody or antigen-binding fragment thereof according to any one of Embodiments 1-15; a nucleic acid according to Embodiment 16; a construct according to Embodiment 17; and a cell according to Embodiment 18.

Embodiment 21. A method of treating a subject in need of such treatment, the method comprising administering the antibody or antigen-binding fragment thereof of any one of Embodiments 5-15.

Embodiment 22. The method of Embodiment 21, wherein the subject comprises a target antibody, wherein the target antibody comprises an antigen-binding domain that binds CD3, and wherein administering the antibody or antigen-binding fragment thereof neutralizes the target antibody.

Embodiment 23. The method of Embodiment 21, wherein the antibody or antigen-binding fragment thereof is administered in combination with a target antibody, wherein the target antibody comprises an antigen-binding domain that binds CD3.

Embodiment 24. The method of Embodiment 23, wherein the antibody or antigen-binding fragment thereof is conjugated with the target antibody.

Embodiment 25. The method of Embodiment 24, wherein the antibody or antigen-binding fragment thereof is conjugated with the target antibody via a linker.

Embodiment 26. The method of Embodiment 25, wherein the linker comprises a cleavable linker.

Embodiment 27. The method of any one of Embodiments 23-26, wherein the antibody or antigen-binding fragment thereof reduces an effect of the target antibody in at least one cell or tissue.

Embodiment 28. The method of any one of Embodiments 23-27, wherein the target antibody is administered to treat a target cell or target tissue and wherein the antibody or antigen-binding fragment thereof reduces an effect of the target antibody in a non-target cell or non-target tissue.

Embodiment 29. The method of any one of Embodiments 23-28, wherein the antibody or antigen-binding fragment thereof is used as a masking agent of the target antibody.

Embodiment 30. The method of any one of Embodiments 23-29, wherein the antibody or antigen-binding fragment thereof is bound to the target antibody prior to administration.

Embodiment 31. The method of Embodiment 30, wherein the antibody or antigen-binding fragment thereof dissociates from the target antibody after administration.

Embodiment 32. The method of Embodiment 31, wherein dissociation of the antibody or antigen-binding fragment thereof from the target antibody is induced by one or more of change in pH, association with an inhibitor, presence of a competitive inhibitor, and presence of a protease.

Embodiment 33. The method of any one of Embodiments 21-32, wherein the antibody or antigen-binding fragment thereof is administered to the subject to treat a disorder.

Embodiment 34. The method of Embodiment 33, wherein the disorder is related to a prior treatment of the subject with a target antibody, wherein the target antibody comprises an antigen-binding domain that binds CD3.

Embodiment 35. The method of Embodiment 33 or 34, wherein the disorder comprises an inflammatory disorder.

Embodiment 36. The method of any one of Embodiments 33-35, wherein the disorder comprises cytokine release syndrome.

Embodiment 37. The method of any one of Embodiments 21-36 comprising administering an additional therapeutic agent.

Embodiment 38. The method of any one of Embodiments 21-37, wherein the subject is a mammal, wherein the mammal is optionally a human.

Embodiment 39. A method of isolating a target antibody, the method comprising contacting the target antibody with the antibody or antigen-binding fragment thereof of any one of Embodiments 5-15.

Embodiment 40. A method of characterizing a target antibody, wherein the target antibody comprises an antigen-binding domain that binds CD3, the method comprising the use of an antibody or antigen-binding fragment thereof according to any one of Embodiments 5-15.

Embodiment 41. The method of Embodiment 40, wherein the antibody or antigen-binding fragment thereof is used to characterize a pharmacokinetic and/or pharmacodynamic property associated with the target antibody.

Embodiment 42. A method of determining the presence or absence of an anti-drug antibody in a sample, the method comprising the use of an antibody or antigen-binding fragment thereof according to any one of Embodiments 5-15.

TABLE 1A
Anti-CD3 VH sequences

| Antibody | | |
|---|---|---|
| Contain- | | SEQ |
| ing ADI | VH | ID |
| Name | Sequence | NO: |
| ADI-22523 | QVQLVQSGAEVKKPGASVKVSCKASGFNIKDYYMHWVRQAPGQRLEWMGWIDLENGNTIYDPKFQGRVTI | 110 |
| | TRDTSASTAYMELSSLRSEDTAVYYCARDGYGRYFYDVWGQGTLVTVSS | |
| ADI-26927 | QVQLVQSGAEVKKPGASVKVSCKASGFNIKDYYMHWVRQAPGQRLEWMGWIDLEEGNTIYDAKFQGRVTI | 120 |
| | TRDTSASTAYMELSSLRSEDTAVYYCARDRYGAYFYDVWGQGTLVTVSS | |
| ADI-26928 | QVQLVQSGAEVKKPGASVKVSCKASGFNIKDYYMHWVRQAPGQRLEWMGWIDLEEGNTIYDAKFQGRVTI | 130 |
| | TRDTSASTAYMELSSLRSEDTAVYYCGRDAYGLYFYDVWGQGTLVTVSS | |
| ADI-26941 | QVQLVQSGAEVKKPGASVKVSCKASGFNIKDYYMHWVRQAPGQRLEWMGWIDLEEGNTIYDAKFQGRVTI | 140 |
| | TRDTSASTAYMELSSLRSEDTAVYYCARDQYGRYFYDVWGQGTLVTVSS | |
| ADI-26954 | QVQLVQSGAEVKKPGASVKVSCKASGFNIKDYYMHWVRQAPGQRLEWMGWIDLENANTIYDAKFQGRVTI | 150 |
| | TRDTSASTAYMELSSLRSEDTAVYYCARDNYGGYFYDVWGQGTLVTVSS | |
| CTL-19613 | EVQLVESGGGLVQPGGSLKLSCAASGFTFNKYAMNWVRQAPGKGLEWVARIRSKYNNYATYYADSVKDRFTI | 160 |
| | SRDDSKNTAYLQMNNLKTEDTAVYYCVRHGNFGNSYISYWAYWGQGTLVTVSS | |
| CTL-19672 | EVQLVQSGAEVKKPGASVKVSCKASGFTFTSYYIHWVRQAPGQGLEWIGWIYPENDNTKYNEKFKDRVTITA | 170 |
| | DTSTSTAYLELSSLRSEDTAVYYCARDGYSRYYFDYWGQGTLVTVSS | |
| ADI-50024 | QVQLVQSGAEVKKPGASVKVSCKASGFNIKDYYMHWVRQAPGQRLEWIGWIDLENANTVYDAKFQGRVTI | 180 |
| | TRDTSASTAYMELSSLRSEDTAVYYCARDAYGRYFYDVWGQGTLVTVSS | |
| ADI-70326 | QVQLVQSGAEVKKPGASVKVSCKASGFDIKDYYMHWVRQAPGQRLEWIGWIELENADTVYDAKFQGRVTIT | 190 |
| | RDTSASTAYMELSSLRSEDTAVYYCARDAYGRYFYDVWGQGTLVTVSS | |
| ADI-70327 | QVQLVQSGAEVKKPGASVKVSCKASGFDIKDYYMHWVRQAPGQRLEWIGWIELENDDTVYDAKFQGRVTIT | 200 |
| | RDTSASTAYMELSSLRSEDTAVYYCARDAYGRYFYDVWGQGTLVTVSS | |
| ADI-70330 | QVQLVQSGAEVKKPGASVKVSCKASGFNIDDYYMHWVRQAPGQRLEWIGWIDLENANTVDDAKFQGRVTI | 210 |
| | TRDTSASTAYMELSSLRSEDTAVYYCARDAYGRYFYDVWGQGTLVTVSS | |
| ADI-26906 | QVQLVQSGAEVKKPGASVKVSCKASGFNIKDYYMHWVRQAPGQRLEWMGWIDLENANTIYDAKFQGRVTI | 220 |
| | TRDTSASTAYMELSSLRSEDTAVYYCARDAYGRYFYDVWGQGTLVTVSS | |
| ADI-67450 | QVQLVQSGAEVKKPGASVKVSCKASGFNIDDYYMHWVRQAPGQRLEWMGWIDLENANTIDDAKFQGRVT | 230 |
| | ITRDTSASTAYMELSSLRSEDTAVYYCARDAYGRYFYDVWGQGTLVTVSS | |
| ADI-67445 | QVQLVQSGAEVKKPGASVKVSCKASGFNIKDYYMHWVRQAPGQRLEWMGWIDLENAETIYDAKFQDRVTI | 240 |
| | TRDTSASTAYMELSSLRSEDTAVYYCARDAYGRYFYDEWGQGTLVTVSS | |
| ADI-67447 | QVQLVQSGAEVKKPGASVKVSCKASGFNIDDYYMHWVRQAPGQRLEWMGWIDLENDNTIYDAKFQGRVTI | 250 |
| | TRDTSASTAYMELSSLRSEDTAVYYCARDAYGRYFYDVWGQGTLVTVSS | |
| ADI-26909 | QVQLVQSGAEVKKPGASVKVSCKASGFNIKDYYMHWVRQAPGQRLEWMGWIDLEEGNTIYDAKFQGRVTI | 260 |
| | TRDTSASTAYMELSSLRSEDTAVYYCGRDAYGRYFYDVWGQGTLVTVSS | |
| ADI-26913 | QVQLVQSGAEVKKPGASVKVSCKASGFNIKDYYMHWVRQAPGQRLEWMGWIDLENANTIYDAKFQGRVTI | 270 |
| | TRDTSASTAYMELSSLRSEDTAVYYCARDQYGRYFYDVWGQGTLVTVSS | |
| ADI-26919 | QVQLVQSGAEVKKPGASVKVSCKASGFNIKDYYMHWVRQAPGQRLEWMGWIDLENANTIYDAKFQGRVTI | 280 |
| | TRDTSASTAYMELSSLRSEDTAVYYCARDAYGRYFYDVWGQGTLVTVSS | |
| ADI-26920 | QVQLVQSGAEVKKPGASVKVSCKASGFNIKDYYMHWVRQAPGQRLEWMGWIDLENANTIYDAKFQGRVTI | 290 |
| | TRDTSASTAYMELSSLRSEDTAVYYCGRDAYGRYFYDVWGQGTLVTVSS | |
| ADI-26921 | QVQLVQSGAEVKKPGASVKVSCKASGFNIKDYYMHWVRQAPGQRLEWMGWIDLEEGNTIYDAKFQGRVTI | 300 |
| | TRDTSASTAYMELSSLRSEDTAVYYCARDAYGRYFYDVWGQGTLVTVSS | |
| ADI-26916 | QVQLVQSGAEVKKPGASVKVSCKASGFNIKDYYMHWVRQAPGQRLEWMGWIDLENANTIYDAKFQGRVTI | 310 |
| | TRDTSASTAYMELSSLRSEDTAVYYCARDVYGRYFYDLWGQGTLVTVSS | |
| ADI-26912 | QVQLVQSGAEVKKPGASVKVSCKASGFNIKDYYMHWVRQAPGQRLEWMGWIDLENANTIYDAKFQGRVTI | 320 |
| | TRDTSASTAYMELSSLRSEDTAVYYCARDVLGRYFYDVWGQGTLVTVSS | |
| ADI-26924 | QVQLVQSGAEVKKPGASVKVSCKASGFNIKDYYMHWVRQAPGQRLEWMGWIDLENANTIYDAKFQGRVTI | 330 |
| | TRDTSASTAYMELSSLRSEDTAVYYCGRDAYGLYFYDVWGQGTLVTVSS | |
| ADI-26915 | QVQLVQSGAEVKKPGASVKVSCKASGFNIKDYYMHWVRQAPGQRLEWMGWIDLEEGNTIYDAKFQGRVTI | 340 |
| | TRDTSASTAYMELSSLRSEDTAVYYCARDVLGRYFYDVWGQGTLVTVSS | |
| ADI-26943 | QVQLVQSGAEVKKPGASVKVSCKASGFNIKDYYMHWVRQAPGQRLEWMGWIDLENANTIYDAKFQGRVTI | 350 |
| | TRDTSASTAYMELSSLRSEDTAVYYCARDAYGGYFYDVWGQGTLVTVSS | |
| ADI-48576 | QVQLVQSGAEVKKPGASVKVSCKASGFNIKDYYMHWVRQAPGQRLEWMGWIDLENANTIYDAKFQGRVTI | 360 |
| | TRDTSASTAYMELSSLRSEDTAVYYCARDAYHRYFYDHWGQGTLVTVSS | |
| ADI-48587 | QVQLVQSGAEVKKPGASVKVSCKASGFNIKDYYMHWVRQAPGQRLEWMGWIDLENANTIYDAKFQGRVTI | 370 |
| | TRDTSASTAYMELSSLRSEDTAVYYCARDHYHRYFYDVWGQGTLVTVSS | |
| ADI-48592 | QVQLVQSGAEVKEPGASVKVSCKASGFNIKDYYMHWVRQAPGQRLEWMGWIDLENANTIYDAKFQGRVTI | 380 |
| | TRDTSASTAYMELSSLRSEDTAVYYCARDAHHRYFYDVWGQGTLVTVSS | |
| ADI-48650 | QVQLVQSGAEVKKPGASVKVSCKASGFNIKDYYMHWVRQAPGQRLEWMGWIDLENANTIYDAKFQGRVTI | 390 |
| | TRDTSASTAYMELSSLRSEDTAVYYCARDATHRYFYDVWGQGTLVTVSS | |
| ADI-74965 | QVQLVQSGAEVKKPGASVKVSCKASGFNIKDYYMHWVRQAPGQRLEWMGWIDLENANTIYDAKFQGRVTI | 400 |
| | TRDTSASTAYMELSSLRSEDTAVYYCARDAYHRYFYDVWGQGTLVTVSS | |
| ADI-74966 | QVQLVQSGAEVKKPGASVKVSCKASGFNIKDYYMHWVRQAPGQRLEWMGWIDLENANTIYDAKFQGRVTI | 410 |
| | TRDTSASTAYMELSSLRSEDTAVYYCARDHYHRYFYDVWGQGTLVTVSS | |
| ADI-74967 | QVQLVQSGAEVKKPGASVKVSCKASGFNIKDYYMHWVRQAPGQRLEWMGWIDLENANTIYDAKFQGRVTI | 420 |
| | TRDTSASTAYMELSSLRSEDTAVYYCARDHYHRYFYDVWGQGTLVTVSS | |
| ADI-74968 | QVQLVQSGAEVKKPGASVKVSCKASGFNIKDYYMHWVRQAPGQRLEWMGWIDLENANTIYDAKFQGRVTI | 430 |
| | TRDTSASTAYMELSSLRSEDTAVYYCARDAYHRYFYDVWGQGTLVTVSS | |
| ADI-76357 | QVQLVQSGAEVKKPGASVKVSCKASGFDIKDYYMHWVRQAPGQRLEWMGWIDLENANTIYDAKFQGRVTI | 440 |
| | TRDTSASTAYMELSSLRSEDTAVYYCARDQYGRYFYDVWGQGTLVTVSS | |
| ADI-76358 | QVQLVQSGAEVKKPGASVKVSCKASGFNIKDYYMHWVRQAPGQRLEWMGWIDLENADTIYDAKFQGRVTI | 450 |
| | TRDTSASTAYMELSSLRSEDTAVYYCARDQYGRYFYDVWGQGTLVTVSS | |
| ADI-76359 | QVQLVQSGAEVKKPGASVKVSCKASGFDIKDYYMHWVRQAPGQRLEWMGWIDLENANTIYDAKFQGRVTI | 460 |
| | TRDTSASTAYMELSSLRSEDTAVYYCGRDAYGRYFYDVWGQGTLVTVSS | |
| ADI-76360 | QVQLVQSGAEVKKPGASVKVSCKASGFNIKDYYMHWVRQAPGQRLEWMGWIDLENADTIYDAKFQGRVTI | 470 |
| | TRDTSASTAYMELSSLRSEDTAVYYCGRDAYGRYFYDVWGQGTLVTVSS | |
| ADI-76361 | QVQLVQSGAEVKKPGASVKVSCKASGFDIKDYYMHWVRQAPGQRLEWMGWIDLEEGNTIYDAKFQGRVTI | 480 |
| | TRDTSASTAYMELSSLRSEDTAVYYCARDAYGRYFYDVWGQGTLVTVSS | |
| ADI-76362 | QVQLVQSGAEVKKPGASVKVSCKASGFNIKDYYMHWVRQAPGQRLEWMGWIDLEEGDTIYDAKFQGRVTI | 490 |
| | TRDTSASTAYMELSSLRSEDTAVYYCARDAYGRYFYDVWGQGTLVTVSS | |
| CTL-62223 | EVQLVQSGAEVKKPGASVKVSCKASGYTFTNYYIHWVRQAPGQGLEWIGWIYPGDGNTKYNEKFKGRATLTA | 500 |
| | DTSTSTAYLELSSLRSEDTAVYYCARDSYSNYYFDYWGQGTLVTVSS | |
| CTL-62227 | EVQLVESGGGLVQPGRSLRLSCAASGFTFDDYTMHWVRQAPGKGLEWVSGISWNSGSIGYADSVKGRFTISR | 510 |
| | DNAKKSLYLQMNSLRAEDTALYYCAKDNSGYGHYYYGMDVWGQGTTVTVAS | |
| CTL-62228 | EVQLLESGGGLVQPGGSLRLSCAASGFTFSTYAMNWVRQAPGKGLEWVSRIRSKYNNYATYYADSVKGRFTI | 520 |
| | SRDDSKNTLYLQMNSLRAEDTAVYYCVRHGNFGNSYVSWFAYWGQGTLVTVSS | |
| CTL-62240 | QVQLVQSGGGVVQPGRSLRLSCVASGFTFSSYGMHWVRQAPGKGLEWVAQIWYNARKQEYSDSVKGRFTISR | 530 |
| | DNSKNTLYLQMNSLRAEDTAVYYCTRGTGYNWFDPWGQGTLVTVSS | |

Underlined sequence spans correspond to CDRH1, CDRH2, and CDRH3 in the order of appearance. Sequence spans without an underline correspond to FRH1, FRH2, FRH3, and FRH4 in the order of appearance.

TABLE 1B
Anti-CD3 VL sequences

| Antibody | | |
|---|---|---|
| Contain- | | SEQ |
| ing ADI | VL | ID |
| Name | Sequence | NO: |
| ADI-22523 | DIVMTQSPDSLAVSLGERATINCKSSQSLLNSRTGKNYLAWYQQKPGQPPKLLIYWASTRESGVPDRFSGSGSGTDF | 115 |
| | TLTISSLQAEDVAVYYCKQSYSRRTFGGGTKVEIK | |
| ADI-26927 | DIVMTQSPDSLAVSLGERATINCKSSQSLLNARTGKNYLAWYQQKPGQPPKLLIYWASTRESGVPDRFSGSGSGTDF | 125 |
| | TLTISSLQAEDVAVYYCKQSYSLRTFGGGTKVEIK | |
| ADI-26928 | DIVMTQSPDSLAVSLGERATINCKSSQSLLNARTGKNYLAWYQQKPGQPPKLLIYWASTRESGVPDRFSGSGSGTDF | 135 |
| | TLTISSLQAEDVAVYYCKQSYSLRTFGGGTKVEIK | |
| ADI-26941 | DIVMTQSPDSLAVSLGERATINCKSSQSLLNARTGKNYLAWYQQKPGQPPKLLIYWASTRESGVPDRFSGSGSGTDF | 145 |
| | TLTISSLQAEDVAVYYCKQSYSIRTFGGGTKVEIK | |
| ADI-26954 | DIVMTQSPDSLAVSLGERATINCKSSQSLLESRTGKNYLAWYQQKPGQPPKLLIYWASTRESGVPDRFSGSGSGTDF | 155 |
| | TLTISSLQAEDVAVYYCKQSYSLRTFGGGTKVEIK | |
| CTL-19613 | QTVVTQEPSLTVSPGGTVTLTCGSSTGAVTSGNYPNWVQQKPGQAPRGLIGGTKFLAPGTPARFSGSLLGGKAALT | 165 |
| | LSGVQPEDEAEYYCVLWYSNRWVFGGGTKLTVL | |
| CTL-19672 | DIVMTQSPDSLAVSLGERATINCKSSQSLLNSRTRKNYLAWYQQKPGQSPKLLIYWTSTRKSGVPDRFSGSGSGTDF | 175 |
| | TLTISSLQAEDVAVYYCKQSFILRTFGQGTKVEIK | |
| ADI-50024 | DIVMSQSPDSLAVSLGERATINCKSSQSLLNARTGKNYLAWYQQKPGQPPKLLIYWASTRSSGVPDRFSGSGSGTDF | 185 |
| | TLTISSLQAEDVAVYYCVQSYFRRTFGGGTKVEIK | |
| ADI-70326 | DIVMSQSPDSLAVSLGERATINCKSSQSLLNARTGKNYLAWYQQKPGQPPKLLIYWASTRSSGVPDRFSGSGSGTDF | 195 |
| | TLTISSLQAEDVAVYYCVQSYFRRTFGGGTKVEIK | |
| ADI-70327 | DIVMSQSPDSLAVSLGERATINCKSSQSLLNARTGKNYLAWYQQKPGQPPKLLIYWASTRSSGVPDRFSGSGSGTDF | 205 |
| | TLTISSLQAEDVAVYYCVQSYFRRTFGGGTKVEIK | |
| ADI-70330 | DIVMSQSPDSLAVSLGERATINCKSSQSLLNARTGKNYLAWYQQKPGQPPKLLIYWASTRSSGVPDRFSGSGSGTDF | 215 |
| | TLTISSLQAEDVAVYYCVQSYFRRTFGGGTKVEIK | |
| ADI-26906 | DIVMTQSPDSLAVSLGERATINCKSSQSLLNARTGKNYLAWYQQKPGQPPKLLIYWASTRESGVPDRFSGSGSGTDF | 225 |
| | TLTISSLQAEDVAVYYCKQSYSRRTFGGGTKVEIK | |
| ADI-67450 | DIVMTQSPDSLAVSLGERATINCKSSQSLLNARTGKNYLAWYQQKPGQPPKLLIYWASTRESGVPDRFSGSGSGTDF | 235 |
| | TLTISSLQAEDVAVYYCKQSYSRRTFGGGTKVEIK | |
| ADI-67445 | DIVMTQSPDSLAVSLGERATINCKSSQSLLNARTGKNYLAWYQQKPGQPPKLLIYWASTRESGVPDRFSGSGSGTDF | 245 |
| | TLTISSLQAEDVAVYYCKQSYSRRTFGGGTKVEIK | |
| ADI-67447 | DIVMTQSPDSLAVSLGERATINCKSSQSLLNARTGKNYLAWYQQKPGQPPKLLIYWASTRESGVPDRFSGSGSGTDF | 255 |
| | TLTISSLQAEDVAVYYCKQSYSRRTFGGGTKVEIK | |
| ADI-26909 | DIVMTQSPDSLAVSLGERATINCKSSQSLLNARTGKNYLAWYQQKPGQPPKLLIYWASTRESGVPDRFSGSGSGTDF | 265 |
| | TLTISSLQAEDVAVYYCKQSYSRRTFGGGTKVEIK | |
| ADI-26913 | DIVMTQSPDSLAVSLGERATINCKSSQSLLNARTGKNYLAWYQQKPGQPPKLLIYWASTRESGVPDRFSGSGSGTDF | 275 |
| | TLTISSLQAEDVAVYYCKQSYSRRTFGGGTKVEIK | |
| ADI-26919 | DIVMTQSPDSLAVSLGERATINCKSSQSLLNARTGKNYLAWYQQKPGQPPKLLIYWASTRESGVPDRFSGSGSGTDF | 285 |
| | TLTISSLQAEDVAVYYCKQSYSLRTFGGGTKVEIK | |
| ADI-26920 | DIVMTQSPDSLAVSLGERATINCKSSQSLLNARTGKNYLAWYQQKPGQPPKLLIYWASTRESGVPDRFSGSGSGTDF | 295 |
| | TLTISSLQAEDVAVYYCKQSYSLRTFGGGTKVEIK | |
| ADI-26921 | DIVMTQSPDSLAVSLGERATINCKSSQSLLNARTGKNYLAWYQQKPGQPPKLLIYWASTRESGVPDRFSGSGSGTDF | 305 |
| | TLTISSLQAEDVAVYYCKQSYSLRTFGGGTKVEIK | |
| ADI-26916 | DIVMTQSPDSLAVSLGERATINCKSSQSLLNARTGKNYLAWYQQKPGQPPKLLIYWASTRESGVPDRFSGSGSGTDF | 315 |
| | TLTISSLQAEDVAVYYCKQSYSRRTFGGGTKVEIK | |
| ADI-26912 | DIVMTQSPDSLAVSLGERATINCKSSQSLLNARTGKNYLAWYQQKPGQPPKLLIYWASTRESGVPDRFSGSGSGTDF | 325 |
| | TLTISSLQAEDVAVYYCKQSYSRRTFGGGTKVEIK | |
| ADI-26924 | DIVMTQSPDSLAVSLGERATINCKSSQSLLNARTGKNYLAWYQQKPGQPPKLLIYWASTRESGVPDRFSGSGSGTDF | 335 |
| | TLTISSLQAEDVAVYYCKQSYSLRTFGGGTKVEIK | |
| ADI-26915 | DIVMTQSPDSLAVSLGERATINCKSSQSLLNARTGKNYLAWYQQKPGQPPKLLIYWASTRESGVPDRFSGSGSGTDF | 345 |
| | TLTISSLQAEDVAVYYCKQSYSRRTFGGGTKVEIK | |
| ADI-26943 | DIVMTQSPDSLAVSLGERATINCKSSQSLLNARTGKNYLAWYQQKPGQPPKLLIYWASTRESGVPDRFSGSGSGTDF | 355 |
| | TLTISSLQAEDVAVYYCKQSYSIRTFGGGTKVEIK | |
| ADI-48576 | DIVMTQSPDSLAVSLGERATINCKSSQSLLNAHTGKNHLAWYQQKPGQPPKLLIYWASTRESGVPDRFSGSGSGTD | 365 |
| | FTLTISSLQAEDVAVYYCKQSHSHRTFGGGTKVEIK | |
| ADI-48587 | DIVMTQSPDSLAVSLGERATINCKSSQSLLNARTGHNYLAWYQQKPGQPPKLLIYWASTRESGVPDRFSGSGSGTD | 375 |
| | FTLTISSLQAEDVAVYYCKQSHSHRTFGGGTKVEIK | |
| ADI-48592 | DIVMTQSPDSLAVSLGERATINCKSSQSLLNAHTGHNYLAWYQQKPGQPPKLLIYWASTRESGVPDRFSGSGSGTD | 385 |
| | FTLTISSLQAEDVAVYYCKQSYSRRTFGGGTKVEIK | |
| ADI-48650 | DIVMTQSPDSLAVSLGERATINCKSSQSLLNARTGKNYLAWYQQKPGQPPKLLIYWASTRESGVPDRFSGSGSGTDF | 395 |
| | TLTISSLQAEDVAVYYCKQSHTRRTFGGGTKVEIK | |
| ADI-74965 | DIVMTQSPDSLAVSLGERATINCKSSQSLLNARTGHNYLAWYQQKPGQPPKLLIYWASTRESGVPDRFSGSGSGTD | 405 |
| | FTLTISSLQAEDVAVYYCKQSHSHRTFGGGTKVEIK | |
| ADI-74966 | DIVMTQSPDSLAVSLGERATINCKSSQSLLNARTGHNYLAWYQQKPGQPPKLLIYWASTRESGVPDRFSGSGSGTD | 415 |
| | FTLTISSLQAEDVAVYYCKQSYSHRTFGGGTKVEIK | |
| ADI-74967 | GIVMTQSPDSLAVSLGERATINCKSSQSLLNARTGKNYLAWYQQKPGQPPKLLIYWASTRESGVPDRFSGSGSGTDF | 425 |
| | TLTISSLQAEDVAVYYCKQSHSHRTFGGGTKVEIK | |
| ADI-74968 | DIVMTQSPDSLAVSLGERATINCKSSQSLLNARTGHNYLAWYQQKPGQPPKLLIYWASTRESGVPDRFSGSGSGTD | 435 |
| | FTLTISSLQAEDVAVYYCKQSYSHRTFGGGTKVEIK | |
| ADI-76357 | DIVMTQSPDSLAVSLGERATINCKSSQSLLNARTGKNYLAWYQQKPGQPPKLLIYWASTRESGVPDRFSGSGSGTDF | 445 |
| | TLTISSLQAEDVAVYYCKQSYSRRTFGGGTKVEIK | |
| ADI-76358 | DIVMTQSPDSLAVSLGERATINCKSSQSLLNARTGKNYLAWYQQKPGQPPKLLIYWASTRESGVPDRFSGSGSGTDF | 455 |
| | TLTISSLQAEDVAVYYCKQSYSRRTFGGGTKVEIK | |
| ADI-76359 | DIVMTQSPDSLAVSLGERATINCKSSQSLLNARTGKNYLAWYQQKPGQPPKLLIYWASTRESGVPDRFSGSGSGTDF | 465 |
| | TLTISSLQAEDVAVYYCKQSYSLRTFGGGTKVEIK | |
| ADI-76360 | DIVMTQSPDSLAVSLGERATINCKSSQSLLNARTGKNYLAWYQQKPGQPPKLLIYWASTRESGVPDRFSGSGSGTDF | 475 |
| | TLTISSLQAEDVAVYYCKQSYSLRTFGGGTKVEIK | |
| ADI-76361 | DIVMTQSPDSLAVSLGERATINCKSSQSLLNARTGKNYLAWYQQKPGQPPKLLIYWASTRESGVPDRFSGSGSGTDF | 485 |
| | TLTISSLQAEDVAVYYCKQSYSLRTFGGGTKVEIK | |
| ADI-76362 | DIVMTQSPDSLAVSLGERATINCKSSQSLLNARTGKNYLAWYQQKPGQPPKLLIYWASTRESGVPDRFSGSGSGTDF | 495 |
| | TLTISSLQAEDVAVYYCKQSYSLRTFGGGTKVEIK | |
| CTL-62223 | DIVMTQSPDSLAVSLGERATINCKSSQSLLNSRTRKNYLAWYQQKPGQPPKLLIYWASTRESGVPDRFSGSGSGTDFT | 505 |
| | LTISSLQAEDVAVYYCTQSFILRTFGQGTKVEIK | |
| CTL-62227 | EIVMTQSPATLSVSPGERATLSCRASQSVSSNLAWYQQKPGQAPRLLIYGASTRATGIPARFSGSGSGTEFTLTISS | 515 |
| | LQSEDFAVYYCQHYINWPLTFGGGTKVEIK | |
| CTL-62228 | QAVVTQEPSLTVSPGGTVTLTCGSSTGAVTTSNYANWVQEKPGQAFRGLIGGTNKRAPGTPARFSGSLLGGKAALTL | 525 |
| | SGAQPEDEAEYYCALWYSNLWVFGGGTKLTVL | |
| CTL-62240 | DIQMTQSPSSLSASVGDRVTITCRASQSISSYLNWYQQKPGKAPKLLIYAASSLQSGVPSRFSGSGSGTDFTLTISS | 535 |
| | LQPEDFATYYCQQSYSTPPTFGQGTKVEIK | |

Underlined sequence spans correspond to CDRL1, CDRL2, and CDRL3 in the order of appearance. Sequence spans without an underline correspond to FRL1, FRL2, FRL3, and FRL4 in the order of appearance.

TABLE 2A
Anti-CD3 VH CDR sequences

| | CDRH1 | | CDRH2 | | CDRH3 | |
|---|---|---|---|---|---|---|
| Antibody | | SEQ | | SEQ | | SEQ |
| Containing | | ID | | ID | | ID |
| ADI Name | Sequence | NO: | Sequence | NO: | Sequence | NO: |
| ADI-22523 | FNIKDYYMH | 111 | WIDLENGNTIYDPKFQG | 112 | ARDGYGRYFYDV | 113 |
| ADI-26927 | FNIKDYYMH | 121 | WIDLEEGNTIYDAKFQG | 122 | ARDRYGAYFYDV | 123 |
| ADI-26928 | FNIKDYYMH | 131 | WIDLEEGNTIYDAKFQG | 132 | GRDAYGLYFYDV | 133 |
| ADI-26941 | FNIKDYYMH | 141 | WIDLEEGNTIYDAKFQG | 142 | ARDQYGRYFYDV | 143 |
| ADI-26954 | FNIKDYYMH | 151 | WIDLENANTIYDAKFQG | 152 | ARDNYGGYFYDV | 153 |
| CTL-19613 | FTFNKYAMN | 161 | RIRSKYNNYATYYADSVKD | 162 | VRHGNFGNSYIS | 163 |
| | | | | | YWAY | |
| CTL-19672 | FTFTSYYIH | 171 | WIYPENDNTKYNEKFKD | 172 | ARDGYSRYYFDY | 173 |
| ADI-50024 | FNIKDYYMH | 181 | WIDLENANTVYDAKFQG | 182 | ARDAYGRYFYDV | 183 |
| ADI-70326 | FDIKDYYMH | 191 | WIELENADTVYDAKFQG | 192 | ARDAYGRYFYDV | 193 |
| ADI-70327 | FDIKDYYMH | 201 | WIELENDDTVYDAKFQG | 202 | ARDAYGRYFYDV | 203 |
| ADI-70330 | FNIDDYYMH | 211 | WIDLENANTVDDAKFQG | 212 | ARDAYGRYFYDV | 213 |
| ADI-26906 | FNIKDYYMH | 221 | WIDLENANTIYDAKFQG | 222 | ARDAYGRYFYDV | 223 |
| ADI-67450 | FNIDDYYMH | 231 | WIDLENANTIDDAKFQG | 232 | ARDAYGRYFYDV | 233 |
| ADI-67445 | FNIKDYYMH | 241 | WIDLENAETIYDAKFQD | 242 | ARDAYGRYFYDE | 243 |
| ADI-67447 | FNIDDYYMH | 251 | WIDLENDNTIYDAKFQG | 252 | ARDAYGRYFYDV | 253 |
| ADI-26909 | FNIKDYYMH | 261 | WIDLEEGNTIYDAKFQG | 262 | GRDAYGRYFYDV | 263 |
| ADI-26913 | FNIKDYYMH | 271 | WIDLENANTIYDAKFQG | 272 | ARDQYGRYFYDV | 273 |
| ADI-26919 | FNIKDYYMH | 281 | WIDLENANTIYDAKFQG | 282 | ARDAYGRYFYDV | 283 |
| ADI-26920 | FNIKDYYMH | 291 | WIDLENANTIYDAKFQG | 292 | GRDAYGRYFYDV | 293 |
| ADI-26921 | FNIKDYYMH | 301 | WIDLEEGNTIYDAKFQG | 302 | ARDAYGRYFYDV | 303 |
| ADI-26916 | FNIKDYYMH | 311 | WIDLENANTIYDAKFQG | 312 | ARDVYGRYFYDL | 313 |
| ADI-26912 | FNIKDYYMH | 321 | WIDLENANTIYDAKFQG | 322 | ARDVLGRYFYDV | 323 |
| ADI-26924 | FNIKDYYMH | 331 | WIDLENANTIYDAKFQG | 332 | GRDAYGLYFYDV | 333 |
| ADI-26915 | FNIKDYYMH | 341 | WIDLEEGNTIYDAKFQG | 342 | ARDVLGRYFYDV | 343 |
| ADI-26943 | FNIKDYYMH | 351 | WIDLENANTIYDAKFQG | 352 | ARDAYGGYFYDV | 353 |
| ADI-48576 | FNIKDYYMH | 361 | WIDLENANTIYDAKFQG | 362 | ARDAYHRYFYDH | 363 |
| ADI-48587 | FNIKDYYMH | 371 | WIDLENANTIYDAKFQG | 372 | ARDHYHRYFYDV | 373 |
| ADI-48592 | FNIKDYYMH | 381 | WIDLENANTIYDAKFQG | 382 | ARDAHHRYFYDV | 383 |
| ADI-48650 | FNIKDYYMH | 391 | WIDLENANTIYDAKFQG | 392 | ARDATHRYFYDV | 393 |
| ADI-74965 | FNIKDYYMH | 401 | WIDLENANTIYDAKFQG | 402 | ARDAYHRYFYDV | 403 |
| ADI-74966 | FNIKDYYMH | 411 | WIDLENANTIYDAKFQG | 412 | ARDHYHRYFYDV | 413 |
| ADI-74967 | FNIKDYYMH | 421 | WIDLENANTIYDAKFQG | 422 | ARDHYHRYFYDV | 423 |
| ADI-74968 | FNIKDYYMH | 431 | WIDLENANTIYDAKFQG | 432 | ARDAYHRYFYDV | 433 |
| ADI-76357 | FDIKDYYMH | 441 | WIDLENANTIYDAKFQG | 442 | ARDQYGRYFYDV | 443 |
| ADI-76358 | FNIKDYYMH | 451 | WIDLENADTIYDAKFQG | 452 | ARDQYGRYFYDV | 453 |
| ADI-76359 | FDIKDYYMH | 461 | WIDLENANTIYDAKFQG | 462 | GRDAYGRYFYDV | 463 |
| ADI-76360 | FNIKDYYMH | 471 | WIDLENADTIYDAKFQG | 472 | GRDAYGRYFYDV | 473 |
| ADI-76361 | FDIKDYYMH | 481 | WIDLEEGNTIYDAKFQG | 482 | ARDAYGRYFYDV | 483 |
| ADI-76362 | FNIKDYYMH | 491 | WIDLEEGDTIYDAKFQG | 492 | ARDAYGRYFYDV | 493 |
| CTL-62223 | YTFTNYYIH | 501 | WIYPGDGNTKYNEKFKG | 502 | ARDSYSNYYFDY | 503 |
| CTL-62227 | FTFDDYTMH | 511 | GISWNSGSIGYADSVKG | 512 | AKDNSGYGHYYYG | 513 |
| | | | | | MDV | |
| CTL-62228 | FTFSTYAMN | 521 | RIRSKYNNYATYYADSV | 522 | VRHGNFGNSYVSW | 523 |
| | | | KG | | FAY | |
| CTL-62240 | FTFSSYGMH | 531 | QIWYNARKQEYSDSVKG | 532 | TRGTGYNWFDP | 533 |

TABLE 2B
Anti-CD3 VL CDR sequences

| | CDRL1 | | CDRL2 | | CDRL3 | |
|---|---|---|---|---|---|---|
| Antibody | | SEQ | | SEQ | | SEQ |
| Containing | | ID | | ID | | ID |
| ADI Name | Sequence | NO: | Sequence | NO: | Sequence | NO: |
| ADI-22523 | KSSQSLLNSRTGKNYLA | 116 | WASTRES | 117 | KQSYSRRT | 118 |
| ADI-26927 | KSSQSLLNARTGKNYLA | 126 | WASTRES | 127 | KQSYSLRT | 128 |
| ADI-26928 | KSSQSLLNARTGKNYLA | 136 | WASTRES | 137 | KQSYSLRT | 138 |
| ADI-26941 | KSSQSLLNARTGKNYLA | 146 | WASTRES | 147 | KQSYSIRT | 148 |
| ADI-26954 | KSSQSLLESRTGKNYLA | 156 | WASTRES | 157 | KQSYSLRT | 158 |
| CTL-19613 | GSSTGAVTSGNYPN | 166 | GTKFLAP | 167 | VLWYSNRWV | 168 |
| CTL-19672 | KSSQSLLNSRTRKNYLA | 176 | WTSTRKS | 177 | KQSFILRT | 178 |
| ADI-50024 | KSSQSLLNARTGKNYLA | 186 | WASTRSS | 187 | VQSYFRRT | 188 |
| ADI-70326 | KSSQSLLNARTGKNYLA | 196 | WASTRSS | 197 | VQSYFRRT | 198 |
| ADI-70327 | KSSQSLLNARTGKNYLA | 206 | WASTRSS | 207 | VQSYFRRT | 208 |
| ADI-70330 | KSSQSLLNARTGKNYLA | 216 | WASTRSS | 217 | VQSYFRRT | 218 |
| ADI-26906 | KSSQSLLNARTGKNYLA | 226 | WASTRES | 227 | KQSYSRRT | 228 |
| ADI-67450 | KSSQSLLNARTGKNYLA | 236 | WASTRES | 237 | KQSYSRRT | 238 |
| ADI-67445 | KSSQSLLNARTGKNYLA | 246 | WASTRES | 247 | KQSYSRRT | 248 |
| ADI-67447 | KSSQSLLNARTGKNYLA | 256 | WASTRES | 257 | KQSYSRRT | 258 |
| ADI-26909 | KSSQSLLNARTGKNYLA | 266 | WASTRES | 267 | KQSYSRRT | 268 |
| ADI-26913 | KSSQSLLNARTGKNYLA | 276 | WASTRES | 277 | KQSYSRRT | 278 |
| ADI-26919 | KSSQSLLNARTGKNYLA | 286 | WASTRES | 287 | KQSYSLRT | 288 |
| ADI-26920 | KSSQSLLNARTGKNYLA | 296 | WASTRES | 297 | KQSYSLRT | 298 |
| ADI-26921 | KSSQSLLNARTGKNYLA | 306 | WASTRES | 307 | KQSYSLRT | 308 |
| ADI-26916 | KSSQSLLNARTGKNYLA | 316 | WASTRES | 317 | KQSYSRRT | 318 |
| ADI-26912 | KSSQSLLNARTGKNYLA | 326 | WASTRES | 327 | KQSYSRRT | 328 |
| ADI-26924 | KSSQSLLNARTGKNYLA | 336 | WASTRES | 337 | KQSYSLRT | 338 |
| ADI-26915 | KSSQSLLNARTGKNYLA | 346 | WASTRES | 347 | KQSYSRRT | 348 |
| ADI-26943 | KSSQSLLNARTGKNYLA | 356 | WASTRES | 357 | KQSYSIRT | 358 |
| ADI-48576 | KSSQSLLNAHTGKNHLA | 366 | WASTRES | 367 | KQSHSHRT | 368 |
| ADI-48587 | KSSQSLLNARTGHNYLA | 376 | WASTRES | 377 | KQSHSHRT | 378 |
| ADI-48592 | KSSQSLLNAHTGHNYLA | 386 | WASTRES | 387 | KQSYSRRT | 388 |
| ADI-48650 | KSSQSLLNARTGKNYLA | 396 | WASTRES | 397 | KQSHTRRT | 398 |
| ADI-74965 | KSSQSLLNARTGHNYLA | 406 | WASTRES | 407 | KQSHSHRT | 408 |
| ADI-74966 | KSSQSLLNARTGHNYLA | 416 | WASTRES | 417 | KQSYSHRT | 418 |
| ADI-74967 | KSSQSLLNARTGKNYLA | 426 | WASTRES | 427 | KQSHSHRT | 428 |
| ADI-74968 | KSSQSLLNARTGHNYLA | 436 | WASTRES | 437 | KQSYSHRT | 438 |
| ADI-76357 | KSSQSLLNARTGKNYLA | 446 | WASTRES | 447 | KQSYSRRT | 448 |
| ADI-76358 | KSSQSLLNARTGKNYLA | 456 | WASTRES | 457 | KQSYSRRT | 458 |
| ADI-76359 | KSSQSLLNARTGKNYLA | 466 | WASTRES | 467 | KQSYSLRT | 468 |
| ADI-76360 | KSSQSLLNARTGKNYLA | 476 | WASTRES | 477 | KQSYSLRT | 478 |
| ADI-76361 | KSSQSLLNARTGKNYLA | 486 | WASTRES | 487 | KQSYSLRT | 488 |
| ADI-76362 | KSSQSLLNARTGKNYLA | 496 | WASTRES | 497 | KQSYSLRT | 498 |
| CTL-62223 | KSSQSLLNSRTRKNYLA | 506 | WASTRES | 507 | TQSFILRT | 508 |
| CTL-62227 | RASQSVSSNLA | 516 | GASTRAT | 517 | QHYINWPLT | 518 |
| CTL-62228 | GSSTGAVTTSNYAN | 526 | GTNKRAP | 527 | ALWYSNLWV | 528 |
| CTL-62240 | RASQSISSYLN | 536 | AASSLOS | 537 | QQSYSTPPT | 538 |

TABLE 2C
Anti-CD3 variable domain germline origin

| ADI Name | VH Germline | VL Germline | ADI Name | VH Germline | VL Germline |
|---|---|---|---|---|---|
| ADI-22523 | VH1-03 | VK4-01 | ADI-48576 | VH1-03 | VK4-01 |
| ADI-26927 | VH1-03 | VK4-01 | ADI-48587 | VH1-03 | VK4-01 |
| ADI-26928 | VH1-03 | VK4-01 | ADI-48592 | VH1-03 | VK4-01 |
| ADI-26941 | VH1-03 | VK4-01 | ADI-48650 | VH1-03 | VK4-01 |
| ADI-26954 | VH1-03 | VK4-01 | ADI-74965 | VH1-03 | VK4-01 |
| CTL-19613 | VH3-72 | VL7-43 | ADI-74966 | VH1-03 | VK4-01 |
| CTL-19672 | VH1-03 | VK4-01 | ADI-74967 | VH1-03 | VK4-01 |
| ADI-50024 | VH1-03 | VK4-01 | ADI-74968 | VH1-03 | VK4-01 |
| ADI-70326 | VH1-03 | VK4-01 | ADI-76357 | VH1-03 | VK4-01 |
| ADI-70327 | VH1-03 | VK4-01 | ADI-76358 | VH1-03 | VK4-01 |
| ADI-70330 | VH1-03 | VK4-01 | ADI-76359 | VH1-03 | VK4-01 |
| ADI-26906 | VH1-03 | VK4-01 | ADI-76360 | VH1-03 | VK4-01 |
| ADI-67450 | VH1-03 | VK4-01 | ADI-76361 | VH1-03 | VK4-01 |
| ADI-67445 | VH1-03 | VK4-01 | ADI-76362 | VH1-03 | VK4-01 |
| ADI-67447 | VH1-03 | VK4-01 | CTL-62223 | VH1-03 | VK4-01 |
| ADI-26909 | VH1-03 | VK4-01 | CTL-62227 | VH3-09 | VK3-15 |
| ADI-26913 | VH1-03 | VK4-01 | CTL-62228 | VH3-23 | VL7-46 |
| ADI-26919 | VH1-03 | VK4-01 | CTL-62240 | VH3-33 | VK1-39 |
| ADI-26920 | VH1-03 | VK4-01 | | | |
| ADI-26921 | VH1-03 | VK4-01 | | | |
| ADI-26916 | VH1-03 | VK4-01 | | | |
| ADI-26912 | VH1-03 | VK4-01 | | | |
| ADI-26924 | VH1-03 | VK4-01 | | | |
| ADI-26915 | VH1-03 | VK4-01 | | | |
| ADI-26943 | VH1-03 | VK4-01 | | | |

TABLE 3A
Anti-ID VH sequences

| | VH | |
|---|---|---|
| Antibody | | SEQ |
| Containing | | ID |
| ADI Name | Sequence | NO: |
| ADI-30242 | QVQLVQSGAEVKKPGSSVKVSCKASGGTFSSYAISWVRQAPGQGLEWMGGIIPIFGTANYAQKFQGRVTITADEST | 560 |
| | STAYMELSSLRSEDTAVYYCARGAPNYYGSGGYDDGWFDPWGQGTLVTVSS | |
| ADI-34131 | QVQLVQSGAEVKKPGSSVKVSCKASGGTFSSYAISWVRQAPGQGLEWMGGIIPIFGTANYAQKFQGRVTITADEST | 580 |
| | STAYMELSSLRSEDTAVYYCARGAPNYYGSGGYDDGWFDPWGQGTLVTVSS | |
| ADI-34132 | QVQLVQSGAEVKKPGSSVKVSCKASGGTFSSYAISWVRQAPGQGLEWMGGIIPIFGTANYAQKFQGRVTITADEST | 600 |
| | STAYMELSSLRSEDTAVYYCARGAPNYYGSGGYDDGWFDPWGQGTLVTVSS | |
| ADI-34133 | QVQLVQSGAEVKKPGSSVKVSCKASGGTFSSYAISWVRQAPGQGLEWMGGIIPIFGTANYAQKFQGRVTITADEST | 620 |
| | STAYMELSSLRSEDTAVYYCARGAPNYYGSGGYDDGWFDPWGQGTLVTVSS | |
| ADI-34134 | QVQLVQSGAEVKKPGSSVKVSCKASGGTFSSYAISWVRQAPGQGLEWMGGIIPIFGTANYAQKFQGRVTITADEST | 640 |
| | STAYMELSSLRSEDTAVYYCARGAPNYYGSGGYDDGWFDPWGQGTLVTVSS | |
| ADI-34135 | QVQLVQSGAEVKKPGSSVKVSCKASGGTFSSYAISWVRQAPGQGLEWMGGIIPIFGTANYAQKFQGRVTITADEST | 660 |
| | STAYMELSSLRSEDTAVYYCARGAPNYYGSGGYDDGWFDPWGQGTLVTVSS | |
| ADI-34136 | QVQLVQSGAEVKKPGSSVKVSCKASGGTFSSYAISWVRQAPGQGLEWMGGIIPIFGTANYAQKFQGRVTITADEST | 680 |
| | STAYMELSSLRSEDTAVYYCARGAPNYYGSGGYDDGWFDPWGQGTLVTVSS | |
| ADI-34137 | QVQLVQSGAEVKKPGSSVKVSCKASGGTFSSYAISWVRQAPGQGLEWMGGIIPIFGTANYAQKFQGRVTITADEST | 700 |
| | STAYMELSSLRSEDTAVYYCARGAPNYYGSGGYDDGWFDPWGQGTLVTVSS | |
| ADI-34138 | QVQLVQSGAEVKKPGSSVKVSCKASGGTFSSYAISWVRQAPGQGLEWMGGIIPIFGTANYAQKFQGRVTITADEST | 720 |
| | STAYMELSSLRSEDTAVYYCARGAPNYYGSGGYDDGWFDPWGQGTLVTVSS | |
| ADI-30254 | EVQLLESGGGLVQPGGSLRLSCAASGFTFSSYAMSWVRQAPGKGLEWVSTISGSGGSTYYADSVKGRFTISRDNSKN | 740 |
| | TLYLQMNSLRAEDTAVYYCARLGGRRGSGSSYYYYYGMDVWGQGTTVTVSS | |
| ADI-34139 | EVQLLESGGGLVQPGGSLRLSCAASGFTFSSYAMSWVRQAPGKGLEWVSTISGSGGSTYYADSVKGRFTISRDNSKN | 760 |
| | TLYLQMNSLRAEDTAVYYCARLGGRRGSGSSYYYYYGMDVWGQGTTVTVSS | |
| ADI-34140 | EVQLLESGGGLVQPGGSLRLSCAASGFTFSSYAMSWVRQAPGKGLEWVSTISGSGGSTYYADSVKGRFTISRDNSKN | 780 |
| | TLYLQMNSLRAEDTAVYYCARLGGRRGSGSSYYYYYGMDVWGQGTTVTVSS | |
| ADI-34141 | EVQLLESGGGLVQPGGSLRLSCAASGFTFSSYAMSWVRQAPGKGLEWVSTISGSGGSTYYADSVKGRFTISRDNSKN | 800 |
| | TLYLQMNSLRAEDTAVYYCARLGGRRGSGSSYYYYYGMDVWGQGTTVTVSS | |
| ADI-34142 | EVQLLESGGGLVQPGGSLRLSCAASGFTFSSYAMSWVRQAPGKGLEWVSTISGSGGSTYYADSVKGRFTISRDNSKN | 820 |
| | TLYLQMNSLRAEDTAVYYCARLGGRRGSGSSYYYYYGMDVWGQGTTVTVSS | |
| ADI-34143 | EVQLLESGGGLVQPGGSLRLSCAASGFTFSSYAMSWVRQAPGKGLEWVSTISGSGGSTYYADSVKGRFTISRDNSKN | 840 |
| | TLYLQMNSLRAEDTAVYYCARLGGRRGSGSSYYYYYGMDVWGQGTTVTVSS | |
| ADI-34144 | EVQLLESGGGLVQPGGSLRLSCAASGFTFSSYAMSWVRQAPGKGLEWVSTISGSGGSTYYADSVKGRFTISRDNSKN | 860 |
| | TLYLQMNSLRAEDTAVYYCARLGGRRGSGSSYYYYYGMDVWGQGTTVTVSS | |
| ADI-34145 | EVQLLESGGGLVQPGGSLRLSCAASGFTFSSYAMSWVRQAPGKGLEWVSTISGSGGSTYYADSVKGRFTISRDNSKN | 880 |
| | TLYLQMNSLRAEDTAVYYCARLGGRRGSGSSYYYYYGMDVWGQGTTVTVSS | |
| ADI-34146 | EVQLLESGGGLVQPGGSLRLSCAASGFTFSSYAMSWVRQAPGKGLEWVSTISGSGGSTYYADSVKGRFTISRDNSKN | 900 |
| | TLYLQMNSLRAEDTAVYYCARLGGRRGSGSSYYYYYGMDVWGQGTTVTVSS | |

Underlined sequence spans correspond to CDRH1, CDRH2, and CDRH3 in the order of appearance.
Sequence spans without an underline correspond to FRH1, FRH2, FRH3, and FRH4 in the order of appearance.

TABLE 3B
Anti-ID VL sequences

| | VL | |
|---|---|---|
| Antibody | | SEQ |
| Containing | | ID |
| ADI Name | Sequence | NO: |
| ADI-30242 | EIVLTQSPGTLSLSPGERATLSCRASQSVSSSYLAWYQQKPGQAPRLLIYGASSRATGIPDRFSGSGSGTDFTLTISRLE | 570 |
| | PEDFAVYYCQQFGRDPTFGGGTKVEIK | |
| ADI-34131 | EIVLTQSPGTLSLSPGERATLSCRASQSVSSSYLAWYQQKPGQAPRLLIYGASSRATGIPDRFSGSGSGTDFTLTISRLE | 590 |
| | PEDFAVYYCLQFGSSPTFGGGTKVEIK | |
| ADI-34132 | DIQLTQSPSSLSASVGDRVTITCRASQSISSYLNWYQQKPGKAPKLLIYAASNLQSGVPSRFSGSGSGTDFTLTISSLQP | 610 |
| | EDFATYYCQQTLHTPITFGGGTKVEIK | |
| ADI-34133 | EIVLTQSPGTLSLSPGERATLSCRASQSVSSSYLAWYQQKPGQAPRLLIYGASSRATGIPDRFSGSGSGTDFTLTISRLE | 630 |
| | PEDFAVYYCQQFGRSPTFGGGTKVEIK | |
| ADI-34134 | EIVLTQSPGTLSLSPGERATLSCRASQSVSSDYLAWYQQKPGQAPRLLIYGASSRATGIPDRFSGSGSGTDFTLTISRLE | 650 |
| | PEDFAVYYCQQFGRVPTFGGGTKVEIK | |
| ADI-34135 | EIVMTQSPGTLSLSPGERATLSCRASQSVSSSYLAWYQQKPGQAPRLLIYGASSRATGIPDRFSGSGSGTDFTLTISRL | 670 |
| | EPEDFAVYYCQQFGRYPTFGGGTKVEIK | |
| ADI-34136 | EIVLTQSPGTLSLSPGERATLSCRASQSVSSSYLAWYQQKPGQAPRLLIYGASSRATGIPDRFSGSGSGTDFTLTISRLE | 690 |
| | PEDFAVYYCQQFGPSPTFGGGTKVEIK | |
| ADI-34137 | EIVLTQSPGTLSLSPGERATLSCRASQSVSSSYLAWYQQKPGQAPRLLIYGASSRATGIPDRFSGSGSGTDFTLTISRLE | 710 |
| | PEDFAVYYCQQFGRFPTFGGGTKVEIK | |
| ADI-34138 | EIVMTQSPGTLSLSPGERATLSCRASQSVSSSYLAWYQQKPGQAPRLLIYGASNRATGIPDRFSGSGSGTDFTLTISRL | 730 |
| | EPEDFAVYYCQQAHTALWTFGGGTKVEIK | |
| ADI-30254 | DIVMTQSPLSLPVTPGEPASISCRSSQSLLHSNGYNYLDWYLQKPGQSPQLLIYLGSNRASGVPDRFSGSGSGTDFTL | 750 |
| | KISRVEAEDVGVYYCMQVRQTPWTFGGGTKVEIK | |
| ADI-34139 | DIVMTQSPLSLPVTPGEPASISCRSSQSLLHSNGYNYLDWYLQKPGQSPQLLIYLGSNRASGVPDRFSGSGSGTDFTL | 770 |
| | KISRVEAEDVGVYYCMQVLDLPWTFGGGTKVEIK | |
| ADI-34140 | DIVMTQSPLSLPVTPGEPASISCRSSQSLLHSNGYNYLDWYLQKPGQSPQLLIYLGSNRASGVPDRFSGSGSGTDFTL | 790 |
| | KISRVEAEDVGVYYCMQVLELPWTFGGGTKVEIK | |
| ADI-34141 | DIVMTQSPLSLPVTPGEPASISCRSSQSLLHSNGYNYLDWYLQKPGQSPQLLIYLGSNRASGVPDRFSGSGSGTDFTL | 810 |
| | KISRVEAEDVGVYYCMQVKDTPWTFGGGTKVEIK | |
| ADI-34142 | DIVMTQSPLSLPVTPGEPASISCRSSQSLLYSNGYNYLDWYLQKPGQSPQLLIYLGSNRASGVPDRFSGSGSGTDFTL | 830 |
| | KISRVEAEDVGVYYCMQVLGLPWTFGGGTKVEIK | |
| ADI-34143 | DIVMTQSPLSLPVTPGEPASISCRSSQSLLHSNGYNYLDWYLQKPGQSPQLLIYLGSNRASGVPDRFSGSGSGTDFTL | 850 |
| | KISRVEAEDVGVYYCMQVSDTPWTFGGGTKVEIK | |
| ADI-34144 | DIVMTQSPLSLPVTPGEPASISCRSSQSLLHSNGYNYLDWYLQKPGQSPQLLIYLGSNRASGVPDRFSGSGSGTDFTL | 870 |
| | KISRVEAEDVGVYYCMQVLEIPWTFGGGTKVEIK | |
| ADI-34145 | DIVMTQSPLSLPVTPGEPASISCRSSQSLLHSNGYNYLDWYLQKPGQSPQLLIYLGSNRASGVPDRFSGSGSGTDFTL | 890 |
| | KISRVEAEDVGVYYCMQVLELPWTFGGGTKVEIK | |
| ADI-34146 | DIVMTQSPLSLPVTPGEPASISCRSSQSLLHSNGYNYLDWYLQKPGQSPQLLIYLGSNRASGVPDRFSGSGSGTDFTL | 910 |
| | KISRVEAEDVGVYYCMQVLDLPWTFGGGTKVEIK | |

Underlined sequence spans correspond to CDRL1, CDRL2, and CDRL3 in the order of appearance.
Sequence spans without an underline correspond to FRL1, FRL2, FRL3, and FRL4 in the order of appearance.

TABLE 4A
Anti-ID VH CDR sequences

| Antibody | | SEQ | | SEQ | | SEQ |
|---|---|---|---|---|---|---|
| Containing | CDRH1 | ID | CDRH2 | ID | CDRH3 | ID |
| ADI Name | Sequence | NO: | Sequence | NO: | Sequence | NO: |
| ADI-30242 | GTFSSYAIS | 562 | GIIPIFGTANYAQKFQG | 564 | ARGAPNYYGSGGYDDGWFDP | 566 |
| ADI-34131 | GTFSSYAIS | 582 | GIIPIFGTANYAQKFQG | 584 | ARGAPNYYGSGGYDDGWFDP | 586 |
| ADI-34132 | GTFSSYAIS | 602 | GIIPIFGTANYAQKFQG | 604 | ARGAPNYYGSGGYDDGWFDP | 606 |
| ADI-34133 | GTFSSYAIS | 622 | GIIPIFGTANYAQKFQG | 624 | ARGAPNYYGSGGYDDGWFDP | 626 |
| ADI-34134 | GTFSSYAIS | 642 | GIIPIFGTANYAQKFQG | 644 | ARGAPNYYGSGGYDDGWFDP | 646 |
| ADI-34135 | GTFSSYAIS | 662 | GIIPIFGTANYAQKFQG | 664 | ARGAPNYYGSGGYDDGWFDP | 666 |
| ADI-34136 | GTFSSYAIS | 682 | GIIPIFGTANYAQKFQG | 684 | ARGAPNYYGSGGYDDGWFDP | 686 |
| ADI-34137 | GTFSSYAIS | 702 | GIIPIFGTANYAQKFQG | 704 | ARGAPNYYGSGGYDDGWFDP | 706 |
| ADI-34138 | GTFSSYAIS | 722 | GIIPIFGTANYAQKFQG | 724 | ARGAPNYYGSGGYDDGWFDP | 726 |
| Consensus | GTFSSYAIS | 922 | GIIPIFGTANYAQKFQG | 924 | ARGAPNYYGSGGYDDGWFDP | 926 |
| sequence | | | | | | |
| ADI-30254 | FTFSSYAMS | 742 | TISGSGGSTYYADSVKG | 744 | ARLGGRRGSGSSYYYYYGMDV | 746 |
| ADI-34139 | FTFSSYAMS | 762 | TISGSGGSTYYADSVKG | 764 | ARLGGRRGSGSSYYYYYGMDV | 766 |
| ADI-34140 | FTFSSYAMS | 782 | TISGSGGSTYYADSVKG | 784 | ARLGGRRGSGSSYYYYYGMDV | 786 |
| ADI-34141 | FTFSSYAMS | 802 | TISGSGGSTYYADSVKG | 804 | ARLGGRRGSGSSYYYYYGMDV | 806 |
| ADI-34142 | FTFSSYAMS | 822 | TISGSGGSTYYADSVKG | 824 | ARLGGRRGSGSSYYYYYGMDV | 826 |
| ADI-34143 | FTFSSYAMS | 842 | TISGSGGSTYYADSVKG | 844 | ARLGGRRGSGSSYYYYYGMDV | 846 |
| ADI-34144 | FTFSSYAMS | 862 | TISGSGGSTYYADSVKG | 864 | ARLGGRRGSGSSYYYYYGMDV | 866 |
| ADI-34145 | FTFSSYAMS | 882 | TISGSGGSTYYADSVKG | 884 | ARLGGRRGSGSSYYYYYGMDV | 886 |
| ADI-34146 | FTFSSYAMS | 902 | TISGSGGSTYYADSVKG | 904 | ARLGGRRGSGSSYYYYYGMDV | 906 |
| Consensus | FTFSSYAMS | 942 | TISGSGGSTYYADSVKG | 944 | ARLGGRRGSGSSYYYYYGMDV | 946 |
| sequence | | | | | | |

TABLE 4B
Anti-ID VL CDR sequences

| Antibody | | SEQ | | SEQ | | SEQ |
|---|---|---|---|---|---|---|
| Containing | CDRL1 | ID | CDRL2 | ID | CDRL3 | ID |
| ADI Name | Sequence | NO: | Sequence | NO: | Sequence | NO: |
| ADI-30242 | RASQSVSSSYLA | 572 | GASSRAT | 574 | QQFGRDPT | 576 |
| ADI-34131 | RASQSVSSSYLA | 592 | GASSRAT | 594 | LQFGSSPT | 596 |
| ADI-34132 | RASQSISSYLN | 612 | AASNLQS | 614 | QQTLHTPIT | 616 |
| ADI-34133 | RASQSVSSSYLA | 632 | GASSRAT | 634 | QQFGRSPT | 636 |
| ADI-34134 | RASQSVSSDYLA | 652 | GASSRAT | 654 | QQFGRVPT | 656 |
| ADI-34135 | RASQSVSSSYLA | 672 | GASSRAT | 674 | QQFGRYPT | 676 |
| ADI-34136 | RASQSVSSSYLA | 692 | GASSRAT | 694 | QQFGPSPT | 696 |
| ADI-34137 | RASQSVSSSYLA | 712 | GASSRAT | 714 | QQFGRFPT | 716 |
| ADI-34138 | RASQSVSSSYLA | 732 | GASNRAT | 734 | QQAHTALWT | 736 |
| Consensus | RASQSX₁SS | 932 | X₁ASX₂ | 934 | X₁QX₂X₃X₄ | 936 |
| sequence | X₂YLX₃ | | X₃X₄X₅ | | X₅X₆X₇T | |
| | X₁ = V or I; | | X₁ = G or A; | | X₁ = Q or L; | |
| | X₂ = D, S, | | X₂ = S or N; | | X₂ = F, T, | |
| | or deletion; | | X₃ = R or L; | | or A; | |
| | and X₃ = | | X₄ = A or Q; | | X₃ = G, L, | |
| | A or N | | and | | or H; | |
| | | | X₅ = T or S | | X₄ = R, S, H, | |
| | | | | | P, or T; | |
| | | | | | X₅ = V, D, S, | |
| | | | | | T, Y, F, or A; | |
| | | | | | X₆ = P or L; | |
| | | | | | and | |
| | | | | | X₇ = deletion, | |
| | | | | | I, or W | |
| ADI-30254 | RSSQSLLHSNGYNYLD | 752 | LGSNRAS | 754 | MQVRQTPWT | 756 |
| ADI-34139 | RSSQSLLHSNGYNYLD | 772 | LGSNRAS | 774 | MQVLDLPWT | 776 |
| ADI-34140 | RSSQSLLHSNGYNYLD | 792 | LGSNRAS | 794 | MQVLELPWT | 796 |
| ADI-34141 | RSSQSLLHSNGYNYLD | 812 | LGSNRAS | 814 | MQVKDTPWT | 816 |
| ADI-34142 | RSSQSLLYSNGYNYLD | 832 | LGSNRAS | 834 | MQVLGLPWT | 836 |
| ADI-34143 | RSSQSLLHSNGYNYLD | 852 | LGSNRAS | 854 | MQVSDTPWT | 856 |
| ADI-34144 | RSSQSLLHSNGYNYLD | 872 | LGSNRAS | 874 | MQVLEIPWT | 876 |
| ADI-34145 | RSSQSLLHSNGYNYLD | 892 | LGSNRAS | 894 | MQVLELPWT | 896 |
| ADI-34146 | RSSQSLLHSNGYNYLD | 912 | LGSNRAS | 914 | MQVLDLPWT | 916 |
| Consensus | RSSQSLLX₁SNGYNYLD | 952 | LGSNRAS | 954 | MQVX₁X₂X₃PWT | 956 |
| sequence | X₁ = H or Y | | | | X₁ = L, R, K, | |
| | | | | | or S; | |
| | | | | | X₂ = E, Q, D, | |
| | | | | | or G; | |
| | | | | | and X₃ = L, | |
| | | | | | T, or I | |

TABLE 4C
Anti-ID VH FR sequences

| | FRH1 | | FRH2 | | FRH3 | | FRH4 | |
|---|---|---|---|---|---|---|---|---|
| Antibody | | SEQ | | SEQ | | SEQ | | SEQ |
| Containing | | ID | | ID | | ID | | ID |
| ADI Name | Sequence | NO: | Sequence | NO: | Sequence | NO: | Sequence | NO: |
| ADI-30242 | QVQLVQSGAEVKKPGSSV | 561 | WVRQAPGQGL | 563 | RVTITADESTSTAYMELSSLRS | 565 | WGQGTLV | 567 |
| | KVSCKASG | | EWMG | | EDTAVYYC | | TVSS | |
| ADI-34131 | QVQLVQSGAEVKKPGSSV | 581 | WVRQAPGQGL | 583 | RVTITADESTSTAYMELSSLRS | 585 | WGQGTLV | 587 |
| | KVSCKASG | | EWMG | | EDTAVYYC | | TVSS | |
| ADI-34132 | QVQLVQSGAEVKKPGSSV | 601 | WVRQAPGQGL | 603 | RVTITADESTSTAYMELSSLRS | 605 | WGQGTLV | 607 |
| | KVSCKASG | | EWMG | | EDTAVYYC | | TVSS | |
| ADI-34133 | QVQLVQSGAEVKKPGSSV | 621 | WVRQAPGQGL | 623 | RVTITADESTSTAYMELSSLRS | 625 | WGQGTLV | 627 |
| | KVSCKASG | | EWMG | | EDTAVYYC | | TVSS | |
| ADI-34134 | QVQLVQSGAEVKKPGSSV | 641 | WVRQAPGQGL | 643 | RVTITADESTSTAYMELSSLRS | 645 | WGQGTLV | 647 |
| | KVSCKASG | | EWMG | | EDTAVYYC | | TVSS | |
| ADI-34135 | QVQLVQSGAEVKKPGSSV | 661 | WVRQAPGQGL | 663 | RVTITADESTSTAYMELSSLRS | 665 | WGQGTLV | 667 |
| | KVSCKASG | | EWMG | | EDTAVYYC | | TVSS | |
| ADI-34136 | QVQLVQSGAEVKKPGSSV | 681 | WVRQAPGQGL | 683 | RVTITADESTSTAYMELSSLRS | 685 | WGQGTLV | 687 |
| | KVSCKASG | | EWMG | | EDTAVYYC | | TVSS | |
| ADI-34137 | QVQLVQSGAEVKKPGSSV | 701 | WVRQAPGQGL | 703 | RVTITADESTSTAYMELSSLRS | 705 | WGQGTLV | 707 |
| | KVSCKASG | | EWMG | | EDTAVYYC | | TVSS | |
| ADI-34138 | QVQLVQSGAEVKKPGSSV | 721 | WVRQAPGQGL | 723 | RVTITADESTSTAYMELSSLRS | 725 | WGQGTLV | 727 |
| | KVSCKASG | | EWMG | | EDTAVYYC | | TVSS | |
| Consensus | QVQLVQSGAEVKKPGSSV | 921 | WVRQAPGQGL | 923 | RVTITADESTSTAYMELSSLRS | 925 | WGQGTLV | 927 |
| sequence | KVSCKASG | | EWMG | | EDTAVYYC | | TVSS | |
| ADI-30254 | EVQLLESGGGLVQPGGSL | 741 | WVRQAPGKGL | 743 | RFTISRDNSKNTLYLQMNSLR | 745 | WGQGTTV | 747 |
| | RLSCAASG | | EWVS | | AEDTAVYYC | | TVSS | |
| ADI-34139 | EVQLLESGGGLVQPGGSL | 761 | WVRQAPGKGL | 763 | RFTISRDNSKNTLYLQMNSLR | 765 | WGQGTTV | 767 |
| | RLSCAASG | | EWVS | | AEDTAVYYC | | TVSS | |
| ADI-34140 | EVQLLESGGGLVQPGGSL | 781 | WVRQAPGKGL | 783 | RFTISRDNSKNTLYLQMNSLR | 785 | WGQGTTV | 787 |
| | RLSCAASG | | EWVS | | AEDTAVYYC | | TVSS | |
| ADI-34141 | EVQLLESGGGLVQPGGSL | 801 | WVRQAPGKGL | 803 | RFTISRDNSKNTLYLQMNSLR | 805 | WGQGTTV | 807 |
| | RLSCAASG | | EWVS | | AEDTAVYYC | | TVSS | |
| ADI-34142 | EVQLLESGGGLVQPGGSL | 821 | WVRQAPGKGL | 823 | RFTISRDNSKNTLYLQMNSLR | 825 | WGQGTTV | 827 |
| | RLSCAASG | | EWVS | | AEDTAVYYC | | TVSS | |
| ADI-34143 | EVQLLESGGGLVQPGGSL | 841 | WVRQAPGKGL | 843 | RFTISRDNSKNTLYLQMNSLR | 845 | WGQGTTV | 847 |
| | RLSCAASG | | EWVS | | AEDTAVYYC | | TVSS | |
| ADI-34144 | EVQLLESGGGLVQPGGSL | 861 | WVRQAPGKGL | 863 | RFTISRDNSKNTLYLQMNSLR | 865 | WGQGTTV | 867 |
| | RLSCAASG | | EWVS | | AEDTAVYYC | | TVSS | |
| ADI-34145 | EVQLLESGGGLVQPGGSL | 881 | WVRQAPGKGL | 883 | RFTISRDNSKNTLYLQMNSLR | 885 | WGQGTTV | 887 |
| | RLSCAASG | | EWVS | | AEDTAVYYC | | TVSS | |
| ADI-34146 | EVQLLESGGGLVQPGGSL | 901 | WVRQAPGKGL | 903 | RFTISRDNSKNTLYLQMNSLR | 905 | WGQGTTV | 907 |
| | RLSCAASG | | EWVS | | AEDTAVYYC | | TVSS | |
| Consensus | EVQLLESGGGLVQPGGSL | 941 | WVRQAPGKGL | 943 | RFTISRDNSKNTLYLQMNSLR | 945 | WGQGTTV | 947 |
| sequence | RLSCAASG | | EWVS | | AEDTAVYYC | | TVSS | |

TABLE 4D
Anti-ID VL FR sequences

| | FRL1 | | FRL2 | | FRL3 | | FRL4 | |
|---|---|---|---|---|---|---|---|---|
| Antibody | | SEQ | | SEQ | | SEQ | | SEQ |
| Containing | | ID | | ID | | ID | | ID |
| ADI Name | Sequence | NO: | Sequence | NO: | Sequence | NO: | Sequence | NO: |
| ADI-30242 | EIVLTQSPGTLSLSPGERATLSC | 571 | WYQQKPGQ | 573 | GIPDRFSGSGSGTDFTL | 575 | FGGGTKVEIK | 577 |
| | | | APRLLIY | | TISRLEPEDFAVYYC | | | |
| ADI-34131 | EIVLTQSPGTLSLSPGERATLSC | 591 | WYQQKPGQ | 593 | GIPDRFSGSGSGTDFTL | 595 | FGGGTKVEIK | 597 |
| | | | APRLLIY | | TISRLEPEDFAVYYC | | | |
| ADI-34132 | DIQLTQSPSSLSASVGDRVTITC | 611 | WYQQKPGK | 613 | GVPSRFSGSGSGTDFTL | 615 | FGGGTKVEIK | 617 |
| | | | APKLLIY | | TISSLQPEDFATYYC | | | |
| ADI-34133 | EIVLTQSPGTLSLSPGERATLSC | 631 | WYQQKPGQ | 633 | GIPDRFSGSGSGTDFTL | 635 | FGGGTKVEIK | 637 |
| | | | APRLLIY | | TISRLEPEDFAVYYC | | | |
| ADI-34134 | EIVLTQSPGTLSLSPGERATLSC | 651 | WYQQKPGQ | 653 | GIPDRFSGSGSGTDFTL | 655 | FGGGTKVEIK | 657 |
| | | | APRLLIY | | TISRLEPEDFAVYYC | | | |
| ADI-34135 | EIVMTQSPGTLSLSPGERATLSC | 671 | WYQQKPGQ | 673 | GIPDRFSGSGSGTDFTL | 675 | FGGGTKVEIK | 677 |
| | | | APRLLIY | | TISRLEPEDFAVYYC | | | |
| ADI-34136 | EIVLTQSPGTLSLSPGERATLSC | 691 | WYQQKPGQ | 693 | GIPDRFSGSGSGTDFTL | 695 | FGGGTKVEIK | 697 |
| | | | APRLLIY | | TISRLEPEDFAVYYC | | | |
| ADI-34137 | EIVLTQSPGTLSLSPGERATLSC | 711 | WYQQKPGQ | 713 | GIPDRFSGSGSGTDFTL | 715 | FGGGTKVEIK | 717 |
| | | | APRLLIY | | TISRLEPEDFAVYYC | | | |
| ADI-34138 | EIVMTQSPGTLSLSPGERATLSC | 731 | WYQQKPGQ | 733 | GIPDRFSGSGSGTDFTL | 735 | FGGGTKVEIK | 737 |
| | | | APRLLIY | | TISRLEPEDFAVYYC | | | |
| Consensus | X₁X₂X₃TQSPX₄X₅LSX₆ | 931 | WYQQKPGX₁ | 933 | GX₁PX₂RFSGSGSGTDFT | 935 | FGGGTKVEIK | 937 |
| sequence | SX₇GX₈RX₉TX₁₀X₁₁C | | APX₂LLIY | | LTISX₃LX₄PEDFAX₅ | | | |
| | X₁ = E or D; | | X₁ = Q or K; | | YYC | | | |
| | X₂ = V or Q; | | and X₂ = R | | X₁ = I or V; | | | |
| | X₃ = L or M; | | or K | | X₂ = D or S; | | | |
| | X₄ = G or S; | | | | X₃ = R or | | | |
| | X₅ = T or S; | | | | S; | | | |
| | X₆ = L or A; | | | | X₄ = E or Q; | | | |
| | X₇ = P or V; | | | | and | | | |
| | X₈ = E or D; | | | | X₅ = V or T | | | |
| | X₉ = A or V; | | | | | | | |
| | X₁₀ = L or I; | | | | | | | |
| | and X₁₁ = S or T | | | | | | | |
| ADI-30254 | DIVMTQSPLSLPVTPGEPASISC | 751 | WYLAKPGQ | 753 | GVPDRFSGSGSGTDFTL | 755 | FGGGTKVEIK | 757 |
| | | | SPQLLIY | | KISRVEAEDVGVYYC | | | |
| ADI-34139 | DIVMTQSPLSLPVTPGEPASISC | 771 | WYLAKPGQ | 773 | GVPDRFSGSGSGTDFTL | 775 | FGGGTKVEIK | 777 |
| | | | SPQLLIY | | KISRVEAEDVGVYYC | | | |
| ADI-34140 | DIVMTQSPLSLPVTPGEPASISC | 791 | WYLQKPGQ | 793 | GVPDRFSGSGSGTDFTL | 795 | FGGGTKVEIK | 797 |
| | | | SPQLLIY | | KISRVEAEDVGVYYC | | | |
| ADI-34141 | DIVMTQSPLSLPVTPGEPASISC | 811 | WYLQKPGQ | 813 | GVPDRFSGSGSGTDFTL | 815 | FGGGTKVEIK | 817 |
| | | | SPQLLIY | | KISRVEAEDVGVYYC | | | |
| ADI-34142 | DIVMTQSPLSLPVTPGEPASISC | 831 | WYLQKPGQ | 833 | GVPDRFSGSGSGTDFTL | 835 | FGGGTKVEIK | 837 |
| | | | SPQLLIY | | KISRVEAEDVGVYYC | | | |
| ADI-34143 | DIVMTQSPLSLPVTPGEPASISC | 851 | WYLAKPGQ | 853 | GVPDRFSGSGSGTDFTL | 855 | FGGGTKVEIK | 857 |
| | | | SPQLLIY | | KISRVEAEDVGVYYC | | | |
| ADI-34144 | DIVMTQSPLSLPVTPGEPASISC | 871 | WYLAKPGQ | 873 | GVPDRFSGSGSGTDFTL | 875 | FGGGTKVEIK | 877 |
| | | | SPQLLIY | | KISRVEAEDVGVYYC | | | |
| ADI-34145 | DIVMTQSPLSLPVTPGEPASISC | 891 | WYLQKPGQ | 893 | GVPDRFSGSGSGTDFTL | 895 | FGGGTKVEIK | 897 |
| | | | SPQLLIY | | KISRVEAEDVGVYYC | | | |
| ADI-34146 | DIVMTQSPLSLPVTPGEPASISC | 911 | WYLAKPGQ | 913 | GVPDRFSGSGSGTDFTL | 915 | FGGGTKVEIK | 917 |
| | | | SPQLLIY | | KISRVEAEDVGVYYC | | | |
| Consensus | DIVMTQSPLSLPVTPGEPASISC | 951 | WYLAKPGQ | 953 | GVPDRFSGSGSGTDFTL | 955 | FGGGTKVEIK | 957 |
| sequence | | | SPQLLIY | | KISRVEAEDVGVYYC | | | |

TABLE 5
Anti-ID antibody kinetic values

| | Anti-ID IgG/ | Anti-ID IgG/ |
|---|---|---|
| | ADI-22523 IgG | ADI-22523 Fab |
| ADI Name | K_{D} (nM) | K_{D} (nM) |
| ADI-30242 | 0.57 | 37.43 |
| ADI-34131 | 0.73 | 13.54 |
| ADI-34132 | 84.91 | No observable binding |
| ADI-34133 | 0.59 | 9.59 |
| ADI-34134 | 0.46 | 5.62 |
| ADI-34135 | 0.54 | 7.81 |
| ADI-34136 | 0.75 | 30.44 |
| ADI-34137 | 0.63 | 14.84 |
| ADI-34138 | 0.57 | 9.69 |
| ADI-30254 | 3.34 | 50.28 |
| ADI-34139 | 2.99 | 12.52 |
| ADI-34140 | 2.90 | 18.85 |
| ADI-34141 | 2.83 | 16.49 |
| ADI-34142 | 4.11 | 14.70 |
| ADI-34143 | 2.98 | 18.16 |
| ADI-34144 | 2.60 | 6.17 |
| ADI-34145 | 2.72 | 4.13 |

TABLE 6
Anti-ID antibody kinetic values

| | anti-CD3 IgG/ | ADI-34134 IgG/ | anti-CD3 IgG/ | anti-CD3 IgG/ | ADI-34145 IgG/ | anti-CD3 IgG/ |
|---|---|---|---|---|---|---|
| | ADI-34134 IgG | anti-CD3 Fab | ADI-34134 Fab | ADI-34145 IgG | anti-CD3 Fab | ADI-34145 Fab |
| ADI Name | K_{D} (nM) | K_{D} (nM) | K_{D} (nM) | K_{D} (nM) | K_{D} (nM) | K_{D} (nM) |
| ADI-22523 | 0.37 | 4.46 | 5.68 | 2.60 | 3.16 | 3.22 |
| ADI-26906 | 0.43 | 36.00 | 17.80 | 8.39 | Poor fit | 3.25 |
| ADI-26913 | 0.77 | 86.50 | 79.80 | 1.03 | Poor fit | 3.15 |
| ADI-26916 | 1.04 | 110.00 | 157.00 | 7.75 | Poor fit | 19.40 |
| ADI-26920 | 0.46 | 29.80 | 31.00 | 3.50 | 3.71 | 8.97 |
| ADI-26921 | 0.57 | 92.70 | 56.50 | 12.70 | No observable | Poor fit |
| | | | | | binding | |
| ADI-26927 | 0.45 | 25.60 | 38.20 | 26.80 | No observable | No observable |
| | | | | | binding | binding |
| ADI-26928 | 0.85 | 182.00 | 185.00 | 34.20 | No observable | No observable |
| | | | | | binding | binding |
| ADI-26941 | 1.13 | 112.00 | 138.00 | 13.40 | No observable | Poor fit |
| | | | | | binding | |
| ADI-26954 | No observable | No observable | No observable | 1.82 | No observable | Poor fit |
| | binding | binding | binding | | binding | |
| CTL-19672 | 9.21 | No observable | No observable | No observable | No observable | No observable |
| | | binding | binding | binding | binding | binding |
| CTL-19613 | No observable | No observable | No observable | No observable | No observable | No observable |
| | binding | binding | binding | binding | binding | binding |

TABLE 7
Anti-ID antibody kinetic values

| | ADI-34134 IgG/ | ADI-34145 IgG/ | ADI-34134 IgG/ | ADI-34145 IgG/ |
|---|---|---|---|---|
| | anti-CD3 IgG | anti-CD3 IgG | anti-CD3 Fab | anti-CD3 Fab |
| ADI Name | K_{D} (nM) | K_{D} (nM) | K_{D} (nM) | K_{D} (nM) |
| ADI-22523 | 0.46 | 2.72 | 5.62 | 4.13 |
| ADI-26906 | 0.43 | 8.39 | 17.78 | 3.55 |
| ADI-26913 | 0.77 | 8.10 | 79.79 | 7.52 |
| ADI-26916 | 0.57 | 48.04 | 56.46 | No observable binding |
| ADI-26920 | No observable binding | 14.33 | No observable binding | Poor fit |
| ADI-26921 | 0.85 | No observable binding | 184.79 | No observable binding |
| ADI-26927 | 0.45 | 39.01 | 38.22 | No observable binding |
| ADI-26928 | 0.46 | 4.44 | 30.97 | 8.97 |
| ADI-26941 | 1.13 | No observable binding | 137.92 | Poor fit |
| ADI-26954 | 1.04 | 7.75 | 157.47 | 19.39 |
| CTL-19672 | No observable binding | No observable binding | No observable binding | No observable binding |
| CTL-19613 | 9.21 | No observable binding | No observable binding | No observable binding |

TABLE 8
Anti-ID antibody kinetic values

| | anti-CD3 bsIgG/ | anti-CD3 bsIgG/ | anti-CD3 bsIgG/ | anti-CD3 bsIgG/ |
|---|---|---|---|---|
| | ADI-34134 Fab | ADI-34134 IgG | ADI-34145 Fab | ADI-34145 IgG |
| ADI Name | K_{D} (nM) | K_{D} (nM) | K_{D} (nM) | K_{D} (nM) |
| ADI-22523 | 10.86 | 0.47 | Poor fit | 9.13 |
| ADI-26906 | 27.24 | 0.75 | 24.45 | 13.45 |
| ADI-26913 | 74.22 | 0.98 | 159.18 | 14.65 |
| ADI-26916 | Poor fit | 1.94 | No observable binding | 57.09 |
| ADI-26920 | 28.70 | 0.47 | 93.79 | 6.72 |
| ADI-26921 | 37.07 | 0.94 | No observable binding | 33.21 |
| ADI-26927 | 48.90 | 1.02 | No observable binding | 48.53 |
| ADI-26928 | 110.36 | 1.39 | No observable binding | Poor fit |
| ADI-26954 | No observable binding | No observable binding | No observable binding | 27.84 |
| CTL-19672 | No observable binding | 16.39 | No observable binding | No observable binding |
| CTL-19613 | No observable binding | No observable binding | No observable binding | No observable binding |

TABLE 9
PSR score and HIC retention time

| ADI Name | PSR Score | HIC retention time (min) |
|---|---|---|
| ADI-30242 | 0.00 | 10.07 |
| ADI-34131 | 0.00 | 10.43 |
| ADI-34132 | 0.00 | 11.52 |
| ADI-34133 | 0.00 | 10.50 |
| ADI-34134 | 0.00 | 10.14 |
| ADI-34135 | 0.00 | 10.38 |
| ADI-34136 | 0.00 | 10.70 |
| ADI-34137 | 0.00 | 10.61 |
| ADI-34138 | 0.01 | 10.70 |
| ADI-30254 | 0.00 | 9.18 |
| ADI-34139 | 0.00 | 9.60 |
| ADI-34140 | 0.06 | 9.62 |
| ADI-34141 | 0.01 | 9.27 |
| ADI-34142 | 0.13 | 10.46 |
| ADI-34143 | 0.02 | 9.22 |
| ADI-34144 | 0.01 | 9.50 |
| ADI-34145 | 0.10 | 9.61 |
| ADI-34146 | 0.01 | 9.58 |

TABLE 10A
Anti-ID antibody avidity K_{D} (M) values by SPR using CARTERRA ® (yeast-produced ADI-30242 and ADI-30242 variants)

| ADI Name | ADI-30242 | ADI-34131 | ADI-34132 | ADI-34133 | ADI-34134 | ADI-34135 | ADI-34136 | ADI-34137 | ADI-34138 |
|---|---|---|---|---|---|---|---|---|---|
| ADI-50024 | 1.1E−08 | 1.8E−08 | 3.5E−10 | 1.1E−09 | P.F. | *1.7E−10 | *4.2E−10 | 3.9E−10 | 1.9E−08 |
| ADI-70326 | 4.5E−08 | 1.3E−07 | P.F. | P.F. | P.F. | 4.1E−09 | 2.1E−09 | P.F. | 4.2E−08 |
| ADI-70327 | 4.5E−08 | 1.3E−07 | P.F. | P.F. | 1.4E−08 | P.F. | 2.0E−09 | 9.5E−10 | 1.6E−07 |
| ADI-70330 | 4.0E−08 | 8.0E−08 | P.F. | 1.9E−09 | 1.3E−08 | 4.7E−10 | 4.2E−10 | 3.0E−10 | 4.5E−08 |
| ADI-26906 | 5.9E−09 | 2.7E−08 | 1.6E−10 | *3.0E−10 | P.F. | *1.3E−10 | *2.5E−10 | 3.3E−10 | 2.0E−08 |
| ADI-67450 | 5.6E−08 | 6.9E−08 | P.F. | 2.0E−09 | 1.2E−08 | 6.3E−10 | 5.6E−10 | 9.2E−10 | *2.7E−08 |
| ADI-67445 | 1.1E−08 | *2.5E−08 | 6.5E−10 | 1.1E−09 | P.F. | 3.4E−10 | 3.4E−10 | 3.7E−10 | *2.7E−08 |
| ADI-67447 | 8.4E−09 | *1.3E−08 | 5.6E−10 | 1.3E−09 | P.F. | 2.6E−10 | 1.8E−10 | 2.2E−10 | *1.2E−08 |
| ADI-26909 | 1.7E−08 | *1.3E−08 | 9.0E−10 | 1.3E−09 | P.F. | 1.0E−10 | 3.0E−10 | 3.2E−10 | *1.2E−08 |
| ADI-26913 | 2.4E−08 | 2.0E−08 | 9.3E−10 | 1.9E−09 | 2.1E−08 | 1.0E−10 | 1.8E−10 | 3.0E−10 | *1.6E−08 |
| ADI-26919 | *3.5E−09 | 2.5E−08 | *4.4E−11 | *3.5E−10 | 9.1E−09 | *8.2E−11 | 1.6E−10 | 1.4E−10 | 3.3E−08 |
| ADI-26920 | *4.2E−09 | *1.1E−08 | 1.5E−10 | *2.4E−10 | 1.6E−08 | *1.1E−10 | *1.4E−10 | *8.8E−11 | *1.0E−08 |
| ADI-26921 | 2.1E−08 | *1.8E−08 | 3.6E−10 | 9.6E−10 | 3.6E−09 | *7.3E−11 | 1.8E−10 | *4.8E−11 | *1.7E−08 |
| ADI-26916 | 3.5E−08 | 8.4E−08 | 6.0E−10 | P.F. | *6.3E−09 | 1.4E−10 | 3.5E−10 | 3.1E−10 | 6.4E−08 |
| ADI-26912 | 3.3E−08 | N.B. | P.F. | 5.2E−08 | 3.4E−08 | P.F. | P.F. | P.F | N.B. |
| ADI-26924 | *5.3E−09 | *1.7E−08 | 2.0E−10 | 1.1E−09 | 2.0E−08 | 1.4E−10 | 1.7E−10 | 1.9E−10 | *1.6E−08 |
| ADI-26915 | 5.6E−08 | N.B. | P.F. | P.F. | 1.9E−08 | 1.5E−08 | P.F | 1.4E−08 | N.B. |
| ADI-26943 | *6.2E−09 | *2.2E−08 | 1.5E−10 | 6.5E−10 | 1.3E−08 | *9.9E−11 | 2.5E−10 | 1.7E−10 | *2.2E−08 |
| ADI-48576 | N.B. | N.B. | W.B. | N.B. | N.B. | 3.1E−08 | 2.9E−08 | W.B. | W.B. |
| ADI-48587 | 4.3E−08 | W.B. | P.F. | 2.0E−08 | W.B. | 1.8E−09 | 1.9E−09 | 8.8E−10 | 2.9E−08 |
| ADI-48592 | W.B. | N.B. | W.B. | N.B. | N.B. | 1.9E−08 | W.B. | P.F | W.B. |
| ADI-48650 | 4.4E−08 | N.B. | P.F. | 4.0E−08 | N.B. | P.F. | 3.3E−09 | P.F. | 1.1E−07 |
| ADI-74965 | 4.5E−08 | N.B. | P.F. | 1.8E−08 | N.B. | P.F. | 9.4E−09 | P.F | 3.4E−08 |
| ADI-74966 | 2.5E−08 | 3.1E−08 | 5.8E−10 | P.F. | 5.2E−08 | 3.5E−10 | 6.4E−10 | 2.1E−10 | *1.5E−08 |
| ADI-74967 | *5.6E−09 | 2.2E−08 | 2.8E−10 | 1.9E−09 | P.F. | 2.1E−10 | *1.5E−10 | *1.2E−10 | *1.8E−08 |
| ADI-74968 | 3.6E−08 | W.B. | P.F. | 8.2E−09 | W.B. | 8.3E−10 | 1.2E−09 | 7.6E−10 | 2.9E−08 |
| ADI-76357 | 2.2E−08 | 3.5E−08 | 5.7E−10 | 9.6E−10 | 9.5E−09 | 9.7E−11 | *1.2E−10 | 8.6E−11 | 1.7E−08 |
| ADI-76358 | 4.2E−08 | 9.1E−08 | P.F | P.F. | P.F. | 1.1E−09 | 7.3E−10 | 9.6E−10 | 2.9E−08 |
| ADI-76359 | *3.7E−09 | *8.7E−09 | 1.2E−10 | 2.4E−10 | P.F. | *5.3E−11 | 1.2E−10 | *5.9E−11 | *9.5E−09 |
| ADI-76360 | 1.1E−08 | 4.3E−08 | 6.1E−10 | 1.7E−09 | P.F. | 1.5E−10 | 3.5E−10 | 8.2E−11 | 5.7E−08 |
| ADI-76361 | 1.5E−08 | *1.9E−08 | 1.5E−10 | 9.1E−10 | 7.1E−09 | *4.4E−11 | *1.0E−10 | 5.4E−11 | 2.1E−08 |
| ADI-76362 | 2.7E−08 | *1.6E−08 | 8.2E−10 | 2.0E−09 | 6.3E−09 | 4.1E−10 | 1.4E−10 | 2.2E−10 | *1.7E−08 |
| CTL-62223 | 3.1E−08 | 5.4E−08 | 9.9E−10 | P.F | 1.8E−08 | 1.2E−09 | 1.2E−09 | 1.8E−09 | 4.6E−08 |
| CTL-62227 | N.B. | N.B. | N.B. | N.B. | N.B. | N.B. | N.B. | N.B. | N.B. |
| CTL-62228 | N.B. | N.B. | N.B. | N.B. | N.B. | N.B. | N.B. | N.B. | N.B. |
| CTL-62240 | N.B. | N.B. | N.B. | N.B. | N.B. | N.B. | N.B. | N.B. | N.B. |

*Reached koff limit for 300 second dissociation.

TABLE 10B
Anti-ID antibody avidity K_{D} (M) values by SPR using CARTERRA ® (yeast-produced
ADI-30254 and ADI-30254 variants)

| ADI Name | ADI-30254 | ADI-34146 | ADI-34145 | ADI-34141 | ADI-34142 | ADI-34143 | ADI-34144 |
|---|---|---|---|---|---|---|---|
| ADI-50024 | N.B. | 1.1E−09 | N.B. | W.B. | W.B. | N.B. | W.B. |
| ADI-70326 | N.B. | 3.9E−09 | N.B. | W.B. | N.B. | N.B. | N.B. |
| ADI-70327 | N.B. | 1.7E−09 | N.B. | N.B. | N.B. | N.B. | N.B. |
| ADI-70330 | N.B. | 1.2E−09 | N.B. | N.B. | N.B. | N.B. | N.B. |
| ADI-26906 | W.B. | 6.3E−10 | 1.4E−08 | *1.3E−08 | *1.2E−08 | *1.3E−08 | *1.2E−08 |
| ADI-67450 | N.B. | 1.8E−09 | N.B. | P.F. | W.B. | W.B. | W.B. |
| ADI-67445 | N.B. | 9.3E−10 | N.B. | N.B. | N.B. | N.B. | N.B. |
| ADI-67447 | N.B. | *3.5E−10 | 2.7E−08 | 1.7E−08 | 2.1E−08 | 3.3E−08 | 2.2E−08 |
| ADI-26909 | N.B. | 6.4E−10 | N.B. | W.B. | N.B. | N.B. | N.B. |
| ADI-26913 | W.B. | 5.5E−10 | 1.8E−08 | *7.0E−09 | 1.1E−08 | 2.3E−08 | *8.6E−09 |
| ADI-26919 | *3.3E−08 | 3.5E−10 | 1.8E−08 | *1.0E−08 | 1.8E−08 | 2.4E−08 | 3.2E−08 |
| ADI-26920 | *2.1E−08 | *2.9E−10 | 8.6E−09 | *7.1E−09 | *6.1E−09 | 3.5E−08 | *7.5E−09 |
| ADI-26921 | N.B. | 2.4E−10 | N.B. | W.B. | N.B. | W.B. | N.B. |
| ADI-26916 | 5.6E−08 | 1.8E−09 | 2.0E−08 | *1.0E−08 | 2.1E−08 | 4.3E−08 | 1.6E−08 |
| ADI-26912 | N.B. | P.F. | N.B. | P.F. | P.F. | W.B. | W.B. |
| ADI-26924 | *2.1E−08 | *2.5E−10 | 1.7E−08 | *5.4E−09 | 3.2E−08 | 2.0E−08 | 4.9E−09 |
| ADI-26915 | N.B. | P.F. | N.B. | N.B. | N.B. | N.B. | N.B. |
| ADI-26943 | 2.7E−08 | 8.7E−10 | 9.5E−09 | *5.7E−09 | 6.5E−09 | 1.1E−08 | *5.1E−09 |
| ADI-48576 | N.B. | 1.2E−08 | W.B. | 2.2E−08 | 7.6E−08 | W.B. | P.F. |
| ADI-48587 | N.B. | 7.9E−10 | N.B. | W.B. | N.B. | N.B. | N.B. |
| ADI-48592 | W.B. | 1.6E−08 | P.F. | P.F. | P.F. | P.F. | P.F. |
| ADI-48650 | N.B. | 3.3E−09 | N.B. | W.B. | W.B. | N.B. | W.B. |
| ADI-74965 | N.B. | 7.5E−10 | W.B. | 3.3E−08 | P.F. | P.F. | P.F. |
| ADI-74966 | W.B. | 8.6E−10 | P.F. | 5.1E−08 | P.F. | 3.6E−08 | 5.9E−08 |
| ADI-74967 | N.B. | 5.0E−10 | N.B. | 3.2E−08 | W.B. | W.B. | N.B. |
| ADI-74968 | N.B. | 4.1E−10 | 6.7E−08 | 6.3E−08 | 4.5E−08 | 1.3E−07 | 7.2E−08 |
| ADI-76357 | W.B. | 5.4E−10 | 2.3E−08 | *7.6E−09 | *5.7E−09 | *6.6E−09 | *6.3E−09 |
| ADI-76358 | N.B. | 2.9E−09 | N.B. | P.F. | W.B. | W.B. | W.B. |
| ADI-76359 | 2.7E−08 | 3.9E−10 | 1.4E−08 | 1.4E−08 | 7.3E−09 | 2.0E−08 | 1.7E−08 |
| ADI-76360 | N.B. | 5.7E−10 | W.B. | 5.4E−08 | 3.6E−08 | P.F. | W.B. |
| ADI-76361 | N.B. | 2.4E−10 | N.B. | W.B. | W.B. | W.B. | P.F. |
| ADI-76362 | N.B. | 1.4E−09 | N.B. | P.F. | W.B. | W.B. | W.B. |
| CTL-62223 | N.B. | 4.5E−09 | N.B. | N.B. | N.B. | N.B. | N.B. |
| CTL-62227 | N.B. | N.B. | N.B. | N.B. | N.B. | N.B. | N.B. |
| CTL-62228 | N.B. | N.B. | N.B. | N.B. | N.B. | N.B. | N.B. |
| CTL-62240 | N.B. | N.B. | N.B. | N.B. | N.B. | N.B. | N.B. |

*Reached koff limit for 300 second dissociation.

TABLE 10C
Anti-ID antibody avidity K_{D} (M) values
by SPR using CARTERRA ® (CHO-produced
ADI-34134 and ADI-34145)

| ADI Name | ADI-34134 | ADI-34145 |
|---|---|---|
| ADI-50024 | *2.6E−10 | P.F. |
| ADI-70326 | 1.2E−09 | N.B. |
| ADI-70327 | 1.3E−09 | N.B. |
| ADI-70330 | 6.4E−10 | P.F. |
| ADI-26906 | *1.8E−10 | *6.4E−09 |
| ADI-67450 | 6.8E−10 | *1.3E−08 |
| ADI-67445 | *1.3E−10 | N.B. |
| ADI-67447 | *1.2E−10 | 1.4E−08 |
| ADI-26909 | *1.1E−10 | P.F |
| ADI-26913 | 1.3E−10 | *4.4E−09 |
| ADI-26919 | *1.1E−10 | 1.2E−08 |
| ADI-26920 | *1.1E−10 | 1.5E−08 |
| ADI-26921 | *1.1E−10 | P.F |
| ADI-26916 | 1.7E−10 | 1.7E−08 |
| ADI-26912 | P.F. | P.F. |
| ADI-26924 | *1.0E−10 | 3.5E−09 |
| ADI-26915 | P.F. | N.B. |
| ADI-26943 | *1.0E−10 | 4.2E−09 |
| ADI-48576 | P.F. | P.F |
| ADI-48587 | 1.3E−09 | P.F. |
| ADI-48592 | P.F. | P.F. |
| ADI-48650 | P.F. | P.F. |
| ADI-74965 | P.F. | P.F. |
| ADI-74966 | 3.9E−10 | P.F. |
| ADI-74967 | *1.1E−10 | P.F. |
| ADI-74968 | 7.8E−10 | 4.1E−08 |
| ADI-76357 | *8.8E−11 | 5.6E−09 |
| ADI-76358 | 5.4E−10 | P.F. |
| ADI-76359 | 1.5E−10 | 8.0E−09 |
| ADI-76360 | 2.1E−10 | P.F. |
| ADI-76361 | *9.4E−11 | P.F. |
| ADI-76362 | 2.3E−10 | W.B. |
| CTL-62223 | *1.9E−10 | N.B. |
| CTL-62227 | N.B. | N.B. |
| CTL-62228 | N.B. | N.B. |
| CTL-62240 | N.B. | N.B. |

*Reached koff limit for 300 second dissociation.

TABLE 11
Anti-CD3 antibody variable region SEQ ID NO summary

| ADI Name | VH | CDRH1 | CDRH2 | CDRH3 | VL | CDRL1 | CDRL2 | CDRL3 |
|---|---|---|---|---|---|---|---|---|
| ADI-22523 | 110 | 111 | 112 | 113 | 115 | 116 | 117 | 118 |
| ADI-26927 | 120 | 121 | 122 | 123 | 125 | 126 | 127 | 128 |
| ADI-26928 | 130 | 131 | 132 | 133 | 135 | 136 | 137 | 138 |
| ADI-26941 | 140 | 141 | 142 | 143 | 145 | 146 | 147 | 148 |
| ADI-26954 | 150 | 151 | 152 | 153 | 155 | 156 | 157 | 158 |
| CTL-19613 | 160 | 161 | 162 | 163 | 165 | 166 | 167 | 168 |
| CTL-19672 | 170 | 171 | 172 | 173 | 175 | 176 | 177 | 178 |
| ADI-50024 | 180 | 181 | 182 | 183 | 185 | 186 | 187 | 188 |
| ADI-70326 | 190 | 191 | 192 | 193 | 195 | 196 | 197 | 198 |
| ADI-70327 | 200 | 201 | 202 | 203 | 205 | 206 | 207 | 208 |
| ADI-70330 | 210 | 211 | 212 | 213 | 215 | 216 | 217 | 218 |
| ADI-26906 | 220 | 221 | 222 | 223 | 225 | 226 | 227 | 228 |
| ADI-67450 | 230 | 231 | 232 | 233 | 235 | 236 | 237 | 238 |
| ADI-67445 | 240 | 241 | 242 | 243 | 245 | 246 | 247 | 248 |
| ADI-67447 | 250 | 251 | 252 | 253 | 255 | 256 | 257 | 258 |
| ADI-26909 | 260 | 261 | 262 | 263 | 265 | 266 | 267 | 268 |
| ADI-26913 | 270 | 271 | 272 | 273 | 275 | 276 | 277 | 278 |
| ADI-26919 | 280 | 281 | 282 | 283 | 285 | 286 | 287 | 288 |
| ADI-26920 | 290 | 291 | 292 | 293 | 295 | 296 | 297 | 298 |
| ADI-26921 | 300 | 301 | 302 | 303 | 305 | 306 | 307 | 308 |
| ADI-26916 | 310 | 311 | 312 | 313 | 315 | 316 | 317 | 318 |
| ADI-26912 | 320 | 321 | 322 | 323 | 325 | 326 | 327 | 328 |
| ADI-26924 | 330 | 331 | 332 | 333 | 335 | 336 | 337 | 338 |
| ADI-26915 | 340 | 341 | 342 | 343 | 345 | 346 | 347 | 348 |
| ADI-26943 | 350 | 351 | 352 | 353 | 355 | 356 | 357 | 358 |
| ADI-48576 | 360 | 361 | 362 | 363 | 365 | 366 | 367 | 368 |
| ADI-48587 | 370 | 371 | 372 | 373 | 375 | 376 | 377 | 378 |
| ADI-48592 | 380 | 381 | 382 | 383 | 385 | 386 | 387 | 388 |
| ADI-48650 | 390 | 391 | 392 | 393 | 395 | 396 | 397 | 398 |
| ADI-74965 | 400 | 401 | 402 | 403 | 405 | 406 | 407 | 408 |
| ADI-74966 | 410 | 411 | 412 | 413 | 415 | 416 | 417 | 418 |
| ADI-74967 | 420 | 421 | 422 | 423 | 425 | 426 | 427 | 428 |
| ADI-74968 | 430 | 431 | 432 | 433 | 435 | 436 | 437 | 438 |
| ADI-76357 | 440 | 441 | 442 | 443 | 445 | 446 | 447 | 448 |
| ADI-76358 | 450 | 451 | 452 | 453 | 455 | 456 | 457 | 458 |
| ADI-76359 | 460 | 461 | 462 | 463 | 465 | 466 | 467 | 468 |
| ADI-76360 | 470 | 471 | 472 | 473 | 475 | 476 | 477 | 478 |
| ADI-76361 | 480 | 481 | 482 | 483 | 485 | 486 | 487 | 488 |
| ADI-76362 | 490 | 491 | 492 | 493 | 495 | 496 | 497 | 498 |
| CTL-62223 | 500 | 501 | 502 | 503 | 505 | 506 | 507 | 508 |
| CTL-62227 | 510 | 511 | 512 | 513 | 515 | 516 | 517 | 518 |
| CTL-62228 | 520 | 521 | 522 | 523 | 525 | 526 | 527 | 528 |
| CTL-62240 | 530 | 531 | 532 | 533 | 535 | 536 | 537 | 538 |

TABLE 12
Anti-ID antibody variable region SEQ ID NO summary

| ADI | | | | | | | | | | | | | | | | |
|---|---|---|---|---|---|---|---|---|---|---|---|---|---|---|---|---|
| Name | VH | FRH1 | CDRH1 | FRH2 | CDRH2 | FRH3 | CDRH3 | FRH4 | VL | FRL1 | CDRL1 | FRL2 | CDRL2 | FRL3 | CDRL3 | FRL4 |
| ADI- | 560 | 561 | 562 | 563 | 564 | 565 | 566 | 567 | 570 | 571 | 572 | 573 | 574 | 575 | 576 | 577 |
| 30242 | | | | | | | | | | | | | | | | |
| ADI- | 580 | 581 | 582 | 583 | 584 | 585 | 586 | 587 | 590 | 591 | 592 | 593 | 594 | 595 | 596 | 597 |
| 34131 | | | | | | | | | | | | | | | | |
| ADI- | 600 | 601 | 602 | 603 | 604 | 605 | 606 | 607 | 610 | 611 | 612 | 613 | 614 | 615 | 616 | 617 |
| 34132 | | | | | | | | | | | | | | | | |
| ADI- | 620 | 621 | 622 | 623 | 624 | 625 | 626 | 627 | 630 | 631 | 632 | 633 | 634 | 635 | 636 | 637 |
| 34133 | | | | | | | | | | | | | | | | |
| ADI- | 640 | 641 | 642 | 643 | 644 | 645 | 646 | 647 | 650 | 651 | 652 | 653 | 654 | 655 | 656 | 657 |
| 34134 | | | | | | | | | | | | | | | | |
| ADI- | 660 | 661 | 662 | 663 | 664 | 665 | 666 | 667 | 670 | 671 | 672 | 673 | 674 | 675 | 676 | 677 |
| 34135 | | | | | | | | | | | | | | | | |
| ADI- | 680 | 681 | 682 | 683 | 684 | 685 | 686 | 687 | 690 | 691 | 692 | 693 | 694 | 695 | 696 | 697 |
| 34136 | | | | | | | | | | | | | | | | |
| ADI- | 700 | 701 | 702 | 703 | 704 | 705 | 706 | 707 | 710 | 711 | 712 | 713 | 714 | 715 | 716 | 717 |
| 34137 | | | | | | | | | | | | | | | | |
| ADI- | 720 | 721 | 722 | 723 | 724 | 725 | 726 | 727 | 730 | 731 | 732 | 733 | 734 | 735 | 736 | 737 |
| 34138 | | | | | | | | | | | | | | | | |
| Consensus | | 921 | 922 | 923 | 924 | 925 | 926 | 927 | | 931 | 932 | 933 | 934 | 935 | 936 | 937 |
| sequence | | | | | | | | | | | | | | | | |
| ADI- | 740 | 741 | 742 | 743 | 744 | 745 | 746 | 747 | 750 | 751 | 752 | 753 | 754 | 755 | 756 | 757 |
| 30254 | | | | | | | | | | | | | | | | |
| ADI- | 760 | 761 | 762 | 763 | 764 | 765 | 766 | 767 | 770 | 771 | 772 | 773 | 774 | 775 | 776 | 777 |
| 34139 | | | | | | | | | | | | | | | | |
| ADI- | 780 | 781 | 782 | 783 | 784 | 785 | 786 | 787 | 790 | 791 | 792 | 793 | 794 | 795 | 796 | 797 |
| 34140 | | | | | | | | | | | | | | | | |
| ADI- | 800 | 801 | 802 | 803 | 804 | 805 | 806 | 807 | 810 | 811 | 812 | 813 | 814 | 815 | 816 | 817 |
| 34141 | | | | | | | | | | | | | | | | |
| ADI- | 820 | 821 | 822 | 823 | 824 | 825 | 826 | 827 | 830 | 831 | 832 | 833 | 834 | 835 | 836 | 837 |
| 34142 | | | | | | | | | | | | | | | | |
| ADI- | 840 | 841 | 842 | 843 | 844 | 845 | 846 | 847 | 850 | 851 | 852 | 853 | 854 | 855 | 856 | 857 |
| 34143 | | | | | | | | | | | | | | | | |
| ADI- | 860 | 861 | 862 | 863 | 864 | 865 | 866 | 867 | 870 | 871 | 872 | 873 | 874 | 875 | 876 | 877 |
| 34144 | | | | | | | | | | | | | | | | |
| ADI- | 880 | 881 | 882 | 883 | 884 | 885 | 886 | 887 | 890 | 891 | 892 | 893 | 894 | 895 | 896 | 897 |
| 34145 | | | | | | | | | | | | | | | | |
| ADI- | 900 | 901 | 902 | 903 | 904 | 905 | 906 | 907 | 910 | 911 | 912 | 913 | 914 | 915 | 916 | 917 |
| 34146 | | | | | | | | | | | | | | | | |
| Consensus | | 941 | 942 | 943 | 944 | 945 | 946 | 947 | | 951 | 952 | 953 | 954 | 955 | 956 | 957 |
| sequence | | | | | | | | | | | | | | | | |

TABLE 13
Exemplary Constant Region amino acid sequences

| | | SEQ |
|---|---|---|
| | | ID |
| | Sequences | NO: |
| Human IgG1 CH1, | ASTKGPSVFPLAPSSKSTSGGTAALGCLVKDYFPEPVTVSWNSGALTSGVHTFPAVL | 541 |
| reference 1 | QSSGLYSLSSVVTVPSSSLGTQTYICNVNHKPSNTKVDKKV | |
| Human IgG1 CH1, | ASTKGPSVFPLAPSSKSTSGGTAALGCLVKDYFPEPVTVSWNSGALTSGVHTFPAVL | 542 |
| reference 2 | QSSGLYSLSSVVTVPSSSLGTQTYICNVNHKPSNTKVDKRV | |
| Human IgG1 hinge, | EPKSCDKTHTCPPCP | 543 |
| reference | | |
| Human IgG1 CH2, | APELLGGPSVFLFPPKPKDTLMISRTPEVTCVVVDVSHEDPEVKFNWYVDGVEVHN | 544 |
| reference | AKTKPREEQYNSTYRVVSVLTVLHQDWLNGKEYKCKVSNKALPAPIEKTISKAK | |
| Human IgG1 CH3 | GQPREPQVYTLPPSRDELTKNQVSLTCLVKGFYPSDIAVEWESNGQPENNYKTTPPV | 545 |
| (356D and 358L) | LDSDGSFFLYSKLTVDKSRWQQGNVFSCSVMHEALHNHYTQKSLSLSPG | |
| Human IgG1 CH3 | GQPREPQVYTLPPSREEMTKNQVSLTCLVKGFYPSDIAVEWESNGQPENNYKTTPP | 546 |
| (356E and 358M) | VLDSDGSFFLYSKLTVDKSRWQQGNVFSCSVMHEALHNHYTQKSLSLSPG | |
| Human IgG1 CH3 | GQPREPQVYTLPPSRDELTKNQVSLTCLVKGFYPSDIAVEWESNGQPENNYKTTPPV | 547 |
| (356D, 358L, | LDSDGSFFLYSKLTVDKSRWQQGNIFSCSVMHEALHNHYTQKSLSLSPG | |
| and V4221) | | |
| Human IgG1 CH3 | GQPREPQVYTLPPSRDELTKNQVSLTCLVKGFYPSDIAVEWESNGQPENNYKTTPPV | 548 |
| (356D, 358L, | LDSDGSFFLYSKLTVDKSRWQQGNVFSCSVMHEGLHNHYTQKSLSLSPG | |
| and A431G) | | |
| Human IgG CK | RTVAAPSVFIFPPSDEQLKSGTASVVCLLNNFYPREAKVQWKVDNALQSGNSQESV | 551 |
| | TEQDSKDSTYSLSSTLTLSKADYEKHKVYACEVTHQGLSSPVTKSFNRGEC | |
| Human IgG CL | GQPKAAPSVTLFPPSSEELQANKATLVCLISDFYPGAVTVAWKADSSPVKAGVETTT | 552 |
| | PSKQSNNKYAASSYLSLTPEQWKSHRSYSCQVTHEGSTVEKTVAPTECS | |

TABLE 14
Other amino acid sequences (anti-CD3 VH VL)

| | | SEQ |
|---|---|---|
| | | ID |
| | Sequence | NO |
| VH | QVQLVQSGAEVKKPGASVKVSCKASGFNIKDYYMHWVRQAPGQRLEWMGWIDLENGNTIYDPKFQGRVTITRDTSAS | 1 |
| | TAYMELSSLRSEDTAVYYCARDGYGRYFYDVWGQGTLVTVSS | |
| VH | QVQLVQSGAEVKKPGASVKVSCKASGFNIKDYYMHWVRQAPGQRLEWMGWIDLENANTIYDAKFQGRVTITRDTSAS | 2 |
| | TAYMELSSLRSEDTAVYYCARDAYGRYFYDVWGQGTLVTVSS | |
| VH | QVQLVQSGAEVKKPGASVKVSCKASGFNIKDYYMHWVRQAPGQRLEWMGWIDLENANTIYDAKFQGRVTITRDTSAS | 3 |
| | TAYMELSSLRSEDTAVYYCARDQYGRYFYDVWGQGTLVTVSS | |
| VH | QVQLVQSGAEVKKPGASVKVSCKASGFNIKDYYMHWVRQAPGQRLEWMGWIDLENANTIYDAKFQGRVTITRDTSAS | 4 |
| | TAYMELSSLRSEDTAVYYCARDVYGRYFYDLWGQGTLVTVSS | |
| VH | QVQLVQSGAEVKKPGASVKVSCKASGFNIKDYYMHWVRQAPGQRLEWMGWIDLENANTIYDAKFQGRVTITRDTSAS | 5 |
| | TAYMELSSLRSEDTAVYYCGRDAYGRYFYDVWGQGTLVTVSS | |
| VH | QVQLVQSGAEVKKPGASVKVSCKASGFNIKDYYMHWVRQAPGQRLEWMGWIDLEEGNTIYDAKFQGRVTITRDTSAST | 6 |
| | AYMELSSLRSEDTAVYYCARDAYGRYFYDVWGQGTLVTVSS | |
| VH | QVQLVQSGAEVKKPGASVKVSCKASGFNIKDYYMHWVRQAPGQRLEWMGWIDLEEGNTIYDAKFQGRVTITRDTSAST | 7 |
| | AYMELSSLRSEDTAVYYCARDRYGAYFYDVWGQGTLVTVSS | |
| VH | QVQLVQSGAEVKKPGASVKVSCKASGFNIKDYYMHWVRQAPGQRLEWMGWIDLEEGNTIYDAKFQGRVTITRDTSAST | 8 |
| | AYMELSSLRSEDTAVYYCGRDAYGLYFYDVWGQGTLVTVSS | |
| VH | QVQLVQSGAEVKKPGASVKVSCKASGFNIKDYYMHWVRQAPGQRLEWMGWIDLEEGNTIYDAKFQGRVTITRDTSAST | 9 |
| | AYMELSSLRSEDTAVYYCARDQYGRYFYDVWGQGTLVTVSS | |
| VH | QVQLVQSGAEVKKPGASVKVSCKASGFNIKDYYMHWVRQAPGQRLEWMGWIDLENANTIYDAKFQGRVTITRDTSAST | 10 |
| | AYMELSSLRSEDTAVYYCARDNYGGYFYDVWGQGTLVTVSS | |
| VH | EVQLVESGGGLVQPGGSLKLSCAASGFTFNKYAMNWVRQAPGKGLEWVARIRSKYNNYATYYADSVKDRFTISRDDSK | 11 |
| | NTAYLQMNNLKTEDTAVYYCVRHGNFGNSYISYWAYWGQGTLVTVSS | |
| VH | EVQLVQSGAEVKKPGASVKVSCKASGFTFTSYYIHWVRQAPGQGLEWIGWIYPENDNTKYNEKFKDRVTITADTSTST | 12 |
| | AYLELSSLRSEDTAVYYCARDGYSRYYFDYWGQGTLVTVSS | |
| VL | DIVMTQSPDSLAVSLGERATINCKSSQSLLNSRTGKNYLAWYQQKPGQPPKLLIYWASTRESGVPDRFSGSGSGTDFTLT | 13 |
| | ISSLQAEDVAVYYCKQSYSRRTFGGGTKVEIK | |
| VL | DIVMTQSPDSLAVSLGERATINCKSSQSLLNARTGKNYLAWYQQKPGQPPKLLIYWASTRESGVPDRFSGSGSGTDFTLT | 14 |
| | ISSLQAEDVAVYYCKQSYSRRTFGGGTKVEIK | |
| VL | DIVMTQSPDSLAVSLGERATINCKSSQSLLNARTGKNYLAWYQQKPGQPPKLLIYWASTRESGVPDRFSGSGSGTDFTLT | 15 |
| | ISSLQAEDVAVYYCKQSYSLRTFGGGTKVEIK | |
| VL | DIVMTQSPDSLAVSLGERATINCKSSQSLLNARTGKNYLAWYQQKPGQPPKLLIYWASTRESGVPDRFSGSGSGTDFTLT | 16 |
| | ISSLQAEDVAVYYCKQSYSIRTFGGGTKVEIK | |
| VL | DIVMTQSPDSLAVSLGERATINCKSSQSLLESRTGKNYLAWYQQKPGQPPKLLIYWASTRESGVPDRFSGSGSGTDFTLT | 17 |
| | ISSLQAEDVAVYYCKQSYSLRTFGGGTKVEIK | |
| VL | QTVVTQEPSLTVSPGGTVTLTCGSSTGAVTSGNYPNWVQQKPGQAPRGLIGGTKFLAPGTPARFSGSLLGGKAALTLSGV | 18 |
| | QPEDEAEYYCVLWYSNRWVFGGGTKLTVL | |
| VL | DIVMTQSPDSLAVSLGERATINCKSSQSLLNSRTRKNYLAWYQQKPGQSPKLLIYWTSTRKSGVPDRFSGSGSGTDFTLT | 19 |
| | ISSLQAEDVAVYYCKQSFILRTFGQGTKVEIK | |

In VH sequences, underlined sequence spans correspond to CDRH1, CDRH2, and CDRH3 in the order of appearance. Sequence spans without an underline correspond to FRH1, FRH2, FRH3, and FRH4 in the order of appearance.
In VL sequences, underlined sequence spans correspond to CDRL1, CDRL2, and CDRL3 in the order of appearance. Sequence spans without an underline correspond to FRL1, FRL2, FRL3, and FRL4 in the order of appearance.
For example, an anti-CD3 antibody may comprise the VH and VL of: SEQ ID NOS: 1 and 13, respectively; SEQ ID NOS: 2 and 14, respectively; SEQ ID NOS: 3 and 14, respectively; SEQ ID NOS: 4 and 14, respectively; SEQ ID NOS: 5 and 15, respectively; SEQ ID NOS: 6 and 15, respectively; SEQ ID NOS: 7 and 15, respectively; SEQ ID NOS: 8 and 15, respectively; SEQ ID NOS: 9 and 16, respectively; SEQ ID NOS: 10 and 17, respectively; SEQ ID NOS: 11 and 18, respectively; or SEQ ID NOS: 12 and 19, respectively.

TABLE 15
Other amino acid sequences (anti-CD3 CDRs)

| | | SEQ |
|---|---|---|
| | | ID |
| | Sequence | NO |
| CDRH1 | FNIKDYYMH | 20 |
| CDRH1 | FTFNKYAMN | 21 |
| CDRH1 | FTFTSYYIH | 22 |
| CDRH2 | WIDLENGNTI | 23 |
| | YDPKFQG | |
| CDRH2 | WIDLENANTI | 24 |
| | YDAKFQG | |
| CDRH2 | WIDLEEGNTI | 25 |
| | YDAKFQG | |
| CDRH2 | RIRSKYNNYA | 26 |
| | TYYADSVKD | |
| CDRH2 | WIYPENDNTK | 27 |
| | YNEKFKD | |
| CDRH3 | ARDGYGRYFY | 28 |
| | DV | |
| CDRH3 | ARDAYGRYFY | 29 |
| | DV | |
| CDRH3 | ARDQYGRYFY | 30 |
| | DV | |
| CDRH3 | ARDVYGRYFY | 31 |
| | DL | |
| CDRH3 | GRDAYGRYFY | 32 |
| | DV | |
| CDRH3 | ARDRYGAYFY | 33 |
| | DV | |
| CDRH3 | GRDAYGLYFY | 34 |
| | DV | |
| CDRH3 | ARDNYGGYFY | 35 |
| | DV | |
| CDRH3 | VRHGNFGNSY | 36 |
| | ISYWAY | |
| CDRH3 | ARDGYSRYYFDY | 37 |
| CDRL1 | KSSQSLLNSR | 38 |
| | TGKNYLA | |
| CDRL1 | KSSQSLLNAR | 39 |
| | TGKNYLA | |
| CDRL1 | KSSQSLLESR | 40 |
| | TGKNYLA | |
| CDRL1 | GSSTGAVTSG | 41 |
| | NYPN | |
| CDRL1 | KSSQSLLNSR | 42 |
| | TRKNYLA | |
| CDRL2 | WASTRES | 43 |
| CDRL2 | GTKFLAP | 44 |
| CDRL2 | WTSTRKS | 45 |
| CDRL3 | KQSYSRRT | 46 |
| CDRL3 | KQSYSLRT | 47 |
| CDRL3 | KQSYSIRT | 48 |
| CDRL3 | VLWYSNRWV | 49 |
| CDRL3 | KQSFILRT | 50 |

TABLE 16
Other amino acid sequences (anti-ID VH VL)

| | | SEQ |
|---|---|---|
| | | ID |
| | Sequence | NO |
| VH | QVQLVQSGAEVKKPGSSVKVSCKASGGTFSSYAISWVRQAPGQGLEWMGGIIPIFGTANYAQKFQGRVTITADE | 51 |
| | STSTAYMELSSLRSEDTAVYYCARGAPNYYGSGGYDDGWFDPWGQGTLVTVSS | |
| VH | EVQLLESGGGLVQPGGSLRLSCAASGFTFSSYAMSWVRQAPGKGLEWVSTISGSGGSTYYADSVKGRFTISRDNS | 52 |
| | KNTLYLQMNSLRAEDTAVYYCARLGGRRGSGSSYYYYYGMDVWGQGTTVTVSS | |
| VL | EIVLTQSPGTLSLSPGERATLSCRASQSVSSSYLAWYQQKPGQAPRLLIYGASSRATGIPDRFSGSGSGTDFTLT | 53 |
| | ISRLEPEDFAVYYCQQFGRDPTFGGGTKVEIK | |
| VL | EIVLTQSPGTLSLSPGERATLSCRASQSVSSSYLAWYQQKPGQAPRLLIYGASSRATGIPDRFSGSGSGTDFTLTI | 54 |
| | SRLEPEDFAVYYCLQFGSSPTFGGGTKVEIK | |
| VL | DIQLTQSPSSLSASVGDRVTITCRASQSISSYLNWYQQKPGKAPKLLIYAASNLQSGVPSRFSGSGSGTDFTLTIS | 55 |
| | SLQPEDFATYYCQQTLHTPITFGGGTKVEIK | |
| VL | EIVLTQSPGTLSLSPGERATLSCRASQSVSSSYLAWYQQKPGQAPRLLIYGASSRATGIPDRFSGSGSGTDFTLTI | 56 |
| | SRLEPEDFAVYYCQQFGRSPTFGGGTKVEIK | |
| VL | EIVLTQSPGTLSLSPGERATLSCRASQSVSSDYLAWYQQKPGQAPRLLIYGASSRATGIPDRFSGSGSGTDFTLTI | 57 |
| | SRLEPEDFAVYYCQQFGRVPTFGGGTKVEIK | |
| VL | EIVMTQSPGTLSLSPGERATLSCRASQSVSSSYLAWYQQKPGQAPRLLIYGASSRATGIPDRFSGSGSGTDFTLTI | 58 |
| | SRLEPEDFAVYYCQQFGRYPTFGGGTKVEIK | |
| VL | EIVLTQSPGTLSLSPGERATLSCRASQSVSSSYLAWYQQKPGQAPRLLIYGASSRATGIPDRFSGSGSGTDFTLTI | 59 |
| | SRLEPEDFAVYYCQQFGPSPTFGGGTKVEIK | |
| VL | EIVLTQSPGTLSLSPGERATLSCRASQSVSSSYLAWYQQKPGQAPRLLIYGASSRATGIPDRFSGSGSGTDFTLTI | 60 |
| | SRLEPEDFAVYYCQQFGRFPTFGGGTKVEIK | |
| VL | EIVMTQSPGTLSLSPGERATLSCRASQSVSSSYLAWYQQKPGQAPRLLIYGASNRATGIPDRFSGSGSGTDFTLTI | 61 |
| | SRLEPEDFAVYYCQQAHTALWTFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 62 |
| | LKISRVEAEDVGVYYCMQVRQTPWTFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 63 |
| | LKISRVEAEDVGVYYCMQVLDLPWTFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 64 |
| | LKISRVEAEDVGVYYCMQVLELPWTFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 65 |
| | LKISRVEAEDVGVYYCMQVKDTPWTFGGGTKVEIK | |
| VL | DIVMTQSPLSLPVTPGEPASISCRSSQSLLYSNGYNYLDWYLQKPGQSPQLLIYLGSNRASGVPDRFSGSGSGTDFT | 66 |
| | LKISRVEAEDVGVYYCMQVLGLPWTFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 67 |
| | LKISRVEAEDVGVYYCMQVSDTPWTFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 68 |
| | LKISRVEAEDVGVYYCMQVLEIPWTFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 69 |
| | LKISRVEAEDVGVYYCMQVLELPWAFGGGTKVEIK | |
| VL | DIVMTQSPLSLPVTPGEPASISCRSSQSLLHSNGYNYLDWYLQKPGQSPQLLIYLGSNRASGVPDRFSGSGSGTDFT | 70 |
| | LKISRVEAEDVGVYYCMQVLDLPWAFGGGTKVEIK | |

In VH sequences, underlined sequence spans correspond to CDRH1, CDRH2, and CDRH3 in the order of appearance. Sequence spans without an underline correspond to FRH1, FRH2, FRH3, and FRH4 in the order of appearance.
In VL sequences, underlined sequence spans correspond to CDRL1, CDRL2, and CDRL3 in the order of appearance. Sequence spans without an underline correspond to FRL1, FRL2, FRL3, and FRL4 in the order of appearance.
For example, an anti-ID antibody may comprise: a VH of SEQ ID NO: 51 and a VL of SEQ ID NO: 53, 54, 55, 56, 57, 58, 59, 60, or 61; or a VH of SEQ ID NO: 52 and a VL of 62, 63, 64, 65, 66, 67, 68, 69, or 70.

TABLE 17
Other amino acid sequences (anti-ID CDRs)

| | | SEQ |
|---|---|---|
| | | ID |
| | Sequence | NO |
| CDRH1 | GTFSSYAIS | 71 |
| CDRH1 | FTFSSYAMS | 72 |
| CDRH2 | GIIPIFGTAN | 73 |
| | YAQKFQG | |
| CDRH2 | TISGSGGSTY | 74 |
| | YADSVKG | |
| CDRH3 | ARGAPNYYGSG | 75 |
| | WGYDDGFDP | |
| CDRH3 | ARLGGRRGSGSS | 76 |
| | YYYYYGMDV | |
| CDRL1 | RASQSVSSSYLA | 77 |
| CDRL1 | RASQSISSYLN | 78 |
| CDRL1 | RASQSVSSDYLA | 79 |
| CDRL1 | RSSQSLLHSNGY | 80 |
| | NYLD | |
| CDRL1 | RSSQSLLYSNGY | 81 |
| | NYLD | |
| CDRL2 | GASSRAT | 82 |
| CDRL2 | AASNLQS | 83 |
| CDRL2 | GASNRAT | 84 |
| CDRL2 | LGSNRAS | 85 |
| CDRL3 | QQFGRDPT | 86 |
| CDRL3 | LQFGSSPT | 87 |
| CDRL3 | QQTLHTPIT | 88 |
| CDRL3 | QQFGRSPT | 89 |
| CDRL3 | QQFGRVPT | 90 |
| CDRL3 | QQFGRYPT | 91 |
| CDRL3 | QQFGPSPT | 92 |
| CDRL3 | QQFGRFPT | 93 |
| CDRL3 | QQAHTALWT | 94 |
| CDRL3 | MQVRQTPWT | 95 |
| CDRL3 | MQVLDLPWT | 96 |
| CDRL3 | MQVLELPWT | 97 |
| CDRL3 | MQVKDTPWT | 98 |
| CDRL3 | MQVLGLPWT | 99 |
| CDRL3 | MQVSDTPWT | 100 |
| CDRL3 | MQVLEIPWT | 101 |
| CDRL3 | MQVLEIPWA | 102 |
| CDRL3 | MQVLDIPWT | 104 |

TABLE 18
Other amino acid sequences

| | Sequences | SEQ ID NO: |
|---|---|---|
| CD3eN27 peptide | QDGNEEMGSITQTP | 104 |
| H2N-QDGNEEMGSITQTPY | YQVSISGTTVILTK | |
| QVSISGTTVILT[K/ | | |
| SCBiot(dPEG4)]-amide | | |
| CD3eN13 peptide | QDGNEEMGGITQTK | 105 |
| H2N-QDGNEEMGGITQT | | |
| [K/SCBiot(dPEG4)]- | | |
| amide | | |

Both peptides have: Lys/SCBiot(dPEG4); and C-term amide

What is claimed is:

1. An antibody or antigen-binding fragment thereof comprising:
(A) a heavy chain variable domain (VH) polypeptide comprising a set of VH complementarity determining regions: VH (CDR)1 (CDRH1), VH CDR2 (CDRH2), and VH CDR3 (CDRH3); and
(B) a light chain variable domain (VL) polypeptide comprising a set of VL complementarity determining regions: VL CDR1 (CDRL1), VL CDR2 (CDRL2), and VL CDR3 (CDRL3),
wherein at least three, four, five or all six of said CDRs comprises:
(i) the amino acid sequence of a corresponding VH or VL CDR contained in any of the variable domain sequences listed in Table 3A or 3B; and/or
(ii) the amino acid sequence of a corresponding VH or VL CDR selected from any of the VH or VL CDRs listed in Table 4A or 4B,
optionally wherein:
(A) the VH polypeptide comprises a set of VH CDRs: CDRH1, CDRH2, and CDRH3 of a specific antibody selected from those listed in Table 4A; and/or
(B) the VL polypeptide comprises a set of VL CDRs: CDRL1, CDRL2, and CDRL3 of a specific antibody selected from those listed in Table 4B.

2. An antibody or antigen-binding fragment thereof comprising:
(A) a VH polypeptide comprising:
(a) a CDRH1 comprising the amino acid sequence of:
(i) the CDRH1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the CDRH1 contained in any one of SEQ ID NOS: 640, 880, 560, 580, 600, 620, 660, 680, 700, 720, 740, 760, 780, 800, 820, 840, 860, and 900; and/or
(iii) any one of SEQ ID NOS: 642, 882, 562, 582, 602, 622, 662, 682, 702, 722, 742, 762, 782, 802, 822, 842, 862, 902, 922, and 942;
(b) a CDRH2 comprising the amino acid sequence of:
(i) the CDRH2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the CDRH2 contained in any one of SEQ ID NOS: 640, 880, 560, 580, 600, 620, 660, 680, 700, 720, 740, 760, 780, 800, 820, 840, 860, and 900; and/or
(iii) any one of SEQ ID NOS: 644, 884, 564, 584, 604, 624, 664, 684, 704, 724, 744, 764, 784, 804, 824, 844, 864, 904, 924, and 944; and/or
(c) a CDRH3 comprising the amino acid sequence of:
(i) the CDRH3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the CDRH3 contained in any one of SEQ ID NOS: 640, 880, 560, 580, 600, 620, 660, 680, 700, 720, 740, 760, 780, 800, 820, 840, 860, and 900; and/or
(iii) any one of SEQ ID NOS: 646, 886, 566, 586, 606, 626, 666, 686, 706, 726, 926, 746, 766, 786, 806, 826, 846, 866, 906, and 946; and/or
(B) a VL polypeptide comprising:
(a) a CDRL1 comprising the amino acid sequence of:
(i) the CDRL1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the CDRL1 contained in any one of SEQ ID NOS: 650, 890, 570, 590, 610, 630, 670, 690, 710, 730, 750, 770, 790, 810, 830, 850, 870, and 910;
(iii) (iii-1) RASQSX₁SSX₂YLX₃ (SEQ ID NO: 932), wherein X₁ is V or I, X₂ is D, S, or deletion (no amino acid), and X₃ is A or N, or (iii-2) RSSQSLLX₁SNGYNYLD (SEQ ID NO: 952), wherein X₁=H or Y; and/or
(iv) any one of SEQ ID NOS: 652, 892, 572, 592, 612, 632, 672, 692, 712, 732, 752, 772, 792, 812, 832, 852, 872, and 912;
(b) a CDRL2 comprising the amino acid sequence of:
(i) the CDRL2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the CDRL2 contained in any one of SEQ ID NOS: 650, 890, 570, 590, 610, 630, 670, 690, 710, 730, 750, 770, 790, 810, 830, 850, 870, and 910;
(iii) X₁ASX₂X₃X₄X₅ (SEQ ID NO: 934), wherein X₁ is G or A, X₂ is S or N, X₃ is R or L, X₄ is A or Q, and X₅ is T or S; and/or
(iv) any one of SEQ ID NOS: 654, 894, 574, 594, 614, 634, 674, 694, 714, 734, 754, 774, 794, 814, 834, 854, 874, 914, and 954;
(c) a CDRL3 comprising the amino acid sequence of:
(i) the CDRL3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the CDRL3 contained in any one of SEQ ID NOS: 650, 890, 570, 590, 610, 630, 670, 690, 710, 730, 750, 770, 790, 810, 830, 850, 870, and 910;
(iii) (iii-1) X₁QX₂X₃X₄X₅X₆X₇T (SEQ ID NO: 936), wherein X₁ is Q or L, X₂ is F, T, or A, X₃ is G, L, or H, X₄ is R, S, H, P, or T, X₅ is V, D, S, T, Y, F, or A, X₆ is P or L, and X₇ is deletion (no amino acid), I, or W, or (iii-2) MQVX₁X₂X₃PWT (SEQ ID NO: 956), wherein X₁ is L, R, K, or S, X₂ is E, Q, D, or G, and X₃ is L, T, or I; and/or
(iv) any one of SEQ ID NOS: 656, 896, 576, 596, 616, 636, 676, 696, 716, 736, 756, 776, 796, 816, 836, 856, 876, and 916,
or an antibody or antigen-binding fragment thereof which comprises a combination of one or more of the foregoing CDRs.

3. The antibody or antigen-binding fragment thereof of claim 2, comprising:
(A) a VH polypeptide comprising said CDRH1, said CDRH2, and said CDRH3; and/or
(B) a VL polypeptide comprising said CDRL1, said CDRL2, and said CDRL3.

4. The antibody or antigen-binding fragment thereof of claim 2, comprising:
(A) a VH polypeptide comprising said CDRH1, said CDRH2, and said CDRH3; and
(B) a VL polypeptide comprising said CDRL1, said CDRL2, and said CDRL3.

5. The antibody or antigen-binding fragment thereof of any one of claims claim 2-4, comprising:
(I) (i) the CDRL1, the CDRL2, and the CDRL3 contained in ADI-34134; or
(ii) a CDRL1 comprising SEQ ID NO: 652, a CDRL2 comprising SEQ ID NO: 654, and a CDRL3 comprising SEQ ID NO: 656; or
(II) (i) the CDRL1, the CDRL2, and the CDRL3 contained in ADI-34145; or
(ii) a CDRL1 comprising SEQ ID NO: 892, a CDRL2 comprising SEQ ID NO: 894, and a CDRL3 comprising SEQ ID NO: 896.

6. The antibody or antigen-binding fragment thereof of any one of claims claim 2-4, comprising:
(I) (i) the CDRH1, the CDRH2, the CDRH3, the CDRL1, the CDRL2, and the CDRL3 contained in ADI-34134; or
(ii) a CDRH1 comprising SEQ ID NO: 642, a CDRH2 comprising SEQ ID NO: 644, a CDRH3 comprising SEQ ID NO: 646, a CDRL1 comprising SEQ ID NO: 652, a CDRL2 comprising SEQ ID NO: 654, and a CDRL3 comprising SEQ ID NO: 656;
(II) (i) the CDRH1, the CDRH2, the CDRH3, the CDRL1, the CDRL2, and the CDRL3 contained in ADI-34145; or
(ii) a CDRH1 comprising SEQ ID NO: 882, a CDRH2 comprising SEQ ID NO: 884, a CDRH3 comprising SEQ ID NO: 886, a CDRL1 comprising SEQ ID NO: 892, a CDRL2 comprising SEQ ID NO: 894, and a CDRL3 comprising SEQ ID NO: 896;
(III) (i) the CDRH1, the CDRH2, the CDRH3, the CDRL1, the CDRL2, and the CDRL3 contained in ADI-30242; or
(ii) a CDRH1 comprising SEQ ID NO: 562, a CDRH2 comprising SEQ ID NO: 564, a CDRH3 comprising SEQ ID NO: 566, a CDRL1 comprising SEQ ID NO: 572, a CDRL2 comprising SEQ ID NO: 574, and a CDRL3 comprising SEQ ID NO: 576;
(IV) (i) the CDRH1, the CDRH2, the CDRH3, the CDRL1, the CDRL2, and the CDRL3 contained in ADI-34131; or
(ii) a CDRH1 comprising SEQ ID NO: 582, a CDRH2 comprising SEQ ID NO: 584, a CDRH3 comprising SEQ ID NO: 586, a CDRL1 comprising SEQ ID NO: 592, a CDRL2 comprising SEQ ID NO: 594, and a CDRL3 comprising SEQ ID NO: 596;
(V) (i) the CDRH1, the CDRH2, the CDRH3, the CDRL1, the CDRL2, and the CDRL3 contained in ADI-34132; or
(ii) a CDRH1 comprising SEQ ID NO: 602, a CDRH2 comprising SEQ ID NO: 604, a CDRH3 comprising SEQ ID NO: 606, a CDRL1 comprising SEQ ID NO: 612, a CDRL2 comprising SEQ ID NO: 614, and a CDRL3 comprising SEQ ID NO: 616;
(VI) (i) the CDRH1, the CDRH2, the CDRH3, the CDRL1, the CDRL2, and the CDRL3 contained in ADI-34133; or
(ii) a CDRH1 comprising SEQ ID NO: 622, a CDRH2 comprising SEQ ID NO: 624, a CDRH3 comprising SEQ ID NO: 626, a CDRL1 comprising SEQ ID NO: 632, a CDRL2 comprising SEQ ID NO: 634, and a CDRL3 comprising SEQ ID NO: 636;
(VII) (i) the CDRH1, the CDRH2, the CDRH3, the CDRL1, the CDRL2, and the CDRL3 contained in ADI-34135; or
(ii) a CDRH1 comprising SEQ ID NO: 662, a CDRH2 comprising SEQ ID NO: 664, a CDRH3 comprising SEQ ID NO: 666, a CDRL1 comprising SEQ ID NO: 672, a CDRL2 comprising SEQ ID NO: 674, and a CDRL3 comprising SEQ ID NO: 676;
(VIII) (i) the CDRH1, the CDRH2, the CDRH3, the CDRL1, the CDRL2, and the CDRL3 contained in ADI-34136; or
(ii) a CDRH1 comprising SEQ ID NO: 682, a CDRH2 comprising SEQ ID NO: 684, a CDRH3 comprising SEQ ID NO: 686, a CDRL1 comprising SEQ ID NO: 692, a CDRL2 comprising SEQ ID NO: 694, and a CDRL3 comprising SEQ ID NO: 696;
(IX) (i) the CDRH1, the CDRH2, the CDRH3, the CDRL1, the CDRL2, and the CDRL3 contained in ADI-34137; or
(ii) a CDRH1 comprising SEQ ID NO: 702, a CDRH2 comprising SEQ ID NO: 704, a CDRH3 comprising SEQ ID NO: 706, a CDRL1 comprising SEQ ID NO: 712, a CDRL2 comprising SEQ ID NO: 714, and a CDRL3 comprising SEQ ID NO: 716;
(X) (i) the CDRH1, the CDRH2, the CDRH3, the CDRL1, the CDRL2, and the CDRL3 contained in ADI-34138; or
(ii) a CDRH1 comprising SEQ ID NO: 722, a CDRH2 comprising SEQ ID NO: 724, a CDRH3 comprising SEQ ID NO: 726, a CDRL1 comprising SEQ ID NO: 732, a CDRL2 comprising SEQ ID NO: 734, and a CDRL3 comprising SEQ ID NO: 736;
(XI) (i) the CDRH1, the CDRH2, the CDRH3, the CDRL1, the CDRL2, and the CDRL3 contained in ADI-30254; or
(ii) a CDRH1 comprising SEQ ID NO: 742, a CDRH2 comprising SEQ ID NO: 744, a CDRH3 comprising SEQ ID NO: 746, a CDRL1 comprising SEQ ID NO: 752, a CDRL2 comprising SEQ ID NO: 754, and a CDRL3 comprising SEQ ID NO: 756;
(XII) (i) the CDRH1, the CDRH2, the CDRH3, the CDRL1, the CDRL2, and the CDRL3 contained in ADI-34139; or
(ii) a CDRH1 comprising SEQ ID NO: 762, a CDRH2 comprising SEQ ID NO: 764, a CDRH3 comprising SEQ ID NO: 766, a CDRL1 comprising SEQ ID NO: 772, a CDRL2 comprising SEQ ID NO: 774, and a CDRL3 comprising SEQ ID NO: 776;
(XIII) (i) the CDRH1, the CDRH2, the CDRH3, the CDRL1, the CDRL2, and the CDRL3 contained in ADI-34140; or
(ii) a CDRH1 comprising SEQ ID NO: 782, a CDRH2 comprising SEQ ID NO: 784, a CDRH3 comprising SEQ ID NO: 786, a CDRL1 comprising SEQ ID NO: 792, a CDRL2 comprising SEQ ID NO: 794, and a CDRL3 comprising SEQ ID NO: 796;
(XIV) (i) the CDRH1, the CDRH2, the CDRH3, the CDRL1, the CDRL2, and the CDRL3 contained in ADI-34141; or
(ii) a CDRH1 comprising SEQ ID NO: 802, a CDRH2 comprising SEQ ID NO: 804, a CDRH3 comprising SEQ ID NO: 806, a CDRL1 comprising SEQ ID NO: 812, a CDRL2 comprising SEQ ID NO: 814, and a CDRL3 comprising SEQ ID NO: 816;
(XV) (i) the CDRH1, the CDRH2, the CDRH3, the CDRL1, the CDRL2, and the CDRL3 contained in ADI-34142; or
(ii) a CDRH1 comprising SEQ ID NO: 822, a CDRH2 comprising SEQ ID NO: 824, a CDRH3 comprising SEQ ID NO: 826, a CDRL1 comprising SEQ ID NO: 832, a CDRL2 comprising SEQ ID NO: 834, and a CDRL3 comprising SEQ ID NO: 836;
(XVI) (i) the CDRH1, the CDRH2, the CDRH3, the CDRL1, the CDRL2, and the CDRL3 contained in ADI-34143; or
(ii) a CDRH1 comprising SEQ ID NO: 842, a CDRH2 comprising SEQ ID NO: 844, a CDRH3 comprising SEQ ID NO: 846, a CDRL1 comprising SEQ ID NO: 852, a CDRL2 comprising SEQ ID NO: 854, and a CDRL3 comprising SEQ ID NO: 856;
(XVII) (i) the CDRH1, the CDRH2, the CDRH3, the CDRL1, the CDRL2, and the CDRL3 contained in ADI-34144; or
(ii) a CDRH1 comprising SEQ ID NO: 862, a CDRH2 comprising SEQ ID NO: 864, a CDRH3 comprising SEQ ID NO: 866, a CDRL1 comprising SEQ ID NO: 872, a CDRL2 comprising SEQ ID NO: 874, and a CDRL3 comprising SEQ ID NO: 876;
(XVIII) (i) the CDRH1, the CDRH2, the CDRH3, the CDRL1, the CDRL2, and the CDRL3 contained in ADI-34146; or
(ii) a CDRH1 comprising SEQ ID NO: 902, a CDRH2 comprising SEQ ID NO: 904, a CDRH3 comprising SEQ ID NO: 906, a CDRL1 comprising SEQ ID NO: 912, a CDRL2 comprising SEQ ID NO: 914, and a CDRL3 comprising SEQ ID NO: 916.

7. The antibody or antigen-binding fragment thereof of any one of claims claim 2-6, wherein:
(A) the VH polypeptide comprises:
(a) a VH framework region 1 (FRH1) comprising the amino acid sequence of:
(i) the FRH1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRH1 contained in any one of SEQ ID NOS: 640, 880, 560, 580, 600, 620, 660, 680, 700, 720, 740, 760, 780, 800, 820, 840, 860, and 900; and/or
(iii) any one of SEQ ID NOS: 641, 881, 561, 581, 601, 621, 661, 681, 701, 721, 741, 761, 781, 801, 821, 841, 861, 901, 921, and 941;
(b) a VH framework region 2 (FRH2) comprising the amino acid sequence of:
(i) the FRH2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRH2 contained in any one of SEQ ID NOS: 640, 880, 560, 580, 600, 620, 660, 680, 700, 720, 740, 760, 780, 800, 820, 840, 860, and 900; and/or
(iii) any one of SEQ ID NOS: 643, 883, 563, 583, 603, 623, 663, 683, 703, 723, 743, 763, 783, 803, 823, 843, 863, 903, 923, and 943;
(c) a VH framework region 3 (FRH3) comprising the amino acid sequence of:
(i) the FRH3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRH3 contained in any one of SEQ ID NOS: 640, 880, 560, 580, 600, 620, 660, 680, 700, 720, 740, 760, 780, 800, 820, 840, 860, and 900; and/or
(iii) any one of SEQ ID NOS: 645, 885, 565, 585, 605, 625, 665, 685, 705, 725, 745, 765, 785, 805, 825, 845, 865, 905, 925, and 945; and/or
(d) a VH framework region 4 (FRH4) comprising the amino acid sequence of:
(i) the FRH4 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRH4 contained in any one of SEQ ID NOS: 640, 880, 560, 580, 600, 620, 660, 680, 700, 720, 740, 760, 780, 800, 820, 840, 860, and 900; and/or
(iii) any one of SEQ ID NOS: 647, 887, 567, 587, 607, 627, 667, 687, 707, 727, 747, 767, 787, 807, 827, 847, 867, 907, 927, and 947; and/or
(B) the VL polypeptide comprises:
(a) a VL framework region 1 (FRL1) comprising the amino acid sequence of:
(i) the FRL1 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRL1 contained in any one of SEQ ID NOS: 650, 890, 570, 590, 610, 630, 670, 690, 710, 730, 750, 770, 790, 810, 830, 850, 870, and 910;
(iii) X₁IX₂X₃TQSPX₄X₅LSX₆SX₇GX₅RX₉TX₁₀X₁₁C (SEQ ID NO: 931), wherein X₁ is E or D, X₂ is V or Q, X₃ is L or M, X₄ is G or S, X₅ is T or S, X₆ is L or A, X₇ is P or V, X₅ is E or D, X₉ is A or V, X₁₀ is L or I, and X₁₁ is S or T; and/or
(iv) any one of SEQ ID NOS: 651, 891, 571, 591, 611, 631, 671, 691, 711, 731, 751, 771, 791, 811, 831, 851, 871, 911, and 951;
(b) a VL framework region 2 (FRL2) comprising the amino acid sequence of:
(i) the FRL2 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRL2 contained in any one of SEQ ID NOS: 650, 890, 570, 590, 610, 630, 670, 690, 710, 730, 750, 770, 790, 810, 830, 850, 870, and 910;
(iii) WYQQKPGX₁APX₂LLIY (SEQ ID NO: 933), wherein X₁ is Q or K, and X₂ is R or K; and/or
(iv) any one of SEQ ID NOS: 653, 893, 573, 593, 613, 633, 673, 693, 713, 733, 753, 773, 793, 813, 833, 853, 873, 913, and 953;
(c) a VL framework region 3 (FRL3) comprising the amino acid sequence of:
(i) the FRL3 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRL3 contained in any one of SEQ ID NOS: 650, 890, 570, 590, 610, 630, 670, 690, 710, 730, 750, 770, 790, 810, 830, 850, 870, and 910;
(iii) GX₁PX₂RFSGSGSGTDFTLTISX₃LX₄PEDFAX5YYC (SEQ ID NO: 935), wherein X₁ is I or V, X₂ is D or S, X₃ is R or S, X₄ is E or Q, and X₅ is V or T; and/or
(iv) any one of SEQ ID NOS: 655, 895, 575, 595, 615, 635, 675, 695, 715, 735, 755, 775, 795, 815, 835, 855, 875, 915, and 955; and/or
(d) a VL framework region 4 (FRL4) comprising the amino acid sequence of:
(i) the FRL4 contained in any one of ADI-34134, ADI-34145, ADI-30242, ADI-34131, ADI-34132, ADI-34133, ADI-34135, ADI-34136, ADI-34137, ADI-34138, ADI-30254, ADI-34139, ADI-34140, ADI-34141, ADI-34142, ADI-34143, ADI-34144, and ADI-34146;
(ii) the FRL4 contained in any one of SEQ ID NOS: 650, 890, 570, 590, 610, 630, 670, 690, 710, 730, 750, 770, 790, 810, 830, 850, 870, and 910; and/or
(iii) any one of SEQ ID NOS: 657, 897, 577, 597, 617, 637, 677, 697, 717, 737, 757, 777, 797, 817, 837, 857, 877, 917, 937, and 957,
or the antibody or antigen-binding fragment thereof comprising a VH and/or a VL that comprises any combination of the foregoing VH and VL framework regions.

8. The antibody or antigen-binding fragment thereof of any one of claims claim 2-7, comprising:
(I) (i) the FRH1, the FRH2, the FRH3, the FLRH4, the FRL1, the FRL2, the FRL3, and the FRL4 contained in any one of ADI-34134, ADI-30242, ADI-34131, ADI-34133, ADI-34136, and ADI-34137; and/or
(ii) a FRH1 comprising any one of SEQ ID NOS: 641, 561, 581, 621, 681, and 701, a FRH2 comprising any one of SEQ ID NOS: 643, 563, 583, 623, 683, and 703, a FRH3 comprising any one of SEQ ID NOS: 645, 565, 585, 625, 685, and 705, a FRH4 comprising any one of SEQ ID NOS: 647, 567, 587, 627, 687, and 707, a FRL1 comprising any one of SEQ ID NOS: 651, 571, 591, 631, 691, and 711, a FRL2 comprising any one of SEQ ID NOS: 653, 573, 593, 633, 693, and 713, a FRL3 comprising any one of SEQ ID NOS: 655, 575, 595, 635, 695, and 715, and a FRL4 comprising any one of SEQ ID NOS: 657, 577, 597, 637, 697, and 717;
(II) (i) the FRH1, the FRH2, the FRH3, the FLRH4, the FRL1, the FRL2, the FRL3, and the FRL4 contained in any one of ADI-34145, ADI-30254, ADI-34139, ADI-34140, ADI-34141, ADI-34142, ADI-34143, ADI-34144, and ADI-34146; and/or
(ii) a FRH1 comprising any one of SEQ ID NOS: 881, 741, 761, 781, 801, 821, 841, 861, and 901, a FRH2 comprising any one of SEQ ID NOS: 883, 743, 763, 783, 803, 823, 843, 863, and 903, a FRH3 comprising any one of SEQ ID NOS: 885, 745, 765, 785, 805, 825, 845, 865, and 905, a FRH4 comprising any one of SEQ ID NOS: 887, 747, 767, 787, 807, 827, 847, 867, and 907, a FRL1 comprising any one of SEQ ID NOS: 891, 751, 771, 791, 811, 831, 851, 871, and 911, a FRL2 comprising any one of SEQ ID NOS: 893, 753, 773, 793, 813, 833, 853, 873, and 913, a FRL3 comprising any one of SEQ ID NOS: 895, 755, 775, 795, 815, 835, 855, 875, and 915, and a FRL4 comprising any one of SEQ ID NOS: 897, 757, 777, 797, 817, 837, 857, 877, and 917;
(III) (i) the FRH1, the FRH2, the FRH3, the FLRH4, the FRL1, the FRL2, the FRL3, and the FRL4 contained in ADI-34135 or ADI-34138; and/or
(ii) a FRH1 comprising SEQ ID NO: 661 or 721, a FRH2 comprising SEQ ID NO: 663 or 723, a FRH3 comprising SEQ ID NO: 665 or 725, a FRH4 comprising SEQ ID NO: 667 or 727, a FRL1 comprising SEQ ID NO: 671 or 731, a FRL2 comprising SEQ ID NO: 673 or 733, a FRL3 comprising SEQ ID NO: 675 or 735, and a FRL4 comprising SEQ ID NO: 677 or 737; or
(IV) (i) the FRH1, the FRH2, the FRH3, the FLRH4, the FRL1, the FRL2, the FRL3, and the FRL4 contained in ADI-34132; and/or
(ii) a FRH1 comprising SEQ ID NO: 601, a FRH2 comprising SEQ ID NO: 603, a FRH3 comprising SEQ ID NO: 605, a FRH4 comprising SEQ ID NO: 607, a FRL1 comprising SEQ ID NO: 611, a FRL2 comprising SEQ ID NO: 613, a FRL3 comprising SEQ ID NO: 615, and a FRL4 comprising SEQ ID NO: 617.

9. The antibody or antigen-binding fragment thereof of any one of claims claim 2-8, comprising:
(I) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34134; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 641, 642, 643, 644, 645, 646, 647, 651, 652, 653, 654, 655, 656, and 657, respectively;
(II) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34145; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 881, 882, 883, 884, 885, 886, 887, 891, 892, 893, 894, 895, 896, and 897, respectively;
(III) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-30242; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 561, 562, 563, 564, 565, 566, 567, 571, 572, 573, 574, 575, 576, and 577, respectively;
(IV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34131; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 581, 582, 583, 584, 585, 586, 587, 591, 592, 593, 594, 595, 596, and 597, respectively;
(V) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34132; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 601, 602, 603, 604, 605, 606, 607, 611, 612, 613, 614, 615, 616, and 617, respectively;
(VI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34133; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 621, 622, 623, 624, 625, 626, 627, 631, 632, 633, 634, 635, 636, and 637, respectively;
(VII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34135; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 661, 662, 663, 664, 665, 666, 667, 671, 672, 673, 674, 675, 676, and 677, respectively;
(VIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34136; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 681, 682, 683, 684, 685, 686, 687, 691, 692, 693, 694, 695, 696, and 697, respectively;
(IX) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34137; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 701, 702, 703, 704, 705, 706, 707, 711, 712, 713, 714, 715, 716, and 717, respectively;
(X) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34138; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 721, 722, 723, 724, 725, 726, 727, 731, 732, 733, 734, 735, 736, and 737, respectively;
(XI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-30254; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 741, 742, 743, 744, 745, 746, 747, 751, 752, 753, 754, 755, 756, and 757, respectively;
(XII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34139; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 761, 762, 763, 764, 765, 766, 767, 771, 772, 773, 774, 775, 776, and 777, respectively;
(XIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34140; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 781, 782, 783, 784, 785, 786, 787, 791, 792, 793, 794, 795, 796, and 797, respectively;
(XIV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34141; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 801, 802, 803, 804, 805, 806, 807, 811, 812, 813, 814, 815, 816, and 817, respectively;
(XV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34142; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 821, 822, 823, 824, 825, 826, 827, 831, 832, 833, 834, 835, 836, and 837, respectively;
(XVI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34143; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 841, 842, 843, 844, 845, 846, 847, 851, 852, 853, 854, 855, 856, and 857, respectively;
(XVII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34144; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 861, 862, 863, 864, 865, 866, 867, 871, 872, 873, 874, 875, 876, and 877, respectively;
(XVIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in ADI-34146; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 901, 902, 903, 904, 905, 906, 907, 911, 912, 913, 914, 915, 916, and 917, respectively.

10. The antibody or antigen-binding fragment thereof of claim 6, wherein:
in (I), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 640; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 650;
in (II), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 880; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 890;
in (III), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 560; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 570;
in (IV), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 580; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 590;
in (V), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 600; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 610;
in (VI), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 620; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 630;
in (VII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 660; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 670;
in (VIII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 680; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 690;
in (IX), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 700; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 710;
in (X), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 720; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 730;
in (XI), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 740; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 750;
in (XII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 760; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 770;
in (XIII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 780; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 790;
in (XIV), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 800; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 810;
in (XV), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 820; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 830;
in (XVI), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 840; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 850;
in (XVII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 860; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 870;
in (XVIII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 900; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 910.

11. The antibody or antigen-binding fragment thereof of any one of claims 2-10 wherein the VH polypeptide and the VL polypeptide comprise the amino acid sequences of:
(I) SEQ ID NOS: 640 and 650, respectively;
(II) SEQ ID NOS: 880 and 890, respectively;
(III) SEQ ID NOS: 560 and 570, respectively;
(IV) SEQ ID NOS: 580 and 590, respectively;
(V) SEQ ID NOS: 600 and 610, respectively;
(VI) SEQ ID NOS: 620 and 630, respectively;
(VII) SEQ ID NOS: 660 and 670, respectively;
(VIII) SEQ ID NOS: 680 and 690, respectively;
(IX) SEQ ID NOS: 700 and 710, respectively;
(X) SEQ ID NOS: 720 and 730, respectively;
(XI) SEQ ID NOS: 740 and 750, respectively;
(XII) SEQ ID NOS: 760 and 770, respectively;
(XIII) SEQ ID NOS: 780 and 790, respectively;
(XIV) SEQ ID NOS: 800 and 810, respectively;
(XV) SEQ ID NOS: 820 and 830, respectively;
(XVI) SEQ ID NOS: 840 and 850, respectively;
(XVII) SEQ ID NOS: 860 and 870, respectively; or
(XVIII) SEQ ID NOS: 900 and 910, respectively.

12. The antibody or antigen-binding fragment thereof of any one of claims 1-11, which comprises any one or more of the following:
(i) one or more antibody constant regions, a CH1 domain, a hinge, a CH2 domain, and/or a CH3 domain, which optionally is/are individually of, or derived from an IgG or human IgG, further optionally of or derived from a human IgG1, IgG4, IgG2, or IgG3;
(ii) a fragment crystallizable (Fc) region, optionally of, or derived from:
(1) a human IgG1, further optionally comprising one or more of the following amino acid modifications: N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, or any combination thereof, according to EU numbering;
(2) a human IgG4, further optionally comprising one or more of the following amino acid modifications: E233P, F234V, L235A, G237A, E318A, S228P, L236E, S241P, L248E, T394D, M252Y, S254T, T256E, N297A, N297Q, or any combination thereof, according to EU numbering;
(3) a human IgG2, further optionally comprising one or more of the following amino acid modifications: P238S, V234A, G237A, H268A, H268Q, H268E, V309L, N297A, N297Q, A330S, P331S, C232S, C233S, M252Y, S254T, T256E, or any combination thereof, according to EU numbering; and/or
(4) a human IgG3, further optionally comprising E235Y, according to EU numbering;
(iii) an IgG, IgA, IgE, IgD, or IgM, optionally IgG1, IgG4, IgG2, or IgG3; and/or
(iv) an antibody fragment selected from the group consisting of: a fragment antigen-binding (Fab); an Fab₂; an Fab₃; an Fab′ fragment; an F(ab′)₂; a variable fragment (Fv); a single-chain Fv (scFv) fragment; a diabody; a triabody; a minibody; a scFv-Fc; a scFv2-Fc2; scFv-IgG; and/or a monovalent IgG (or a half IgG).

13. The antibody or antigen-binding fragment thereof of any one of claims 1-12, wherein the antibody or antigen-binding fragment thereof binds to a target antibody, optionally wherein the target antibody:
(i) comprises an antigen-binding domain that binds Cluster of Differentiation 3 (CD3); and/or
(ii) is or comprises (ii-1) an IgG, optionally IgG1, or (ii-2) a Fab, further optionally wherein the antibody or antigen-binding fragment thereof:
(I) binds to the target antibody with an equilibrium dissociation constant (Kd) value of about 1.0×10⁶ (M) or lower, about 5.0×10⁷ (M) or lower, about 10×10⁷ (M) or lower, about 5.0×10⁸ (M) or lower, about 1.0×10⁸ (M) or lower, about 9.0×10⁹ (M) or lower, about 8.0×10⁹ (M) or lower, about 7.0×10⁹ (M) or lower, about 6.0×10⁹ (M) or lower, about 5.0×10⁹ (M) or lower, about 4.0×10⁹ (M) or lower, about 3.0×10⁹ (M) or lower, about 2.0×10⁹ (M) or lower, about 1.0×10⁹ (M) or lower, about 9.0×10¹⁰ (M) or lower, about 8.0×10¹⁰ (M) or lower, about 7.0×10¹⁰ (M) or lower, about 6.0×10¹⁰ (M) or lower, about 5.0×10¹⁰ (M) or lower, about 4.0×10¹⁰ (M) or lower, about 3.0×10¹⁰ (M) or lower, about 2.0×10¹⁰ (M) or lower, about 1.0×10¹⁰ (M) or lower, about 9.0×10¹¹ (M) or lower, about 8.0×10¹¹ (M) or lower, about 7.0×10¹¹ (M) or lower, about 6.0×10¹¹ (M) or lower, about 5.0×10¹¹ (M) or lower, or about 4.0×10¹¹ (M) or lower, optionally measured via bio-layer interferometry (BLI), optionally using an OCTET® system or via surface plasmon resonance (SPR), optionally using a CARTERRA® system;
(II) exhibits a PSR score lower than 0.33, lower than about 0.30, lower than about 0.20, lower than about 0.15, lower than about 0.12, lower than about 0.10, lower than about 0.08, lower than about 0.06, lower than about 0.05, lower than about 0.04, lower than about 0.03, lower than about 0.02, lower than about 0.01, or of about 0.00; and/or a PSR score of about >0.10 and <0.33 or a PSR score of about <0.10; and/or
(III) exhibits a hydrophobic interaction chromatography (HIC) retention time of <about 10.5 minutes, <about 10.0 minutes, or <about 9.5 minutes.

14. The antibody or antigen-binding fragment thereof of claim 13, wherein the target antibody comprises:
(A) a VH polypeptide comprising:
(a) a FRH1 comprising the amino acid sequence of:
(i) the FRH1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRH1 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530;
(b) a CDRH1 comprising the amino acid sequence of:
(i) the CDRH1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240;
(ii) the CDRH1 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; and/or
(iii) any one of SEQ ID NOS: 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, and 531;
(c) a FRH2 comprising the amino acid sequence of:
(i) the FRH2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRH2 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530;
(d) a CDRH2 comprising the amino acid sequence of:
(i) the CDRH2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240;
(ii) the CDRH2 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; and/or
(iii) any one of SEQ ID NOS: 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, and 532;
(e) a FRH3 comprising the amino acid sequence of:
(i) the FRH3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRH3 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530;
(f) a CDRH3 comprising the amino acid sequence of:
(i) the CDRH3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240;
(ii) the CDRH3 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; and/or
(iii) any one of SEQ ID NOS: 183, 193, 203, 213, 223, 233, 243, 253, 263, 273, 283, 293, 303, 313, 323, 333, 343, 353, 363, 373, 383, 393, 403, 413, 423, 433, 443, 453, 463, 473, 483, 493, 503, 513, 523, and 533; and/or
(g) a FRH4 comprising the amino acid sequence of:
(i) the FRH4 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRH4 contained in any one of SEQ ID NOS: 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530; and/or
(B) a VL polypeptide comprising:
(a) a FRL1 comprising the amino acid sequence of:
(i) the FRL1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRL1 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535;
(b) a CDRL1 comprising the amino acid sequence of:
(i) the CDRL1 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240;
(ii) the CDRL1 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; and/or
(iii) any one of SEQ ID NOS: 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, and 536;
(c) a FRL2 comprising the amino acid sequence of:
(i) the FRL2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRL2 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535;
(d) a CDRL2 comprising the amino acid sequence of:
(i) the CDRL2 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240;
(ii) the CDRL2 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435,445, 455, 465, 475, 485, 495, 505, 515, 525, and 535; and/or
(iii) any one of SEQ ID NOS: 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, and 537;
(e) a FRL3 comprising the amino acid sequence of:
(i) the FRL3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRL3 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535;
(f) a CDRL3 comprising the amino acid sequence of:
(i) the CDRL3 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240;
(ii) the CDRL3 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, and 535; and/or
(iii) any one of SEQ ID NOS: 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, 528, and 538; and/or
(g) a FRL4 comprising the amino acid sequence of:
(i) the FRL4 contained in any one of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240; and/or
(ii) the FRL4 contained in any one of SEQ ID NOS: 185, 195, 205, 215, 225, 235, 245, 255, 265, 275, 285, 295, 305, 315, 325, 335, 345, 355, 365, 375, 385, 395, 405, 415, 425, 435,445, 455, 465, 475, 485, 495, 505, 515, 525, and 535.

15. The antibody or antigen-binding fragment thereof of claim 13, wherein the target antibody comprises:
(A) a VH polypeptide comprising a set of a CDRH1, a CDRH2, and a CDRH3 according to any of those listed in Table 2A; and/or
(B) a VL polypeptide comprising a set of a CDRL1, a CDRL2, and a CDRL3 according to any of those listed in Table 2B.

16. The antibody or antigen-binding fragment thereof of any one of claims 13-15, wherein the target antibody comprises:
(A) a VH polypeptide comprising an amino acid sequence at least 80%, at least 85%, at least 90%, at least 92%, at least 94%, at least 95%, at least 96%, at least 97%, at least 98%, at least 99%, or 100% identical to any of those listed in Table 1A; and/or
(B) a VL polypeptide comprising an amino acid sequence at least 80%, at least 85%, at least 90%, at least 92%, at least 94%, at least 95%, at least 96%, at least 97%, at least 98%, at least 99%, or 100% identical to any of those listed in Table 1B.

17. The antibody or antigen-binding fragment thereof of any one of claims 13-16, wherein the target antibody comprises a VH polypeptide and a VL polypeptide comprising the amino acid sequences of: 110 and 115, respectively; 120 and 125, respectively; 130 and 135, respectively; 140 and 145, respectively; 150 and 155, respectively; 160 and 165, respectively; 170 and 175, respectively; 180 and 185, respectively; 190 and 195, respectively; 200 and 205, respectively; 210 and 215, respectively; 220 and 225, respectively; 230 and 235, respectively; 240 and 245, respectively; 250 and 255, respectively; 260 and 265, respectively; 270 and 275, respectively; 280 and 285, respectively; 290 and 295, respectively; 300 and 305, respectively; 310 and 315, respectively; 320 and 325, respectively; 330 and 335, respectively; 340 and 345, respectively; 350 and 355, respectively; 360 and 365, respectively; 370 and 375, respectively; 380 and 385, respectively; 390 and 395, respectively; 400 and 405; respectively, 410 and 415, respectively; 420 and 425, respectively; 430 and 435, respectively; 440 and 445, respectively; 450 and 455, respectively; 460 and 465, respectively; 470 and 475, respectively; 480 and 485, respectively; 490 and 495, respectively; 500 and 505, respectively; 510 and 515, respectively; 520 and 525, respectively; 530 and 535, respectively, optionally wherein the target antibody comprises any one or more of ADI-22523, ADI-26927, ADI-26928, ADI-26941, ADI-26954, CTL-19613, CTL-19672, ADI-50024, ADI-70326, ADI-70327, ADI-70330, ADI-26906, ADI-67450, ADI-67445, ADI-67447, ADI-26909, ADI-26913, ADI-26919, ADI-26920, ADI-26921, ADI-26916, ADI-26912, ADI-26924, ADI-26915, ADI-26943, ADI-48576, ADI-48587, ADI-48592, ADI-48650, ADI-ADI-74965, ADI-74966, ADI-74967, ADI-74968, ADI-76357, ADI-76358, ADI-76359, ADI-76360, ADI-76361, ADI-76362, CTL-62223, CTL-62227, CTL-62228, and CTL-62240 or an antigen-binding fragment thereof.

18. The antibody or antigen-binding fragment thereof of any one of claims 13-17, wherein the target antibody comprises or is comprised in a multispecific antibody or antibody fragment comprising at least:
(a) a first antigen-binding region that binds to CD3; and
(b) a second antigen-binding region that binds to a second antigen, optionally wherein the multispecific antibody or antibody fragment is a bispecific antibody or antibody fragment.

19. The antibody or antigen-binding fragment thereof of claim 18, wherein the second antigen is or comprises one or more of: an oncology target; a target molecule expressed on cancer cells; an immune-oncology target; a target molecule expressed on immune cells; a neurodegenerative disease target; an autoimmune disorder target (optionally a self-reactive immune molecule or a target molecule expressed on an immune cell expressing a self-reactive immune molecule); an inflammatory disease target (optionally an inflammatory cytokine or chemokine or a receptor thereof); an infectious disease target (optionally a target molecule of a virus, bacterium, or a fungus); a target molecule expressed on infected cells (optionally infected with a virus, a bacterium, or fungus); a metabolic disease target; a cognitive disorder target; a blood-brain barrier target; and/or a blood disease target.

20. The antibody or antigen-binding fragment thereof of claim 18, wherein the second antigen is or comprises one or more of: 17-IA, 4-1BB, 4Dc, 6-keto-PGFla, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-l-antitrypsin, alpha-V/beta-1 antagonist, ANG, Ang, APAF-1, APE, APJ, APP, APRIL, AR, ARC, ART, Artemin, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen (CCA), Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD3ε, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor Ila, Factor VII, Factor VIIIc, Factor IX, fibroblast activation protein (FAP), Fas, FcRl, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alphal, GFR-alpha2, GFR-alpha3, GITR, Glucagon, Glut 4, glycoprotein Ilb/IIIa (GP Ilb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309 antigen, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCK-2, MCP, M-CSF, MDC, Mer, metalloproteases, MGDF receptor, MGMT, MHC (HLA-DR), MIF, MIG, MIP, MIP-l-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian inhibiting substance, Mug, MuSK, NAIP, NAP, NCAD, N-Cadherin, NCA 90, NCAM, NCAM, Neprilysin, Neurotrophin-3,-4, or-6, Neurturin, Neuronal growth factor (NGF), NGFR, NGF-beta, nNOS, NO, NOS, Npn, NRG-3, NT, NTN, OB, OGGI, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PlGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta Rllb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF1OA (TRAIL R1 Apo-2, DR4), TNFRSFIOB (TRAIL R2 DR5, KILLER, TRICK-2A, TRICK-B), TNFRSF1OC (TRAIL R3 DcRl, LIT, TRID), TNFRSF1OD (TRAIL R4 DcR2, TRUNDD), TNFRSF11 A (RANK ODF R, TRANCE R), TNFRSFllB (OPG OCIF, TRI), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-lBB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF1O (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSFIA (TNF-a Conectin, DIF, TNFSF2), TNFSFlB (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD3β Ligand CD153), TNFSF9 (4-lBB Ligand CD137 Ligand), TP-1, t-PA, Tpo, TRAIL, TRAIL R, TRAIL-R1, TRAIL-R2, TRANCE, transferrin receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (fit-4), VEGI, VIM, Viral antigens, VLA, VLA-1, VLA-4, VNR integrin, von Willebrand's factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCRI, XCRI, XEDAR, XIAP, XPD, CTLA4 (cytotoxic T lymphocyte antigen-4), PD-1 (programmed cell death protein 1), PD-L1 (programmed cell death ligand 1), LAG-3 (lymphocyte activation gene-3), TIM-3 (T cell immunoglobulin and mucin protein-3), a hormone receptor, and a growth factor.

21. The antibody or antigen-binding fragment thereof of claim 18, wherein the second antigen is or comprises one or more of: BCMA, CTLA4 (cytotoxic T lymphocyte antigen-4), PD-1 (programmed cell death protein 1), PD-L1 (programmed cell death ligand 1), LAG-3 (lymphocyte activation gene-3), TIM-3, CD20, CD2, CD19, Her2, EGFR, EpCAM, FcγRIIIa (CD16), FcγRIIa (CD32a), FcγRIIb (CD32b), FcγRI (CD64), Toll-like receptors (TLRs), TLR4, TLR9, cytokines, IL-2, IL-5, IL-13, IL-6, IL-17, IL-12, IL-23, TNFα, TGFO, a cytokine receptor, IL-2R, a chemokine, a chemokine receptor, a growth factor, VEGF, HGF, and a hormone receptor.

22. The antibody or antigen-binding fragment thereof of any one of claims 18-21, wherein the multispecific antibody or antibody fragment comprises a multispecific format according to one or more of a Fab-Fc-scFv, scFv2-Fc2, scFv-IgG “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

23. The antibody or antigen-binding fragment thereof of any one of claims 13-22, wherein the target antibody comprises:
(i) a chimeric antigen receptor (CAR), optionally wherein the CAR comprises a transmembrane domain, an intracellular domain from a T-cell receptor, a CD3 subunit, and/or a co-stimulatory domain;
(ii) an scFv, an scFv2-Fc2, and/or scFv-IgG; and/or
(iii) an IgG constant domain.

24. A nucleic acid encoding an antibody or antigen-binding fragment thereof according to any one of claims 1-23.

25. A vector comprising the nucleic acid according to claim 24.

26. A cell comprising the nucleic acid according to claim 24 and/or a vector according to claim 25, wherein the cell is optionally a mammalian cell or a yeast cell.

27. A composition comprising:
(a) an excipient; and
(b) one or more of:
(i) the antibody or antigen-binding fragment thereof according to any one of claims 1-23;
(ii) the nucleic acid according to claim 24;
(iii) the vector according to claim 25; and/or
(iv) the cell according to claim 26.

28. A pharmaceutical composition comprising:
(a) a pharmaceutically acceptable excipient; and
(b) one or more of:
(i) the antibody or antigen-binding fragment thereof according to any one of claims 1-23;
(ii) the nucleic acid according to claim 24;
(iii) the vector according to claim 25; and
(iv) the cell according to claim 26.

29. A method of treating a subject in need of such treatment, the method comprising administering the antibody or antigen-binding fragment thereof of any one of claims 1-23 or the composition according to claim 27 or 28.

30. The method of claim 29, wherein the subject comprises a target antibody, wherein the target antibody comprises an antigen-binding region that binds CD3 (anti-CD3 antibody), and wherein administering the antibody or antigen-binding fragment thereof partially or completely neutralizes the target anti-CD3 antibody and/or inhibits or prevents the binding of the target anti-CD3 antibody to CD3 or CD3-expressing cells.

31. The method of claim 29, wherein the antibody or antigen-binding fragment thereof is administered in combination with a target antibody, wherein the target antibody comprises an antigen-binding region that binds CD3.

32. The method of claim 31, wherein the antibody or antigen-binding fragment thereof is conjugated with the target antibody, optionally wherein the antibody or antigen-binding fragment thereof is conjugated with the target antibody via a linker, optionally a cleavable linker.

33. The method of claim 31 or 32, wherein:
(i) the antibody or antigen-binding fragment thereof reduces an effect of the target antibody in at least one cell or tissue;
(ii) the target antibody is administered to treat a target cell or target tissue, wherein the antibody or antigen-binding fragment thereof reduces an effect of the target antibody in a non-target cell or non-target tissue; and/or
(iii) the antibody or antigen-binding fragment thereof is used as a masking agent of the target antibody.

34. The method of any one of claims 31-33, wherein the antibody or antigen-binding fragment thereof is bound to the target antibody prior to administration, optionally wherein the antibody or antigen-binding fragment thereof dissociates from the target antibody after administration, further optionally wherein dissociation of the antibody or antigen-binding fragment thereof from the target antibody is induced by one or more of change in pH, association with an inhibitor, presence of a competitive inhibitor, and presence of a protease.

35. The method of any one of claims 29-34, wherein the antibody or antigen-binding fragment thereof is administered to the subject to treat a disorder, optionally wherein the disorder:
(i) is related to a prior treatment of the subject with a/the target antibody, wherein the target antibody comprises an antigen-binding domain that binds CD3;
(ii) comprises an inflammatory disorder; and/or
(iii) comprises cytokine release syndrome.

36. The method of any one of claims 29-35, comprising administering an additional therapeutic agent.

37. The method of any one of claims 29-36, wherein the subject is a mammal, wherein the mammal is optionally a human.

38. A method of isolating a target antibody, the method comprising contacting the target antibody with the antibody or antigen-binding fragment thereof of any one of claims 1-23.

39. A method of detecting a target antibody, the method comprising contacting the target antibody with the antibody or antigen-binding fragment thereof of any one of claims 1-23, optionally wherein the contacting occurs (i) in vitro, further optionally wherein the target antibody is contained in or derived from a biological sample, further optionally from a subject, or (ii) in vivo, optionally wherein the method comprises administering the antibody or antigen-binding fragment thereof to a subject, optionally a subject administered with or comprising the target antibody.

40. A method of characterizing a target antibody, wherein the target antibody comprises an antigen-binding domain that binds CD3, the method comprising the use of an antibody or antigen-binding fragment thereof according to any one of claims 1-23, optionally wherein the antibody or antigen-binding fragment thereof is used to characterize a pharmacokinetic and/or pharmacodynamic property of or associated with the target antibody.

41. A method of determining the presence or absence of an anti-drug antibody in a sample, the method comprising the use of an antibody or antigen-binding fragment thereof according to any one of claims 1-23, optionally wherein the method is for determining the presence or absence of an anti-drug antibody which competes with said antibody or antigen-binding fragment thereof for binding to the target antibody.

42. A method of making the antibody or antigen-binding fragment thereof according to any one of claims 1-23, comprising:
(a) culturing cells comprising the nucleic acid of claim 24 in a condition that allows for expression of said antibody or antigen-binding fragment, and
(b) harvesting and/or purifying the antibody or antigen-binding fragment from the cell culture from (a).

43. A method of manufacturing the cell of claim 26 or a population of such cells, comprising introducing the nucleic acid of claim 24 and/or the vector of claim 25 into one or more cells, optionally wherein the introducing occurs in vitro, ex vivo, or in vivo.

44. The antibody or antigen-binding fragment thereof according to any one of claims 1-23, for use in the method according to any one of claims 29-43.

45. The antibody or antigen-binding fragment thereof according to any one of claims 1-23, for use in the manufacture of a medicament, optionally wherein the medicament is for a treatment method according to any one of claims 29-37.

46. Use of the antibody or antigen-binding fragment thereof according to any one of claims 1-23 in the method according to any one of claims 29-43.

47. A kit comprising:
(a) the antibody or antigen-binding fragment thereof according to any one of claims 13-23; and
(b) the target antibody.


SEQUENCE LISTING (957 sequences)

SEQ ID NO: 1 - protein, 119 aa, synthetic construct
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYFYDVWGQGTLVTVSS

SEQ ID NO: 2 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 3 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDQYGRYFYDVWGQGTLVTVSS

SEQ ID NO: 4 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDVYGRYFYDLWGQGTLVTVSS

SEQ ID NO: 5 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCGRDAYGRYFYDVWGQGTLVTVSS

SEQ ID NO: 6 - protein, 119 aa, synthetic construct
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSS

SEQ ID NO: 7 - protein, 119 aa, synthetic construct
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDRYGAYFYDVWGQGTLVTVSS

SEQ ID NO: 8 - protein, 119 aa, synthetic construct
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCGRDAYGLYFYDVWGQGTLVTVSS

SEQ ID NO: 9 - protein, 119 aa, synthetic construct
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDQYGRYFYDVWGQGTLVTVSS

SEQ ID NO: 10 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDNYGGYFYDVWGQGTLVTVSS

SEQ ID NO: 11 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 12 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 13 - protein, 112 aa, synthetic construct
DIVMTQSPDSLAVSLGERATINCKSSQSLLNSRTGKNYLAWYQQKPGQPPKLLIYWASTRESGVPDRFSGSGSGTDFTLTISSLQAEDVAVYYCKQSYSRRTFGGGTKVEIK

SEQ ID NO: 14 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 15 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSLRTFGGGTKVEIK

SEQ ID NO: 16 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSIRTFGGGTKVEIK

SEQ ID NO: 17 - protein, 112 aa, synthetic construct
DIVMTQSPDSLAVSLGERATINCKSSQSLLESRTGKNYLAWYQQKPGQPPKLLIYWASTRESGVPDRFSGSGSGTDFTLTISSLQAEDVAVYYCKQSYSLRTFGGGTKVEIK

SEQ ID NO: 18 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 19 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 20 - protein, 9 aa, synthetic construct
FNIKDYYMH

SEQ ID NO: 21 - protein, 9 aa, synthetic construct
FTFNKYAMN

SEQ ID NO: 22 - protein, 9 aa, synthetic construct
FTFTSYYIH

SEQ ID NO: 23 - protein, 17 aa, synthetic construct
WIDLENGNTIYDPKFQG

SEQ ID NO: 24 - protein, 17 aa, synthetic construct
WIDLENANTIYDAKFQG

SEQ ID NO: 25 - protein, 17 aa, synthetic construct
WIDLEEGNTIYDAKFQG

SEQ ID NO: 26 - protein, 19 aa, synthetic construct
RIRSKYNNYATYYADSVKD

SEQ ID NO: 27 - protein, 17 aa, synthetic construct
WIYPENDNTKYNEKFKD

SEQ ID NO: 28 - protein, 12 aa, synthetic construct
ARDGYGRYFYDV

SEQ ID NO: 29 - protein, 12 aa, synthetic construct
ARDAYGRYFYDV

SEQ ID NO: 30 - protein, 12 aa, synthetic construct
ARDQYGRYFYDV

SEQ ID NO: 31 - protein, 12 aa, synthetic construct
ARDVYGRYFYDL

SEQ ID NO: 32 - protein, 12 aa, synthetic construct
GRDAYGRYFYDV

SEQ ID NO: 33 - protein, 12 aa, synthetic construct
ARDRYGAYFYDV

SEQ ID NO: 34 - protein, 12 aa, synthetic construct
GRDAYGLYFYDV

SEQ ID NO: 35 - protein, 12 aa, synthetic construct
ARDNYGGYFYDV

SEQ ID NO: 36 - protein, 16 aa, synthetic construct
VRHGNFGNSYISYWAY

SEQ ID NO: 37 - protein, 12 aa, synthetic construct
ARDGYSRYYFDY

SEQ ID NO: 38 - protein, 17 aa, synthetic construct
KSSQSLLNSRTGKNYLA

SEQ ID NO: 39 - protein, 17 aa, synthetic construct
KSSQSLLNARTGKNYLA

SEQ ID NO: 40 - protein, 17 aa, synthetic construct
KSSQSLLESRTGKNYLA

SEQ ID NO: 41 - protein, 14 aa, synthetic construct
GSSTGAVTSGNYPN

SEQ ID NO: 42 - protein, 17 aa, synthetic construct
KSSQSLLNSRTRKNYLA

SEQ ID NO: 43 - protein, 7 aa, synthetic construct
WASTRES

SEQ ID NO: 44 - protein, 7 aa, synthetic construct
GTKFLAP

SEQ ID NO: 45 - protein, 7 aa, synthetic construct
WTSTRKS

SEQ ID NO: 46 - protein, 8 aa, synthetic construct
KQSYSRRT

SEQ ID NO: 47 - protein, 8 aa, synthetic construct
KQSYSLRT

SEQ ID NO: 48 - protein, 8 aa, synthetic construct
KQSYSIRT

SEQ ID NO: 49 - protein, 9 aa, synthetic construct
VLWYSNRWV

SEQ ID NO: 50 - protein, 8 aa, synthetic construct
KQSFILRT

SEQ ID NO: 51 - protein, 127 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGAPNYYGSGGYDDGWFDPWGQGTLVTVSS

SEQ ID NO: 52 - protein, 128 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARLGGRRGSGSSYYYYYGMDVWGQGTTVTVSS

SEQ ID NO: 53 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQFGRDPTFGGGTKVEIK

SEQ ID NO: 54 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCLQFGSSPTFGGGTKVEIK

SEQ ID NO: 55 - protein, 107 aa, synthetic construct
DIQLTQSPSSLSASVGDRVTITCRASQSISSYLNWYQQKPGKAPKLLIYAASNLQSGVPSRFSGSGSGTDFTLTISSLQPEDFATYYCQQTLHTPITFGGGTKVEIK

SEQ ID NO: 56 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQFGRSPTFGGGTKVEIK

SEQ ID NO: 57 - protein, 107 aa, synthetic construct
EIVLTQSPGTLSLSPGERATLSCRASQSVSSDYLAWYQQKPGQAPRLLIYGASSRATGIPDRFSGSGSGTDFTLTISRLEPEDFAVYYCQQFGRVPTFGGGTKVEIK

SEQ ID NO: 58 - protein, 107 aa, synthetic construct
EIVMTQSPGTLSLSPGERATLSCRASQSVSSSYLAWYQQKPGQAPRLLIYGASSRATGIPDRFSGSGSGTDFTLTISRLEPEDFAVYYCQQFGRYPTFGGGTKVEIK

SEQ ID NO: 59 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQFGPSPTFGGGTKVEIK

SEQ ID NO: 60 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQFGRFPTFGGGTKVEIK

SEQ ID NO: 61 - protein, 108 aa, synthetic construct
EIVMTQSPGTLSLSPGERATLSCRASQSVSSSYLAWYQQKPGQAPRLLIYGASNRATGIPDRFSGSGSGTDFTLTISRLEPEDFAVYYCQQAHTALWTFGGGTKVEIK

SEQ ID NO: 62 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVRQTPWTFGGGTKVEIK

SEQ ID NO: 63 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLDLPWTFGGGTKVEIK

SEQ ID NO: 64 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLELPWTFGGGTKVEIK

SEQ ID NO: 65 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVKDTPWTFGGGTKVEIK

SEQ ID NO: 66 - protein, 112 aa, synthetic construct
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQVLGLPWTFGGGTKVEIK

SEQ ID NO: 67 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVSDTPWTFGGGTKVEIK

SEQ ID NO: 68 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLEIPWTFGGGTKVEIK

SEQ ID NO: 69 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLELPWAFGGGTKVEIK

SEQ ID NO: 70 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLDLPWAFGGGTKVEIK

SEQ ID NO: 71 - protein, 9 aa, synthetic construct
GTFSSYAIS

SEQ ID NO: 72 - protein, 9 aa, synthetic construct
FTFSSYAMS

SEQ ID NO: 73 - protein, 17 aa, synthetic construct
GIIPIFGTANYAQKFQG

SEQ ID NO: 74 - protein, 17 aa, synthetic construct
TISGSGGSTYYADSVKG

SEQ ID NO: 75 - protein, 20 aa, synthetic construct
ARGAPNYYGSGGYDDGWFDP

SEQ ID NO: 76 - protein, 21 aa, synthetic construct
ARLGGRRGSGSSYYYYYGMDV

SEQ ID NO: 77 - protein, 12 aa, synthetic construct
RASQSVSSSYLA

SEQ ID NO: 78 - protein, 11 aa, synthetic construct
RASQSISSYLN

SEQ ID NO: 79 - protein, 12 aa, synthetic construct
RASQSVSSDYLA

SEQ ID NO: 80 - protein, 16 aa, synthetic construct
RSSQSLLHSNGYNYLD

SEQ ID NO: 81 - protein, 16 aa, synthetic construct
RSSQSLLYSNGYNYLD

SEQ ID NO: 82 - protein, 7 aa, synthetic construct
GASSRAT

SEQ ID NO: 83 - protein, 7 aa, synthetic construct
AASNLQS

SEQ ID NO: 84 - protein, 7 aa, synthetic construct
GASNRAT

SEQ ID NO: 85 - protein, 7 aa, synthetic construct
LGSNRAS

SEQ ID NO: 86 - protein, 8 aa, synthetic construct
QQFGRDPT

SEQ ID NO: 87 - protein, 8 aa, synthetic construct
LQFGSSPT

SEQ ID NO: 88 - protein, 9 aa, synthetic construct
QQTLHTPIT

SEQ ID NO: 89 - protein, 8 aa, synthetic construct
QQFGRSPT

SEQ ID NO: 90 - protein, 8 aa, synthetic construct
QQFGRVPT

SEQ ID NO: 91 - protein, 8 aa, synthetic construct
QQFGRYPT

SEQ ID NO: 92 - protein, 8 aa, synthetic construct
QQFGPSPT

SEQ ID NO: 93 - protein, 8 aa, synthetic construct
QQFGRFPT

SEQ ID NO: 94 - protein, 9 aa, synthetic construct
QQAHTALWT

SEQ ID NO: 95 - protein, 9 aa, synthetic construct
MQVRQTPWT

SEQ ID NO: 96 - protein, 9 aa, synthetic construct
MQVLDLPWT

SEQ ID NO: 97 - protein, 9 aa, synthetic construct
MQVLELPWT

SEQ ID NO: 98 - protein, 9 aa, synthetic construct
MQVKDTPWT

SEQ ID NO: 99 - protein, 9 aa, synthetic construct
MQVLGLPWT

SEQ ID NO: 100 - protein, 9 aa, synthetic construct
MQVSDTPWT

SEQ ID NO: 101 - protein, 9 aa, synthetic construct
MQVLEIPWT

SEQ ID NO: 102 - protein, 9 aa, synthetic construct
MQVLELPWA

SEQ ID NO: 103 - protein, 9 aa, synthetic construct
MQVLDLPWA

SEQ ID NO: 104 - protein, 28 aa, synthetic construct
  SITE 28: note = Lysine short chain biotin discrete polyethylene glycol amide
QDGNEEMGSITQTPYQVSISGTTVILTK

SEQ ID NO: 105 - protein, 14 aa, synthetic construct
  SITE 14: note = Lysine short chain biotin discrete polyethylene glycol amide
QDGNEEMGGITQTK

SEQ ID NO: 106-109 - skipped (4 entries)

SEQ ID NO: 110 - protein, 119 aa, synthetic construct; identical to SEQ ID NO: 1

SEQ ID NO: 111-119 - skipped (9 entries)

SEQ ID NO: 120 - protein, 119 aa, synthetic construct; identical to SEQ ID NO: 7

SEQ ID NO: 121-129 - skipped (9 entries)

SEQ ID NO: 130 - protein, 119 aa, synthetic construct; identical to SEQ ID NO: 8

SEQ ID NO: 131-139 - skipped (9 entries)

SEQ ID NO: 140 - protein, 119 aa, synthetic construct; identical to SEQ ID NO: 9

SEQ ID NO: 141 - protein, 9 aa, synthetic construct
FNIKDYYMH

SEQ ID NO: 142 - protein, 17 aa, synthetic construct
WIDLEEGNTIYDAKFQG

SEQ ID NO: 143 - protein, 12 aa, synthetic construct
ARDQYGRYFYDV

SEQ ID NO: 144-149 - skipped (6 entries)

SEQ ID NO: 150 - protein, 119 aa, synthetic construct; identical to SEQ ID NO: 10

SEQ ID NO: 151 - protein, 9 aa, synthetic construct
FNIKDYYMH

SEQ ID NO: 152 - protein, 17 aa, synthetic construct
WIDLENANTIYDAKFQG

SEQ ID NO: 153 - protein, 12 aa, synthetic construct
ARDNYGGYFYDV

SEQ ID NO: 154-159 - skipped (6 entries)

SEQ ID NO: 160 - protein, 125 aa, synthetic construct; identical to SEQ ID NO: 11

SEQ ID NO: 161 - protein, 9 aa, synthetic construct
FTFNKYAMN

SEQ ID NO: 162 - protein, 19 aa, synthetic construct
RIRSKYNNYATYYADSVKD

SEQ ID NO: 163 - protein, 16 aa, synthetic construct
VRHGNFGNSYISYWAY

SEQ ID NO: 164-169 - skipped (6 entries)

SEQ ID NO: 170 - protein, 119 aa, synthetic construct; identical to SEQ ID NO: 12

SEQ ID NO: 171 - protein, 9 aa, synthetic construct
FTFTSYYIH

SEQ ID NO: 172 - protein, 17 aa, synthetic construct
WIYPENDNTKYNEKFKD

SEQ ID NO: 173 - protein, 12 aa, synthetic construct
ARDGYSRYYFDY

SEQ ID NO: 174-179 - skipped (6 entries)

SEQ ID NO: 180 - protein, 119 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 181 - protein, 9 aa, synthetic construct
FNIKDYYMH

SEQ ID NO: 182 - protein, 17 aa, synthetic construct
WIDLENANTVYDAKFQG

SEQ ID NO: 183 - protein, 12 aa, synthetic construct
ARDAYGRYFYDV

SEQ ID NO: 184-189 - skipped (6 entries)

SEQ ID NO: 190 - protein, 119 aa, synthetic construct
QVQLVQSGAEVKKPGASVKVSCKASGFDIKDYYMHWVRQAPGQRLEWIGWIELENADTVYDAKFQGRVTITRDTSASTAYMELSSLRSEDTAVYYCARDAYGRYFYDVWGQGTLVTVSS

SEQ ID NO: 191 - protein, 9 aa, synthetic construct
FDIKDYYMH

SEQ ID NO: 192 - protein, 17 aa, synthetic construct
WIELENADTVYDAKFQG

SEQ ID NO: 193 - protein, 12 aa, synthetic construct
ARDAYGRYFYDV

SEQ ID NO: 194-199 - skipped (6 entries)

SEQ ID NO: 200 - protein, 119 aa, synthetic construct
QVQLVQSGAEVKKPGASVKVSCKASGFDIKDYYMHWVRQAPGQRLEWIGWIELENDDTVYDAKFQGRVTITRDTSASTAYMELSSLRSEDTAVYYCARDAYGRYFYDVWGQGTLVTVSS

SEQ ID NO: 201 - protein, 9 aa, synthetic construct
FDIKDYYMH

SEQ ID NO: 202 - protein, 17 aa, synthetic construct
WIELENDDTVYDAKFQG

SEQ ID NO: 203 - protein, 12 aa, synthetic construct
ARDAYGRYFYDV

SEQ ID NO: 204-209 - skipped (6 entries)

SEQ ID NO: 210 - protein, 119 aa, synthetic construct
QVQLVQSGAEVKKPGASVKVSCKASGFNIDDYYMHWVRQAPGQRLEWIGWIDLENANTVDDAKFQGRVTITRDTSASTAYMELSSLRSEDTAVYYCARDAYGRYFYDVWGQGTLVTVSS

SEQ ID NO: 211 - protein, 9 aa, synthetic construct
FNIDDYYMH

SEQ ID NO: 212 - protein, 17 aa, synthetic construct
WIDLENANTVDDAKFQG

SEQ ID NO: 213 - protein, 12 aa, synthetic construct
ARDAYGRYFYDV

SEQ ID NO: 214-219 - skipped (6 entries)

SEQ ID NO: 220 - protein, 119 aa, synthetic construct; identical to SEQ ID NO: 2

SEQ ID NO: 221 - protein, 9 aa, synthetic construct
FNIKDYYMH

SEQ ID NO: 222 - protein, 17 aa, synthetic construct
WIDLENANTIYDAKFQG

SEQ ID NO: 223 - protein, 12 aa, synthetic construct
ARDAYGRYFYDV

SEQ ID NO: 224-229 - skipped (6 entries)

SEQ ID NO: 230 - protein, 119 aa, synthetic construct
QVQLVQSGAEVKKPGASVKVSCKASGFNIDDYYMHWVRQAPGQRLEWMGWIDLENANTIDDAKFQGRVTITRDTSASTAYMELSSLRSEDTAVYYCARDAYGRYFYDVWGQGTLVTVSS

SEQ ID NO: 231 - protein, 9 aa, synthetic construct
FNIDDYYMH

SEQ ID NO: 232 - protein, 17 aa, synthetic construct
WIDLENANTIDDAKFQG

SEQ ID NO: 233 - protein, 12 aa, synthetic construct
ARDAYGRYFYDV

SEQ ID NO: 234-239 - skipped (6 entries)

SEQ ID NO: 240 - protein, 119 aa, synthetic construct
QVQLVQSGAEVKKPGASVKVSCKASGFNIKDYYMHWVRQAPGQRLEWMGWIDLENAETIYDAKFQDRVTITRDTSASTAYMELSSLRSEDTAVYYCARDAYGRYFYDEWGQGTLVTVSS

SEQ ID NO: 241 - protein, 9 aa, synthetic construct
FNIKDYYMH

SEQ ID NO: 242 - protein, 17 aa, synthetic construct
WIDLENAETIYDAKFQD

SEQ ID NO: 243 - protein, 12 aa, synthetic construct
ARDAYGRYFYDE

SEQ ID NO: 244-249 - skipped (6 entries)

SEQ ID NO: 250 - protein, 119 aa, synthetic construct
QVQLVQSGAEVKKPGASVKVSCKASGFNIDDYYMHWVRQAPGQRLEWMGWIDLENDNTIYDAKFQGRVTITRDTSASTAYMELSSLRSEDTAVYYCARDAYGRYFYDVWGQGTLVTVSS

SEQ ID NO: 251 - protein, 9 aa, synthetic construct
FNIDDYYMH

SEQ ID NO: 252 - protein, 17 aa, synthetic construct
WIDLENDNTIYDAKFQG

SEQ ID NO: 253 - protein, 12 aa, synthetic construct
ARDAYGRYFYDV

SEQ ID NO: 254-259 - skipped (6 entries)

SEQ ID NO: 260 - protein, 119 aa, synthetic construct
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCGRDAYGRYFYDVWGQGTLVTVSS

SEQ ID NO: 261 - protein, 9 aa, synthetic construct
FNIKDYYMH

SEQ ID NO: 262 - protein, 17 aa, synthetic construct
WIDLEEGNTIYDAKFQG

SEQ ID NO: 263 - protein, 12 aa, synthetic construct
GRDAYGRYFYDV

SEQ ID NO: 264-269 - skipped (6 entries)

SEQ ID NO: 270 - protein, 119 aa, synthetic construct; identical to SEQ ID NO: 3

SEQ ID NO: 271 - protein, 9 aa, synthetic construct
FNIKDYYMH

SEQ ID NO: 272 - protein, 17 aa, synthetic construct
WIDLENANTIYDAKFQG

SEQ ID NO: 273 - protein, 12 aa, synthetic construct
ARDQYGRYFYDV

SEQ ID NO: 274-279 - skipped (6 entries)

SEQ ID NO: 280 - protein, 119 aa, synthetic construct; identical to SEQ ID NO: 2

SEQ ID NO: 281 - protein, 9 aa, synthetic construct
FNIKDYYMH

SEQ ID NO: 282 - protein, 17 aa, synthetic construct
WIDLENANTIYDAKFQG

SEQ ID NO: 283 - protein, 12 aa, synthetic construct
ARDAYGRYFYDV

SEQ ID NO: 284-289 - skipped (6 entries)

SEQ ID NO: 290 - protein, 119 aa, synthetic construct; identical to SEQ ID NO: 5

SEQ ID NO: 291 - protein, 9 aa, synthetic construct
FNIKDYYMH

SEQ ID NO: 292 - protein, 17 aa, synthetic construct
WIDLENANTIYDAKFQG

SEQ ID NO: 293 - protein, 12 aa, synthetic construct
GRDAYGRYFYDV

SEQ ID NO: 294-295 - skipped (2 entries)

SEQ ID NO: 296 - protein, 17 aa, synthetic construct
KSSQSLLNARTGKNYLA

SEQ ID NO: 297 - protein, 7 aa, synthetic construct
WASTRES

SEQ ID NO: 298 - protein, 8 aa, synthetic construct
KQSYSLRT

SEQ ID NO: 299 - skipped

SEQ ID NO: 300 - protein, 119 aa, synthetic construct; identical to SEQ ID NO: 6

SEQ ID NO: 301 - protein, 9 aa, synthetic construct
FNIKDYYMH

SEQ ID NO: 302 - protein, 17 aa, synthetic construct
WIDLEEGNTIYDAKFQG

SEQ ID NO: 303 - protein, 12 aa, synthetic construct
ARDAYGRYFYDV

SEQ ID NO: 304-305 - skipped (2 entries)

SEQ ID NO: 306 - protein, 17 aa, synthetic construct
KSSQSLLNARTGKNYLA

SEQ ID NO: 307 - protein, 7 aa, synthetic construct
WASTRES

SEQ ID NO: 308 - protein, 8 aa, synthetic construct
KQSYSLRT

SEQ ID NO: 309 - skipped

SEQ ID NO: 310 - protein, 119 aa, synthetic construct; identical to SEQ ID NO: 4

SEQ ID NO: 311 - protein, 9 aa, synthetic construct
FNIKDYYMH

SEQ ID NO: 312 - protein, 17 aa, synthetic construct
WIDLENANTIYDAKFQG

SEQ ID NO: 313 - protein, 12 aa, synthetic construct
ARDVYGRYFYDL

SEQ ID NO: 314 - skipped

SEQ ID NO: 315 - protein, 112 aa, synthetic construct; identical to SEQ ID NO: 14

SEQ ID NO: 316 - protein, 17 aa, synthetic construct
KSSQSLLNARTGKNYLA

SEQ ID NO: 317 - protein, 7 aa, synthetic construct
WASTRES

SEQ ID NO: 318 - protein, 8 aa, synthetic construct
KQSYSRRT

SEQ ID NO: 319 - skipped

SEQ ID NO: 320 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDVLGRYFYDVWGQGTLVTVSS

SEQ ID NO: 321 - protein, 9 aa, synthetic construct
FNIKDYYMH

SEQ ID NO: 322 - protein, 17 aa, synthetic construct
WIDLENANTIYDAKFQG

SEQ ID NO: 323 - protein, 12 aa, synthetic construct
ARDVLGRYFYDV

SEQ ID NO: 324 - skipped

SEQ ID NO: 325 - protein, 112 aa, synthetic construct; identical to SEQ ID NO: 14

SEQ ID NO: 326 - protein, 17 aa, synthetic construct
KSSQSLLNARTGKNYLA

SEQ ID NO: 327 - protein, 7 aa, synthetic construct
WASTRES

SEQ ID NO: 328 - protein, 8 aa, synthetic construct
KQSYSRRT

SEQ ID NO: 329 - skipped

SEQ ID NO: 330 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCGRDAYGLYFYDVWGQGTLVTVSS

SEQ ID NO: 331 - protein, 9 aa, synthetic construct
FNIKDYYMH

SEQ ID NO: 332 - protein, 17 aa, synthetic construct
WIDLENANTIYDAKFQG

SEQ ID NO: 333 - protein, 12 aa, synthetic construct
GRDAYGLYFYDV

SEQ ID NO: 334 - skipped

SEQ ID NO: 335 - protein, 112 aa, synthetic construct; identical to SEQ ID NO: 15

SEQ ID NO: 336 - protein, 17 aa, synthetic construct
KSSQSLLNARTGKNYLA

SEQ ID NO: 337 - protein, 7 aa, synthetic construct
WASTRES

SEQ ID NO: 338 - protein, 8 aa, synthetic construct
KQSYSLRT

SEQ ID NO: 339 - skipped

SEQ ID NO: 340 - protein, 119 aa, synthetic construct
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDVLGRYFYDVWGQGTLVTVSS

SEQ ID NO: 341 - protein, 9 aa, synthetic construct
FNIKDYYMH

SEQ ID NO: 342 - protein, 17 aa, synthetic construct
WIDLEEGNTIYDAKFQG

SEQ ID NO: 343 - protein, 12 aa, synthetic construct
ARDVLGRYFYDV

SEQ ID NO: 344 - skipped

SEQ ID NO: 345 - protein, 112 aa, synthetic construct; identical to SEQ ID NO: 14

SEQ ID NO: 346 - protein, 17 aa, synthetic construct
KSSQSLLNARTGKNYLA

SEQ ID NO: 347 - protein, 7 aa, synthetic construct
WASTRES

SEQ ID NO: 348 - protein, 8 aa, synthetic construct
KQSYSRRT

SEQ ID NO: 349 - skipped

SEQ ID NO: 350 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGGYFYDVWGQGTLVTVSS

SEQ ID NO: 351 - protein, 9 aa, synthetic construct
FNIKDYYMH

SEQ ID NO: 352 - protein, 17 aa, synthetic construct
WIDLENANTIYDAKFQG

SEQ ID NO: 353 - protein, 12 aa, synthetic construct
ARDAYGGYFYDV

SEQ ID NO: 354 - skipped

SEQ ID NO: 355 - protein, 112 aa, synthetic construct; identical to SEQ ID NO: 16

SEQ ID NO: 356 - protein, 17 aa, synthetic construct
KSSQSLLNARTGKNYLA

SEQ ID NO: 357 - protein, 7 aa, synthetic construct
WASTRES

SEQ ID NO: 358 - protein, 8 aa, synthetic construct
KQSYSIRT

SEQ ID NO: 359 - skipped

SEQ ID NO: 360 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYHRYFYDHWGQGTLVTVSS

SEQ ID NO: 361 - protein, 9 aa, synthetic construct
FNIKDYYMH

SEQ ID NO: 362 - protein, 17 aa, synthetic construct
WIDLENANTIYDAKFQG

SEQ ID NO: 363 - protein, 12 aa, synthetic construct
ARDAYHRYFYDH

SEQ ID NO: 364 - skipped

SEQ ID NO: 365 - protein, 112 aa, synthetic construct
DIVMTQSPDSLAVSLGERATINCKSSQSLLNAHTGKNHLAWYQQKPGQPPKLLIYWASTRESGVPDRFSGSGSGTDFTLTISSLQAEDVAVYYCKQSHSHRTFGGGTKVEIK

SEQ ID NO: 366 - protein, 17 aa, synthetic construct
KSSQSLLNAHTGKNHLA

SEQ ID NO: 367 - protein, 7 aa, synthetic construct
WASTRES

SEQ ID NO: 368 - protein, 8 aa, synthetic construct
KQSHSHRT

SEQ ID NO: 369 - skipped

SEQ ID NO: 370 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDHYHRYFYDVWGQGTLVTVSS

SEQ ID NO: 371 - protein, 9 aa, synthetic construct
FNIKDYYMH

SEQ ID NO: 372 - protein, 17 aa, synthetic construct
WIDLENANTIYDAKFQG

SEQ ID NO: 373 - protein, 12 aa, synthetic construct
ARDHYHRYFYDV

SEQ ID NO: 374 - skipped

SEQ ID NO: 375 - protein, 112 aa, synthetic construct
DIVMTQSPDSLAVSLGERATINCKSSQSLLNARTGHNYLAWYQQKPGQPPKLLIYWASTRESGVPDRFSGSGSGTDFTLTISSLQAEDVAVYYCKQSHSHRTFGGGTKVEIK

SEQ ID NO: 376 - protein, 17 aa, synthetic construct
KSSQSLLNARTGHNYLA

SEQ ID NO: 377 - protein, 7 aa, synthetic construct
WASTRES

SEQ ID NO: 378 - protein, 8 aa, synthetic construct
KQSHSHRT

SEQ ID NO: 379 - skipped

SEQ ID NO: 380 - protein, 119 aa, synthetic construct
QVQLVQSGAEVKEPGASVKVSCKASGFNIKDYYMHWVRQAPGQRLEWMGWIDLENANTIYDAKFQGRVTITRDTSASTAYMELSSLRSEDTAVYYCARDAHHRYFYDVWGQGTLVTVSS

SEQ ID NO: 381 - protein, 9 aa, synthetic construct
FNIKDYYMH

SEQ ID NO: 382 - protein, 17 aa, synthetic construct
WIDLENANTIYDAKFQG

SEQ ID NO: 383 - protein, 12 aa, synthetic construct
ARDAHHRYFYDV

SEQ ID NO: 384 - skipped

SEQ ID NO: 385 - protein, 112 aa, synthetic construct
DIVMTQSPDSLAVSLGERATINCKSSQSLLNAHTGHNYLAWYQQKPGQPPKLLIYWASTRESGVPDRFSGSGSGTDFTLTISSLQAEDVAVYYCKQSYSRRTFGGGTKVEIK

SEQ ID NO: 386 - protein, 17 aa, synthetic construct
KSSQSLLNAHTGHNYLA

SEQ ID NO: 387 - protein, 7 aa, synthetic construct
WASTRES

SEQ ID NO: 388 - protein, 8 aa, synthetic construct
KQSYSRRT

SEQ ID NO: 389 - skipped

SEQ ID NO: 390 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDATHRYFYDVWGQGTLVTVSS

SEQ ID NO: 391 - protein, 9 aa, synthetic construct
FNIKDYYMH

SEQ ID NO: 392 - protein, 17 aa, synthetic construct
WIDLENANTIYDAKFQG

SEQ ID NO: 393 - protein, 12 aa, synthetic construct
ARDATHRYFYDV

SEQ ID NO: 394 - skipped

SEQ ID NO: 395 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSHTRRTFGGGTKVEIK

SEQ ID NO: 396 - protein, 17 aa, synthetic construct
KSSQSLLNARTGKNYLA

SEQ ID NO: 397 - protein, 7 aa, synthetic construct
WASTRES

SEQ ID NO: 398 - protein, 8 aa, synthetic construct
KQSHTRRT

SEQ ID NO: 399 - skipped

SEQ ID NO: 400 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYHRYFYDVWGQGTLVTVSS

SEQ ID NO: 401 - protein, 9 aa, synthetic construct
FNIKDYYMH

SEQ ID NO: 402 - protein, 17 aa, synthetic construct
WIDLENANTIYDAKFQG

SEQ ID NO: 403 - protein, 12 aa, synthetic construct
ARDAYHRYFYDV

SEQ ID NO: 404 - skipped

SEQ ID NO: 405 - protein, 112 aa, synthetic construct; identical to SEQ ID NO: 375

SEQ ID NO: 406 - protein, 17 aa, synthetic construct
KSSQSLLNARTGHNYLA

SEQ ID NO: 407 - protein, 7 aa, synthetic construct
WASTRES

SEQ ID NO: 408 - protein, 8 aa, synthetic construct
KQSHSHRT

SEQ ID NO: 409 - skipped

SEQ ID NO: 410 - protein, 119 aa, synthetic construct; identical to SEQ ID NO: 370

SEQ ID NO: 411 - protein, 9 aa, synthetic construct
FNIKDYYMH

SEQ ID NO: 412 - protein, 17 aa, synthetic construct
WIDLENANTIYDAKFQG

SEQ ID NO: 413 - protein, 12 aa, synthetic construct
ARDHYHRYFYDV

SEQ ID NO: 414 - skipped

SEQ ID NO: 415 - protein, 112 aa, synthetic construct
DIVMTQSPDSLAVSLGERATINCKSSQSLLNARTGHNYLAWYQQKPGQPPKLLIYWASTRESGVPDRFSGSGSGTDFTLTISSLQAEDVAVYYCKQSYSHRTFGGGTKVEIK

SEQ ID NO: 416 - protein, 17 aa, synthetic construct
KSSQSLLNARTGHNYLA

SEQ ID NO: 417 - protein, 7 aa, synthetic construct
WASTRES

SEQ ID NO: 418 - protein, 8 aa, synthetic construct
KQSYSHRT

SEQ ID NO: 419 - skipped

SEQ ID NO: 420 - protein, 119 aa, synthetic construct; identical to SEQ ID NO: 370

SEQ ID NO: 421 - protein, 9 aa, synthetic construct
FNIKDYYMH

SEQ ID NO: 422 - protein, 17 aa, synthetic construct
WIDLENANTIYDAKFQG

SEQ ID NO: 423 - protein, 12 aa, synthetic construct
ARDHYHRYFYDV

SEQ ID NO: 424 - skipped

SEQ ID NO: 425 - protein, 112 aa, synthetic construct
GIVMTQSPDSLAVSLGERATINCKSSQSLLNARTGKNYLAWYQQKPGQPPKLLIYWASTRESGVPDRFSGSGSGTDFTLTISSLQAEDVAVYYCKQSHSHRTFGGGTKVEIK

SEQ ID NO: 426 - protein, 17 aa, synthetic construct
KSSQSLLNARTGKNYLA

SEQ ID NO: 427 - protein, 7 aa, synthetic construct
WASTRES

SEQ ID NO: 428 - protein, 8 aa, synthetic construct
KQSHSHRT

SEQ ID NO: 429 - skipped

SEQ ID NO: 430 - protein, 119 aa, synthetic construct; identical to SEQ ID NO: 400

SEQ ID NO: 431 - protein, 9 aa, synthetic construct
FNIKDYYMH

SEQ ID NO: 432 - protein, 17 aa, synthetic construct
WIDLENANTIYDAKFQG

SEQ ID NO: 433 - protein, 12 aa, synthetic construct
ARDAYHRYFYDV

SEQ ID NO: 434 - skipped

SEQ ID NO: 435 - protein, 112 aa, synthetic construct; identical to SEQ ID NO: 415

SEQ ID NO: 436 - protein, 17 aa, synthetic construct
KSSQSLLNARTGHNYLA

SEQ ID NO: 437 - protein, 7 aa, synthetic construct
WASTRES

SEQ ID NO: 438 - protein, 8 aa, synthetic construct
KQSYSHRT

SEQ ID NO: 439 - skipped

SEQ ID NO: 440 - protein, 119 aa, synthetic construct
QVQLVQSGAEVKKPGASVKVSCKASGFDIKDYYMHWVRQAPGQRLEWMGWIDLENANTIYDAKFQGRVTITRDTSASTAYMELSSLRSEDTAVYYCARDQYGRYFYDVWGQGTLVTVSS

SEQ ID NO: 441 - protein, 9 aa, synthetic construct
FDIKDYYMH

SEQ ID NO: 442 - protein, 17 aa, synthetic construct
WIDLENANTIYDAKFQG

SEQ ID NO: 443 - protein, 12 aa, synthetic construct
ARDQYGRYFYDV

SEQ ID NO: 444 - skipped

SEQ ID NO: 445 - protein, 112 aa, synthetic construct; identical to SEQ ID NO: 14

SEQ ID NO: 446 - protein, 17 aa, synthetic construct
KSSQSLLNARTGKNYLA

SEQ ID NO: 447 - protein, 7 aa, synthetic construct
WASTRES

SEQ ID NO: 448 - protein, 8 aa, synthetic construct
KQSYSRRT

SEQ ID NO: 449 - skipped

SEQ ID NO: 450 - protein, 119 aa, synthetic construct
QVQLVQSGAEVKKPGASVKVSCKASGFNIKDYYMHWVRQAPGQRLEWMGWIDLENADTIYDAKFQGRVTITRDTSASTAYMELSSLRSEDTAVYYCARDQYGRYFYDVWGQGTLVTVSS

SEQ ID NO: 451 - protein, 9 aa, synthetic construct
FNIKDYYMH

SEQ ID NO: 452 - protein, 17 aa, synthetic construct
WIDLENADTIYDAKFQG

SEQ ID NO: 453 - protein, 12 aa, synthetic construct
ARDQYGRYFYDV

SEQ ID NO: 454 - skipped

SEQ ID NO: 455 - protein, 112 aa, synthetic construct; identical to SEQ ID NO: 14

SEQ ID NO: 456 - protein, 17 aa, synthetic construct
KSSQSLLNARTGKNYLA

SEQ ID NO: 457 - protein, 7 aa, synthetic construct
WASTRES

SEQ ID NO: 458 - protein, 8 aa, synthetic construct
KQSYSRRT

SEQ ID NO: 459 - skipped

SEQ ID NO: 460 - protein, 119 aa, synthetic construct
QVQLVQSGAEVKKPGASVKVSCKASGFDIKDYYMHWVRQAPGQRLEWMGWIDLENANTIYDAKFQGRVTITRDTSASTAYMELSSLRSEDTAVYYCGRDAYGRYFYDVWGQGTLVTVSS

SEQ ID NO: 461 - protein, 9 aa, synthetic construct
FDIKDYYMH

SEQ ID NO: 462 - protein, 17 aa, synthetic construct
WIDLENANTIYDAKFQG

SEQ ID NO: 463 - protein, 12 aa, synthetic construct
GRDAYGRYFYDV

SEQ ID NO: 464 - skipped

SEQ ID NO: 465 - protein, 112 aa, synthetic construct; identical to SEQ ID NO: 15

SEQ ID NO: 466 - protein, 17 aa, synthetic construct
KSSQSLLNARTGKNYLA

SEQ ID NO: 467 - protein, 7 aa, synthetic construct
WASTRES

SEQ ID NO: 468 - protein, 8 aa, synthetic construct
KQSYSLRT

SEQ ID NO: 469 - skipped

SEQ ID NO: 470 - protein, 119 aa, synthetic construct
QVQLVQSGAEVKKPGASVKVSCKASGFNIKDYYMHWVRQAPGQRLEWMGWIDLENADTIYDAKFQGRVTITRDTSASTAYMELSSLRSEDTAVYYCGRDAYGRYFYDVWGQGTLVTVSS

SEQ ID NO: 471 - protein, 9 aa, synthetic construct
FNIKDYYMH

SEQ ID NO: 472 - protein, 17 aa, synthetic construct
WIDLENADTIYDAKFQG

SEQ ID NO: 473 - protein, 12 aa, synthetic construct
GRDAYGRYFYDV

SEQ ID NO: 474 - skipped

SEQ ID NO: 475 - protein, 112 aa, synthetic construct; identical to SEQ ID NO: 15

SEQ ID NO: 476 - protein, 17 aa, synthetic construct
KSSQSLLNARTGKNYLA

SEQ ID NO: 477 - protein, 7 aa, synthetic construct
WASTRES

SEQ ID NO: 478 - protein, 8 aa, synthetic construct
KQSYSLRT

SEQ ID NO: 479 - skipped

SEQ ID NO: 480 - protein, 119 aa, synthetic construct
QVQLVQSGAEVKKPGASVKVSCKASGFDIKDYYMHWVRQAPGQRLEWMGWIDLEEGNTIYDAKFQGRVTITRDTSASTAYMELSSLRSEDTAVYYCARDAYGRYFYDVWGQGTLVTVSS

SEQ ID NO: 481 - protein, 9 aa, synthetic construct
FDIKDYYMH

SEQ ID NO: 482 - protein, 17 aa, synthetic construct
WIDLEEGNTIYDAKFQG

SEQ ID NO: 483 - protein, 12 aa, synthetic construct
ARDAYGRYFYDV

SEQ ID NO: 484 - skipped

SEQ ID NO: 485 - protein, 112 aa, synthetic construct; identical to SEQ ID NO: 15

SEQ ID NO: 486 - protein, 17 aa, synthetic construct
KSSQSLLNARTGKNYLA

SEQ ID NO: 487 - protein, 7 aa, synthetic construct
WASTRES

SEQ ID NO: 488 - protein, 8 aa, synthetic construct
KQSYSLRT

SEQ ID NO: 489 - skipped

SEQ ID NO: 490 - protein, 119 aa, synthetic construct
QVQLVQSGAEVKKPGASVKVSCKASGFNIKDYYMHWVRQAPGQRLEWMGWIDLEEGDTIYDAKFQGRVTITRDTSASTAYMELSSLRSEDTAVYYCARDAYGRYFYDVWGQGTLVTVSS

SEQ ID NO: 491 - protein, 9 aa, synthetic construct
FNIKDYYMH

SEQ ID NO: 492 - protein, 17 aa, synthetic construct
WIDLEEGDTIYDAKFQG

SEQ ID NO: 493 - protein, 12 aa, synthetic construct
ARDAYGRYFYDV

SEQ ID NO: 494 - skipped

SEQ ID NO: 495 - protein, 112 aa, synthetic construct; identical to SEQ ID NO: 15

SEQ ID NO: 496 - protein, 17 aa, synthetic construct
KSSQSLLNARTGKNYLA

SEQ ID NO: 497 - protein, 7 aa, synthetic construct
WASTRES

SEQ ID NO: 498 - protein, 8 aa, synthetic construct
KQSYSLRT

SEQ ID NO: 499 - skipped

SEQ ID NO: 500 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 501 - protein, 9 aa, synthetic construct
YTFTNYYIH

SEQ ID NO: 502 - protein, 17 aa, synthetic construct
WIYPGDGNTKYNEKFKG

SEQ ID NO: 503 - protein, 12 aa, synthetic construct
ARDSYSNYYFDY

SEQ ID NO: 504 - skipped

SEQ ID NO: 505 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 506 - protein, 17 aa, synthetic construct
KSSQSLLNSRTRKNYLA

SEQ ID NO: 507 - protein, 7 aa, synthetic construct
WASTRES

SEQ ID NO: 508 - protein, 8 aa, synthetic construct
TQSFILRT

SEQ ID NO: 509 - skipped

SEQ ID NO: 510 - protein, 123 aa, synthetic construct
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 511 - protein, 9 aa, synthetic construct
FTFDDYTMH

SEQ ID NO: 512 - protein, 17 aa, synthetic construct
GISWNSGSIGYADSVKG

SEQ ID NO: 513 - protein, 16 aa, synthetic construct
AKDNSGYGHYYYGMDV

SEQ ID NO: 514 - skipped

SEQ ID NO: 515 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 516 - protein, 11 aa, synthetic construct
RASQSVSSNLA

SEQ ID NO: 517 - protein, 7 aa, synthetic construct
GASTRAT

SEQ ID NO: 518 - protein, 9 aa, synthetic construct
QHYINWPLT

SEQ ID NO: 519 - skipped

SEQ ID NO: 520 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 521 - protein, 9 aa, synthetic construct
FTFSTYAMN

SEQ ID NO: 522 - protein, 19 aa, synthetic construct
RIRSKYNNYATYYADSVKG

SEQ ID NO: 523 - protein, 16 aa, synthetic construct
VRHGNFGNSYVSWFAY

SEQ ID NO: 524 - skipped

SEQ ID NO: 525 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 526 - protein, 14 aa, synthetic construct
GSSTGAVTTSNYAN

SEQ ID NO: 527 - protein, 7 aa, synthetic construct
GTNKRAP

SEQ ID NO: 528 - protein, 9 aa, synthetic construct
ALWYSNLWV

SEQ ID NO: 529 - skipped

SEQ ID NO: 530 - protein, 118 aa, synthetic construct
QVQLVQSGGGVVQPGRSLRLSCVASGFTFSSYGMHWVRQAPGKGLEWVAQIWYNARKQEYSDSVKGRFTISRDNSKNTLYLQMNSLRAEDTAVYYCTRGTGYNWFDPWGQGTLVTVSS

SEQ ID NO: 531 - protein, 9 aa, synthetic construct
FTFSSYGMH

SEQ ID NO: 532 - protein, 17 aa, synthetic construct
QIWYNARKQEYSDSVKG

SEQ ID NO: 533 - protein, 11 aa, synthetic construct
TRGTGYNWFDP

SEQ ID NO: 534 - skipped

SEQ ID NO: 535 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 536 - protein, 11 aa, synthetic construct
RASQSISSYLN

SEQ ID NO: 537 - protein, 7 aa, synthetic construct
AASSLQS

SEQ ID NO: 538 - protein, 9 aa, synthetic construct
QQSYSTPPT

SEQ ID NO: 539-540 - skipped (2 entries)

SEQ ID NO: 541 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 542 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 543 - protein, 15 aa, Homo sapiens
EPKSCDKTHTCPPCP

SEQ ID NO: 544 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 545 - protein, 106 aa, Homo sapiens [sequence omitted]

SEQ ID NO: 546 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 547 - protein, 106 aa, Homo sapiens
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPG

SEQ ID NO: 548 - protein, 106 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPG

SEQ ID NO: 549-550 - skipped (2 entries)

SEQ ID NO: 551 - protein, 107 aa, Homo sapiens [sequence omitted]

SEQ ID NO: 552 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 553-559 - skipped (7 entries)

SEQ ID NO: 560 - protein, 127 aa, synthetic construct; identical to SEQ ID NO: 51

SEQ ID NO: 561 - protein, 26 aa, synthetic construct
QVQLVQSGAEVKKPGSSVKVSCKASG

SEQ ID NO: 562 - protein, 9 aa, synthetic construct
GTFSSYAIS

SEQ ID NO: 563 - protein, 14 aa, synthetic construct
WVRQAPGQGLEWMG

SEQ ID NO: 564 - protein, 17 aa, synthetic construct
GIIPIFGTANYAQKFQG

SEQ ID NO: 565 - protein, 30 aa, synthetic construct
RVTITADESTSTAYMELSSLRSEDTAVYYC

SEQ ID NO: 566 - protein, 20 aa, synthetic construct
ARGAPNYYGSGGYDDGWFDP

SEQ ID NO: 567 - protein, 11 aa, synthetic construct
WGQGTLVTVSS

SEQ ID NO: 568-569 - skipped (2 entries)

SEQ ID NO: 570 - protein, 107 aa, synthetic construct; identical to SEQ ID NO: 53

SEQ ID NO: 571 - protein, 23 aa, synthetic construct
EIVLTQSPGTLSLSPGERATLSC

SEQ ID NO: 572 - protein, 12 aa, synthetic construct
RASQSVSSSYLA

SEQ ID NO: 573 - protein, 15 aa, synthetic construct
WYQQKPGQAPRLLIY

SEQ ID NO: 574 - protein, 7 aa, synthetic construct
GASSRAT

SEQ ID NO: 575 - protein, 32 aa, synthetic construct
GIPDRFSGSGSGTDFTLTISRLEPEDFAVYYC

SEQ ID NO: 576 - protein, 8 aa, synthetic construct
QQFGRDPT

SEQ ID NO: 577 - protein, 10 aa, synthetic construct
FGGGTKVEIK

SEQ ID NO: 578-579 - skipped (2 entries)

SEQ ID NO: 580 - protein, 127 aa, synthetic construct; identical to SEQ ID NO: 51

SEQ ID NO: 581 - protein, 26 aa, synthetic construct
QVQLVQSGAEVKKPGSSVKVSCKASG

SEQ ID NO: 582 - protein, 9 aa, synthetic construct
GTFSSYAIS

SEQ ID NO: 583 - protein, 14 aa, synthetic construct
WVRQAPGQGLEWMG

SEQ ID NO: 584 - protein, 17 aa, synthetic construct
GIIPIFGTANYAQKFQG

SEQ ID NO: 585 - protein, 30 aa, synthetic construct; identical to SEQ ID NO: 565

SEQ ID NO: 586 - protein, 20 aa, synthetic construct
ARGAPNYYGSGGYDDGWFDP

SEQ ID NO: 587 - protein, 11 aa, synthetic construct
WGQGTLVTVSS

SEQ ID NO: 588-589 - skipped (2 entries)

SEQ ID NO: 590 - protein, 107 aa, synthetic construct; identical to SEQ ID NO: 54

SEQ ID NO: 591 - protein, 23 aa, synthetic construct
EIVLTQSPGTLSLSPGERATLSC

SEQ ID NO: 592 - protein, 12 aa, synthetic construct
RASQSVSSSYLA

SEQ ID NO: 593 - protein, 15 aa, synthetic construct
WYQQKPGQAPRLLIY

SEQ ID NO: 594 - protein, 7 aa, synthetic construct
GASSRAT

SEQ ID NO: 595 - protein, 32 aa, synthetic construct; identical to SEQ ID NO: 575

SEQ ID NO: 596 - protein, 8 aa, synthetic construct
LQFGSSPT

SEQ ID NO: 597 - protein, 10 aa, synthetic construct
FGGGTKVEIK

SEQ ID NO: 598-599 - skipped (2 entries)

SEQ ID NO: 600 - protein, 127 aa, synthetic construct; identical to SEQ ID NO: 51

SEQ ID NO: 601 - protein, 26 aa, synthetic construct
QVQLVQSGAEVKKPGSSVKVSCKASG

SEQ ID NO: 602 - protein, 9 aa, synthetic construct
GTFSSYAIS

SEQ ID NO: 603 - protein, 14 aa, synthetic construct
WVRQAPGQGLEWMG

SEQ ID NO: 604 - protein, 17 aa, synthetic construct
GIIPIFGTANYAQKFQG

SEQ ID NO: 605 - protein, 30 aa, synthetic construct; identical to SEQ ID NO: 565

SEQ ID NO: 606 - protein, 20 aa, synthetic construct
ARGAPNYYGSGGYDDGWFDP

SEQ ID NO: 607 - protein, 11 aa, synthetic construct
WGQGTLVTVSS

SEQ ID NO: 608-609 - skipped (2 entries)

SEQ ID NO: 610 - protein, 107 aa, synthetic construct; identical to SEQ ID NO: 55

SEQ ID NO: 611 - protein, 23 aa, synthetic construct
DIQLTQSPSSLSASVGDRVTITC

SEQ ID NO: 612 - protein, 11 aa, synthetic construct
RASQSISSYLN

SEQ ID NO: 613 - protein, 15 aa, synthetic construct
WYQQKPGKAPKLLIY

SEQ ID NO: 614 - protein, 7 aa, synthetic construct
AASNLQS

SEQ ID NO: 615 - protein, 32 aa, synthetic construct
GVPSRFSGSGSGTDFTLTISSLQPEDFATYYC

SEQ ID NO: 616 - protein, 9 aa, synthetic construct
QQTLHTPIT

SEQ ID NO: 617 - protein, 10 aa, synthetic construct
FGGGTKVEIK

SEQ ID NO: 618-619 - skipped (2 entries)

SEQ ID NO: 620 - protein, 127 aa, synthetic construct; identical to SEQ ID NO: 51

SEQ ID NO: 621 - protein, 26 aa, synthetic construct
QVQLVQSGAEVKKPGSSVKVSCKASG

SEQ ID NO: 622 - protein, 9 aa, synthetic construct
GTFSSYAIS

SEQ ID NO: 623 - protein, 14 aa, synthetic construct
WVRQAPGQGLEWMG

SEQ ID NO: 624 - protein, 17 aa, synthetic construct
GIIPIFGTANYAQKFQG

SEQ ID NO: 625 - protein, 30 aa, synthetic construct; identical to SEQ ID NO: 565

SEQ ID NO: 626 - protein, 20 aa, synthetic construct
ARGAPNYYGSGGYDDGWFDP

SEQ ID NO: 627 - protein, 11 aa, synthetic construct
WGQGTLVTVSS

SEQ ID NO: 628-629 - skipped (2 entries)

SEQ ID NO: 630 - protein, 107 aa, synthetic construct; identical to SEQ ID NO: 56

SEQ ID NO: 631 - protein, 23 aa, synthetic construct
EIVLTQSPGTLSLSPGERATLSC

SEQ ID NO: 632 - protein, 12 aa, synthetic construct
RASQSVSSSYLA

SEQ ID NO: 633 - protein, 15 aa, synthetic construct
WYQQKPGQAPRLLIY

SEQ ID NO: 634 - protein, 7 aa, synthetic construct
GASSRAT

SEQ ID NO: 635 - protein, 32 aa, synthetic construct; identical to SEQ ID NO: 575

SEQ ID NO: 636 - protein, 8 aa, synthetic construct
QQFGRSPT

SEQ ID NO: 637 - protein, 10 aa, synthetic construct
FGGGTKVEIK

SEQ ID NO: 638-639 - skipped (2 entries)

SEQ ID NO: 640 - protein, 127 aa, synthetic construct; identical to SEQ ID NO: 51

SEQ ID NO: 641 - protein, 26 aa, synthetic construct
QVQLVQSGAEVKKPGSSVKVSCKASG

SEQ ID NO: 642 - protein, 9 aa, synthetic construct
GTFSSYAIS

SEQ ID NO: 643 - protein, 14 aa, synthetic construct
WVRQAPGQGLEWMG

SEQ ID NO: 644 - protein, 17 aa, synthetic construct
GIIPIFGTANYAQKFQG

SEQ ID NO: 645 - protein, 30 aa, synthetic construct; identical to SEQ ID NO: 565

SEQ ID NO: 646 - protein, 20 aa, synthetic construct
ARGAPNYYGSGGYDDGWFDP

SEQ ID NO: 647 - protein, 11 aa, synthetic construct
WGQGTLVTVSS

SEQ ID NO: 648-649 - skipped (2 entries)

SEQ ID NO: 650 - protein, 107 aa, synthetic construct; identical to SEQ ID NO: 57

SEQ ID NO: 651 - protein, 23 aa, synthetic construct
EIVLTQSPGTLSLSPGERATLSC

SEQ ID NO: 652 - protein, 12 aa, synthetic construct
RASQSVSSDYLA

SEQ ID NO: 653 - protein, 15 aa, synthetic construct
WYQQKPGQAPRLLIY

SEQ ID NO: 654 - protein, 7 aa, synthetic construct
GASSRAT

SEQ ID NO: 655 - protein, 32 aa, synthetic construct; identical to SEQ ID NO: 575

SEQ ID NO: 656 - protein, 8 aa, synthetic construct
QQFGRVPT

SEQ ID NO: 657 - protein, 10 aa, synthetic construct
FGGGTKVEIK

SEQ ID NO: 658-659 - skipped (2 entries)

SEQ ID NO: 660 - protein, 127 aa, synthetic construct; identical to SEQ ID NO: 51

SEQ ID NO: 661 - protein, 26 aa, synthetic construct
QVQLVQSGAEVKKPGSSVKVSCKASG

SEQ ID NO: 662 - protein, 9 aa, synthetic construct
GTFSSYAIS

SEQ ID NO: 663 - protein, 14 aa, synthetic construct
WVRQAPGQGLEWMG

SEQ ID NO: 664 - protein, 17 aa, synthetic construct
GIIPIFGTANYAQKFQG

SEQ ID NO: 665 - protein, 30 aa, synthetic construct; identical to SEQ ID NO: 565

SEQ ID NO: 666 - protein, 20 aa, synthetic construct
ARGAPNYYGSGGYDDGWFDP

SEQ ID NO: 667 - protein, 11 aa, synthetic construct
WGQGTLVTVSS

SEQ ID NO: 668-669 - skipped (2 entries)

SEQ ID NO: 670 - protein, 107 aa, synthetic construct; identical to SEQ ID NO: 58

SEQ ID NO: 671 - protein, 23 aa, synthetic construct
EIVMTQSPGTLSLSPGERATLSC

SEQ ID NO: 672 - protein, 12 aa, synthetic construct
RASQSVSSSYLA

SEQ ID NO: 673 - protein, 15 aa, synthetic construct
WYQQKPGQAPRLLIY

SEQ ID NO: 674 - protein, 7 aa, synthetic construct
GASSRAT

SEQ ID NO: 675 - protein, 32 aa, synthetic construct; identical to SEQ ID NO: 575

SEQ ID NO: 676 - protein, 8 aa, synthetic construct
QQFGRYPT

SEQ ID NO: 677 - protein, 10 aa, synthetic construct
FGGGTKVEIK

SEQ ID NO: 678-679 - skipped (2 entries)

SEQ ID NO: 680 - protein, 127 aa, synthetic construct; identical to SEQ ID NO: 51

SEQ ID NO: 681 - protein, 26 aa, synthetic construct
QVQLVQSGAEVKKPGSSVKVSCKASG

SEQ ID NO: 682 - protein, 9 aa, synthetic construct
GTFSSYAIS

SEQ ID NO: 683 - protein, 14 aa, synthetic construct
WVRQAPGQGLEWMG

SEQ ID NO: 684 - protein, 17 aa, synthetic construct
GIIPIFGTANYAQKFQG

SEQ ID NO: 685 - protein, 30 aa, synthetic construct; identical to SEQ ID NO: 565

SEQ ID NO: 686 - protein, 20 aa, synthetic construct
ARGAPNYYGSGGYDDGWFDP

SEQ ID NO: 687 - protein, 11 aa, synthetic construct
WGQGTLVTVSS

SEQ ID NO: 688-689 - skipped (2 entries)

SEQ ID NO: 690 - protein, 107 aa, synthetic construct; identical to SEQ ID NO: 59

SEQ ID NO: 691 - protein, 23 aa, synthetic construct
EIVLTQSPGTLSLSPGERATLSC

SEQ ID NO: 692 - protein, 12 aa, synthetic construct
RASQSVSSSYLA

SEQ ID NO: 693 - protein, 15 aa, synthetic construct
WYQQKPGQAPRLLIY

SEQ ID NO: 694 - protein, 7 aa, synthetic construct
GASSRAT

SEQ ID NO: 695 - protein, 32 aa, synthetic construct; identical to SEQ ID NO: 575

SEQ ID NO: 696 - protein, 8 aa, synthetic construct
QQFGPSPT

SEQ ID NO: 697 - protein, 10 aa, synthetic construct
FGGGTKVEIK

SEQ ID NO: 698-699 - skipped (2 entries)

SEQ ID NO: 700 - protein, 127 aa, synthetic construct; identical to SEQ ID NO: 51

SEQ ID NO: 701 - protein, 26 aa, synthetic construct
QVQLVQSGAEVKKPGSSVKVSCKASG

SEQ ID NO: 702 - protein, 9 aa, synthetic construct
GTFSSYAIS

SEQ ID NO: 703 - protein, 14 aa, synthetic construct
WVRQAPGQGLEWMG

SEQ ID NO: 704 - protein, 17 aa, synthetic construct
GIIPIFGTANYAQKFQG

SEQ ID NO: 705 - protein, 30 aa, synthetic construct; identical to SEQ ID NO: 565

SEQ ID NO: 706 - protein, 20 aa, synthetic construct
ARGAPNYYGSGGYDDGWFDP

SEQ ID NO: 707 - protein, 11 aa, synthetic construct
WGQGTLVTVSS

SEQ ID NO: 708-709 - skipped (2 entries)

SEQ ID NO: 710 - protein, 107 aa, synthetic construct; identical to SEQ ID NO: 60

SEQ ID NO: 711 - protein, 23 aa, synthetic construct
EIVLTQSPGTLSLSPGERATLSC

SEQ ID NO: 712 - protein, 12 aa, synthetic construct
RASQSVSSSYLA

SEQ ID NO: 713 - protein, 15 aa, synthetic construct
WYQQKPGQAPRLLIY

SEQ ID NO: 714 - protein, 7 aa, synthetic construct
GASSRAT

SEQ ID NO: 715 - protein, 32 aa, synthetic construct; identical to SEQ ID NO: 575

SEQ ID NO: 716 - protein, 8 aa, synthetic construct
QQFGRFPT

SEQ ID NO: 717 - protein, 10 aa, synthetic construct
FGGGTKVEIK

SEQ ID NO: 718-719 - skipped (2 entries)

SEQ ID NO: 720 - protein, 127 aa, synthetic construct; identical to SEQ ID NO: 51

SEQ ID NO: 721 - protein, 26 aa, synthetic construct
QVQLVQSGAEVKKPGSSVKVSCKASG

SEQ ID NO: 722 - protein, 9 aa, synthetic construct
GTFSSYAIS

SEQ ID NO: 723 - protein, 14 aa, synthetic construct
WVRQAPGQGLEWMG

SEQ ID NO: 724 - protein, 17 aa, synthetic construct
GIIPIFGTANYAQKFQG

SEQ ID NO: 725 - protein, 30 aa, synthetic construct; identical to SEQ ID NO: 565

SEQ ID NO: 726 - protein, 20 aa, synthetic construct
ARGAPNYYGSGGYDDGWFDP

SEQ ID NO: 727 - protein, 11 aa, synthetic construct
WGQGTLVTVSS

SEQ ID NO: 728-729 - skipped (2 entries)

SEQ ID NO: 730 - protein, 108 aa, synthetic construct; identical to SEQ ID NO: 61

SEQ ID NO: 731 - protein, 23 aa, synthetic construct
EIVMTQSPGTLSLSPGERATLSC

SEQ ID NO: 732 - protein, 12 aa, synthetic construct
RASQSVSSSYLA

SEQ ID NO: 733 - protein, 15 aa, synthetic construct
WYQQKPGQAPRLLIY

SEQ ID NO: 734 - protein, 7 aa, synthetic construct
GASNRAT

SEQ ID NO: 735 - protein, 32 aa, synthetic construct; identical to SEQ ID NO: 575

SEQ ID NO: 736 - protein, 9 aa, synthetic construct
QQAHTALWT

SEQ ID NO: 737 - protein, 10 aa, synthetic construct
FGGGTKVEIK

SEQ ID NO: 738-739 - skipped (2 entries)

SEQ ID NO: 740 - protein, 128 aa, synthetic construct; identical to SEQ ID NO: 52

SEQ ID NO: 741 - protein, 26 aa, synthetic construct
EVQLLESGGGLVQPGGSLRLSCAASG

SEQ ID NO: 742 - protein, 9 aa, synthetic construct
FTFSSYAMS

SEQ ID NO: 743 - protein, 14 aa, synthetic construct
WVRQAPGKGLEWVS

SEQ ID NO: 744 - protein, 17 aa, synthetic construct
TISGSGGSTYYADSVKG

SEQ ID NO: 745 - protein, 30 aa, synthetic construct
RFTISRDNSKNTLYLQMNSLRAEDTAVYYC

SEQ ID NO: 746 - protein, 21 aa, synthetic construct
ARLGGRRGSGSSYYYYYGMDV

SEQ ID NO: 747 - protein, 11 aa, synthetic construct
WGQGTTVTVSS

SEQ ID NO: 748-749 - skipped (2 entries)

SEQ ID NO: 750 - protein, 112 aa, synthetic construct; identical to SEQ ID NO: 62

SEQ ID NO: 751 - protein, 23 aa, synthetic construct
DIVMTQSPLSLPVTPGEPASISC

SEQ ID NO: 752 - protein, 16 aa, synthetic construct
RSSQSLLHSNGYNYLD

SEQ ID NO: 753 - protein, 15 aa, synthetic construct
WYLQKPGQSPQLLIY

SEQ ID NO: 754 - protein, 7 aa, synthetic construct
LGSNRAS

SEQ ID NO: 755 - protein, 32 aa, synthetic construct
GVPDRFSGSGSGTDFTLKISRVEAEDVGVYYC

SEQ ID NO: 756 - protein, 9 aa, synthetic construct
MQVRQTPWT

SEQ ID NO: 757 - protein, 10 aa, synthetic construct
FGGGTKVEIK

SEQ ID NO: 758-759 - skipped (2 entries)

SEQ ID NO: 760 - protein, 128 aa, synthetic construct; identical to SEQ ID NO: 52

SEQ ID NO: 761 - protein, 26 aa, synthetic construct
EVQLLESGGGLVQPGGSLRLSCAASG

SEQ ID NO: 762 - protein, 9 aa, synthetic construct
FTFSSYAMS

SEQ ID NO: 763 - protein, 14 aa, synthetic construct
WVRQAPGKGLEWVS

SEQ ID NO: 764 - protein, 17 aa, synthetic construct
TISGSGGSTYYADSVKG

SEQ ID NO: 765 - protein, 30 aa, synthetic construct; identical to SEQ ID NO: 745

SEQ ID NO: 766 - protein, 21 aa, synthetic construct
ARLGGRRGSGSSYYYYYGMDV

SEQ ID NO: 767 - protein, 11 aa, synthetic construct
WGQGTTVTVSS

SEQ ID NO: 768-769 - skipped (2 entries)

SEQ ID NO: 770 - protein, 112 aa, synthetic construct; identical to SEQ ID NO: 63

SEQ ID NO: 771 - protein, 23 aa, synthetic construct
DIVMTQSPLSLPVTPGEPASISC

SEQ ID NO: 772 - protein, 16 aa, synthetic construct
RSSQSLLHSNGYNYLD

SEQ ID NO: 773 - protein, 15 aa, synthetic construct
WYLQKPGQSPQLLIY

SEQ ID NO: 774 - protein, 7 aa, synthetic construct
LGSNRAS

SEQ ID NO: 775 - protein, 32 aa, synthetic construct; identical to SEQ ID NO: 755

SEQ ID NO: 776 - protein, 9 aa, synthetic construct
MQVLDLPWT

SEQ ID NO: 777 - protein, 10 aa, synthetic construct
FGGGTKVEIK

SEQ ID NO: 778-779 - skipped (2 entries)

SEQ ID NO: 780 - protein, 128 aa, synthetic construct; identical to SEQ ID NO: 52

SEQ ID NO: 781 - protein, 26 aa, synthetic construct
EVQLLESGGGLVQPGGSLRLSCAASG

SEQ ID NO: 782 - protein, 9 aa, synthetic construct
FTFSSYAMS

SEQ ID NO: 783 - protein, 14 aa, synthetic construct
WVRQAPGKGLEWVS

SEQ ID NO: 784 - protein, 17 aa, synthetic construct
TISGSGGSTYYADSVKG

SEQ ID NO: 785 - protein, 30 aa, synthetic construct; identical to SEQ ID NO: 745

SEQ ID NO: 786 - protein, 21 aa, synthetic construct
ARLGGRRGSGSSYYYYYGMDV

SEQ ID NO: 787 - protein, 11 aa, synthetic construct
WGQGTTVTVSS

SEQ ID NO: 788-789 - skipped (2 entries)

SEQ ID NO: 790 - protein, 112 aa, synthetic construct; identical to SEQ ID NO: 64

SEQ ID NO: 791 - protein, 23 aa, synthetic construct
DIVMTQSPLSLPVTPGEPASISC

SEQ ID NO: 792 - protein, 16 aa, synthetic construct
RSSQSLLHSNGYNYLD

SEQ ID NO: 793 - protein, 15 aa, synthetic construct
WYLQKPGQSPQLLIY

SEQ ID NO: 794 - protein, 7 aa, synthetic construct
LGSNRAS

SEQ ID NO: 795 - protein, 32 aa, synthetic construct; identical to SEQ ID NO: 755

SEQ ID NO: 796 - protein, 9 aa, synthetic construct
MQVLELPWT

SEQ ID NO: 797 - protein, 10 aa, synthetic construct
FGGGTKVEIK

SEQ ID NO: 798-799 - skipped (2 entries)

SEQ ID NO: 800 - protein, 128 aa, synthetic construct; identical to SEQ ID NO: 52

SEQ ID NO: 801 - protein, 26 aa, synthetic construct
EVQLLESGGGLVQPGGSLRLSCAASG

SEQ ID NO: 802 - protein, 9 aa, synthetic construct
FTFSSYAMS

SEQ ID NO: 803 - protein, 14 aa, synthetic construct
WVRQAPGKGLEWVS

SEQ ID NO: 804 - protein, 17 aa, synthetic construct
TISGSGGSTYYADSVKG

SEQ ID NO: 805 - protein, 30 aa, synthetic construct; identical to SEQ ID NO: 745

SEQ ID NO: 806 - protein, 21 aa, synthetic construct
ARLGGRRGSGSSYYYYYGMDV

SEQ ID NO: 807 - protein, 11 aa, synthetic construct
WGQGTTVTVSS

SEQ ID NO: 808-809 - skipped (2 entries)

SEQ ID NO: 810 - protein, 112 aa, synthetic construct; identical to SEQ ID NO: 65

SEQ ID NO: 811 - protein, 23 aa, synthetic construct
DIVMTQSPLSLPVTPGEPASISC

SEQ ID NO: 812 - protein, 16 aa, synthetic construct
RSSQSLLHSNGYNYLD

SEQ ID NO: 813 - protein, 15 aa, synthetic construct
WYLQKPGQSPQLLIY

SEQ ID NO: 814 - protein, 7 aa, synthetic construct
LGSNRAS

SEQ ID NO: 815 - protein, 32 aa, synthetic construct; identical to SEQ ID NO: 755

SEQ ID NO: 816 - protein, 9 aa, synthetic construct
MQVKDTPWT

SEQ ID NO: 817 - protein, 10 aa, synthetic construct
FGGGTKVEIK

SEQ ID NO: 818-819 - skipped (2 entries)

SEQ ID NO: 820 - protein, 128 aa, synthetic construct; identical to SEQ ID NO: 52

SEQ ID NO: 821 - protein, 26 aa, synthetic construct
EVQLLESGGGLVQPGGSLRLSCAASG

SEQ ID NO: 822 - protein, 9 aa, synthetic construct
FTFSSYAMS

SEQ ID NO: 823 - protein, 14 aa, synthetic construct
WVRQAPGKGLEWVS

SEQ ID NO: 824 - protein, 17 aa, synthetic construct
TISGSGGSTYYADSVKG

SEQ ID NO: 825 - protein, 30 aa, synthetic construct; identical to SEQ ID NO: 745

SEQ ID NO: 826 - protein, 21 aa, synthetic construct
ARLGGRRGSGSSYYYYYGMDV

SEQ ID NO: 827 - protein, 11 aa, synthetic construct
WGQGTTVTVSS

SEQ ID NO: 828-829 - skipped (2 entries)

SEQ ID NO: 830 - protein, 112 aa, synthetic construct; identical to SEQ ID NO: 66

SEQ ID NO: 831 - protein, 23 aa, synthetic construct
DIVMTQSPLSLPVTPGEPASISC

SEQ ID NO: 832 - protein, 16 aa, synthetic construct
RSSQSLLYSNGYNYLD

SEQ ID NO: 833 - protein, 15 aa, synthetic construct
WYLQKPGQSPQLLIY

SEQ ID NO: 834 - protein, 7 aa, synthetic construct
LGSNRAS

SEQ ID NO: 835 - protein, 32 aa, synthetic construct; identical to SEQ ID NO: 755

SEQ ID NO: 836 - protein, 9 aa, synthetic construct
MQVLGLPWT

SEQ ID NO: 837 - protein, 10 aa, synthetic construct
FGGGTKVEIK

SEQ ID NO: 838-839 - skipped (2 entries)

SEQ ID NO: 840 - protein, 128 aa, synthetic construct; identical to SEQ ID NO: 52

SEQ ID NO: 841 - protein, 26 aa, synthetic construct
EVQLLESGGGLVQPGGSLRLSCAASG

SEQ ID NO: 842 - protein, 9 aa, synthetic construct
FTFSSYAMS

SEQ ID NO: 843 - protein, 14 aa, synthetic construct
WVRQAPGKGLEWVS

SEQ ID NO: 844 - protein, 17 aa, synthetic construct
TISGSGGSTYYADSVKG

SEQ ID NO: 845 - protein, 30 aa, synthetic construct; identical to SEQ ID NO: 745

SEQ ID NO: 846 - protein, 21 aa, synthetic construct
ARLGGRRGSGSSYYYYYGMDV

SEQ ID NO: 847 - protein, 11 aa, synthetic construct
WGQGTTVTVSS

SEQ ID NO: 848-849 - skipped (2 entries)

SEQ ID NO: 850 - protein, 112 aa, synthetic construct; identical to SEQ ID NO: 67

SEQ ID NO: 851 - protein, 23 aa, synthetic construct
DIVMTQSPLSLPVTPGEPASISC

SEQ ID NO: 852 - protein, 16 aa, synthetic construct
RSSQSLLHSNGYNYLD

SEQ ID NO: 853 - protein, 15 aa, synthetic construct
WYLQKPGQSPQLLIY

SEQ ID NO: 854 - protein, 7 aa, synthetic construct
LGSNRAS

SEQ ID NO: 855 - protein, 32 aa, synthetic construct; identical to SEQ ID NO: 755

SEQ ID NO: 856 - protein, 9 aa, synthetic construct
MQVSDTPWT

SEQ ID NO: 857 - protein, 10 aa, synthetic construct
FGGGTKVEIK

SEQ ID NO: 858-859 - skipped (2 entries)

SEQ ID NO: 860 - protein, 128 aa, synthetic construct; identical to SEQ ID NO: 52

SEQ ID NO: 861 - protein, 26 aa, synthetic construct
EVQLLESGGGLVQPGGSLRLSCAASG

SEQ ID NO: 862 - protein, 9 aa, synthetic construct
FTFSSYAMS

SEQ ID NO: 863 - protein, 14 aa, synthetic construct
WVRQAPGKGLEWVS

SEQ ID NO: 864 - protein, 17 aa, synthetic construct
TISGSGGSTYYADSVKG

SEQ ID NO: 865 - protein, 30 aa, synthetic construct; identical to SEQ ID NO: 745

SEQ ID NO: 866 - protein, 21 aa, synthetic construct
ARLGGRRGSGSSYYYYYGMDV

SEQ ID NO: 867 - protein, 11 aa, synthetic construct
WGQGTTVTVSS

SEQ ID NO: 868-869 - skipped (2 entries)

SEQ ID NO: 870 - protein, 112 aa, synthetic construct; identical to SEQ ID NO: 68

SEQ ID NO: 871 - protein, 23 aa, synthetic construct
DIVMTQSPLSLPVTPGEPASISC

SEQ ID NO: 872 - protein, 16 aa, synthetic construct
RSSQSLLHSNGYNYLD

SEQ ID NO: 873 - protein, 15 aa, synthetic construct
WYLQKPGQSPQLLIY

SEQ ID NO: 874 - protein, 7 aa, synthetic construct
LGSNRAS

SEQ ID NO: 875 - protein, 32 aa, synthetic construct; identical to SEQ ID NO: 755

SEQ ID NO: 876 - protein, 9 aa, synthetic construct
MQVLEIPWT

SEQ ID NO: 877 - protein, 10 aa, synthetic construct
FGGGTKVEIK

SEQ ID NO: 878-879 - skipped (2 entries)

SEQ ID NO: 880 - protein, 128 aa, synthetic construct; identical to SEQ ID NO: 52

SEQ ID NO: 881 - protein, 26 aa, synthetic construct
EVQLLESGGGLVQPGGSLRLSCAASG

SEQ ID NO: 882 - protein, 9 aa, synthetic construct
FTFSSYAMS

SEQ ID NO: 883 - protein, 14 aa, synthetic construct
WVRQAPGKGLEWVS

SEQ ID NO: 884 - protein, 17 aa, synthetic construct
TISGSGGSTYYADSVKG

SEQ ID NO: 885 - protein, 30 aa, synthetic construct; identical to SEQ ID NO: 745

SEQ ID NO: 886 - protein, 21 aa, synthetic construct
ARLGGRRGSGSSYYYYYGMDV

SEQ ID NO: 887 - protein, 11 aa, synthetic construct
WGQGTTVTVSS

SEQ ID NO: 888-889 - skipped (2 entries)

SEQ ID NO: 890 - protein, 112 aa, synthetic construct; identical to SEQ ID NO: 64

SEQ ID NO: 891 - protein, 23 aa, synthetic construct
DIVMTQSPLSLPVTPGEPASISC

SEQ ID NO: 892 - protein, 16 aa, synthetic construct
RSSQSLLHSNGYNYLD

SEQ ID NO: 893 - protein, 15 aa, synthetic construct
WYLQKPGQSPQLLIY

SEQ ID NO: 894 - protein, 7 aa, synthetic construct
LGSNRAS

SEQ ID NO: 895 - protein, 32 aa, synthetic construct; identical to SEQ ID NO: 755

SEQ ID NO: 896 - protein, 9 aa, synthetic construct
MQVLELPWT

SEQ ID NO: 897 - protein, 10 aa, synthetic construct
FGGGTKVEIK

SEQ ID NO: 898-899 - skipped (2 entries)

SEQ ID NO: 900 - protein, 128 aa, synthetic construct; identical to SEQ ID NO: 52

SEQ ID NO: 901 - protein, 26 aa, synthetic construct
EVQLLESGGGLVQPGGSLRLSCAASG

SEQ ID NO: 902 - protein, 9 aa, synthetic construct
FTFSSYAMS

SEQ ID NO: 903 - protein, 14 aa, synthetic construct
WVRQAPGKGLEWVS

SEQ ID NO: 904 - protein, 17 aa, synthetic construct
TISGSGGSTYYADSVKG

SEQ ID NO: 905 - protein, 30 aa, synthetic construct; identical to SEQ ID NO: 745

SEQ ID NO: 906 - protein, 21 aa, synthetic construct
ARLGGRRGSGSSYYYYYGMDV

SEQ ID NO: 907 - protein, 11 aa, synthetic construct
WGQGTTVTVSS

SEQ ID NO: 908-909 - skipped (2 entries)

SEQ ID NO: 910 - protein, 112 aa, synthetic construct; identical to SEQ ID NO: 63

SEQ ID NO: 911 - protein, 23 aa, synthetic construct
DIVMTQSPLSLPVTPGEPASISC

SEQ ID NO: 912 - protein, 16 aa, synthetic construct
RSSQSLLHSNGYNYLD

SEQ ID NO: 913 - protein, 15 aa, synthetic construct
WYLQKPGQSPQLLIY

SEQ ID NO: 914 - protein, 7 aa, synthetic construct
LGSNRAS

SEQ ID NO: 915 - protein, 32 aa, synthetic construct; identical to SEQ ID NO: 755

SEQ ID NO: 916 - protein, 9 aa, synthetic construct
MQVLDLPWT

SEQ ID NO: 917 - protein, 10 aa, synthetic construct
FGGGTKVEIK

SEQ ID NO: 918-920 - skipped (3 entries)

SEQ ID NO: 921 - protein, 26 aa, synthetic construct
QVQLVQSGAEVKKPGSSVKVSCKASG

SEQ ID NO: 922 - protein, 9 aa, synthetic construct
GTFSSYAIS

SEQ ID NO: 923 - protein, 14 aa, synthetic construct
WVRQAPGQGLEWMG

SEQ ID NO: 924 - protein, 17 aa, synthetic construct
GIIPIFGTANYAQKFQG

SEQ ID NO: 925 - protein, 30 aa, synthetic construct; identical to SEQ ID NO: 565

SEQ ID NO: 926 - protein, 20 aa, synthetic construct
ARGAPNYYGSGGYDDGWFDP

SEQ ID NO: 927 - protein, 11 aa, synthetic construct
WGQGTLVTVSS

SEQ ID NO: 928-930 - skipped (3 entries)

SEQ ID NO: 931 - protein, 23 aa, synthetic construct
  VARIANT 1: note = E or D
  VARIANT 3: note = V or Q
  VARIANT 4: note = L or M
  VARIANT 9: note = G or S
  VARIANT 10: note = T or S
  VARIANT 13: note = L or A
  VARIANT 15: note = P or V
  VARIANT 17: note = E or D
  VARIANT 19: note = A or V
  VARIANT 21: note = L or I
  VARIANT 22: note = S or T
XIXXTQSPXXLSXSXGXRXTXXC

SEQ ID NO: 932 - protein, 12 aa, synthetic construct
  VARIANT 6: note = V or I
  VARIANT 9: note = D, S, or absent
  VARIANT 12: note = A or N
RASQSXSSXYLX

SEQ ID NO: 933 - protein, 15 aa, synthetic construct
  VARIANT 8: note = Q or K
  VARIANT 11: note = R or K
WYQQKPGXAPXLLIY

SEQ ID NO: 934 - skipped

SEQ ID NO: 935 - protein, 32 aa, synthetic construct
  VARIANT 2: note = I or V
  VARIANT 4: note = D or S
  VARIANT 21: note = R or S
  VARIANT 23: note = E or Q
  VARIANT 29: note = V or T
GXPXRFSGSGSGTDFTLTISXLXPEDFAXYYC

SEQ ID NO: 936 - skipped

SEQ ID NO: 937 - protein, 10 aa, synthetic construct
FGGGTKVEIK

SEQ ID NO: 938-940 - skipped (3 entries)

SEQ ID NO: 941 - protein, 26 aa, synthetic construct
EVQLLESGGGLVQPGGSLRLSCAASG

SEQ ID NO: 942 - protein, 9 aa, synthetic construct
FTFSSYAMS

SEQ ID NO: 943 - protein, 14 aa, synthetic construct
WVRQAPGKGLEWVS

SEQ ID NO: 944 - protein, 17 aa, synthetic construct
TISGSGGSTYYADSVKG

SEQ ID NO: 945 - protein, 30 aa, synthetic construct; identical to SEQ ID NO: 745

SEQ ID NO: 946 - protein, 21 aa, synthetic construct
ARLGGRRGSGSSYYYYYGMDV

SEQ ID NO: 947 - protein, 11 aa, synthetic construct
WGQGTTVTVSS

SEQ ID NO: 948-950 - skipped (3 entries)

SEQ ID NO: 951 - protein, 23 aa, synthetic construct
DIVMTQSPLSLPVTPGEPASISC

SEQ ID NO: 952 - protein, 16 aa, synthetic construct
  VARIANT 8: note = H or Y
RSSQSLLXSNGYNYLD

SEQ ID NO: 953 - protein, 15 aa, synthetic construct
WYLQKPGQSPQLLIY

SEQ ID NO: 954 - protein, 7 aa, synthetic construct
LGSNRAS

SEQ ID NO: 955 - protein, 32 aa, synthetic construct; identical to SEQ ID NO: 755

SEQ ID NO: 956 - protein, 9 aa, synthetic construct
  VARIANT 4: note = L, R, K or S
  VARIANT 5: note = E, Q, D, or G
  VARIANT 6: note = L, T, or I
MQVXXXPWT

SEQ ID NO: 957 - protein, 10 aa, synthetic construct
FGGGTKVEIK